(19) United States
(12) Patent Application Publication
(10) Pub. No.: US 2007/0286822 A1
(43) Pub. Date: Dec. 13, 2007

(54) Compounds for the Treatment of Periodontal Disease

(75) Inventors: Virginia Sanders, San Francisco, CA (US); Kirk R. Maples, San Jose, CA (US); Jacob J. Plattner, Berkeley, CA (US); Carolyn Bellinger-Kawahara, Redwood City, CA (US)

Correspondence Address: MORGAN, LEWIS & BOCKIUS LLP (SF), 2 PALO ALTO SQUARE, 3000 El Camino Real, Suite 700, PALO ALTO, CA, 94306, US

(73) Assignee: ANACOR PHARMACEUTICALS INC., Palo Alto, CA (US)

(21) Appl. No.: 11/762,038

(22) Filed: Jun. 12, 2007

Related U.S. Application Data
(60) Provisional application No. 60/804,504, filed on Jun. 12, 2006.
(60) Provisional application No. 60/823,893, filed on Aug. 29, 2006.

(51) Int. Cl. A61K 8/49; A61K 31/69

(52) U.S. Cl. 424/49; 514/64

(57) ABSTRACT

Compounds, compositions and methods are provided which are useful in the treatment of periodontal disease.

### CROSS-REFERENCE TO RELATED APPLICATIONS

This application claims priority to U.S. Provisional Patent Application Ser. No. 60/823,893 filed on Aug. 29, 2006 and 60/804,504, filed on Jun. 12, 2006, which are herein incorporated by reference in their entirety for all purposes.

### BACKGROUND OF THE INVENTION

Bacterial infections of the mouth include inflammation of the gum, gingivitis, and inflammation of the periodontium, periodontitis. Plaque bacteria and bacterial toxins that accumulate below the gum-line may cause inflammation of the gums, termed gingivitis. Inflammation of the gingiva involves influx of lymphocytes and macrophages into the gum tissue and their release of proinflammatory cytokines (TNFa and IL1b) and matrix metalloproteases (MMPs). Periodontitis, or Pyorrhea, is a disease involving chronic inflammation of the gums (gingiva), often persisting unnoticed for years or decades in a patient, that results in loss of connective tissue and/or bone supporting the teeth. It is the loss of bone around the teeth that differentiates these two oral inflammatory diseases. The loss of the surrounding bone, that holds the teeth in the jaws, may over the years result in the teeth becoming loose and so fall out. While gingivitis is reversible with anti-bacterial and/or anti-inflammatory treatments and good oral hygiene, periodontitis is irreversible. However, progression may be halted or slowed significantly with appropriate treatment. Periodontitis is the second most important cause, after tooth decay, of tooth loss.

The development of new compounds and methods to treat infections of the mouth, such as those involving bacteria, viruses, fungi and/or parasites, would represent a significant advance in the art. This development, and others, have been addressed by the current invention.

### SUMMARY OF THE INVENTION

In a first aspect, the invention provides an oral care composition comprising a compound described herein. In an exemplary embodiment, the oral care composition is a member selected from a mouthwash, dentifrice, liquid whitener, chewing gum, dissolvable, partially dissolvable or non-dissolvable film or strip, wipe or towelette, implant, dental floss. In an exemplary embodiment, the oral care composition is a member selected from a toothpaste, prophylactic paste, tooth polish, gel, professional gel and other related products applied by dentists, as well as mouth wash, mouth rinse, dental floss, chewing gum, lozenge, tablet, edible food product and the like. In an exemplary embodiment, the dentifrice is a member selected from a powder, toothpaste and dental gel. In an exemplary embodiment, the compound is present in a therapeutically effective amount. In an exemplary embodiment, the compound is present in an amount of from about 0.1% wgt of compound/wgt of oral care composition to about 5% wgt of compound/wgt of oral care composition. In an exemplary embodiment, the compound is present in an amount of from about 0.3% wgt of compound/wgt of oral care composition to about 0.6% wgt of compound/wgt of oral care composition. In an exemplary embodiment, the compound is present in the range of about (all percentages are in wgt of compound/wgt of oral care composition) 0.3% to about 5%, including about 0.4%, about 0.6%, about 0.8%, about 1%, about 1.5, about 2%, about 2.5%, about 3%, about 3.5%, about 4%, and the like. In exemplary embodiments, the compound is present in the range of about 2% to about 10%. In exemplary embodiments, the compound is present in the range of about 2% to about 4%. In exemplary embodiments, the compound is present in the range of about 2.5% to about 6%. In exemplary embodiments, the compound is present in the range of about 0.1% to about 1%.

In another exemplary embodiment, the compound has a structure according to one of the following formulas: 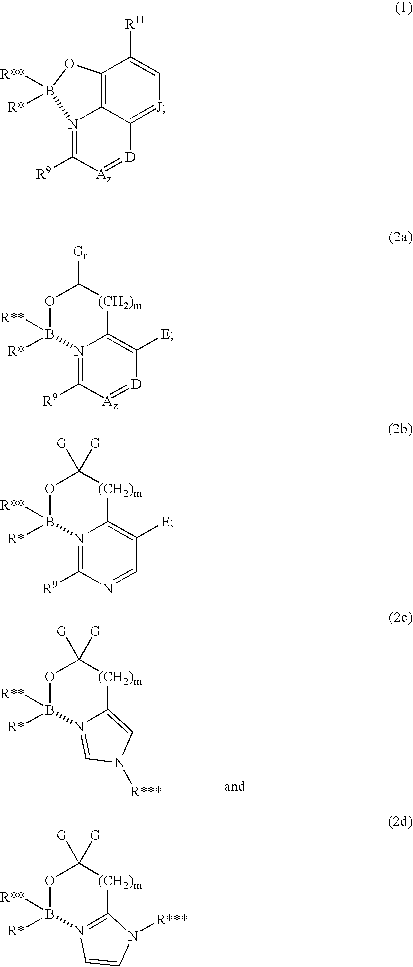
wherein B is boron, O is oxygen, R* and R** are each independently selected from substituted or unsubstituted alkyl (C₁-C₄), substituted or unsubstituted cycloalkyl (C₃-C₇), substituted or unsubstituted alkenyl, substituted or unsubstituted alkynyl, substituted or unsubstituted aralkyl, substituted or unsubstituted phenyl, and substituted or unsubstituted heteroaryl; z is 0 or 1 and when z is 1, A is CH, CR¹⁰ or N; D is N, CH, or CR¹²; E is H, OH, alkoxy or 2-(morpholino)ethoxy, CO₂H or CO₂alkyl; m=0-2; r is 1 or 2, and wherein when r is 1, G is ═O (double-bonded oxygen) and when r is 2, each G is independently H, methyl, ethyl or propyl; R¹² is selected from (CH₂)ₖOH (where k=1, 2 or 3), CH₂NH₂, CH₂NH-alkyl, CH₂N(alkyl)₂, CO₂H, CO₂alkyl, CONH₂, OH, alkoxy, aryloxy, SH, S-alkyl, S-aryl, SO₂N(alkyl)₂, SO₂NHalkyl, SO₂NH₂, SO₂alkyl, SO₃H, SCF₃, CN, halogen, CF₃, NO₂, NH₂, 2^{o}-amino, 3^{o}-amino, NH₂SO₂ and CONH₂, and wherein J is CR¹⁰ or N; R⁹, R¹⁰ and R¹¹ are each independently selected from the group consisting of hydrogen, alkyl, cycloalkyl, (CH₂)ₙOH (n=2 to 3), CH₂NH₂, CH₂NHalkyl, CH₂N(alkyl)₂, halogen, CHO, CH═NOH, CO₂H, CO₂-alkyl, S-alkyl, SO₂-alkyl, S-aryl, SO₂N(alkyl)₂, SO₂NHalkyl, SO₂NH₂, NH₂, alkoxy, CF₃, SCF₃, NO₂, SO₃H and OH, including salts thereof. In another exemplary embodiment, the compound has a structure according to 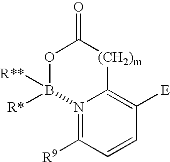
wherein m is 0. In another exemplary embodiment, the compound has a structure according to 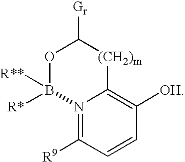
In another exemplary embodiment, E is OH, R⁹ is H and R* and R** are independently selected from substituted or unsubstituted phenyl. In another exemplary embodiment, R* and R** are independently selected from 4-alkyl, 3-halogen phenyl and 4-halogen, 3-alkyl phenyl. In another exemplary embodiment, R* and R** are 4-methyl, 3-chloro phenyl. In another exemplary embodiment, the compound is a member selected from 3-hydroxypyridine-2-carbonyloxy-bis(3-chloro-4-methylphenyl)-borane or (bis(3-Chloro-4-methylphenyl)borinic acid 3-hydroxypicolinate ester), 1,3-dihydro-5-fluoro-1-hydroxy-2,1-benzoxaborole, and 5-(4-cyanophenoxy)-1,3-dihydro-1-hydroxy-2,1-benzoxaborole. In another exemplary embodiment, the compound is 3-hydroxypyridine-2-carbonyloxy-bis(3-chloro-4-methylphenyl)-borane. In another exemplary embodiment, the compound is 3-hydroxypyridine-2-carbonyloxy-bis(3-chloro-4-methylphenyl)-borane and the compound is present in an amount of from about 0.1% wgt/wgt to about 5% wgt/wgt. In another exemplary embodiment, the compound is 3-hydroxypyridine-2-carbonyloxy-bis(3-chloro-4-methylphenyl)-borane and the compound is present in an amount of from about 0.3% wgt/wgt to about 0.6% wgt/wgt.

In an exemplary embodiment, the compound described herein has anti-bacterial properties. In another exemplary embodiment, the compound described herein has anti-inflammatory properties. In an exemplary embodiment, the compound described herein has both anti-bacterial and anti-inflammatory properties. In an exemplary embodiment, the compound described herein has both anti-bacterial and anti-inflammatory properties, and is 3-hydroxypyridine-2-carbonyloxy-bis(3-chloro-4-methylphenyl)-borane.

In a second aspect, the invention provides a method for killing a microorganism or inhibiting the growth of a microorganism, comprising contacting said microorganism with a therapeutically effective amount of a compound described herein, thereby killing or inhibiting the growth of the microorganism. In an exemplary embodiment, the microorganism is a member selected from Actinobacillus species, Porphyromonas species, Tannerella species, Prevotella species, Eubacterium species, Treponema species, Bulleidia species, Mogibacterium species, Slackia species, Campylobacter species, Eikenella species, Peptostreptococcus species, Peptostreptococcus species, Capnocytophaga species, Fusobacterium species, Porphyromonas species and Bacteroides species. In another exemplary embodiment, the microorganism is a member selected from Actinobacillus actinomycetemcomitans, Porphyromonas gingivalis, Tannerella forsythensis, Prevotella intermedia, Eubacterium nodatum, Treponema denticola, Bulleidia extructa, Mogibacterium timidum, Slackia exigua, Campylobacter rectus, Eikenella corrodens, Peptostreptococcus micros, Peptostreptococcus anaerobius, Capnocytophaga ochracea, Fusobacterium nucleatum, Porphyromonas asaccharolytica and Bacteroides forsythus.

In another exemplary embodiment, the compound of use in the method has a structure described above. In another exemplary embodiment, the compound of use in the method is a member selected from 3-hydroxypyridine-2-carbonyloxy-bis(3-chloro-4-methylphenyl)-borane or (bis(3-chloro-4-methylphenyl)borinic acid 3-hydroxypicolinate ester), 1,3-dihydro-5-fluoro-1-hydroxy-2,1-benzoxaborole, and 5-(4-cyanophenoxy)-1,3-dihydro-1-hydroxy-2,1-benzoxaborole. In another exemplary embodiment, the compound of use in the method is 3-hydroxypyridine-2-carbonyloxy-bis(3-chloro-4-methylphenyl)-borane.

In a third aspect, the invention provides a method for treating or preventing periodontal disease in a human or an animal, comprising administering to the human or the animal a therapeutically effective amount of a compound described herein, thereby treating or preventing said periodontal disease. In an exemplary embodiment, the periodontal disease is a member selected from gingivitis, periodontitis and juvenile/acute periodontitis.

In another exemplary embodiment, the compound of use in the method has a structure described above. In another exemplary embodiment, the compound of use in the method is a member selected from 3-hydroxypyridine-2-carbonyloxy-bis(3-chloro-4-methylphenyl)-borane or (bis(3-chloro-4-methylphenyl)borinic acid 3-hydroxypicolinate ester), 1,3-dihydro-5-fluoro-1-hydroxy-2,1-benzoxaborole, and 5-(4-cyanophenoxy)-1,3-dihydro-1-hydroxy-2,1-benzoxaborole. In another exemplary embodiment, the compound of use in the method is 3-hydroxypyridine-2-carbonyloxy-bis(3-chloro-4-methylphenyl)-borane.

In an exemplary embodiment, a compound of use in the compositions and methods described herein has a structure according to Formula I: 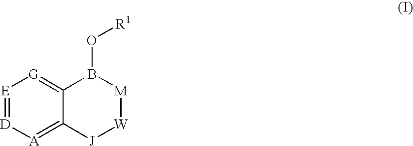
wherein B is boron. R^{1a} is a member selected from a negative charge, a salt counterion, H, cyano, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. M is a member selected from oxygen, sulfur and NR^{2a}. R^{2a} is a member selected from H, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. J is a member selected from (CR^{3a}R^{4a})ₙ₁ and CR^{5a}. R^{3a}, R^{4a}, and R^{5a} are members independently selected from H, cyano, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. The index n1 is an integer selected from 0 to 2. W is a member selected from C═O (carbonyl), (CR^{6a}R^{7a})ₘ₁ and CR^{8a}. R^{6a}, R^{7a}, and R^{8a} are members independently selected from H, cyano, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. The index m1 is an integer selected from 0 and 1. A is a member selected from CR^{9a} and N. D is a member selected from CR^{10a} and N. E is a member selected from CR^{11a} and N. G is a member selected from CR^{12a}, and N. R^{9a}, R^{10a}, R^{11a} and R^{12a} are members independently selected from H, OR*^{a}, NR*^{a}R**^{a}, SR*^{a}, —S(O)R*^{a}, —S(O)₂R*^{a}, —S(O)₂NR*^{a}R**^{a}, —C(O)R*^{a}, —C(O)OR*^{a}, —C(O)NR*^{a}R**^{a}, nitro, halogen, cyano, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. Each R*^{a} and R**^{a} are members independently selected from H, nitro, halogen, cyano, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. The combination of nitrogens (A+D+E+G) is an integer selected from 0 to 3. A member selected from R^{3a}, R^{4a} and R^{5a} and a member selected from R^{6a}, R^{7a} and R^{8a}, together with the atoms to which they are attached, are optionally joined to form a 4 to 7 membered ring. R^{3a} and R^{4a}, together with the atoms to which they are attached, are optionally joined to form a 4 to 7 membered ring. R^{6a} and R^{7a}, together with the atoms to which they are attached, are optionally joined to form a 4 to 7 membered ring. R^{9a} and R^{10a}, together with the atoms to which they are attached, are optionally joined to form a 4 to 7 membered ring. R^{10a} and R^{11a}, together with the atoms to which they are attached, are optionally joined to form a 4 to 7 membered ring. R^{11a} and R^{12a}, together with the atoms to which they are attached, are optionally joined to form a 4 to 7 membered ring.

In an exemplary embodiment, a compound of use in the compositions and methods described herein has a structure according to Formula IX: 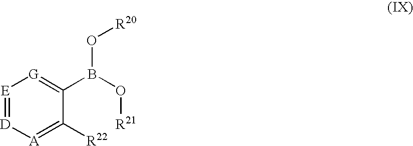
wherein the variables A, D, E and G are described elsewhere herein. R²⁰, R²¹ and R²² are members independently selected from a negative charge, a salt counterion, H, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

In an exemplary embodiment, a compound of use in the compositions and methods described herein has a structure according to Formula XI: 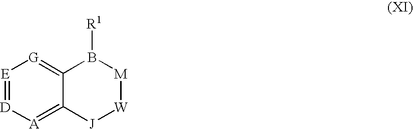
wherein the variables R¹, A, D, E, G, J, W and M are described elsewhere herein.

In an exemplary embodiment, the microorganism is implicated in periodontal disease. In another exemplary embodiment, the microorganism is a member selected from a virus, bacteria, fungus, yeast or parasite. In another exemplary embodiment, the bacteria is a member selected from Actinobacillus species, Porphyromonas species, Tannerella species, Prevotella species, Eubacterium species, Treponema species, Bulleidia species, Mogibacterium species, Slackia species, Campylobacter species, Eikenella species, Peptostreptococcus species, Peptostreptococcus species, Capnocytophaga species, Fusobacterium species, Porphyromonas species and Bacteroides species. In yet another exemplary embodiment, the bacteria is a member selected from Actinobacillus actinomycetemcomitans, Porphyromonas gingivalis, Tannerella forsythensis, Prevotella intermedia, Eubacterium nodatum, Treponema denticola, Bulleidia extructa, Mogibacterium timidum, Slackia exigua, Campylobacter rectus, Eikenella corrodens, Peptostreptococcus micros, Peptostreptococcus anaerobius, Capnocytophaga ochracea, Fusobacterium nucleatum, Porphyromonas asaccharolytica and Bacteroides forsythus.

In a second aspect, the invention provides a method of treating or preventing periodontal disease in an animal, said method comprising administering to the animal a therapeutically effective amount of a boron-containing compound described herein. In an exemplary embodiment, the animal is a human. In another exemplary embodiment, the periodontal disease is a member selected from gingivitis, periodontitis, and juvenile/acute periodontitis.

In a third aspect, the invention provides an oral care composition comprising a boron-containing compound described herein. This oral care composition can be used to treat periodontal disease.

Additional aspects, advantages and objects of the present invention will be apparent from the detailed description that follows.

### BRIEF DESCRIPTION OF THE DRAWINGS

FIG. 1 displays the results of testing several boron-containing compounds of the invention against several bacteria which are implicated in periodontal disease.

FIG. 2 displays exemplary compounds of the invention.

FIG. 3 displays exemplary compounds of the invention.

### DETAILED DESCRIPTION OF THE INVENTION

Definitions and Abbreviations

The abbreviations used herein generally have their conventional meaning within the chemical and biological arts.

“Compound of the invention” and “exemplary compounds of use in methods of the invention,” are used interchangeably and refer to the compounds discussed herein, and pharmaceutically acceptable salts and prodrugs of these compounds.

Where substituent groups are specified by their conventional chemical formulae, written from left to right, they equally encompass the chemically identical substituents, which would result from writing the structure from right to left, e.g., —CH₂O— is intended to also recite —OCH₂—.

The term “poly” as used herein means at least 2. For example, a polyvalent metal ion is a metal ion having a valency of at least 2.

“Moiety” refers to the radical of a molecule that is attached to another moiety.

The symbol [symbol] , whether utilized as a bond or displayed perpendicular to a bond, indicates the point at which the displayed moiety is attached to the remainder of the molecule.

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl.” Alkyl groups that are limited to hydrocarbon groups are termed “homoalkyl”.

The term “alkylene” by itself or as part of another substituent means a divalent radical derived from an alkane, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom. In an exemplary embodiment, the heteroatoms can be selected from the group consisting of B, O, N and S, and wherein the nitrogen and sulfur atoms may optionally be oxidized and the nitrogen heteroatom may optionally be quaternized. The heteroatom(s) B, O, N and S may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —CH₂—CH═N—OCH₃, and —CH═CH—N(CH₃)—CH₃. Up to two heteroatoms may be consecutive, such as, for example, —CH₂—NH—OCH₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, substituent that can be a single ring or multiple rings (preferably from 1 to 3 rings), which are fused together or linked covalently. The term “heteroaryl” refers to aryl groups (or rings) that contain from one to four heteroatoms. In an exemplary embodiment, the heteroatom is selected from B, N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) are meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generically referred to as “alkyl group substitutents,” and they can be one or more of a variety of groups selected from, but not limited to: —OR′, ═O, ═NR′, ═N—OR′, —NR′R″, —SR′, -halogen, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are generically referred to as “aryl group substituents.” The substituents are selected from, for example: halogen, —OR′, ═O, ═NR′, ═N—OR′, —NR′R″, —SR′, -halogen, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, substituted or unsubstituted alkyl, substituted or unsubstituted heteroalkyl, substituted or unsubstituted aryl and substituted or unsubstituted heteroaryl. When a compound of the invention includes more than one R group, for example, each of the R groups is independently selected as are each R′, R″, R′″ and R″″ groups when more than one of these groups is present.

Two of the substituents on adjacent atoms of the aryl or heteroaryl ring may optionally be replaced with a substituent of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X—(CR″R′″)_{d}—, where s and d are independently integers of from 0 to 3, and X is —O—, —NR′—, —S—, —S(O)—, —S(O)₂—, or —S(O)₂NR′—. The substituents R, R′, R″ and R′″ are preferably independently selected from hydrogen or substituted or unsubstituted (C₁-C₆)alkyl.

“Ring” as used herein means a substituted or unsubstituted cycloalkyl, substituted or unsubstituted heterocycloalkyl, substituted or unsubstituted aryl, or substituted or unsubstituted heteroaryl. A ring includes fused ring moieties. The number of atoms in a ring is typically defined by the number of members in the ring. For example, a “5- to 7-membered ring” means there are 5 to 7 atoms in the encircling arrangement. The ring optionally included a heteroatom. Thus, the term “5- to 7-membered ring” includes, for example pyridinyl and piperidinyl. The term “ring” further includes a ring system comprising more than one “ring”, wherein each “ring” is independently defined as above.

As used herein, the term “heteroatom” includes atoms other than carbon (C) and hydrogen (H). Examples include oxygen (O), nitrogen (N) sulfur (S), silicon (Si), germanium (Ge), aluminum (Al) and boron (B).

The symbol “R” is a general abbreviation that represents a substituent group that is selected from substituted or unsubstituted alkyl, substituted or unsubstituted heteroalkyl, substituted or unsubstituted aryl, substituted or unsubstituted heteroaryl, substituted or unsubstituted cycloalkyl and substituted or unsubstituted heterocycloalkyl groups.

By “effective” amount of a drug, formulation, or permeant is meant a sufficient amount of a active agent to provide the desired local or systemic effect. A “Topically effective,” “Cosmetically effective,” “pharmaceutically effective,” or “therapeutically effective” amount refers to the amount of drug needed to effect the desired therapeutic result.

“Topically effective” refers to a material that, when applied to the skin, nail, hair, claw or hoof produces a desired pharmacological result either locally at the place of application or systemically as a result of transdermal passage of an active ingredient in the material.

“Cosmetically effective” refers to a material that, when applied to the skin, nail, hair, claw or hoof, produces a desired cosmetic result locally at the place of application of an active ingredient in the material.

The term “pharmaceutically acceptable salts” is meant to include salts of the compounds of the invention which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., Journal of Pharmaceutical Science 66: 1-19 (1977)). Certain specific compounds of the present invention contain both basic and acidic functionalities that allow the compounds to be converted into either base or acid addition salts.

The neutral forms of the compounds are preferably regenerated by contacting the salt with a base or acid and isolating the parent compounds in the conventional manner. The parent form of the compound differs from the various salt forms in certain physical properties, such as solubility in polar solvents.

In addition to salt forms, the present invention provides compounds which are in a prodrug form. Prodrugs of the compounds or complexes described herein readily undergo chemical changes under physiological conditions to provide the compounds of the present invention. Additionally, prodrugs can be converted to the compounds of the present invention by chemical or biochemical methods in an ex vivo environment.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers are encompassed within the scope of the present invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

The term “pharmaceutically acceptable carrier” or “pharmaceutically acceptable vehicle” refers to any formulation or carrier medium that provides the appropriate delivery of an effective amount of a active agent as defined herein, does not interfere with the effectiveness of the biological activity of the active agent, and that is sufficiently non-toxic to the host or patient. Representative carriers include water, oils, both vegetable and mineral, cream bases, lotion bases, ointment bases and the like. These bases include suspending agents, thickeners, penetration enhancers, and the like. Their formulation is well known to those in the art of cosmetics and topical pharmaceuticals. Additional information concerning carriers can be found in Remington: The Science and Practice of Pharmacy, 21st Ed., Lippincott, Williams & Wilkins (2005) which is incorporated herein by reference.

“Pharmaceutically acceptable topical carrier” and equivalent terms refer to pharmaceutically acceptable carriers, as described herein above, suitable for topical application. An inactive liquid or cream vehicle capable of suspending or dissolving the active agent(s), and having the properties of being nontoxic and non-inflammatory when applied to the skin, nail, hair, claw or hoof is an example of a pharmaceutically-acceptable topical carrier. This term is specifically intended to encompass carrier materials approved for use in topical cosmetics as well.

The term “pharmaceutically acceptable additive” refers to preservatives, antioxidants, fragrances, emulsifiers, dyes and excipients known or used in the field of drug formulation and that do not unduly interfere with the effectiveness of the biological activity of the active agent, and that is sufficiently non-toxic to the host or patient. Additives for topical formulations are well-known in the art, and may be added to the topical composition, as long as they are pharmaceutically acceptable and not deleterious to the epithelial cells or their function. Further, they should not cause deterioration in the stability of the composition. For example, inert fillers, anti-irritants, tackifiers, excipients, fragrances, opacifiers, antioxidants, gelling agents, stabilizers, surfactant, emollients, coloring agents, preservatives, buffering agents, other permeation enhancers, and other conventional components of topical or transdermal delivery formulations as are known in the art.

The terms “enhancement,” “penetration enhancement” or “permeation enhancement” relate to an increase in the permeability of the skin, nail, hair, claw or hoof to a drug, so as to increase the rate at which the drug permeates through the skin, nail, hair, claw or hoof. The enhanced permeation effected through the use of such enhancers can be observed, for example, by measuring the rate of diffusion of the drug through animal or human skin, nail, hair, claw or hoof using a diffusion cell apparatus. A diffusion cell is described by Merritt et al., J of Controlled Release, 1:161-162 (1984). The term “permeation enhancer” or “penetration enhancer” intends an agent or a mixture of agents, which, alone or in combination, act to increase the permeability of the skin, nail, hair or hoof to a drug.

The term “excipients” is conventionally known to mean carriers, diluents and/or vehicles used in formulating drug compositions effective for the desired use.

The term “topical administration” refers to the application of a pharmaceutical agent to the external surface of the skin, nail, hair, claw or hoof, such that the agent crosses the external surface of the skin, nail, hair, claw or hoof and enters the underlying tissues. Topical administration includes application of the composition to intact skin, nail, hair, claw or hoof, or to a broken, raw or open wound of skin, nail, hair, claw or hoof. Topical administration of a pharmaceutical agent can result in a limited distribution of the agent to the skin and surrounding tissues or, when the agent is removed from the treatment area by the bloodstream, can result in systemic distribution of the agent.

The term “transdermal delivery” refers to the diffusion of an agent across the barrier of the skin, nail, hair, claw or hoof resulting from topical administration or other application of a composition. The stratum corneum acts as a barrier and few pharmaceutical agents are able to penetrate intact skin. In contrast, the epidermis and dermis are permeable to many solutes and absorption of drugs therefore occurs more readily through skin, nail, hair, claw or hoof that is abraded or otherwise stripped of the stratum corneum to expose the epidermis. Transdermal delivery includes injection or other delivery through any portion of the skin, nail, hair, claw or hoof or mucous membrane and absorption or permeation through the remaining portion. Absorption through intact skin, nail, hair, claw or hoof can be enhanced by placing the active agent in an appropriate pharmaceutically acceptable vehicle before application to the skin, nail, hair, claw or hoof. Passive topical administration may consist of applying the active agent directly to the treatment site in combination with emollients or penetration enhancers. As used herein, transdermal delivery is intended to include delivery by permeation through or past the integument, i.e. skin, nail, hair, claw or hoof.

The term “microbial infection” refers to any infection of a host tissue by an infectious agent including, but not limited to, viruses, bacteria, mycobacteria, fungus and parasites (see, e.g., Harrison's Principles of Internal Medicine, pp. 93-98 (Wilson et al., eds., 12th ed. 1991); Williams et al., J. of Medicinal Chem. 42:1481-1485 (1999), herein each incorporated by reference in their entirety).

The term “microbial infection” refers to any infection of a host tissue by an infectious agent including, but not limited to, viruses, bacteria, mycobacteria, fungus and parasites (see, e.g., Harrison's Principles of Internal Medicine, pp. 93-98 (Wilson et al., eds., 12th ed. 1991); Williams et al., J. of Medicinal Chem. 42:1481-1485 (1999), herein each incorporated by reference in their entirety).

“Biological medium,” as used herein refers to both in vitro and in vivo biological milieus. Exemplary in vitro “biological media” include, but are not limited to, cell culture, tissue culture, homogenates, plasma and blood. In vivo applications are generally performed in mammals, preferably humans.

MIC, or minimum inhibitory concentration, is the point where compound stops more than 90% of cell growth relative to an untreated control.

Also of use in the present invention are compounds that are poly- or multi-valent species, including, for example, species such as dimers, trimers, tetramers and higher homologs of the compounds of use in the invention or reactive analogues thereof. The poly- and multi-valent species can be assembled from a single species or more than one species of the invention. For example, a dimeric construct can be “homo-dimeric” or “heterodimeric.” Moreover, poly- and multi-valent constructs in which a compound of the invention or a reactive analogue thereof, is attached to an oligomeric or polymeric framework (e.g., polylysine, dextran, hydroxyethyl starch and the like) are within the scope of the present invention. The framework is preferably polyfunctional (i.e. having an array of reactive sites for attaching compounds of use in the invention). Moreover, the framework can be derivatized with a single species of the invention or more than one species of the invention.

Moreover, the present invention includes the use of compounds within the motif set forth in the formulas contained herein, which are functionalized to afford compounds having water-solubility that is enhanced relative to analogous compounds that are not similarly functionalized. Thus, any of the substituents set forth herein can be replaced with analogous radicals that have enhanced water solubility. For example, it is within the scope of the invention to replace a hydroxyl group with a diol, or an amine with a quaternary amine, hydroxy amine or similar more water-soluble moiety. In a preferred embodiment, additional water solubility is imparted by substitution at a site not essential for the activity towards the editing domain of the compounds set forth herein with a moiety that enhances the water solubility of the parent compounds. Methods of enhancing the water-solubility of organic compounds are known in the art. Such methods include, but are not limited to, functionalizing an organic nucleus with a permanently charged moiety, e.g., quaternary ammonium, or a group that is charged at a physiologically relevant pH, e.g. carboxylic acid, amine. Other methods include, appending to the organic nucleus hydroxyl- or amine-containing groups, e.g. alcohols, polyols, polyethers, and the like. Representative examples include, but are not limited to, polylysine, polyethyleneimine, poly(ethyleneglycol) and poly(propyleneglycol). Suitable functionalization chemistries and strategies for these compounds are known in the art. See, for example, Dunn, R. L., et al., Eds. POLYMERIC DRUGS AND DRUG DELIVERY SYSTEMS, ACS Symposium Series Vol. 469, American Chemical Society, Washington, D.C. 1991.

### DESCRIPTION OF THE EMBODIMENTS

I. Boron-Containing Compounds

This invention provides boron-containing compounds which are useful in the treatment of microorganisms located in the oral cavities of animals. The compounds are also useful in treating periodontal disease.

I. a.) Borinic Esters

The invention comprises a compound having the structure according to the following formulae: 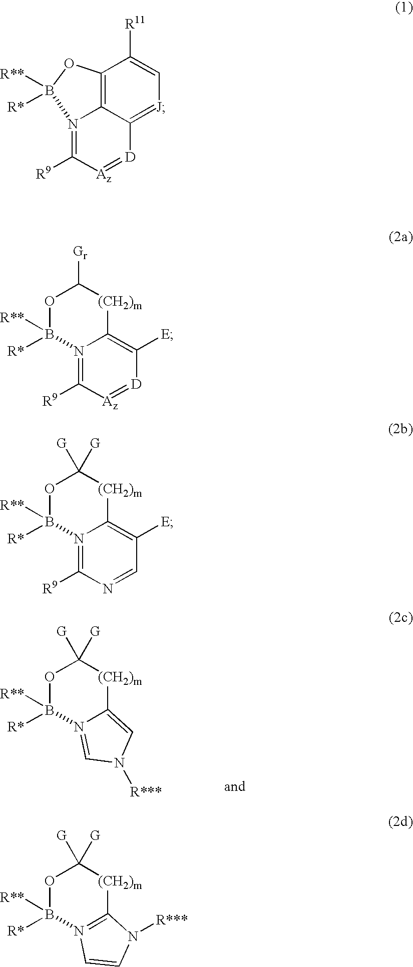
wherein B is boron, O is oxygen, R* and R** are each independently selected from substituted or unsubstituted alkyl (C₁-C₄), substituted or unsubstituted cycloalkyl (C₃-C₇), substituted or unsubstituted alkenyl, substituted or unsubstituted alkynyl, substituted or unsubstituted aralkyl, substituted or unsubstituted phenyl, and substituted or unsubstituted heteroaryl. The index z is 0 or 1 and when z is 1, A is CH, CR¹⁰ or N. D is N, CH, or CR¹². E is H, OH, alkoxy or 2-(morpholino)ethoxy, CO₂H or CO₂alkyl. The index m=0-2, the index r is 1 or 2, and wherein when r is 1, G is ═O (double-bonded oxygen) and when r is 2, each G is independently H, methyl, ethyl or propyl. R¹² is selected from (CH₂)ₖOH (where k=1, 2 or 3), CH₂NH₂, CH₂NH-alkyl, CH₂N(alkyl)₂, CO₂H, CO₂alkyl, CONH₂, OH, alkoxy, aryloxy, SH, S-alkyl, S-aryl, SO₂N(alkyl)₂, SO₂NHalkyl, SO₂NH₂, SO₂alkyl, SO₃H, SCF₃, CN, halogen, CF₃, NO₂, NH₂, 2^{o}-amino, 3^{o}-amino, NH₂SO₂ and CONH₂, and wherein J is CR¹⁰ or N. R⁹, R¹⁰ and R¹¹ are each independently selected from the group consisting of hydrogen, alkyl, cycloalkyl, (CH₂)ₙOH (n=2 to 3), CH₂NH₂, CH₂NHalkyl, CH₂N(alkyl)₂, halogen, CHO, CH═NOH, CO₂H, CO₂-alkyl, S-alkyl, SO₂-alkyl, S-aryl, SO₂N(alkyl)₂, SO₂NHalkyl, SO₂NH₂, NH₂, alkoxy, CF₃, SCF₃, NO₂, SO₃H and OH, including salts thereof.

In preferred embodiments of a formula described herein, such as formula 1, 2a, 2b, 2c or 2d, R* and/or R** are the same or are different, preferably wherein one of R* and R** is a substituted or unsubstituted alkyl (C₁-C₄) or R* and R** are each a substituted or unsubstituted alkyl (C₁-C₄).

In a preferred embodiment of a formula described herein, such as formula 1, 2a, 2b, 2c or 2d, R* and/or R** are the same or are different, preferably wherein one of R* and R** is a substituted or unsubstituted cycloalkyl (C₃-C₇) or R* and R** are each a substituted or unsubstituted cycloalkyl (C₃-C₇).

In a preferred embodiment of a formula described herein, such as formula 1, 2a, 2b, 2c or 2d, R* and/or R** are the same or are different, preferably wherein one of R* and R** is a substituted or unsubstituted alkenyl or R* and R** are each a substituted or unsubstituted alkenyl. In a further preferred embodiment thereof, the alkenyl has the structure 2

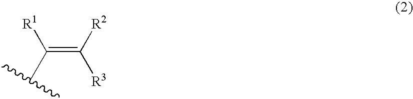

Wherein R¹, R², and R³ are each independently selected from the group consisting of hydrogen, alkyl, aryl, cycloalkyl, substituted aryl, aralkyl, substituted aralkyl, (CH₂)ₖOH (where k=1, 2 or 3), CH₂NH₂, CH₂NH-alkyl, CH₂N(alkyl)₂, CO₂H, CO₂alkyl, CONH₂, S-alkyl, S-aryl, SO₂alkyl, SO₂N(alkyl)₂, SO₂NHalkyl, SO₂NH₂, SO₃H, SCF₃, CN, halogen, CF₃ and NO₂.

In a preferred embodiment of a formula described herein, such as formula 1, 2a, 2b, 2c or 2d, R* and/or R** are the same or are different, preferably wherein one of R* and R** is a substituted or unsubstituted alkynyl or R* and R** are each a substituted or unsubstituted alkynyl. In a further preferred embodiment thereof, the alkynyl has the structure 3 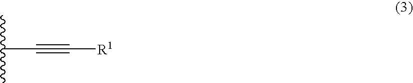
wherein R¹ is selected from the group consisting of hydrogen, alkyl, cycloalkyl, aryl, substituted aryl, aralkyl, substituted aralkyl, (CH₂)ₖOH (where k=1, 2 or 3), CH₂NH₂, CH₂NH-alkyl, CH₂N(alkyl)₂, CO₂H, CO₂alkyl, CONH₂, S-alkyl, S-aryl, SO₂alkyl, SO₂N(alkyl)₂, SO₂NHalkyl, SO₂NH₂, SO₃H, SCF₃, CN, halogen, CF₃ and NO₂.

In a preferred embodiment of a formula described herein, such as formula 1, 2a, 2b, 2c or 2d, R* and/or R** are the same or are different, preferably wherein one of R* and R** is a substituted or unsubstituted phenyl or R* and R** are each a substituted or unsubstituted phenyl but excluding compounds of formula 1 wherein z is 1, A is CR¹⁰, D is CR¹², J is CR¹⁰ and excluding compounds of formula 2 wherein the combination of substituents is such that z is 1, A is CR¹⁰, D is CR¹², m is 2, and G is H or methyl or ethyl. In a separate embodiment of the foregoing, G is also not propyl. However, in specific embodiments such excluded compounds, although not being claimed as novel, may find use in one or more of the methods of the invention, preferably for treatment against infection, most preferably in treatment against fungal infection. In a preferred embodiment, only novel compounds of the invention are contemplated for such uses.

The novel compounds of the invention do not include quinaldine derivatives, such as 2-methylquinoline, wherein R⁹ is methyl, A_{z} is CH, D is CH, J is CH and R¹¹ is hydrogen. However, such compounds may be useful in the methods of the invention.

A preferred embodiment is a compound of a formula described herein, such as formula 1, 2a, 2b, 2c or 2d, wherein R* and R** are each other than a phenyl or substituted phenyl.

Another preferred embodiment is a compound of a formula described herein, such as formula 1, 2a, 2b, 2c or 2d, wherein one of R* or R** is benzyl or substituted benzyl.

An additional preferred embodiment is a compound of a formula described herein, such as formula 1, 2a, 2b, 2c or 2d, wherein r is 1, G is ═O, m is 0 and E is OH.

A preferred embodiment is also a compound of a formula described herein, such as formula 1, 2a, 2b, 2c or 2d, wherein z is 1 and R⁹ is selected from alkyl (greater than C₄), (CH₂)ₙOH (n=1, 2 or 3), CH₂NH₂, CH₂NHalkyl, CH₂N(alkyl)₂, CHO, CH═NOH, CO₂H, CO₂-alkyl, S-alkyl, SO₂-alkyl, S-aryl, alkoxy (greater than C₄), SCF₃, and NO₂.

In one preferred embodiment the compound has a formula described herein, such as formula 1, 2a, 2b, 2c or 2d, wherein z is 1 and R¹⁰ is selected from alkyl (greater than C₄), (CH₂)ₙOH (n=1, 2 or 3), CH₂NH₂, CH₂NHalkyl, CH₂N(alkyl)₂, CHO, CH═NOH, CO₂H, CO₂-alkyl, S-alkyl, SO₂-alkyl, S-aryl, alkoxy (greater than C₄), SCF₃, and NO₂.

In another preferred embodiment the compound has a formula described herein, such as formula 1, 2a, 2b, 2c or 2d, wherein z is 1 and D is CR¹² wherein R¹² is selected from (CH₂)ₖOH (where k=1, 2 or 3), CH₂NH₂, CH₂NH-alkyl, CH₂N(alkyl)₂, CO₂H, CO₂alkyl, CONH₂, OH, alkoxy (greater than C₄), aryloxy, SH, S-alkyl, S-aryl, SO₂alkyl, SO₃H, SCF₃, CN, NO₂, NH₂SO₂ and CONH₂.

In an additional preferred embodiment the compound has a formula described herein, such as formula 1, 2a, 2b, 2c or 2d, wherein z is 1, E is N-(morpholinyl)ethoxy or alkoxy greater than C₄.

Other preferred embodiments are compounds having the structure of a formula described herein, such as formula 1, 2a, 2b, 2c or 2d, wherein A or D is nitrogen, or wherein m is 2.

In another preferred embodiment, the compound has the structure of a formula described herein, such as formula 1, 2a, 2b, 2c or 2d, wherein one of R* or R** is substituted phenyl substituted with 1 to 5 substituents each of which is independently selected from alkyl (greater than C₆), aryl, substituted aryl, benzyl, substituted benzyl, (CH₂)ₖOH (where k=1, 2 or 3), CH₂NH₂, CH₂NH-alkyl, CH₂N(alkyl)₂, CO₂H, CO₂alkyl, CONH₂, CONHalkyl, CON(alkyl)₂, OH, alkoxy (greater than C₆), aryloxy, SH, S-alkyl, S-aryl, SO₂alkyl, SO₃H, SCF₃, CN, NO₂, NH₂, 2^{o}-amino, 3^{o}-amino, NH₂SO₂, OCH₂CH₂NH₂, OCH₂CH₂NHalkyl, OCH₂CH₂N(alkyl)₂, oxazolidin-2-yl, and alkyl substituted oxazolidin-2-yl.

In a further preferred embodiment thereof, the phenyl has the structure 4 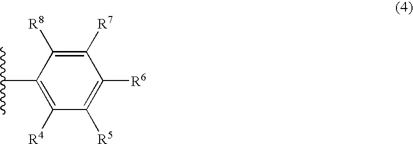
wherein R⁴, R⁵, R⁶, R⁷ and R⁸ are each independently selected from the group consisting of hydrogen, alkyl, cycloalkyl aryl, substituted aryl, aralkyl, substituted aralkyl, (CH₂)ₖOH (where k=1, 2 or 3), CH₂NH₂, CH₂NH-alkyl, CH₂N(alkyl)₂, CO₂H, CO₂alkyl, CONH₂, CONHalkyl, CON(alkyl)₂, OH, alkoxy, aryloxy, SH, S-alkyl, S-aryl, SO₂alkyl, SO₂N(alkyl)₂, SO₂NHalkyl, SO₂NH₂, SO₃H, SCF₃, CN, halogen, CF₃, NO₂, NH₂, 2^{o}-amino, 3^{o}-amino, NH₂SO₂, OCH₂CH₂NH₂, OCH₂CH₂NHalkyl, OCH₂CH₂N(alkyl)₂, oxazolidin-2-yl, or alkyl substituted oxazolidin-2-yl.

One highly preferred embodiment is a compound of a formula described herein, such as formula 1, 2a, 2b, 2c or 2d, wherein R* is 3-fluorophenyl, R** is 4-chlorophenyl, R⁹ is H, R¹¹ is H, A_{z} is CH, D is CH, J is CH and may be called (3-fluorophenyl)(4-chlorophenyl)borinic acid 8-hydroxyquinoline ester.

Another preferred embodiment is a compound of a formula described herein, such as formula 1, 2a, 2b, 2c or 2d, wherein R* and R** are each 3-(4,4-dimethyloxazolidin-2-yl)phenyl, R⁹ is H, R¹¹ is H, A_{z} is CH, D is CH, J is CH and may be called bis(3-(4,4-dimethyloxazolidin-2-yl)phenyl)borinic acid 8-hydroxyquinoline ester.

An additional preferred embodiment is a compound of a formula described herein, such as formula 1, 2a, 2b, 2c or 2d, wherein R* is 3-fluorophenyl, R** is cyclopropyl, R⁹ is H, R¹¹ is H, A_{z} is CH, D is CH, J is CH and referred to as (3-fluorophenyl)(cyclopropyl)borinic acid 8-hydroxyquinoline ester.

A highly preferred embodiment is a compound of a formula described herein, such as formula 1, 2a, 2b, 2c or 2d, wherein R* is 4-(N,N-dimethyl)-aminomethylphenyl, R** is 4-cyanophenyl, R⁹ is H, R¹¹ is H, A_{z} is CH, D is CH, J is CH and is referred to as (4-(N,N-dimethyl)-aminomethylphenyl)(4-cyanophenyl)borinic acid 8-hydroxyquinoline ester.

Another highly preferred embodiment is a compound of a formula described herein, such as formula 1, 2a, 2b, 2c or 2d, wherein R* is the same as R** which is 3-chloro-4-methylphenyl, R⁹ is H, R¹¹ is H, A_{z} is CH, D is CH and E is OH, m=0, r is 1, G is ═O (double bonded oxygen) and is referred to as bis(3-chloro-4-methylphenyl)borinic acid 3-hydroxypicolinate ester.

A further highly preferred embodiment is a compound of a formula described herein, such as formula 1, 2a, 2b, 2c or 2d, wherein R* is the same as R* which is 2-methyl-4-chlorophenyl, R⁹ is H, R¹¹ is H, A_{z} is CH, D is CH and E is OH, m=0, r is 1, G is ═O (double bonded oxygen) and is referred to as bis(2-methyl-4-chlorophenyl)borinic acid 3-hydroxypicolinate ester.

In a preferred embodiment of a formula described herein, such as formula 1, 2a, 2b, 2c or 2d, R* and/or R** are the same or are different, preferably wherein one of R* and R** is a substituted or unsubstituted benzyl or R* and R** are each a substituted or unsubstituted benzyl. In a further preferred embodiment thereof, the benzyl has the structure 5 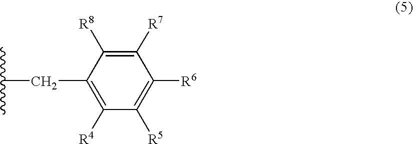
wherein R⁴, R⁵, R⁶, R⁷ and R⁸ are each independently selected from the group consisting of alkyl, cycloalkyl, aryl, substituted aryl, aralkyl, substituted aralkyl, (CH₂)ₖOH (where k=1, 2 or 3), CH₂NH₂, CH₂NH-alkyl, CH₂N(alkyl)₂, CO₂H, CO₂alkyl, CONH₂, CONHalkyl, CON(alkyl)₂, OH, alkoxy, aryloxy, SH, S-alkyl, S-aryl, SO₂alkyl, SO₂N(alkyl)₂, SO₂NHalkyl, SO₂NH₂, SO₃H, SCF₃, CN, halogen, CF₃, NO₂, NH₂, 2^{o}-amino, 3^{o}-amino, NH₂SO₂, OCH₂CH₂NH₂—, OCH₂CH₂NHalkyl, OCH₂CH2N(alkyl)₂, oxazolidin-2-yl, or alkyl substituted oxazolidin-2-yl.

One preferred embodiment is a compound of a formula described herein, such as formula 1, 2a, 2b, 2c or 2d, R* and/or R** are the same or are different, preferably wherein one of R* and R** is a substituted or unsubstituted heterocycle or R* and R** are each a substituted or unsubstituted heterocycle. In a further preferred embodiment thereof, the heterocycle has the structure 6 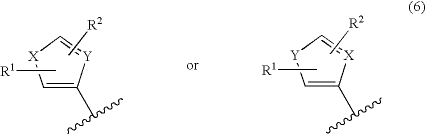
wherein X═CH═CH, N═CH, NR¹³ (wherein R¹³═H, alkyl, aryl or aralkyl), O, or S and wherein Y═CH or N. R¹, R², and R³ are each independently selected from the group consisting of hydrogen, alkyl, cycloalkyl, aryl, substituted aryl, aralkyl, substituted aralkyl, (CH₂)ₖOH (where k=1, 2 or 3), CH₂NH₂, CH₂NH-alkyl, CH₂N(alkyl)₂, CO₂H, CO₂alkyl, CONH₂, S-alkyl, S-aryl, SO₂alkyl, SO₂N(alkyl)₂, SO₂NHalkyl, SO₂NH₂, SO₃H, SCF₃, CN, halogen, CF₃, NO₂, oxazolidin-2-yl, or alkyl substituted oxazolidin-2-yl.

A highly preferred embodiment is a compound of a formula described herein, such as formula 1, 2a, 2b, 2c or 2d, wherein R* is pyrid-3-yl, R** is 4-chlorophenyl, R⁹ is H, R¹¹ is H, A_{z} is CH, D is CH, and J is CH (named (pyrid-3-yl)(4-chlorophenyl)borinic acid 8-hydroxyquinoline ester).

A highly preferred embodiment is a compound of a formula described herein, such as formula 1, 2a, 2b, 2c or 2d, wherein R* is 5-cyanopyrid-3-yl, R** is vinyl, R⁹ is H, R¹¹ is H, A_{z} is CH, D is CH, and J is CH (named (5-cyanopyrid-3-yl)(vinyl)borinic acid 8-hydroxyquinoline ester).

One preferred embodiment is a compound of a formula described herein, such as formula 1, 2a, 2b, 2c or 2d, wherein R⁹ is H, R¹¹ is H, A_{z} is CH, D is CH, and J is CH.

Another preferred embodiment is a compound of a formula described herein, such as formula 1, 2a, 2b, 2c or 2d, wherein R⁹ is H, R¹¹ is H, A_{z} is CH, D is CH and E is OH, m=0, r is 1, and G is ═O (double bonded oxygen).

The structures of the invention also permit solvent interactions that may afford structures (such as Formulas 1b and 2e) 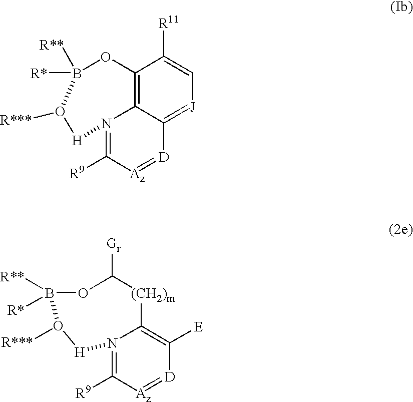
that include atoms derived from the solvent encountered by the compounds of the invention during synthetic procedures and therapeutic uses. Thus, such solvent structures can especially insinuate themselves into at least some of the compounds of the invention, especially between the boron and nitrogen atoms, to increase the ring size of such compounds by one or two atoms. For example, where the boron ring of a structure of the invention comprises 5 atoms, including, for example, the boron, a nitrogen, an oxygen and 2 carbons, insinuation of a solvent atom between the boron and nitrogen would afford a 7 membered ring. In one example, use of hydroxyl and amino solvents may afford structures containing an oxygen or nitrogen between the ring boron and nitrogen atoms to increase the size of the ring. Such structures are expressly contemplated by the present invention, preferably where R*** is H or alkyl.
Methods of Making the Compounds

The synthesis of the compounds of the invention is accomplished in several formats. Reaction scheme A demonstrates the synthesis of the intermediate borinic acids, and their subsequent conversion to the desired borinic acid complexes. When R* and R** are identical, the reaction of two equivalents of an arylmagnesium halide (or aryllithium) with trialkyl borate, followed by acidic hydrolysis affords the desired borinic acid 5. When R* and R** are not identical, the reaction of one equivalent of an arylmagnesium halide (or aryllithium) with appropriate aryl(dialkoxy)borane (4), heteroaryl(dialkoxy)borane or alkyl(dialkoxy)borane (alkoxy group comprised of methoxy, ethoxy, isopropoxy, or propoxy moiety), followed by acidic hydrolysis affords the unsymmetrical borinic acids 6 in excellent yields. Where applicable, the reaction of the alkylene esters (3, T=nothing, CH₂, C(CH₃)₂) with the appropriate organocerium, organolithium, organomagnesium or equivalent reactant is convenient.

As shown in Scheme A, the borinic acid complexes are obtained from the precursor borinic acids by reaction with one equivalent of the desired heterocyclic ligand in suitable solvents (i.e., ethanol, isopropanol, dioxane, ether, toluene, dimethylformamide, N-methylpyrrolidone, or tetrahydrofuran).

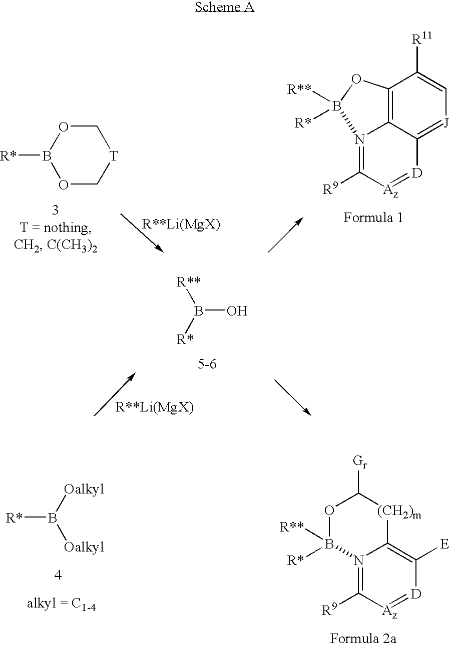

In certain situations, compounds of the invention may contain one or more asymmetric carbon atoms, so that the compounds can exist in different stereoisomeric forms. These compounds can be, for example, racemates or optically active forms. In these situations, the single enantiomers, i.e., optically active forms, can be obtained by asymmetric synthesis or by resolution of the racemates. Resolution of the racemates can be accomplished, for example, by conventional methods such as crystallization in the presence of a resolving agent, or chromatography, using, for example a chiral HPLC column.

I. b.) Boronic Esters

In a first aspect, the invention provides a compound having a structure according to Formula I: 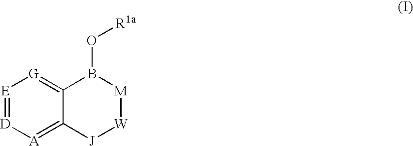
wherein B is boron. R^{1a} is a member selected from a negative charge, a salt counterion, H, cyano, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. M is a member selected from oxygen, sulfur and NR^{2a}. R^{2a} is a member selected from H, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. J is a member selected from (CR^{3a}R^{4a})ₙ₁ and CR^{5a}. R^{3a}, R^{4a}, and R^{5a} are members independently selected from H, cyano, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. The index n1 is an integer selected from 0 to 2. W is a member selected from C═O (carbonyl), (CR^{6a}R^{7a})ₘₗ and CR^{8a}. R^{6a}, R^{7a}, and R^{8a} are members independently selected from H, cyano, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. The index m1 is an integer selected from 0 and 1. A is a member selected from CR^{9a} and N. D is a member selected from CR^{10a} and N. E is a member selected from CR^{11a} and N. G is a member selected from CR^{12a} and N. R^{9a}, R^{10a}, R^{11a} and R^{12a} are members independently selected from H, OR*^{a}, NR*^{a}R**^{a}, SR*^{a}, —S(O)R*^{a}, —S(O)₂R*^{a}, —S(O)₂NR*^{a}R**^{a}, C(O)R*^{a}, —C(O)OR*^{a} C(O)NR*^{a}R**^{a}, nitro, halogen, cyano, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. Each R*^{a} and R**^{a} are members independently selected from H, nitro, halogen, cyano, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. The combination of nitrogens (A+D+E+G) is an integer selected from 0 to 3. A member selected from R^{3a}, R^{4a} and R^{5a} and a member selected from R^{6a}, R^{7a} and R^{8a}, together with the atoms to which they are attached, are optionally joined to form a 4 to 7 membered ring. R^{3a} and R^{4a}, together with the atoms to which they are attached, are optionally joined to form a 4 to 7 membered ring. R^{6a} and R^{7a}, together with the atoms to which they are attached, are optionally joined to form a 4 to 7 membered ring. R^{9a} and R^{10a}, together with the atoms to which they are attached, are optionally joined to form a 4 to 7 membered ring. R^{10a} and R^{11a}, together with the atoms to which they are attached, are optionally joined to form a 4 to 7 membered ring. R^{11a} and R^{12a}, together with the atoms to which they are attached, are optionally joined to form a 4 to 7 membered ring.

In an exemplary embodiment, the compound has a structure according to Formula (Ia):

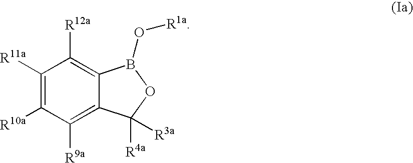

In another exemplary embodiment, each R^{3a} and R^{4a} is a member independently selected from H, cyano, substituted or unsubstituted methyl, substituted or unsubstituted ethyl, trifluoromethyl, substituted or unsubstituted hydroxymethyl, substituted or unsubstituted hydroxyalkyl, substituted or unsubstituted benzyl, substituted or unsubstituted phenyl, substituted or unsubstituted mercaptomethyl, substituted or unsubstituted mercaptoalkyl, substituted or unsubstituted aminomethyl, substituted or unsubstituted alkylaminomethyl, substituted or unsubstituted dialkylaminomethyl, substituted or unsubstituted arylaminomethyl, substituted or unsubstituted indolyl and substituted or unsubstituted amido. In another exemplary embodiment, each R^{3a} and R^{4a} is a member independently selected from cyano, substituted or unsubstituted methyl, substituted or unsubstituted ethyl, trifluoromethyl, substituted or unsubstituted hydroxymethyl, substituted or unsubstituted hydroxyalkyl, substituted or unsubstituted benzyl, substituted or unsubstituted phenyl, substituted or unsubstituted mercaptomethyl, substituted or unsubstituted mercaptoalkyl, substituted or unsubstituted aminomethyl, substituted or unsubstituted alkylaminomethyl, substituted or unsubstituted dialkylaminomethyl, substituted or unsubstituted arylaminomethyl, substituted or unsubstituted indolyl, substituted or unsubstituted amido.

In another exemplary embodiment, each R^{3a} and R^{4a} is a member selected from H, substituted or unsubstituted methyl, substituted or unsubstituted ethyl, substituted or unsubstituted propyl, substituted or unsubstituted isopropyl, substituted or unsubstituted butyl, substituted or unsubstituted t-butyl, substituted or unsubstituted phenyl and substituted or unsubstituted benzyl. In another exemplary embodiment, R^{3a} and R^{4a} is a member selected from methyl, ethyl, propyl, isopropyl, butyl, t-butyl, phenyl and benzyl. In another exemplary embodiment, R^{3a} is H and R^{4a} is a member selected from methyl, ethyl, propyl, isopropyl, butyl, t-butyl, phenyl and benzyl. In another exemplary embodiment, R^{3a} is H and R^{4a} is H.

In another exemplary embodiment, each R^{9a}, R^{10a}, R^{11a} and R^{12a} is a member independently selected from H, OR*^{a}, NR*R**^{a}, SR*^{a}, —S(O)R*^{a}, —S(O)₂R*^{a}, —S(O)₂NR*^{a}R**^{a}, —C(O)R*^{a}, —C(O)OR*^{a}, —C(O)NR*^{a}R**^{a}, halogen, cyano, nitro, substituted or unsubstituted methoxy, substituted or unsubstituted methyl, substituted or unsubstituted ethoxy, substituted or unsubstituted ethyl, trifluoromethyl, substituted or unsubstituted hydroxymethyl, substituted or unsubstituted hydroxyalkyl, substituted or unsubstituted benzyl, substituted or unsubstituted phenyl, substituted or unsubstituted phenyloxy, substituted or unsubstituted phenyl methoxy, substituted or unsubstituted thiophenyloxy, substituted or unsubstituted pyridinyloxy, substituted or unsubstituted pyrimidinyloxy, substituted or unsubstituted benzylfuran, substituted or unsubstituted methylthio, substituted or unsubstituted mercaptomethyl, substituted or unsubstituted mercaptoalkyl, substituted or unsubstituted phenylthio, substituted or unsubstituted thiophenylthio, substituted or unsubstituted phenyl methylthio, substituted or unsubstituted pyridinylthio, substituted or unsubstituted pyrimidinylthio, substituted or unsubstituted benzylthiofuranyl, substituted or unsubstituted phenylsulfonyl, substituted or unsubstituted benzylsulfonyl, substituted or unsubstituted phenylmethylsulfonyl, substituted or unsubstituted thiophenylsulfonyl, substituted or unsubstituted pyridinylsulfonyl, substituted or unsubstituted pyrimidinylsulfonyl, substituted or unsubstituted sulfonamidyl, substituted or unsubstituted phenylsulfinyl, substituted or unsubstituted benzylsulfinyl, substituted or unsubstituted phenylmethylsulfinyl, substituted or unsubstituted thiophenylsulfinyl, substituted or unsubstituted pyridinylsulfinyl, substituted or unsubstituted pyrimidinylsulfinyl, substituted or unsubstituted amino, substituted or unsubstituted alkylamino, substituted or unsubstituted dialkylamino, substituted or unsubstituted trifluoromethylamino, substituted or unsubstituted aminomethyl, substituted or unsubstituted alkylaminomethyl, substituted or unsubstituted dialkylaminomethyl, substituted or unsubstituted arylaminomethyl, substituted or unsubstituted benzylamino, substituted or unsubstituted phenylamino, substituted or unsubstituted thiophenylamino, substituted or unsubstituted pyridinylamino, substituted or unsubstituted pyrimidinylamino, substituted or unsubstituted indolyl, substituted or unsubstituted morpholino, substituted or unsubstituted alkylamido, substituted or unsubstituted arylamido, substituted or unsubstituted ureido, substituted or unsubstituted carbamoyl, and substituted or unsubstituted piperizinyl. In an exemplary embodiment, R^{9a}, R^{10a}, R^{11a} and R^{12a} are selected from the previous list of substituents with the exception of —C(O)R*^{a}, —C(O)OR*^{a}, —C(O)NR*^{a}R**^{a}.

In another exemplary embodiment, R^{9a}, R^{10a}, R^{11a} and R^{12a} are members independently selected from fluoro, chloro, bromo, nitro, cyano, amino, methyl, hydroxylmethyl, trifluoromethyl, methoxy, trifluoromethyoxy, ethyl, diethylcarbamoyl, pyridin-2-yl, pyridin-3-yl, pyridin-4-yl, pyrimidinyl, piperizino, piperizinyl, piperizinocarbonyl, piperizinylcarbonyl, carboxyl, 1-tetrazolyl, 1-ethoxycarbonylmethoxy, carboxymethoxy, thiophenyl, 3-(butylcarbonyl)phenylmethoxy, 1H-tetrazol-5-yl, 1-ethoxycarbonylmethyloxy-, 1-ethoxycarbonylmethyl-, 1-ethoxycarbonyl-, carboxymethoxy-, thiophen-2-yl, thiophen-2-ylthio-, thiophen-3-yl, thiophen-3-ylthio, 4-fluorophenylthio, butylcarbonylphenylmethoxy, butylcarbonylphenylmethyl, butylcarbonylmethyl, 1-(piperidin-1-yl)carbonyl)methyl, 1-(piperidin-1-yl)carbonyl)methoxy, 1-(piperidin-2-yl)carbonyl)methoxy, 1-(piperidin-3-yl)carbonyl)methoxy, 1-(4-(pyrimidin-2-yl)piperazin-1-yl)carbonyl)methoxy, 1-(4-(pyrimidin-2-yl)piperazin-1-yl)carbonyl)methyl, 1-(4-(pyrimidin-2-yl)piperazin-1-yl)carbonyl, 1-4-(pyrimidin-2-yl)piperazin-1-yl, 1-(4-(pyridin-2-yl)piperazin-1-yl)carbonyl), 1-(4-(pyridin-2-yl)piperazin-1-yl)carbonylmethyl, (1-(4-(pyridin-2-yl)piperazin-1-yl)carbonyl)-methoxy), 1-(4-(pyridin-2-yl)piperazin-1-yl, 1H-indol-1-yl, morpholino-, morpholinyl, morpholinocarbonyl, morpholinylcarbonyl, phenylureido, phenylcarbamoyl, acetamido, 3-(phenylthio)-1H-indol-1-yl, 3-(2-cyanoethylthio)-1H-indol-1-yl, benzylamino, 5-methoxy-3-(phenylthio)-1H-indol-1-yl, 5-methoxy-3-(2-cyanoethylthio)-1H-indol-1-yl)), 5-chloro-1H-indol-1-yl, 5-chloro-3-(2-cyanoethylthio)-1H-indol-1-yl)), dibenzylamino, benzylamino, 5-chloro-3-(phenylthio)-1H-indol-1-yl)), 4-(1H-tetrazol-5-yl)phenoxy, 4-(1H-tetrazol-5-yl)phenyl, 4-(1H-tetrazol-5-yl)phenylthio, 2-cyanophenoxy, 3-cyanophenoxy, 4-cyanophenoxy, 2-cyanophenylthio, 3-cyanophenylthio, 4-cyanophenylthio, 2-chlorophenoxy, 3-chlorophenoxy, 4-chlorophenoxy, 2-fluorophenoxy, 3-fluorophenoxy, 4-fluorophenoxy, 2-cyanobenzyloxy, 3-cyanobenzyloxy, 4-cyanobenzyloxy, 2-chlorobenzyloxy, 3-chlorobenzyloxy, 4-chlorobenzyloxy, 2-fluorobenzyloxy, 3-fluorobenzyloxy, 4-fluorobenzyloxy, unsubstituted phenyl, unsubstituted benzyl. In an exemplary embodiment, R^{9a} is H and R^{12a} is H.

In an exemplary embodiment, the compound according to Formula (I) or Formula (Ia) is a member selected from: 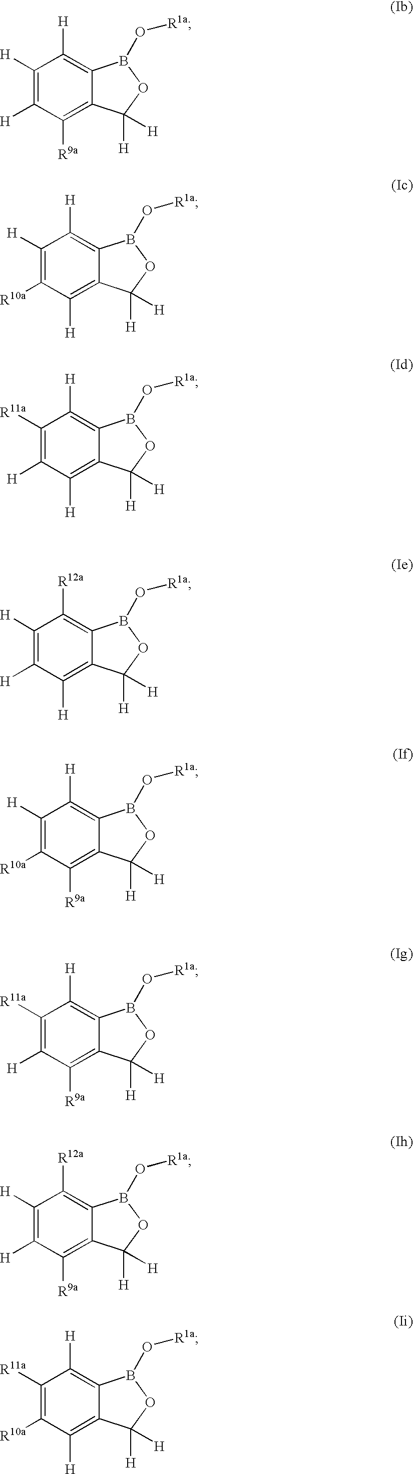 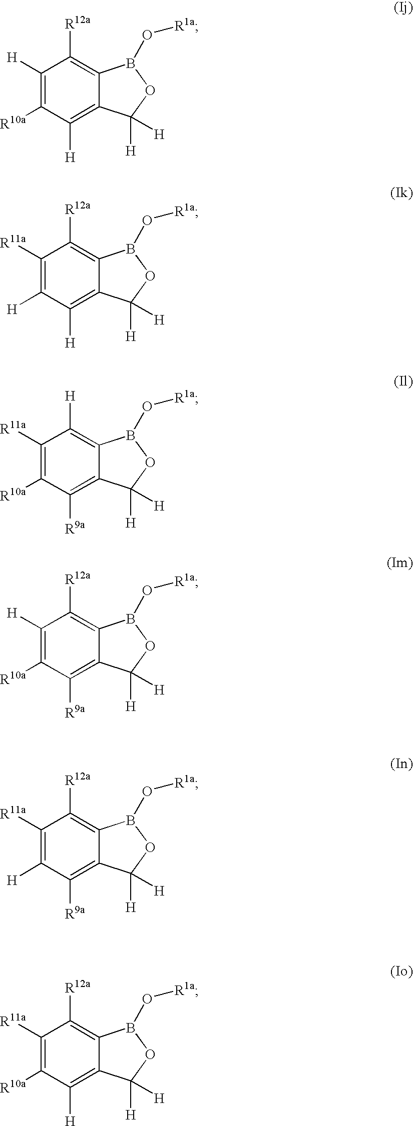
In an exemplary embodiment, the compound has a structure according to one of Formulae I-Io with substituent selections for R^{9a}, R^{10a}, R^{11a} and R^{12a} including all the possibilities contained in paragraph 90 except for H. In an exemplary embodiment, the compound has a structure according to one of Formulae Ib-Io with substituent selections for R^{9a}, R^{10a}, R^{11a} and R^{12a} including all the possibilities contained in paragraph 91 except for H.

In an exemplary embodiment, the compound has a formula according to Formulae (Ib)-(Ie) wherein R^{1a} is a member selected from H, a negative charge and a salt counterion and the remaining R group (R^{9a} in Ib, R^{10a} in Ic, R^{11a} in Id, and R^{12a} in Ie) is a member selected from fluoro, chloro, bromo, nitro, cyano, amino, methyl, hydroxylmethyl, trifluoromethyl, methoxy, trifluoromethyoxy, ethyl, diethylcarbamoyl, pyridin-2-yl, pyridin-3-yl, pyridin-4-yl, pyrimidinyl, piperizino, piperizinyl, piperizinocarbonyl, piperizinylcarbonyl, carboxyl, 1-tetrazolyl, 1-ethoxycarbonylmethoxy, carboxymethoxy, thiophenyl, 3-(butylcarbonyl)phenylmethoxy, 1H-tetrazol-5-yl, 1-ethoxycarbonylmethyloxy-, 1-ethoxycarbonylmethyl-, 1-ethoxycarbonyl-, carboxymethoxy-, thiophen-2-yl, thiophen-2-ylthio-, thiophen-3-yl, thiophen-3-ylthio, 4-fluorophenylthio, butylcarbonylphenylmethoxy, butylcarbonylphenylmethyl, butylcarbonylmethyl, 1-(piperidin-1-yl)carbonyl)methyl, 1-(piperidin-1-yl)carbonyl)methoxy, 1-(piperidin-2-yl)carbonyl)methoxy, 1-(piperidin-3-yl)carbonyl)methoxy, 1-(4-(pyrimidin-2-yl)piperazin-1-yl)carbonyl)methoxy, 1-(4-(pyrimidin-2-yl)piperazin-1-yl)carbonyl)methyl, 1-(4-(pyrimidin-2-yl)piperazin-1-yl)carbonyl, 1-4-(pyrimidin-2-yl)piperazin-1-yl, 1-(4-(pyridin-2-yl)piperazin-1-yl)carbonyl), 1-(4-(pyridin-2-yl)piperazin-1-yl)carbonylmethyl, (1-(4-(pyridin-2-yl)piperazin-1-yl)carbonyl)-methoxy), 1-(4-(pyridin-2-yl)piperazin-1-yl, 1H-indol-1-yl, morpholino-, morpholinyl, morpholinocarbonyl, morpholinylcarbonyl, phenylureido, phenylcarbamoyl, acetamido, 3-(phenylthio)-1H-indol-1-yl, 3-(2-cyanoethylthio)-1H-indol-1-yl, benzylamino, 5-methoxy-3-(phenylthio)-1H-indol-1-yl, 5-methoxy-3-(2-cyanoethylthio)-1H-indol-1-yl)), 5-chloro-1H-indol-1-yl, 5-chloro-3-(2-cyanoethylthio)-1H-indol-1-yl)), dibenzylamino, benzylamino, 5-chloro-3-(phenylthio)-1H-indol-1-yl)), 4-(1H-tetrazol-5-yl)phenoxy, 4-(1H-tetrazol-5-yl)phenyl, 4-(1H-tetrazol-5-yl)phenylthio, 2-cyanophenoxy, 3-cyanophenoxy, 4-cyanophenoxy, 2-cyanophenylthio, 3-cyanophenylthio, 4-cyanophenylthio, 2-chlorophenoxy, 3-chlorophenoxy, 4-chlorophenoxy, 2-fluorophenoxy, 3-fluorophenoxy, 4-fluorophenoxy, 2-cyanobenzyloxy, 3-cyanobenzyloxy, 4-cyanobenzyloxy, 2-chlorobenzyloxy, 3-chlorobenzyloxy, 4-chlorobenzyloxy, 2-fluorobenzyloxy, 3-fluorobenzyloxy and 4-fluorobenzyloxy.

In an exemplary embodiment, the compound has a formula according to Formulae (If)-(Ik) wherein R^{1a} is a member selected from H, a negative charge and a salt counterion and each of the remaining two R groups (R^{9a} and R^{10a} in If, R^{9a} and R^{11a} in Ig, R^{9a} and R^{12a} in Ih, R^{10a} and R^{11a} in Ii, R^{10a} and R^{12a} in Ij, R^{11a} and R^{12a} in Ik) is a member independently selected from fluoro, chloro, bromo, nitro, cyano, amino, methyl, hydroxylmethyl, trifluoromethyl, methoxy, trifluoromethyoxy, ethyl, diethylcarbamoyl, pyridin-2-yl, pyridin-3-yl, pyridin-4-yl, pyrimidinyl, piperizino, piperizinyl, piperizinocarbonyl, piperizinylcarbonyl, carboxyl, 1-tetrazolyl, 1-ethoxycarbonylmethoxy, carboxymethoxy, thiophenyl, 3-(butylcarbonyl)phenylmethoxy, 1H-tetrazol-5-yl, 1-ethoxycarbonylmethyloxy-, 1-ethoxycarbonylmethyl-, 1-ethoxycarbonyl-, carboxymethoxy-, thiophen-2-yl, thiophen-2-ylthio-, thiophen-3-yl, thiophen-3-ylthio, 4-fluorophenylthio, butylcarbonylphenylmethoxy, butylcarbonylphenylmethyl, butylcarbonylmethyl, 1-(piperidin-1-yl)carbonyl)methyl, 1-(piperidin-1-yl)carbonyl)methoxy, 1-(piperidin-2-yl)carbonyl)methoxy, 1-(piperidin-3-yl)carbonyl)methoxy, 1-(4-(pyrimidin-2-yl)piperazin-1-yl)carbonyl)methoxy, 1-(4-(pyrimidin-2-yl)piperazin-1-yl)carbonyl)methyl, 1-(4-(pyrimidin-2-yl)piperazin-1-yl)carbonyl, 1-4-(pyrimidin-2-yl)piperazin-1-yl, 1-(4-(pyridin-2-yl)piperazin-1-yl)carbonyl), 1-(4-(pyridin-2-yl)piperazin-1-yl)carbonylmethyl, (1-(4-(pyridin-2-yl)piperazin-1-yl)carbonyl)-methoxy), 1-(4-(pyridin-2-yl)piperazin-1-yl, 1H-indol-1-yl, morpholino-, morpholinyl, morpholinocarbonyl, morpholinylcarbonyl, phenylureido, phenylcarbamoyl, acetamido, 3-(phenylthio)-1H-indol-1-yl, 3-(2-cyanoethylthio)-1H-indol-1-yl, benzylamino, 5-methoxy-3-(phenylthio)-1H-indol-1-yl, 5-methoxy-3-(2-cyanoethylthio)-1H-indol-1-yl)), 5-chloro-1H-indol-1-yl, 5-chloro-3-(2-cyanoethylthio)-1H-indol-1-yl)), dibenzylamino, benzylamino, 5-chloro-3-(phenylthio)-1H-indol-1-yl)), 4-(1H-tetrazol-5-yl)phenoxy, 4-(1H-tetrazol-5-yl)phenyl, 4-(1H-tetrazol-5-yl)phenylthio, 2-cyanophenoxy, 3-cyanophenoxy, 4-cyanophenoxy, 2-cyanophenylthio, 3-cyanophenylthio, 4-cyanophenylthio, 2-chlorophenoxy, 3-chlorophenoxy, 4-chlorophenoxy, 2-fluorophenoxy, 3-fluorophenoxy, 4-fluorophenoxy, 2-cyanobenzyloxy, 3-cyanobenzyloxy, 4-cyanobenzyloxy, 2-chlorobenzyloxy, 3-chlorobenzyloxy, 4-chlorobenzyloxy, 2-fluorobenzyloxy, 3-fluorobenzyloxy, and 4-fluorobenzyloxy.

In an exemplary embodiment, the compound has a formula according to Formulae (II)-(Io) wherein R^{1a} is a member selected from H, a negative charge and a salt counterion and each of the remaining three R groups (R^{9a}, R^{10a}, R^{11a} in (Il), R^{9a}, R^{10a}, R^{12a} in (Im), R^{9a}, R^{11a}, R^{12a} in (In), R^{10a}, R^{11a}, R^{12a} in (Io)) is a member independently selected from fluoro, chloro, bromo, nitro, cyano, amino, methyl, hydroxylmethyl, trifluoromethyl, methoxy, trifluoromethyoxy, ethyl, diethylcarbamoyl, pyridin-2-yl, pyridin-3-yl, pyridin-4-yl, pyrimidinyl, piperizino, piperizinyl, piperizinocarbonyl, piperizinylcarbonyl, carboxyl, 1-tetrazolyl, 1-ethoxycarbonylmethoxy, carboxymethoxy, thiophenyl, 3-(butylcarbonyl)phenylmethoxy, 1H-tetrazol-5-yl, 1-ethoxycarbonylmethyloxy-, 1-ethoxycarbonylmethyl-, 1-ethoxycarbonyl-, carboxymethoxy-, thiophen-2-yl, thiophen-2-ylthio-, thiophen-3-yl, thiophen-3-ylthio, 4-fluorophenylthio, butylcarbonylphenylmethoxy, butylcarbonylphenylmethyl, butylcarbonylmethyl, 1-(piperidin-1-yl)carbonyl)methyl, 1-(piperidin-1-yl)carbonyl)methoxy, 1-(piperidin-2-yl)carbonyl)methoxy, 1-(piperidin-3-yl)carbonyl)methoxy, 1-(4-(pyrimidin-2-yl)piperazin-1-yl)carbonyl)methoxy, 1-(4-(pyrimidin-2-yl)piperazin-1-yl)carbonyl)methyl, 1-(4-(pyrimidin-2-yl)piperazin-1-yl)carbonyl, 1-4-(pyrimidin-2-yl)piperazin-1-yl, 1-(4-(pyridin-2-yl)piperazin-1-yl)carbonyl), 1-(4-(pyridin-2-yl)piperazin-1-yl)carbonylmethyl, (1-(4-(pyridin-2-yl)piperazin-1-yl)carbonyl)-methoxy), 1-(4-(pyridin-2-yl)piperazin-1-yl, 1H-indol-1-yl, morpholino-, morpholinyl, morpholinocarbonyl, morpholinylcarbonyl, phenylureido, phenylcarbamoyl, acetamido, 3-(phenylthio)-1H-indol-1-yl, 3-(2-cyanoethylthio)-1H-indol-1-yl, benzylamino, 5-methoxy-3-(phenylthio)-1H-indol-1-yl, 5-methoxy-3-(2-cyanoethylthio)-1H-indol-1-yl)), 5-chloro-1H-indol-1-yl, 5-chloro-3-(2-cyanoethylthio)-1H-indol-1-yl)), dibenzylamino, benzylamino, 5-chloro-3-(phenylthio)-1H-indol-1-yl)), 4-(1H-tetrazol-5-yl)phenoxy, 4-(1H-tetrazol-5-yl)phenyl, 4-(1H-tetrazol-5-yl)phenylthio, 2-cyanophenoxy, 3-cyanophenoxy, 4-cyanophenoxy, 2-cyanophenylthio, 3-cyanophenylthio, 4-cyanophenylthio, 2-chlorophenoxy, 3-chlorophenoxy, 4-chlorophenoxy, 2-fluorophenoxy, 3-fluorophenoxy, 4-fluorophenoxy, 2-cyanobenzyloxy, 3-cyanobenzyloxy, 4-cyanobenzyloxy, 2-chlorobenzyloxy, 3-chlorobenzyloxy, 4-chlorobenzyloxy, 2-fluorobenzyloxy, 3-fluorobenzyloxy, and 4-fluorobenzyloxy.

In another exemplary embodiment, there is a proviso that the compound cannot be a member selected from C1-C40.

In another exemplary embodiment, there is a proviso that the compound cannot have a structure according to Formula (IX): 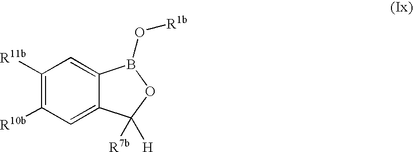
wherein R^{7b} is a member selected from H, methyl, ethyl and phenyl. R^{10b} is a member selected from H, OH, NH₂, SH, halogen, substituted or unsubstituted phenoxy, substituted or unsubstituted phenylalkyloxy, substituted or unsubstituted phenylthio and substituted or unsubstituted phenylalkylthio. R^{11b} is a member selected from H, OH, NH₂, SH, methyl, substituted or unsubstituted phenoxy, substituted or unsubstituted phenylalkyloxy, substituted or unsubstituted phenylthio and substituted or unsubstituted phenylalkylthio. In another exemplary embodiment, there is a proviso that the compound cannot have a structure according to Formula (Ix) wherein R^{1b} is a member selected from a negative charge, H and a salt counterion. In another exemplary embodiment, there is a proviso that the compound cannot have a structure according to Formula (Ix) wherein R^{10b} and R^{11b} are H. In another exemplary embodiment, there is a proviso that the compound cannot have a structure according to Formula (Ix) wherein one member selected from R^{10b} and R^{11b} is H and the other member selected from R^{10b} and R^{11b} is a member selected from halo, methyl, cyano, methoxy, hydroxymethyl and p-cyanophenyloxy. In another exemplary embodiment, there is a proviso that the compound cannot have a structure according to Formula (Ix) wherein R^{10b} and R^{11b} are members independently selected from fluoro, chloro, methyl, cyano, methoxy, hydroxymethyl, and p-cyanophenyl. In another exemplary embodiment, there is a proviso that the compound cannot have a structure according to Formula (Ix) wherein R^{1b} is a member selected from a negative charge, H and a salt counterion; R^{7b} is H; R^{10b} is F and R^{11b} is H. In another exemplary embodiment, there is a proviso that the compound cannot have a structure according to Formula (Ix) wherein R^{11b} and R^{12b}, along with the atoms to which they are attached, are joined to form a phenyl group. In another exemplary embodiment, there is a proviso that the compound cannot have a structure according to Formula (Ix) wherein R^{1b} is a member selected from a negative charge, H and a salt counterion; R^{7b} is H; R^{10b} is 4-cyanophenoxy; and R^{11b} is H.

In another exemplary embodiment, there is a proviso that the compound cannot have a structure according to Formula (Iy) 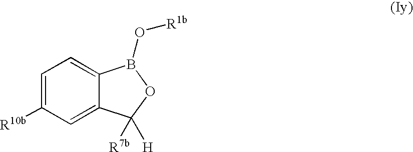
wherein R^{10b} is a member selected from H, halogen, CN and substituted or unsubstituted C₁₋₄ alkyl.

In another exemplary embodiment, there is a proviso that a structure does not have the which is a member selected from Formulae (I) to (Io) at least one member selected from R^{3a}, R^{4a}, R^{5a}, R^{6a}, R^{7a}, R^{8a}, R^{9a}, R^{10a}, R^{11a} and R^{12a} is nitro, cyano or halogen. In another exemplary embodiment, there is a proviso that when M is oxygen, W is a member selected from (CR^{3a}R^{4a})ₙ₁, wherein n1 is 0, J is a member selected from (CR^{6a}R^{7a})ₘₗ, wherein m1 is 1, A is CR^{9a}, D is CR^{10a}, E is CR^{11a}, G is CR^{12a}, the R^{9a} is not halogen, methyl, ethyl, or optionally joined with R^{10a} to form a phenyl ring; R^{10a} is not unsubstituted phenoxy, C(CH₃)₃, halogen, CF₃, methoxy, ethoxy, or optionally joined with R^{9a} to form a phenyl ring; R^{11a} is not halogen or optionally joined with R^{10a} to form a phenyl ring; and R^{12a} is not halogen. In another exemplary embodiment, there is a proviso that when M is oxygen, W is a member selected from (CR^{3a}R^{4a})ₙ₁, wherein n1 is 0, J is a member selected from (CR^{6a}R^{7a})ₘₗ, wherein m1 is 1, A is CR^{9a}, D is CR^{10a}, E is CR^{11a}, G1 is CR^{12a}, then neither R^{6a} nor R^{7a} are halophenyl. In another exemplary embodiment, there is a proviso that when M is oxygen, W is a member selected from (CR^{3a}R^{4a})ₙ₁, wherein n1 is 0, J is a member selected from (CR^{6a}R^{7a})ₘₗ, wherein m1 is 1, A is CR^{9a}, D is CR^{10a}, E is CR^{11a}, G is CR^{12a}, and R^{9a}, R^{10a} and R^{11a} are H, then R^{6a}, R^{7a} and R^{12a} are not H, In another exemplary embodiment, there is a proviso that when M is oxygen wherein n1 is 1, J is a member selected from (CR^{6a}R^{7a})ₘₗ, wherein m1 is 0, A is CR^{9a}, D is CR^{10a}, is CR^{11a}, G is CR^{12a}, R^{9a} is H, R^{10a} is H, R^{11a} is H, R^{6a} is H, R^{7a} is H, R^{12a} is H, then W is not C═O (carbonyl). In another exemplary embodiment, there is a proviso that when M is oxygen, W is CR^{5a}, J is CR^{8a}, A is CR^{9a}, D is CR^{10a}, E is CR^{11a}, G is CR^{12a}, R^{6a}, R^{7a}, R^{9a}, R^{10a}, R^{11a} and R^{12a} are H, then R^{5a} and R^{8a}, together with the atoms to which they are attached, do not form a phenyl ring.

In an exemplary embodiment, the compound of the invention has a structure which is a member selected from: 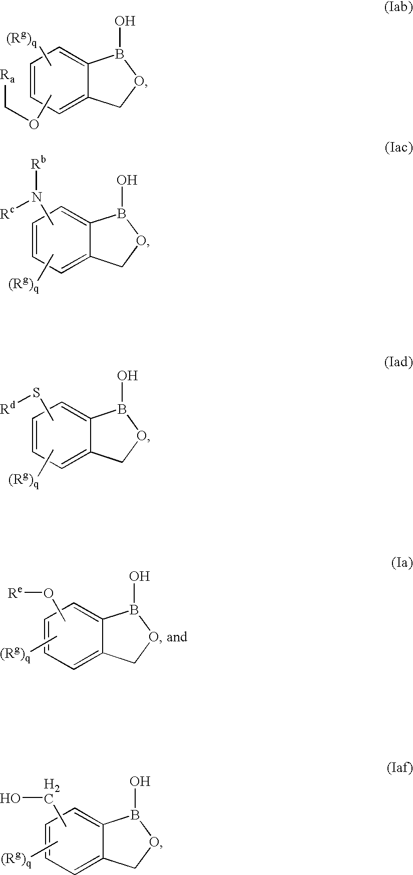
in which q is a number between 0 and 1. R^{g} is halogen. R^{a}, R^{b}, R^{c}, R^{d} and R^{e} are members independently selected from a member selected from H, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. In an exemplary embodiment, there is a proviso that the compound is not a member selected from

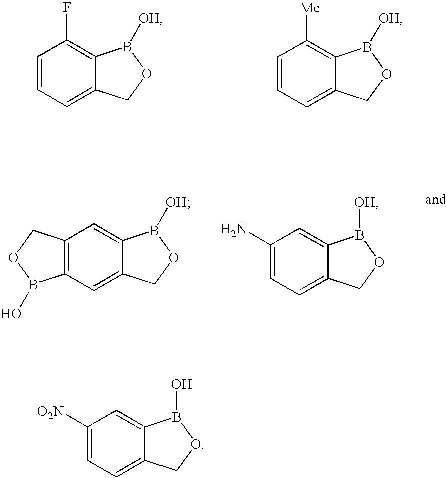

In an exemplary embodiment, the compound has a structure is a member selected from:

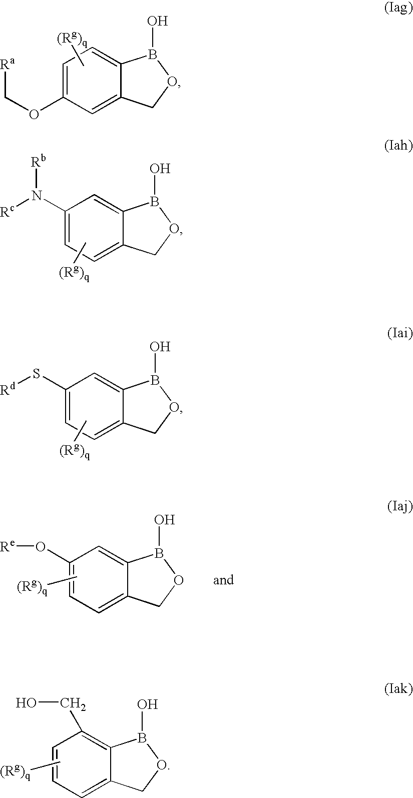

In an exemplary embodiment, R^{a}, R^{d} and R^{e} are each members independently selected from:

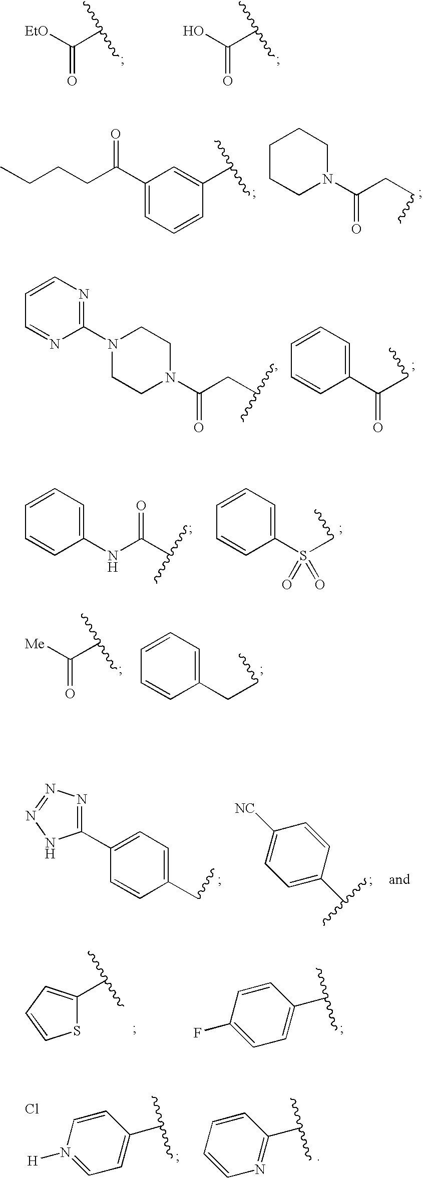

In an exemplary embodiment, R^{b} and R^{c} are members independently selected from H, methyl,

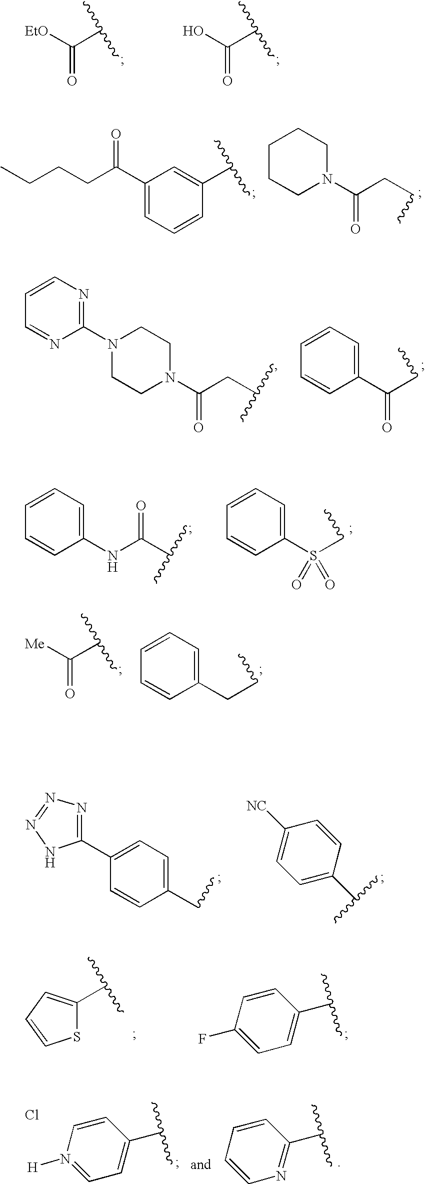

In another exemplary embodiment, R^{b} is H and R^{c} is a member selected from H, methyl, 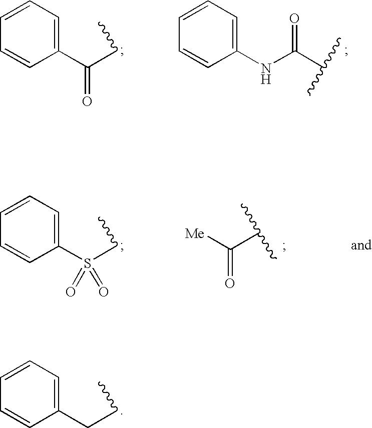
In another exemplary embodiment, R^{b} and R^{c} are, together with the nitrogen to which they are attached, optionally joined to form a member selected from

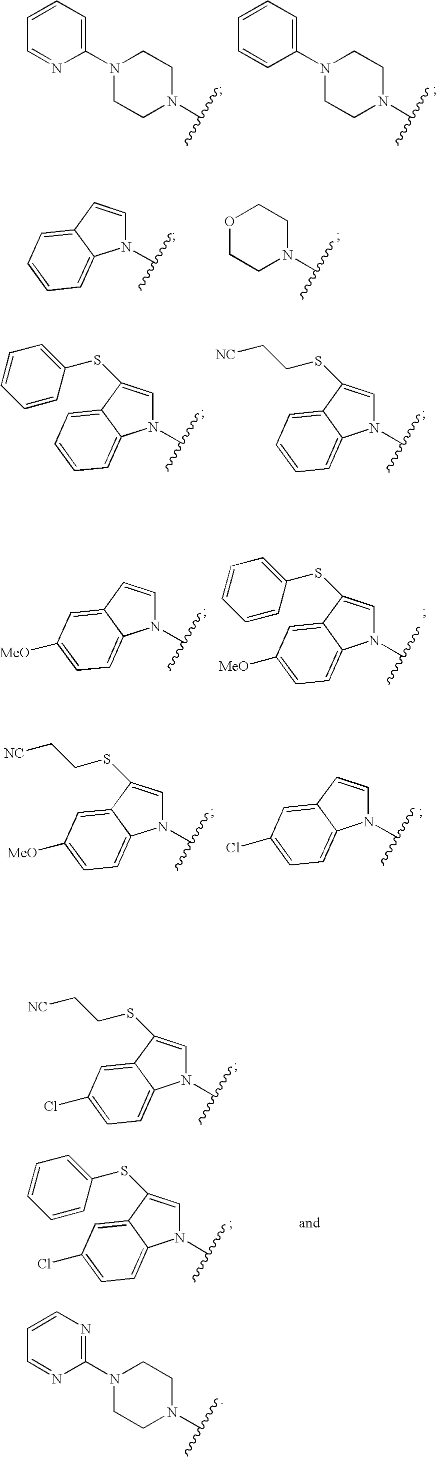

In an exemplary embodiment, R^{a} is a member selected from

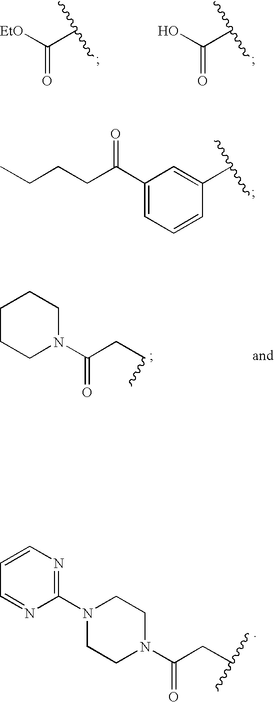

In an exemplary embodiment, R^{d} is a member selected from

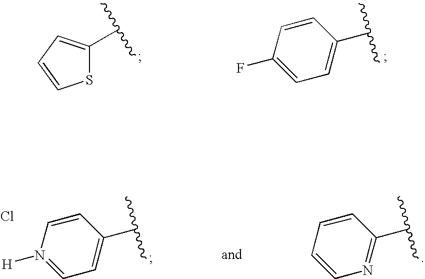

In an exemplary embodiment, R^{e} is a member selected from

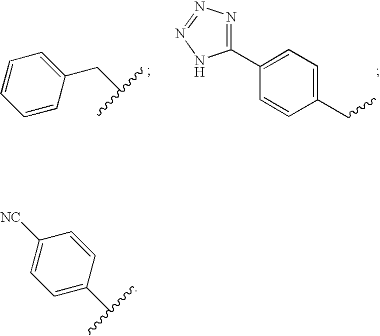

In an exemplary embodiment, the compound is a member selected from

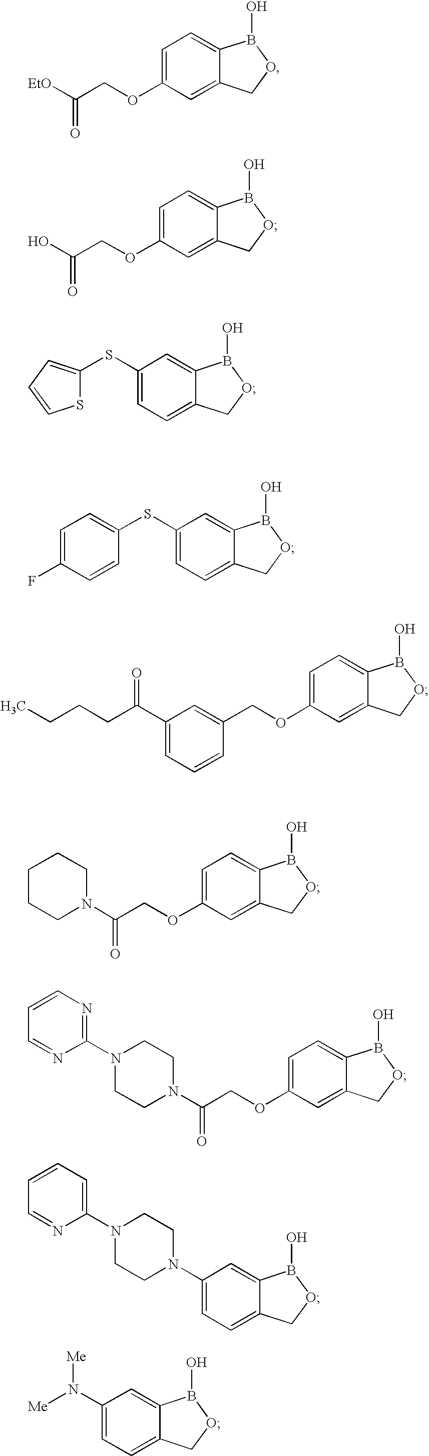

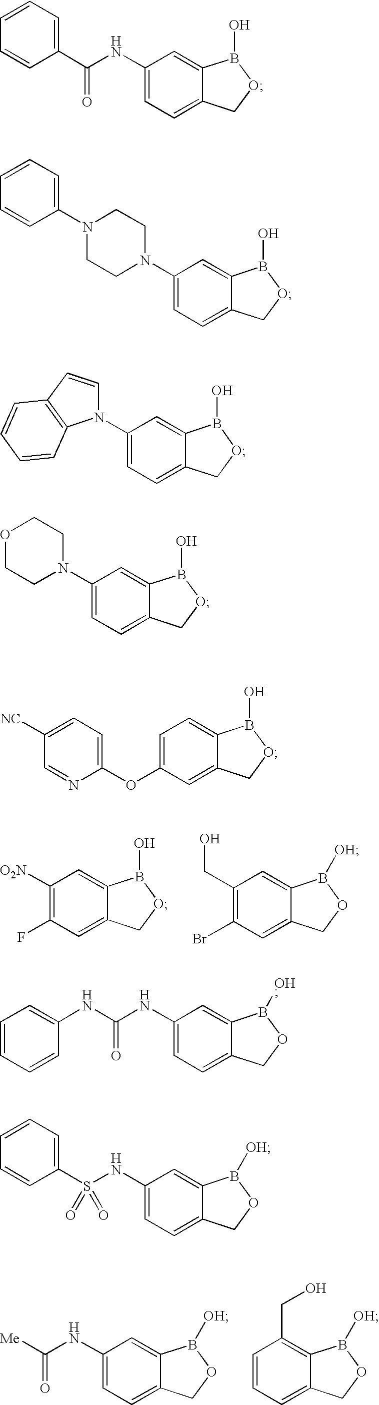

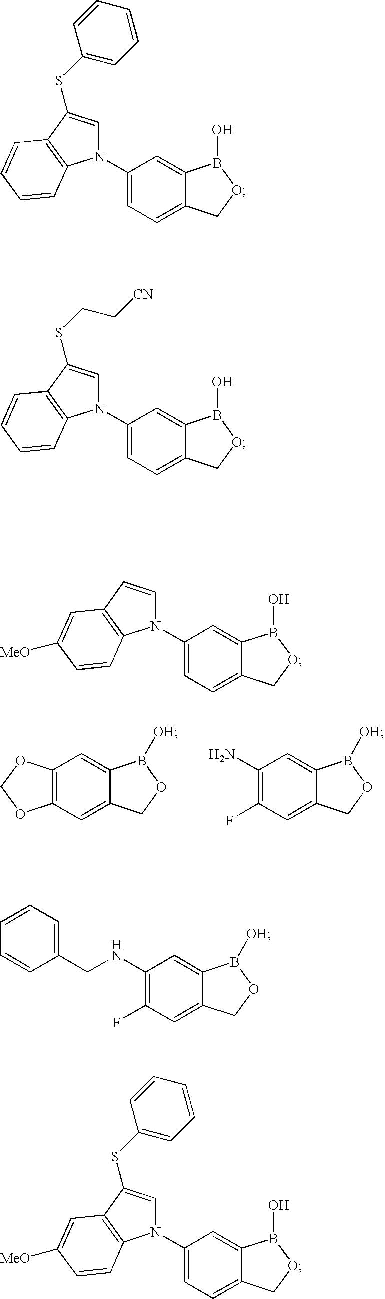

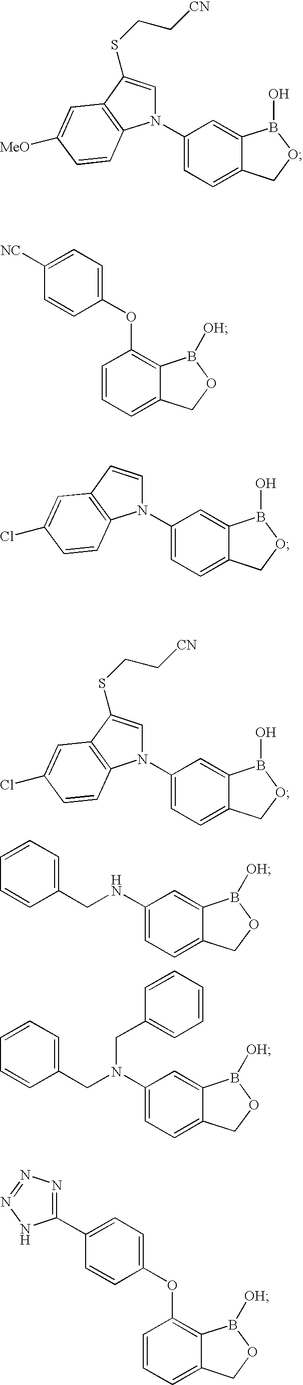

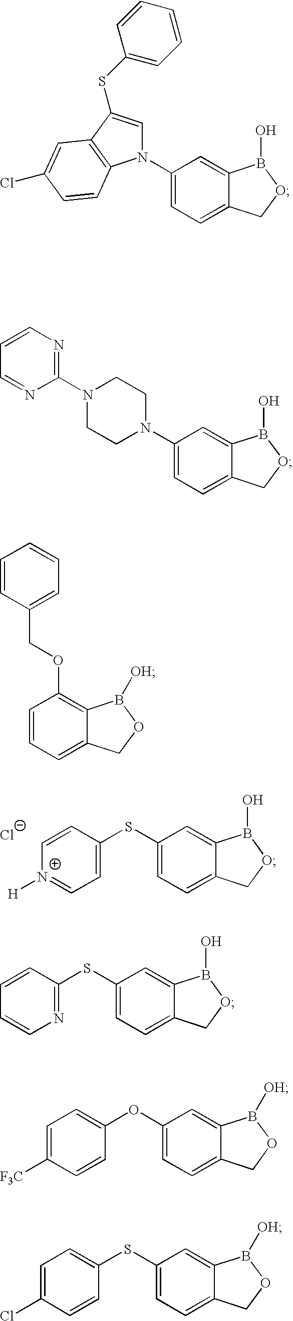

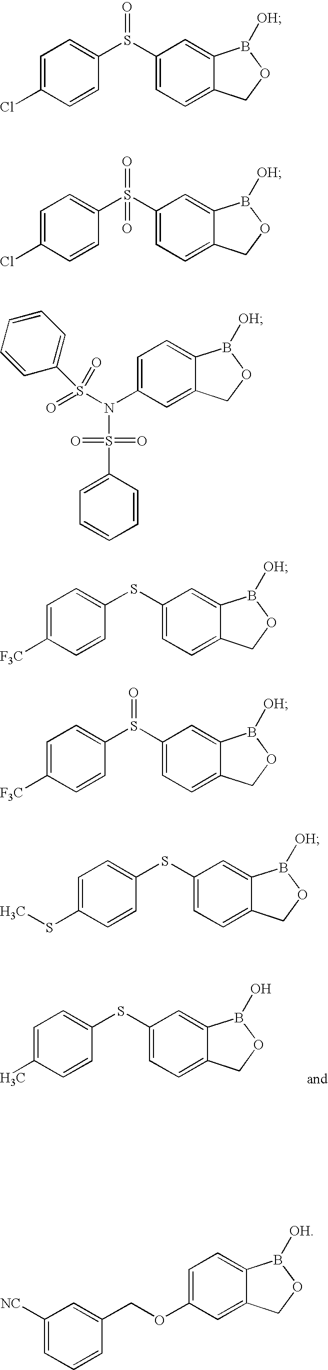

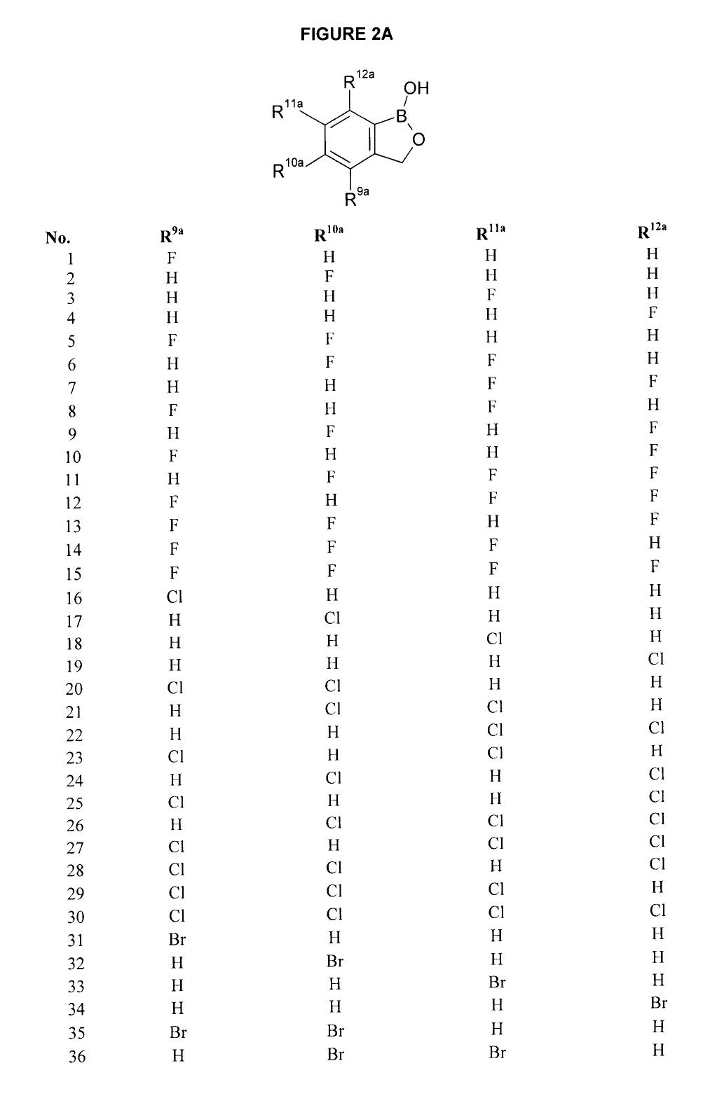

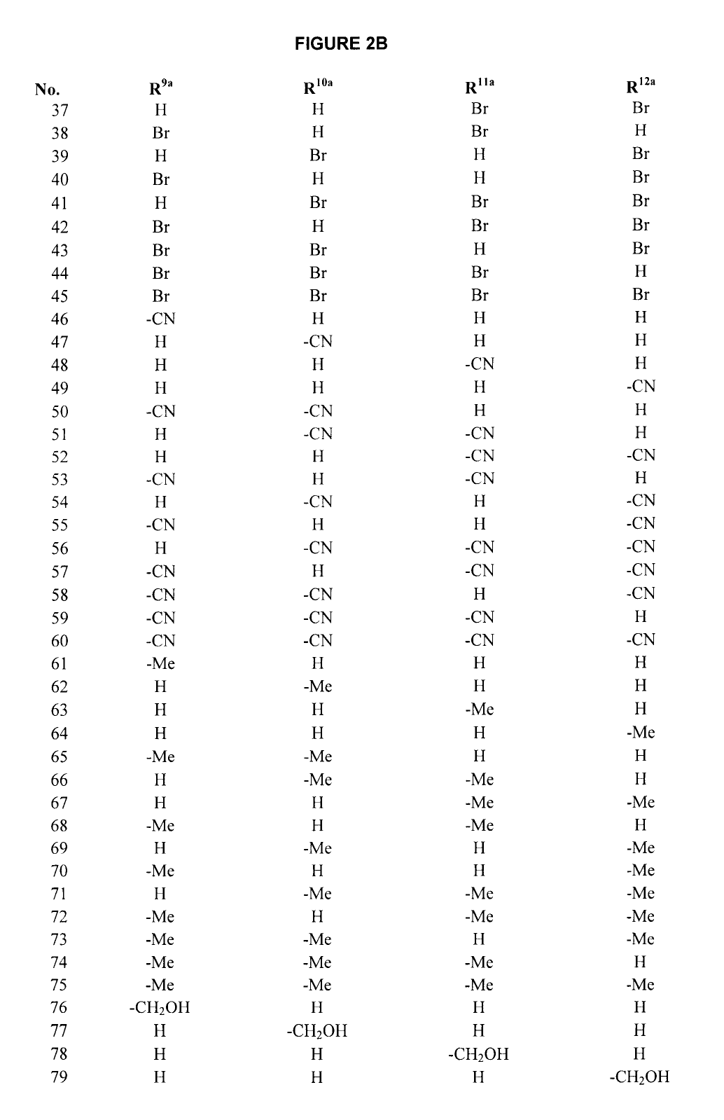

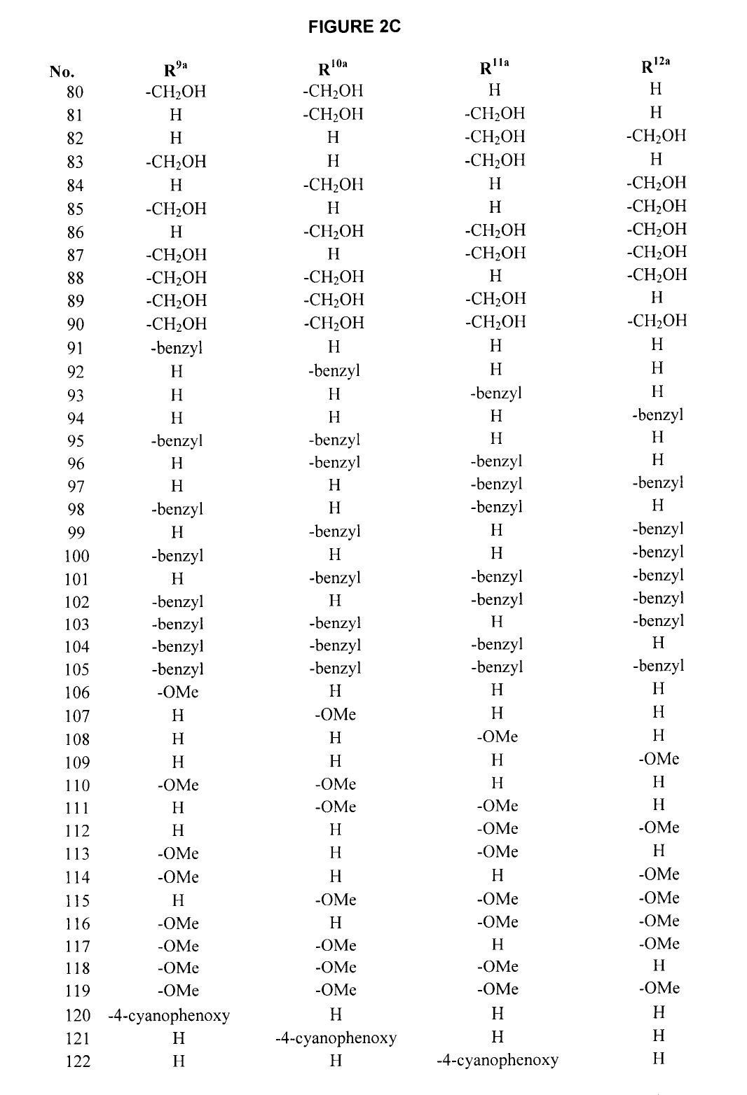

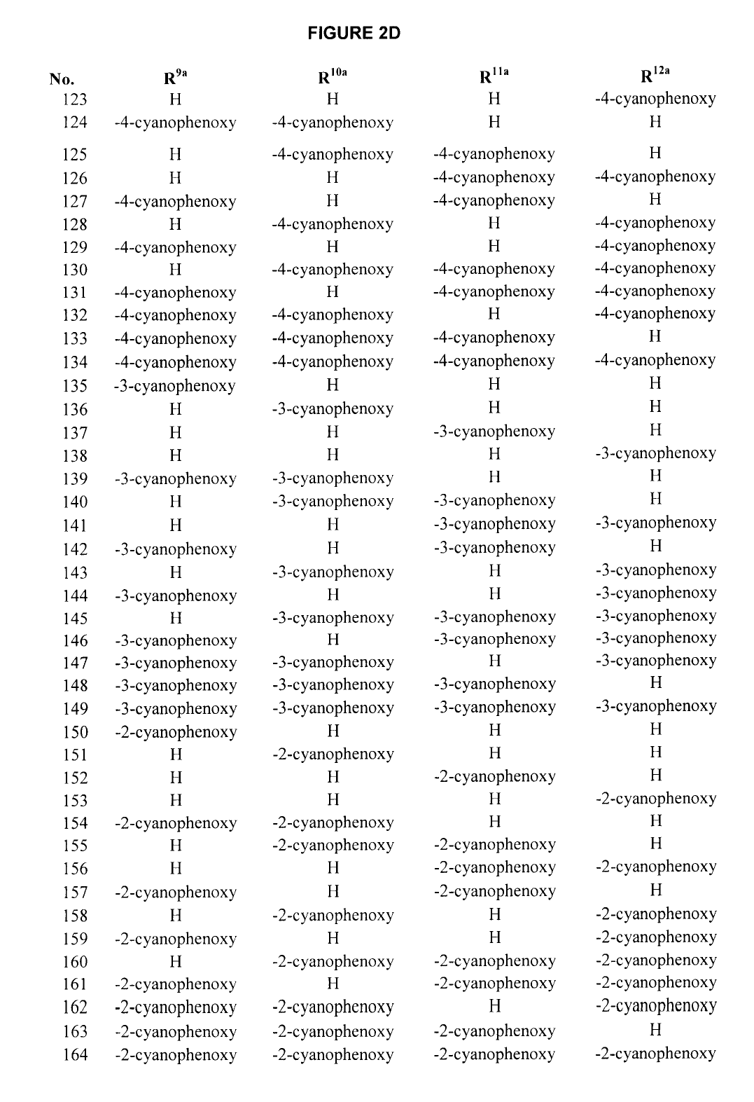

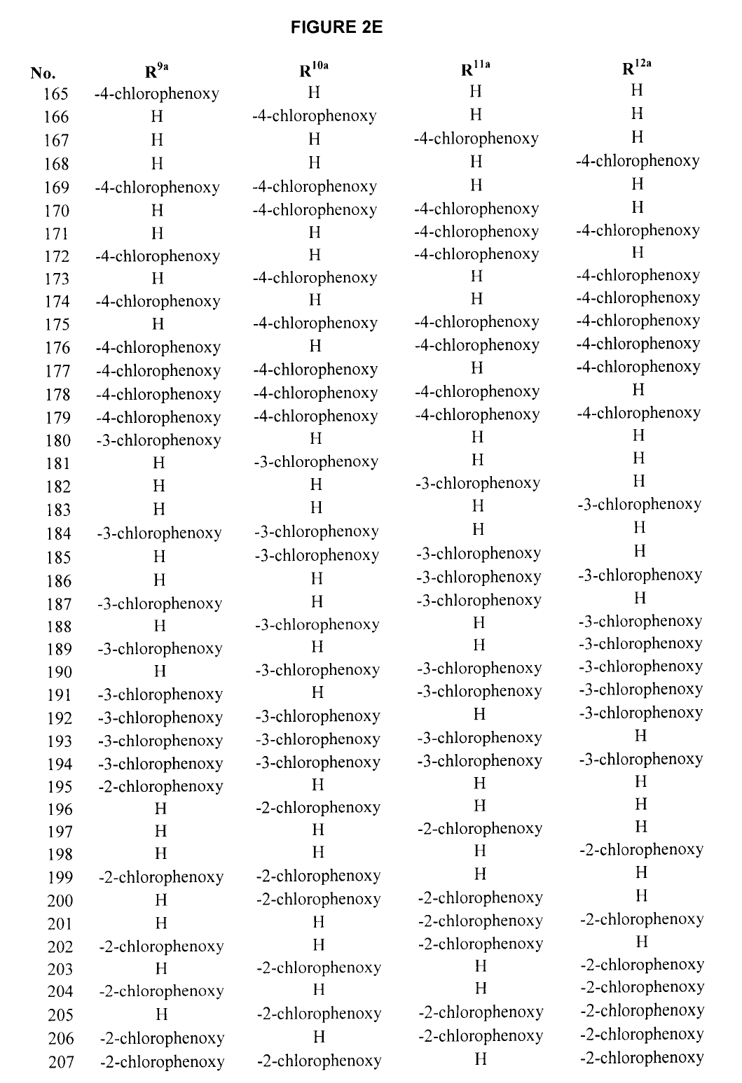

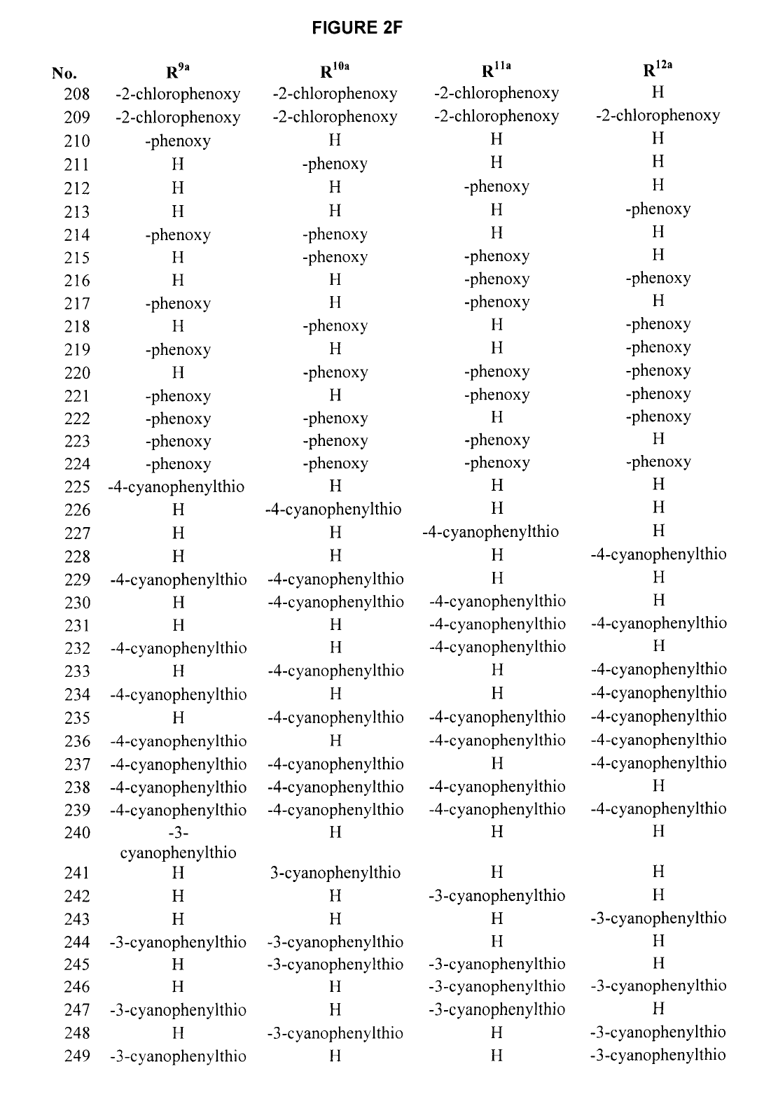

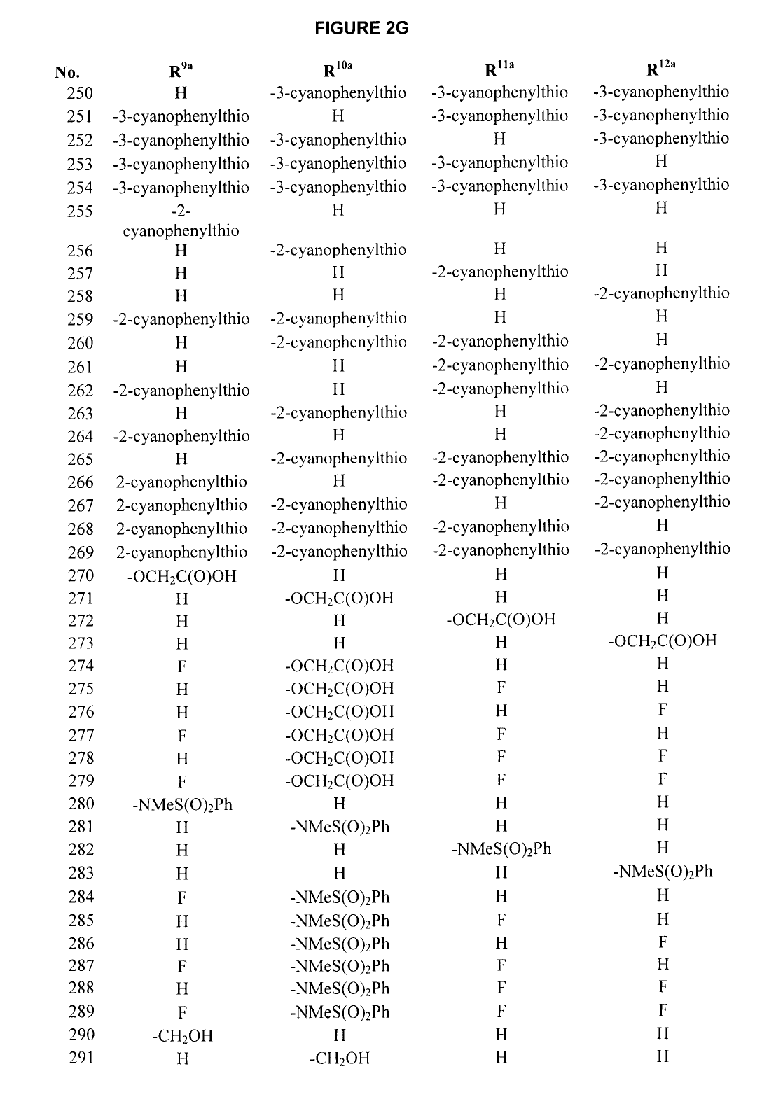

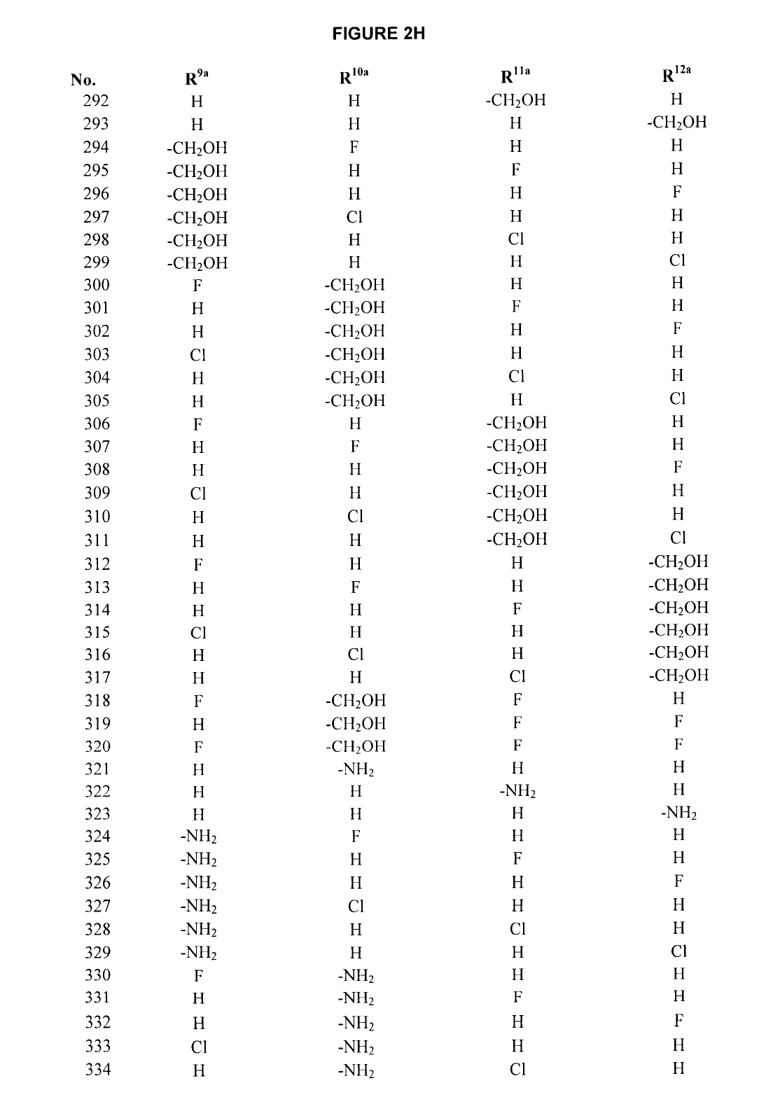

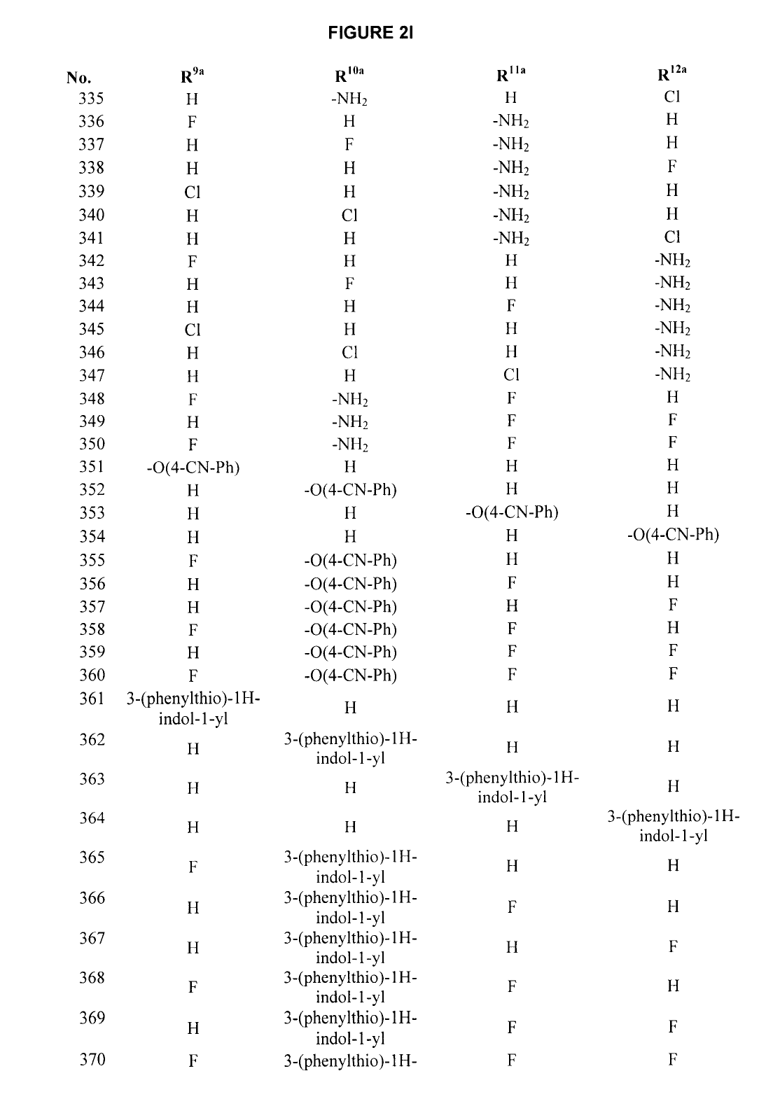

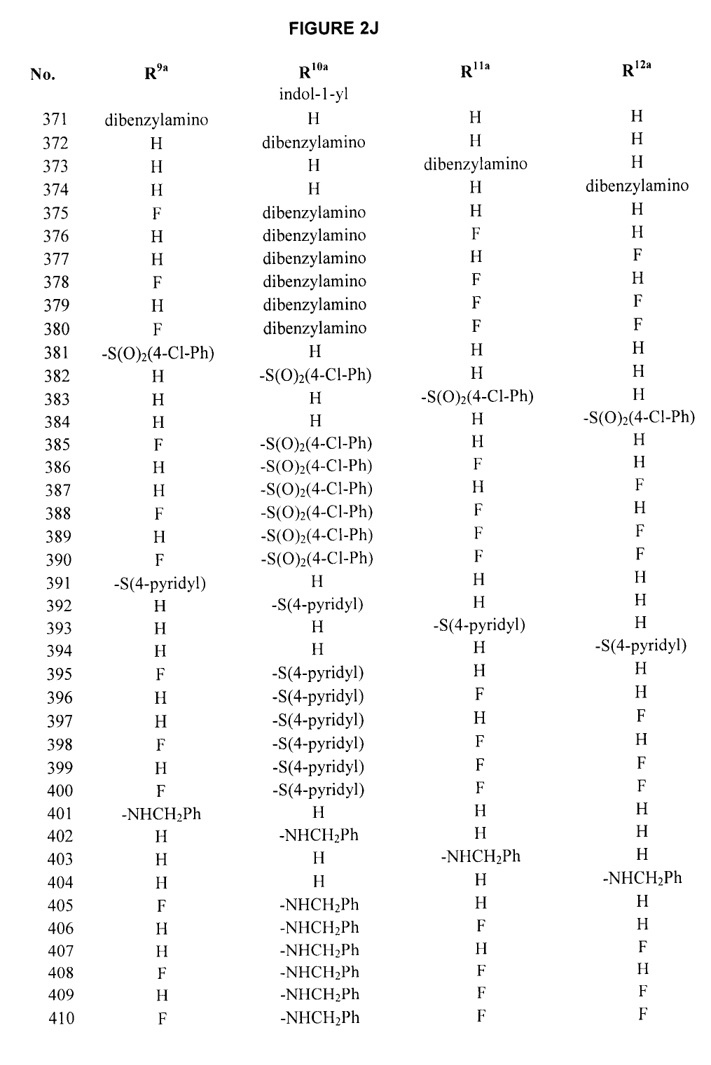

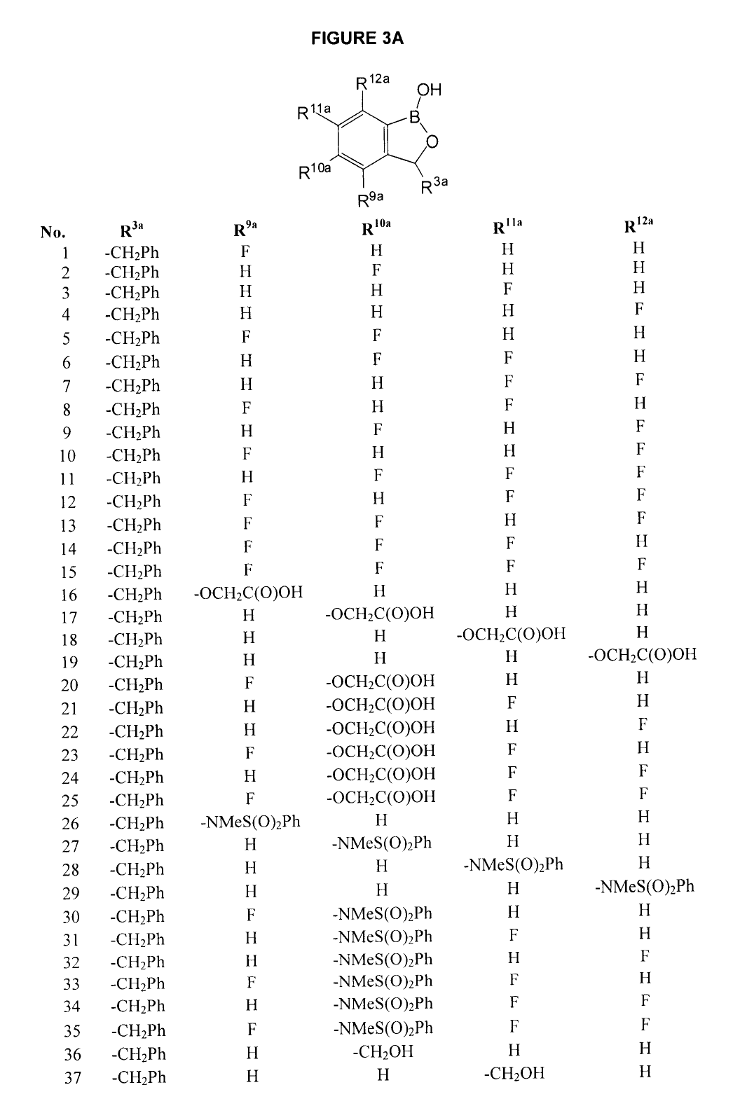

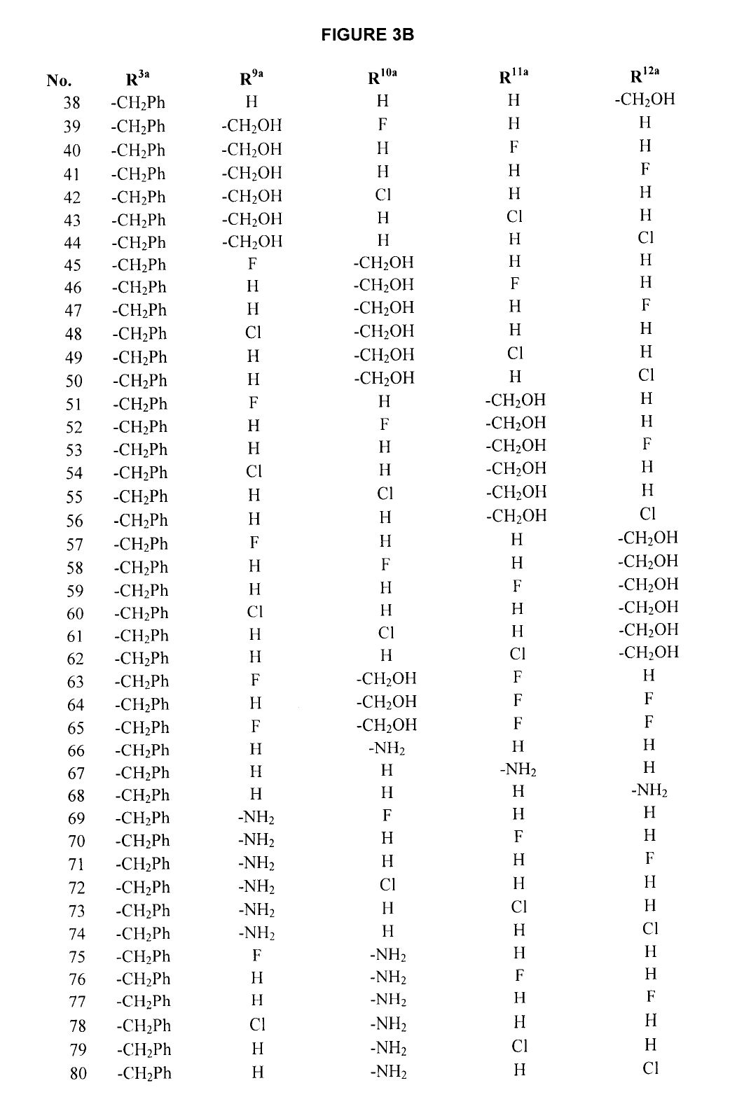

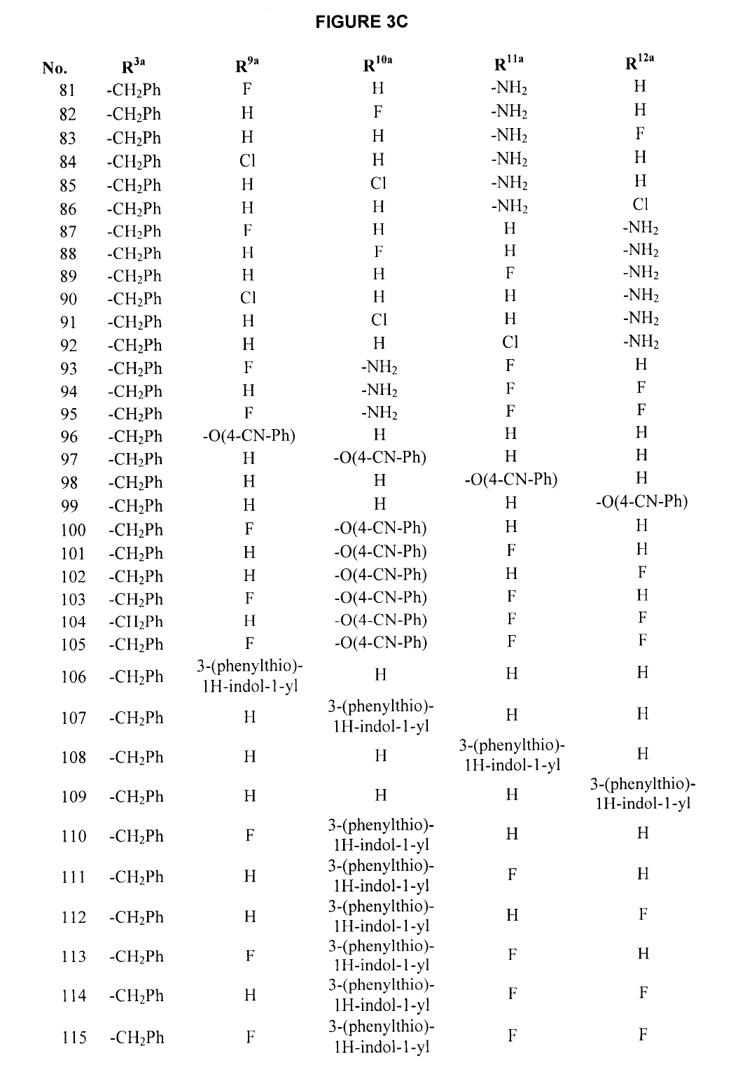

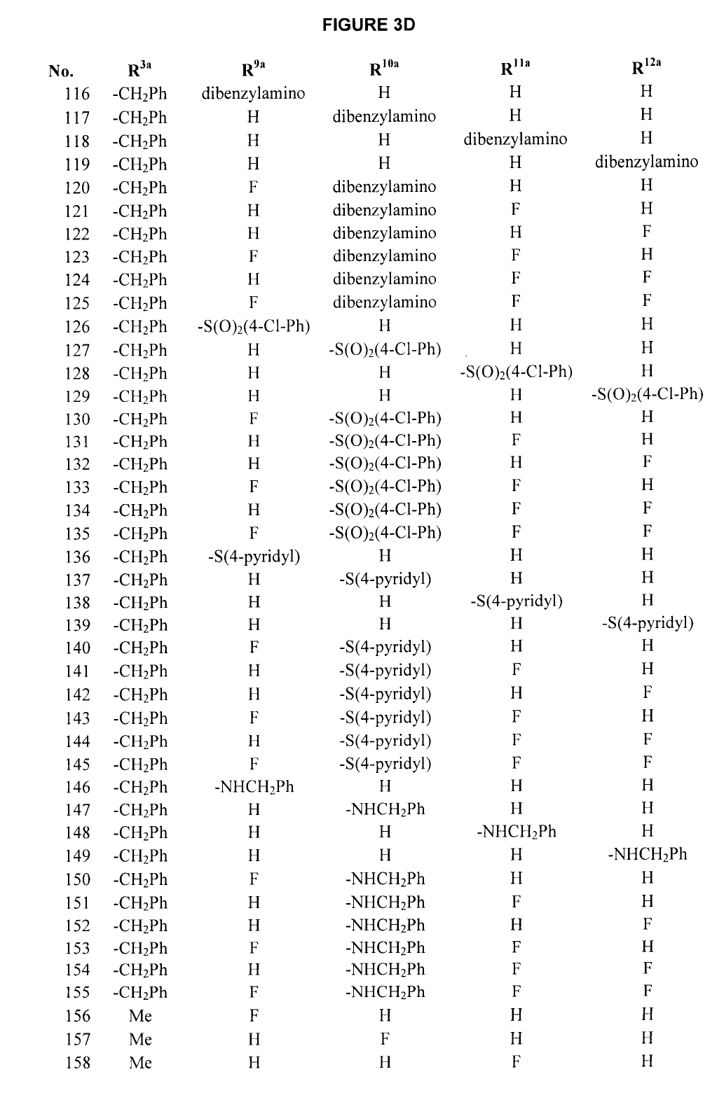

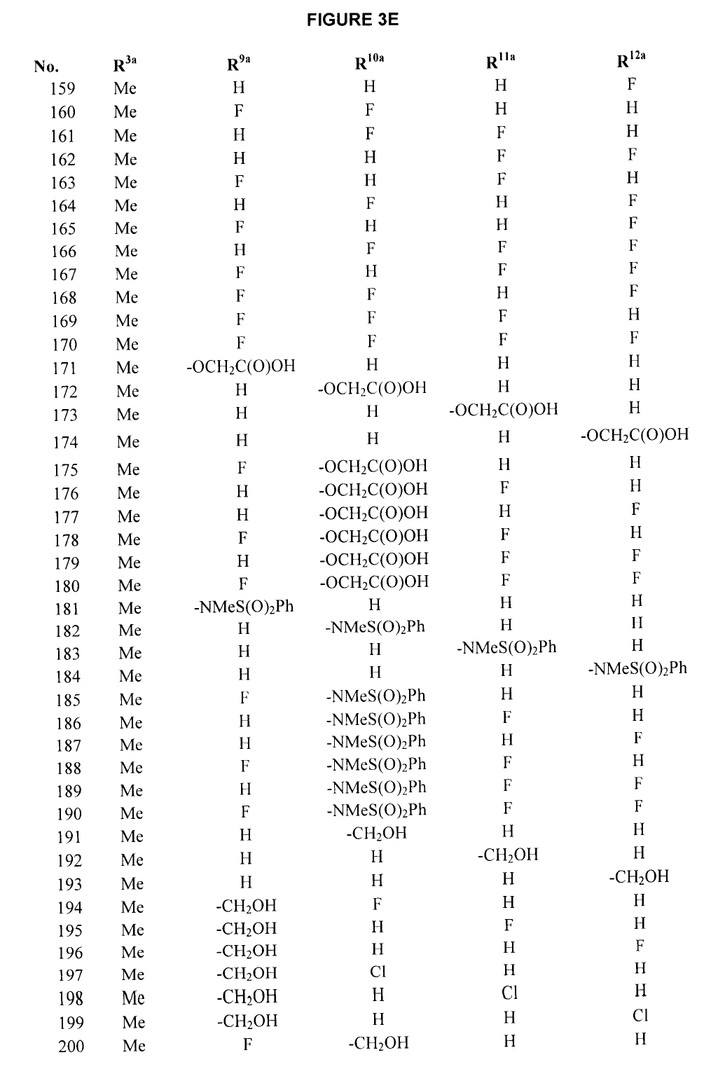

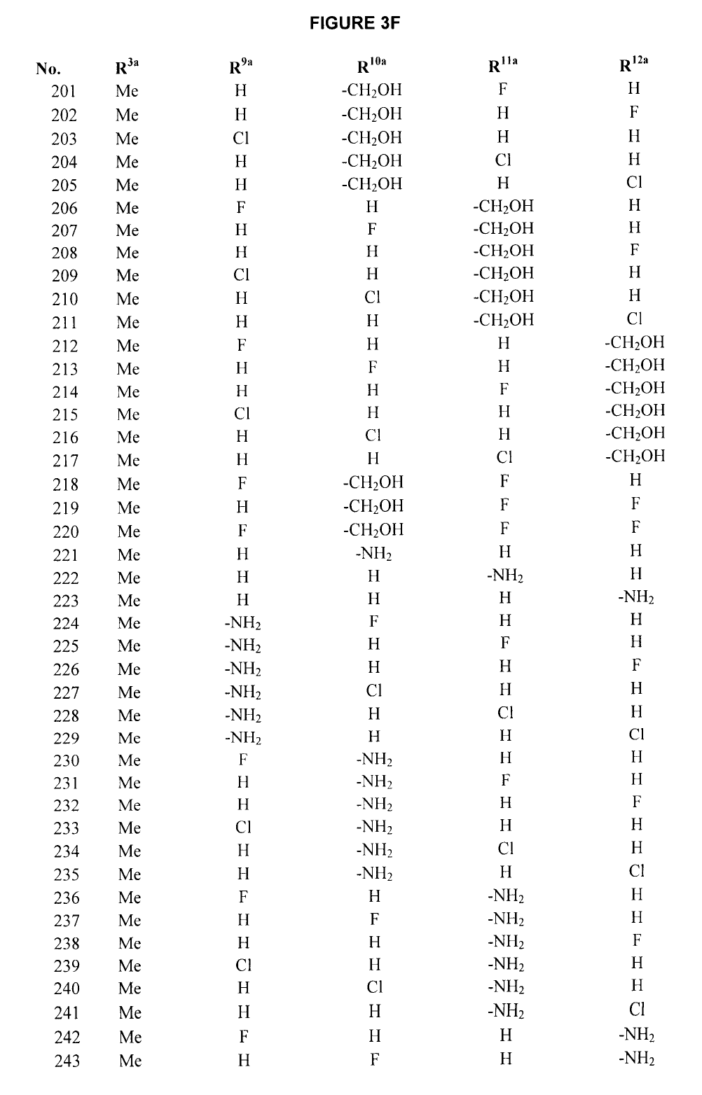

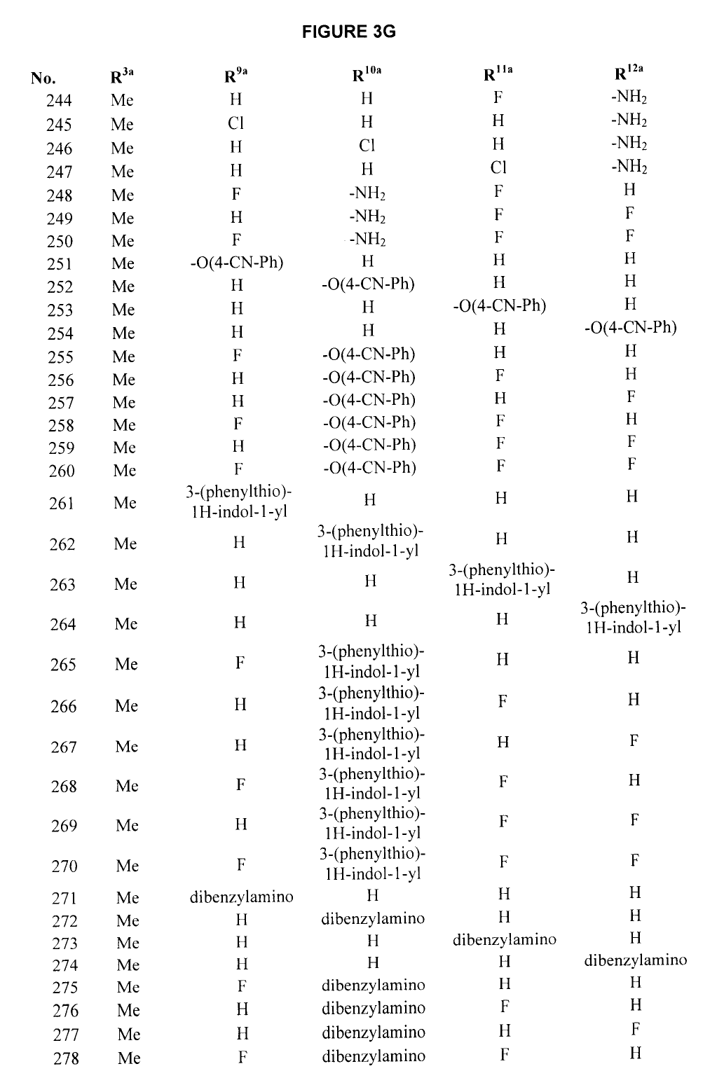

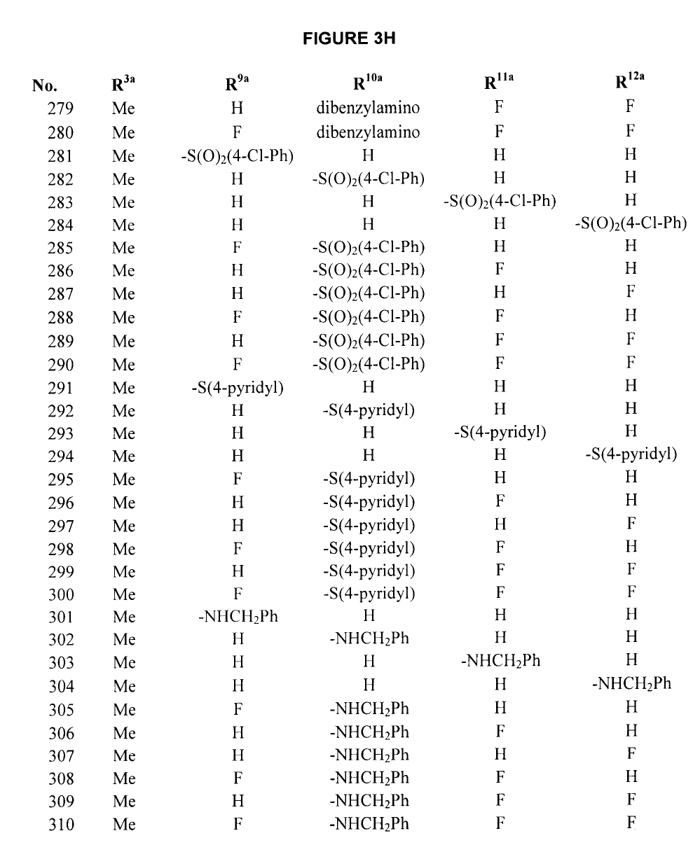

In an exemplary embodiment, the compound has a structure which is described in FIG. 2. In an exemplary embodiment, the compound has a structure which is described in FIG. 3.

In an exemplary embodiment, the compound has a structure according to a member selected from Formulae I(b), I(c), I(d), and I(e) wherein said remaining R group (R^{9a} for I(b), R^{10a} for I(c), R^{11a} for I(d) and R^{12a} for I(e)) is carboxymethoxy.

In an exemplary embodiment, the compound has a structure which is a member selected from Formulae (If)-(Ik), wherein either R^{9a} or R^{10a} for Formula (If), either R^{9a} or R^{11a} for Formula (Ig), either R^{9a} or R^{12a} for Formula (Ih), either R^{10a} or R^{11a} for Formula (Ii), either R^{10a} or R^{12a} for Formula (Ij), either R^{11a} or R^{12a} for Formula (Ik) is halogen, and the other substituent in the pairing (ex. if R^{9a} is F in Formula (If), then R^{10a} is selected from the following substituent listing), is a member selected from NH₂, N(CH₃)H, and N(CH₃)₂.

In another exemplary embodiment, the compound has a structure which is a member selected from: 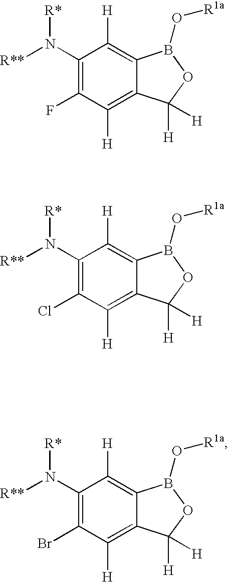
in which R* and R** are members selected from: H, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. In an exemplary embodiment, the compound is a member selected from 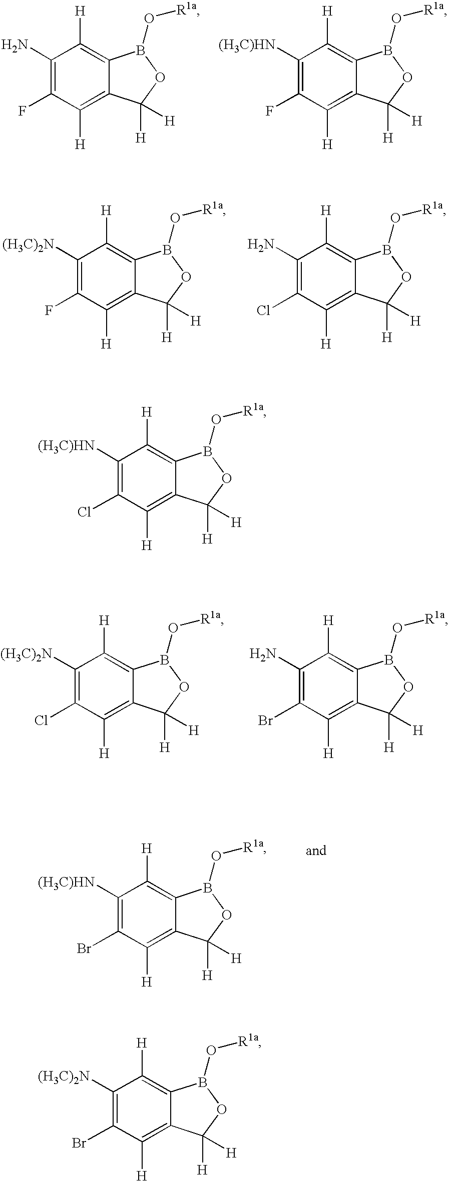
wherein R^{1a} is a member selected from a negative charge, H and a salt counterion.

In another exemplary embodiment, the compound has a structure which is a member selected from: 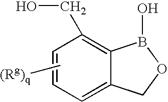
(Iak), wherein q is 1 and R^{g} is a member selected from fluoro, chloro and bromo.

In another exemplary embodiment, the compounds and embodiments described above in Formulae (I)-(Io) can form a hydrate with water, a solvate with an alcohol (e.g. methanol, ethanol, propanol); an adduct with an amino compound (e.g. ammonia, methylamine, ethylamine); an adduct with an acid (e.g. formic acid, acetic acid); complexes with ethanolamine, quinoline, amino acids, and the like.

In another exemplary embodiment, the compound has a structure according to Formula (Ip): 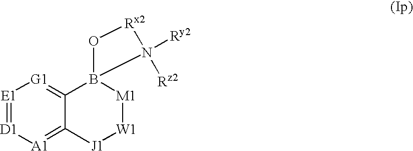
in which R^{x2} is a member selected from substituted or unsubstituted C₁-C₅ alkyl and substituted or unsubstituted C₁-C₅ heteroalkyl. R^{y2} and R^{z2} are members independently selected from H, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. **A1-M1 are undefined?

In another exemplary embodiment, the compound has a structure according to Formula (Iq): 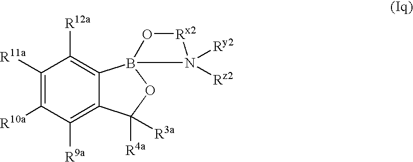
wherein B is boron. R^{x2} is a member selected from substituted or unsubstituted C₁-C₅ alkyl and substituted or unsubstituted C₁-C₅ heteroalkyl. R^{y2} and R^{z2} are members independently selected from H, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. In another exemplary embodiment, at least one member selected from R^{3a}, R^{4a}, R^{5a}, R^{6a}, R^{7a}, R^{8a}, R^{9a}, R^{10a}, R^{11a} la and R^{12a} is a member selected from nitro, cyano and halogen.

In another exemplary embodiment, the compound has a structure which is a member selected from the following Formulae: 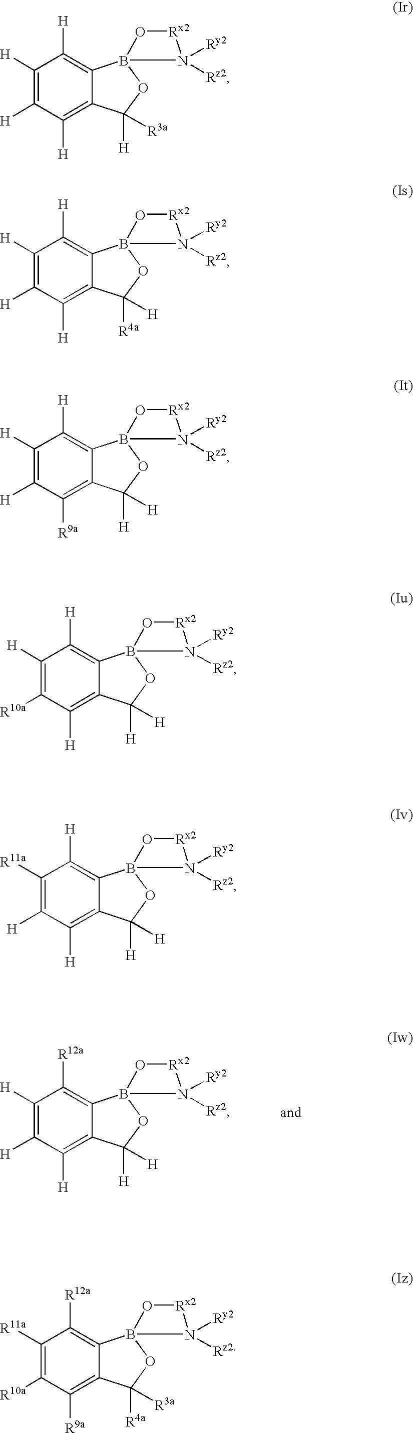
In another exemplary embodiment, the compound has a formula according to Formulae (Ib)-(Ie) wherein at least one member selected from R^{3a}, R^{4a}, R^{5a}, R^{6a}, R^{7a}, R^{8a}, R^{9a}, R^{10a}, R^{11a} and R^{12a} is a member selected from nitro, cyano, fluoro, chloro, bromo and cyanophenoxy. In another exemplary embodiment, the compound is a member selected from

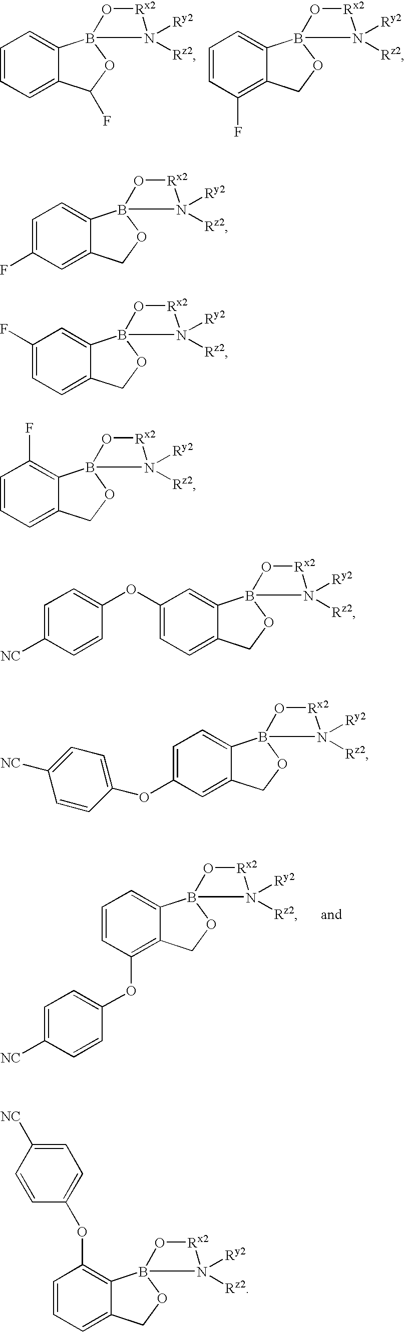

In another exemplary embodiment, the compound is a member selected from

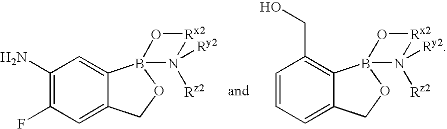

In another exemplary embodiment, there is a proviso that the compound cannot have a structure according to Formula (Iaa): 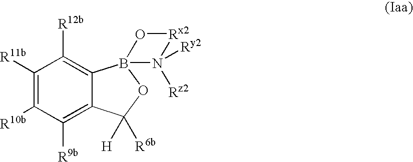
wherein R^{6b}, R^{9b}, R^{10b}, R^{11b} and R^{12b} have the same substituent listings as described for Formulae (Ix) and (Iy) above.

In another exemplary embodiment, the invention provides poly- or multi-valent species of the compounds of the invention. In an exemplary embodiment, the invention provides a dimer of the compounds described herein. In an exemplary embodiment, the invention provides a dimer of the compounds described herein. In an exemplary embodiment, the invention provides a dimer of a compound which is a member selected from C1-C96. In an exemplary embodiment the dimer is a member selected from

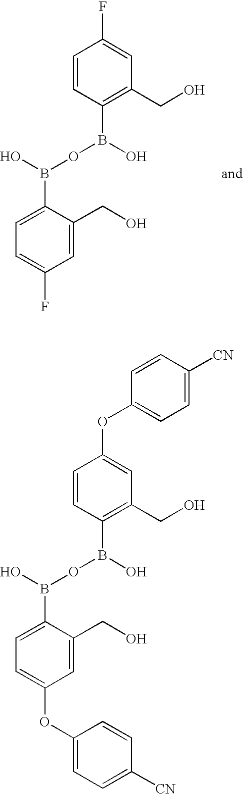

In an exemplary embodiment, the invention provides an anhydride of the compounds described herein. In an exemplary embodiment, the invention provides an anhydride of the compounds described herein. In an exemplary embodiment, the invention provides an anhydride of a compound which is a member selected from C1-C96. In an exemplary embodiment the anhydride is a member selected from

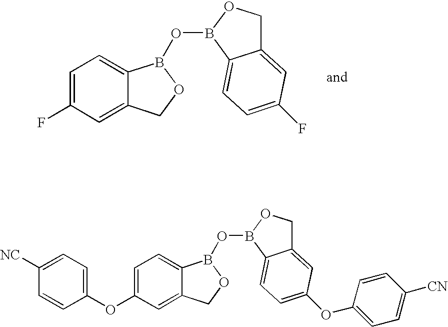

In an exemplary embodiment, the invention provides a trimer of the compounds described herein. In an exemplary embodiment, the invention provides a trimer of the compounds described herein. In an exemplary embodiment, the invention provides a trimer of a compound which is a member selected from C1-C96. In an exemplary embodiment the trimer is a member selected from

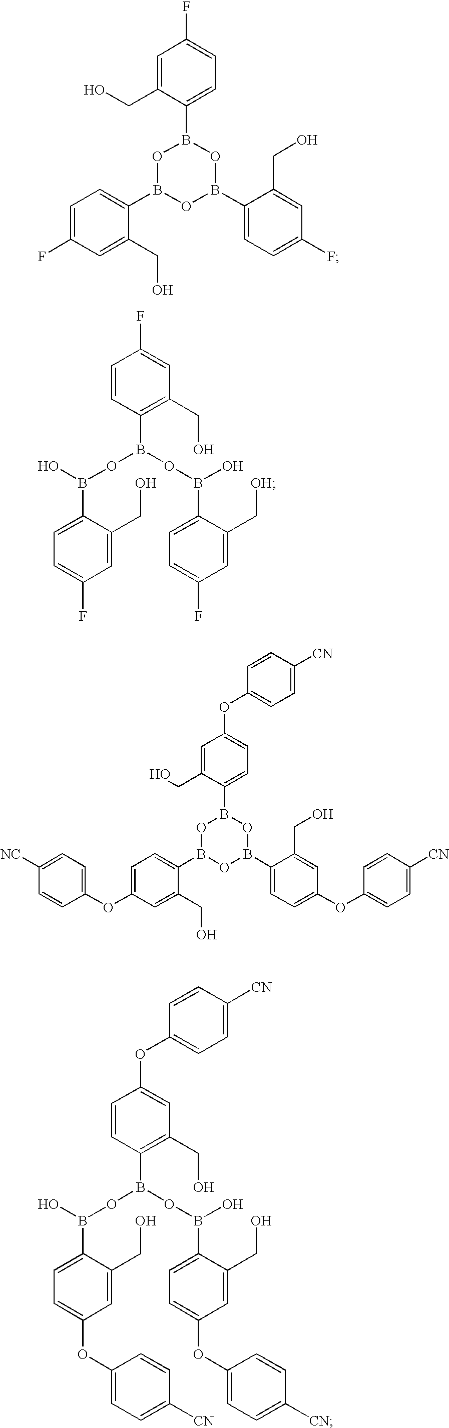

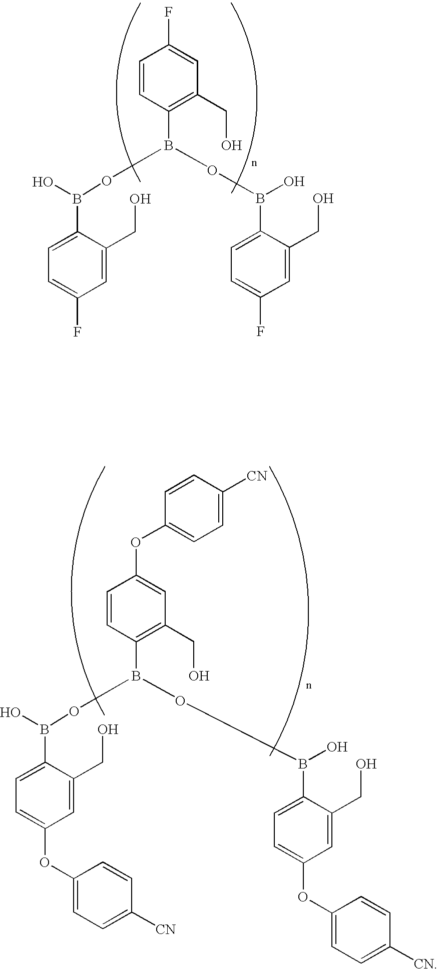

In another exemplary embodiment, R^{10a} is a member selected from 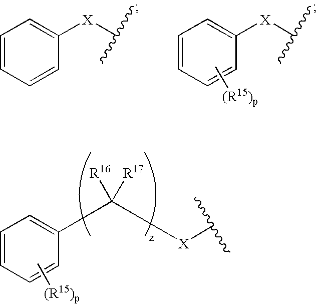
wherein R¹⁵ is a member selected from CN, COOH and 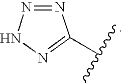
R¹⁶ and R¹⁷ are members independently selected from H, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. The index p is an integer selected from 1 to 5. The index z is an integer selected from 1 to 8. X is a member selected from S and O.

In another exemplary embodiment, the compound has a structure according to Formula (VIII): 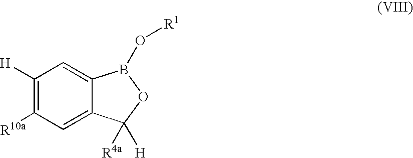
wherein R^{4a} is a member selected from substituted or unsubstituted aryl and substituted or unsubstituted arylalkyl. R^{10a} is a member selected from H, halogen, substituted or unsubstituted aryl, substituted or unsubstituted aryloxy, substituted or unsubstituted arylalkoxy, substituted or unsubstituted arylthio and substituted or unsubstituted arylalkylthio.

In another exemplary embodiment, the compound has a structure compound is a member selected from:

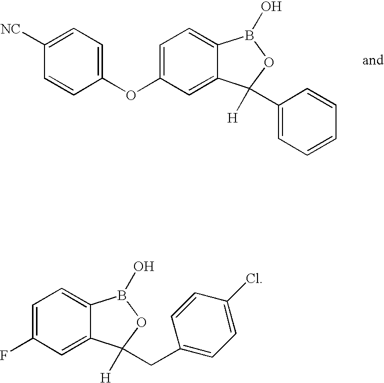

In another exemplary embodiment, the compound is

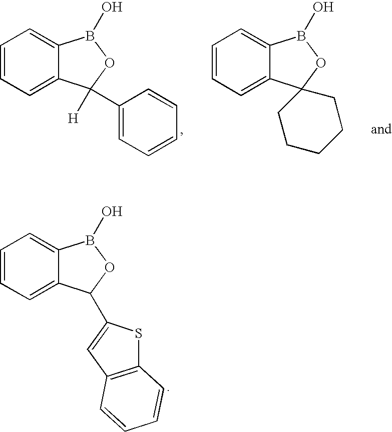

In another aspect, the invention provides compounds useful in the methods which have a structure according to Formula IX: 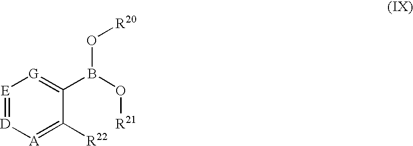
wherein the variables A, D, E and G are described elsewhere herein. R²⁰, R²¹ and R²² are members independently selected from a negative charge, a salt counterion, H, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

In another exemplary embodiment, the compound has a structure according to Formula (X):

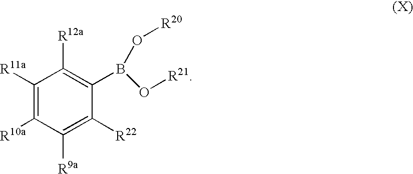

In another exemplary embodiment, is a member selected from:

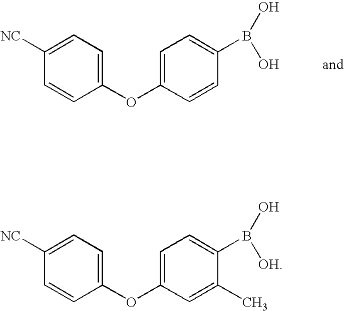

The compounds of the invention can form a hydrate with water, solvates with alcohols such as methanol, ethanol, propanol, and the like; adducts with amino compounds, such as ammonia, methylamine, ethylamine, and the like; adducts with acids, such as formic acid, acetic acid and the like; complexes with ethanolamine, quinoline, amino acids, and the like.

In an exemplary embodiment, the compound has a structure which is a member selected from 5-Chloro-1,3-dihydro-1-hydroxy-2,1-benzoxaborole (C1), 1,3-Dihydro-1-hydroxy-2,1-benzoxaborole (C2), 5-Fluoro-1,3-dihydro-1-hydroxy-3-methyl-2,1-benzoxaborole (C3), 6-Fluoro-1-hydroxy-1,2,3,4-tetrahydro-2,1-benzoxaborine (C4), 5,6-Difluoro-1,3-dihydro-1-hydroxy-2,1-benzoxaborole (C5), 5-Cyano-1,3-dihydro-1-hydroxy-2,1-benzoxaborole (C6), 1,3-Dihydro-1-hydroxy-5-methoxy-2,1-benzoxaborole (C7), 1,3-Dihydro-1-hydroxy-5-methyl-2,1-benzoxaborole (C8), 1,3-Dihydro-1-hydroxy-5-hydroxymethyl-2,1-benzoxaborole (C9), 1,3-Dihydro-5-fluoro-1-hydroxy-2,1-benzoxaborole (C10), 1,3-Dihydro-2-oxa-1-cyclopenta[\acute{α}]naphthalene (C11), 7-Hydroxy-2,1-oxaborolano[5,4-c]pyridine (C12), 1,3-Dihydro-6-fluoro-1-hydroxy-2,1-benzoxaborole (C13), 3-Benzyl-1,3-dihydro-1-hydroxy-3-methyl-2,1-benzoxaborole (C14), 3-Benzyl-1,3-dihydro-1-hydroxy-2,1-benzoxaborole (C15), 1,3-Dihydro-4-fluoro-1-hydroxy-2,1-benzoxaborole (C16), 5-(4-Cyanophenoxy)-1,3-dihydro-1-hydroxy-2,1-benzoxaborole (C17), 6-(4-Cyanophenoxy)-1,3-dihydro-1-hydroxy-2,1-benzoxaborole (C18), 6-(3-Cyanophenoxy)-1,3-dihydro-1-hydroxy-2,1-benzoxaborole (C19), 6-(4-Chlorophenoxy)-1,3-dihydro-1-hydroxy-2,1-benzoxaborole (C20), 6-Phenoxy-1,3-dihydro-1-hydroxy-2,1-benzoxaborole (C21), 5-(4-Cyanobenzyloxy)-1,3-dihydro-1-hydroxy-2,1-benzoxaborole (C22), 5-(2-Cyanophenoxy)-1,3-dihydro-1-hydroxy-2,1-benzoxaborole (C23), 5-Phenoxy-1,3-dihydro-1-hydroxy-2,1-benzoxaborole (C24), 5-[4-(N,N-Diethylcarbamoyl)phenoxy]-1,3-dihydro-1-hydroxy-2,1-benzoxaborole (C25), 1,3-Dihydro-1-hydroxy-5-[4-(morpholinocarbonyl)phenoxy]-2,1-benzoxaborole (C26), 5-(3,4-Dicyanophenoxy)-1,3-dihydro-1-hydroxy-2,1-benzoxaborole (C27), 6-Phenylthio-1,3-dihydro-1-hydroxy-2,1-benzoxaborole (C28), 6-(4-trifluoromethoxyphenoxy)-1,3-dihydro-1-hydroxy-2,1-benzoxaborole (C29), 5-(N-Methyl-N-phenylsulfonylamino)-1,3-dihydro-1-hydroxy-2,1-benzoxaborole (C30), 6-(4-Methoxyphenoxy)-1,3-dihydro-1-hydroxy-2,1-benzoxaborole (C31), 6-(4-Methoxyphenylthio)-1,3-dihydro-1-hydroxy-2,1-benzoxaborole (C32), 6-(4-Methoxyphenylsulfonyl)-1,3-dihydro-1-hydroxy-2,1-benzoxaborole (C33), 6-(4-Methoxyphenylsulfinyl)-1,3-dihydro-1-hydroxy-2,1-benzoxaborole (C34), 5-Trifluoromethyl-1,3-dihydro-1-hydroxy-2,1-benzoxaborole (C35), 4-(4-Cyanophenoxy)-1,3-dihydro-1-hydroxy-2,1-benzoxaborole (C36), 5-(3-Cyanophenoxy)-1,3-dihydro-1-hydroxy-2,1-benzoxaborole (C37), 5-(4-Carboxyphenoxy)-1-hydroxy-2,1-benzoxaborole (C38), 1-Hydroxy-5-[4-(tetrazole-1-yl)phenoxy]-2,1-benzoxaborole (C39), 5-Chloro-1,3-dihydro-1-hydroxy-2,1-benzoxaborole, 1,3-Dihydro-1-hydroxy-2,1-benzoxaborole, 5-Fluoro-1,3-dihydro-1-hydroxy-3-methyl-2,1-benzoxaborole, 6-Fluoro-1-hydroxy-1,2,3,4-tetrahydro-2,1-benzoxaborine, 5,6-Difluoro-1,3-dihydro-1-hydroxy-2,1-benzoxaborole, 5-Cyano-1,3-dihydro-1-hydroxy-2,1-benzoxaborole, 1,3-Dihydro-1-hydroxy-5-methoxy-2,1-benzoxaborole, 1,3-Dihydro-1-hydroxy-5-methyl-2,1-benzoxaborole, 1,3-Dihydro-1-hydroxy-5-hydroxymethyl-2,1-benzoxaborole, 1,3-Dihydro-5-fluoro-1-hydroxy-2,1-benzoxaborole, 1,3-Dihydro-2-oxa-1-cyclopenta[\acute{α}]naphthalene, 7-Hydroxy-2,1-oxaborolano[5,4-c]pyridine, 1,3-Dihydro-6-fluoro-1-hydroxy-2,1-benzoxaborole, 3-Benzyl-1,3-dihydro-1-hydroxy-3-methyl-2,1-benzoxaborole, 3-Benzyl-1,3-dihydro-1-hydroxy-2,1-benzoxaborole, 1,3-Dihydro-4-fluoro-1-hydroxy-2,1-benzoxaborole, 5-(4-Cyanophenoxy)-1,3-dihydro-1-hydroxy-2,1-benzoxaborole, 6-(4-Cyanophenoxy)-1,3-dihydro-1-hydroxy-2,1-benzoxaborole, 6-(3-Cyanophenoxy)-1,3-dihydro-1-hydroxy-2,1-benzoxaborole, 6-(4-Chlorophenoxy)-1,3-dihydro-1-hydroxy-2,1-benzoxaborole, 6-Phenoxy-1,3-dihydro-1-hydroxy-2,1-benzoxaborole, 5-(4-Cyanobenzyloxy)-1,3-dihydro-1-hydroxy-2,1-benzoxaborole, 5-(2-Cyanophenoxy)-1,3-dihydro-1-hydroxy-2,1-benzoxaborole, 5-Phenoxy-1,3-dihydro-1-hydroxy-2,1-benzoxaborole, 5-[4-(N,N-Diethylcarbamoyl)phenoxy]-1,3-dihydro-1-hydroxy-2,1-benzoxaborole, 1,3-Dihydro-1-hydroxy-5-[4-(morpholinocarbonyl)phenoxy]-2,1-benzoxaborole, 5-(3,4-Dicyanophenoxy)-1,3-dihydro-1-hydroxy-2,1-benzoxaborole, 6-Phenylthio-1,3-dihydro-1-hydroxy-2,1-benzoxaborole, 6-(4-trifluoromethoxyphenoxy)-1,3-dihydro-1-hydroxy-2,1-benzoxaborole, 5-(N-Methyl-N-phenylsulfonylamino)-1,3-dihydro-1-hydroxy-2,1-benzoxaborole, 6-(4-Methoxyphenoxy)-1,3-dihydro-1-hydroxy-2,1-benzoxaborole, 6-(4-Methoxyphenylthio)-1,3-dihydro-1-hydroxy-2,1-benzoxaborole, 6-(4-Methoxyphenylsulfonyl)-1,3-dihydro-1-hydroxy-2,1-benzoxaborole, 6-(4-Methoxyphenylsulfinyl)-1,3-dihydro-1-hydroxy-2,1-benzoxaborole, 5-Trifluoromethyl-1,3-dihydro-1-hydroxy-2,1-benzoxaborole, 4-(4-Cyanophenoxy)-1,3-dihydro-1-hydroxy-2,1-benzoxaborole, 5-(3-Cyanophenoxy)-1,3-dihydro-1-hydroxy-2,1-benzoxaborole, 5-(4-Carboxyphenoxy)-1-hydroxy-2,1-benzoxaborole, 1-Hydroxy-5-[4-(tetrazole-1-yl)phenoxy]-2,1-benzoxaborole, 4-(4-Cyanophenoxy)phenylboronic acid, 3-(4-Cyanophenoxy)phenylboronic acid, and 4-(4-Cyanophenoxy)-2-Methylphenylboronic acid.

In an exemplary embodiment, the compound has a structure which is a member selected from the following compounds: 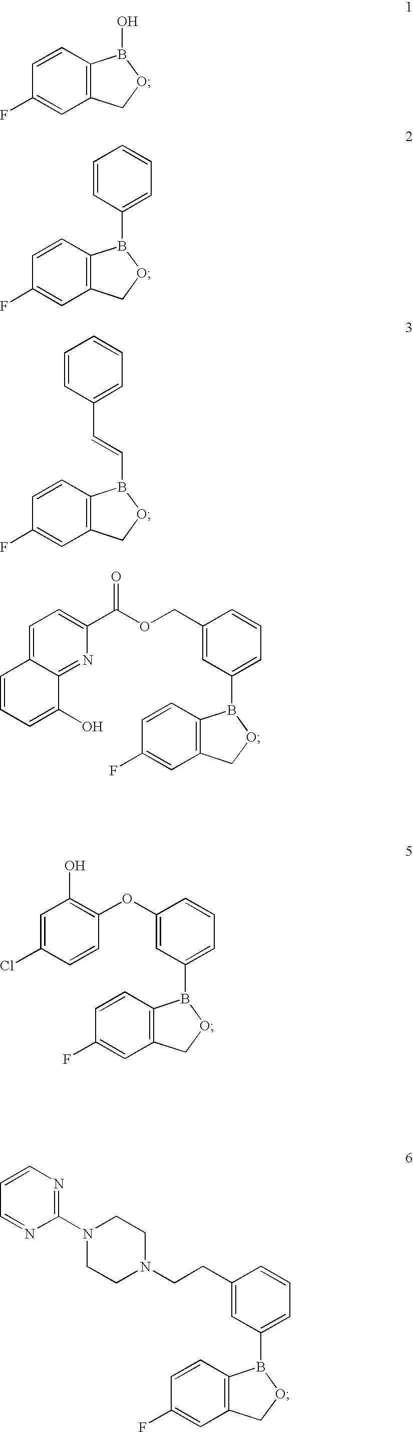 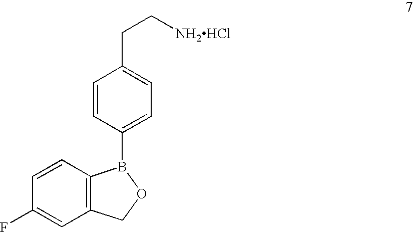
Methods of Making the Compounds

In one aspect, the invention provides compounds useful in the methods

Preparation of Boron-Contain in a Editing Domain Inhibitors

Compounds of use in the present invention can be prepared using commercially available starting materials, known intermediates, or by using the synthetic methods published in references described and incorporated by reference herein.

Boronic Esters

The following exemplary schemes illustrate methods of preparing boron-containing molecules of the present invention. These methods are not limited to producing the compounds shown, but can be used to prepare a variety of molecules such as the compounds and complexes described herein. The compounds of the present invention can also be synthesized by methods not explicitly illustrated in the schemes but are well within the skill of one in the art. The compounds can be prepared using readily available materials of known intermediates.

In the following schemes, the symbol X represents bromo or iodo. The symbol Y is selected from H, lower alkyl, and arylalkyl. The symbol Z is selected from H, alkyl, and aryl. The symbol PG represents protecting group. The symbols A, D, E, G, R^{x}, R^{y}, R^{z}, R^{1a}, R^{2a}, R^{3a}, R^{4a}, R^{5a}, R^{6a}, R^{7a}, R^{8a}, R^{9a}, R^{10a}, R^{11a}, and R^{12a} can be used to refer to the corresponding symbols in the compounds described herein.

Boronic Acid Preparation Strategy #1

In Scheme 1, Step 1 and 2, compounds 1 or 2 are converted into alcohol 3. In step 1, compound 1 is treated with a reducing agent in an appropriate solvent. Suitable reducing agents include borane complexes, such as borane-tetrahydrofuran, borane-dimethylsulfide, combinations thereof and the like. Lithium aluminum hydride, or sodium borohydride can also be used as reducing agents. The reducing agents can be used in quantities ranging from 0.5 to 5 equivalents, relative to compound 1 or 2. Suitable solvents include diethyl ether, tetrahydrofuran, 1,4-dioxane, 1,2-dimethoxyethane, combinations thereof and the like. Reaction temperatures range from 0° C. to the boiling point of the solvent used; reaction completion times range from 1 to 24 h.

In Step 2, the carbonyl group of compound 2 is treated with a reducing agent in an appropriate solvent. Suitable reducing agents include borane complexes, such as borane-tetrahydrofuran, borane-dimethylsulfide, combinations thereof and the like. Lithium aluminum hydride, or sodium borohydride can also be used as reducing agents. The reducing agents can be used in quantities ranging from 0.5 to 5 equivalents, relative to compound 2. Suitable solvents include lower alcohol, such as methanol, ethanol, and propanol, diethyl ether, tetrahydrofuran, 1,4-dioxane and 1,2-dimethoxyethane, combinations thereof and the like. Reaction temperatures range from 0° C. to the boiling point of the solvent used; reaction completion times range from 1 to 24 h.

In Step 3, the hydroxyl group of compound 3 is protected with a protecting group which is stable under neutral or basic conditions. The protecting group is typically selected from methoxymethyl, ethoxyethyl, tetrahydropyran-2-yl, trimethylsilyl, tert-butyldimethylsilyl, tributylsilyl, combinations thereof and the like. In the case of methoxymethyl, compound 3 is treated with 1 to 3 equivalents of chloromethyl methyl ether in the presence of a base. Suitable bases include sodium hydride, potassium tert-butoxide, tertiary amines, such as diisopropylethylamine, triethylamine, 1,8-diazabicyclo[5,4,0]undec-7-ene, and inorganic bases, such as sodium hydroxide, sodium carbonate, potassium hydroxide, potassium carbonate, combinations thereof and the like. The bases can be used in quantities ranging from 1 to 3 equivalents, relative to compound 3. Reaction temperatures range from 0° C. to the boiling point of the solvent used; preferably between 0 and 40° C.; reaction completion times range from 1 h to 5 days.

In the case of tetrahydropyran-2-yl, compound 3 is treated with 1 to 3 equivalents of 3,4-dihydro-2H-pyran in the presence of 1 to 10 mol % of acid catalyst. Suitable acid catalysts include pyridinium p-toluenesulfonic acid, p-toluenesulfonic acid, camphorsulfonic acid, methanesulfonic acid, hydrogen chloride, sulfuric acid, combinations thereof and the like. Suitable solvents include dichloromethane, chloroform, tetrahydrofuran, 1,4-dioxane, 1,2-dimethoxyethane, toluene, benzene, and acetonitrile combinations thereof and the like. Reaction temperatures range from 0° C. to the boiling point of the solvent used; preferably between 0 and 60° C., and is complete in 1 h to 5 days.

In the case of trialkylsilyl, compound 3 is treated with 1 to 3 equivalents of chlorotrialkylsilyane in the presence of 1 to 3 equivalents of base. Suitable bases include tertiary amines, such as imidazole, diisopropylethylamine, triethylamine, 1,8-diazabicyclo[5,4,0]undec-7-ene, combinations thereof and the like. Reaction temperatures range from 0° C. to the boiling point of the solvent used; preferably between 0 and 40° C.; reaction completion times range from 1 to 48 h.

In Step 4, compound 4 is converted into boronic acid (5) through halogen metal exchange reaction. Compound 4 is treated with 1 to 3 equivalents of alkylmetal reagent relative to compound 4, such as n-butyllithium, sec-butyllithium, tert-butyllithium, isopropylmagnesium chloride or Mg turnings with or without an initiator such as diisobutylaluminum hydride (DiBAl), followed by the addition of 1 to 3 equivalents of trialkyl borate relative to compound 4, such as trimethyl borate, triisopropyl borate, or tributyl borate. Suitable solvents include tetrahydrofuran, ether, 1,4-dioxane, 1,2-dimethoxyethane, toluene, hexanes, combinations thereof and the like. Alkylmetal reagent may also be added in the presence of trialkyl borate. The addition of butyllithium is carried out at between −100 and 0° C., preferably at between −80 and −40° C. The addition of isopropylmagnesium chloride is carried out at between −80 and 40° C., preferably at between −20 and 30° C. The addition of Mg turnings, with or without the addition of DiBAl, is carried out at between −80 and 40° C., preferably at between −35 and 30° C. The addition of the trialkyl borate is carried out at between −100 and 20° C. After the addition of trialkyl borate, the reaction is allowed to warm to room temperature, which is typically between −30 and 30° C. When alkylmetal reagent is added in the presence of trialkyl borate, the reaction mixture is allowed to warm to room temperature after the addition. Reaction completion times range from 1 to 12 h. Compound 5 may not be isolated and may be used for the next step without purification or in one pot.

In Step 5, the protecting group of compound 5 is removed under acidic conditions to give compound of the invention. Suitable acids include acetic acid, trifluoroacetic acid, hydrochloric acid, hydrobromic acid, sulfuric acid, p-toluenesulfonic acid and the like. The acids can be used in quantities ranging from 0.1 to 20 equivalents, relative to compound 5. When the protecting group is trialkylsilyl, basic reagents, such as tetrabutylammonium fluoride, can also be used. Suitable solvents include tetrahydrofuran, 1,4-dioxane, 1,2-dimethoxyethane, methanol, ethanol, propanol, acetonitrile, acetone, combination thereof and the like. Reaction temperatures range from 0° C. to the boiling point of the solvent used; preferably between 10° C. and reflux temperature of the solvent; reaction completion times range from 0.5 to 48 h. The product can be purified by methods known to those of skill in the art.

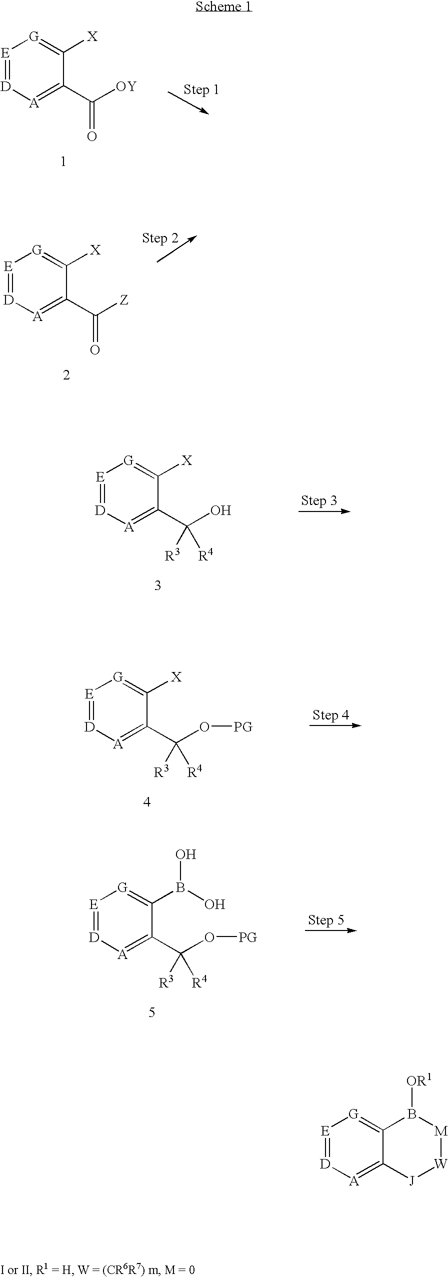

In another aspect, the invention provides a method of making a tetrahydropyran-containing boronic ester, said ester having a structure according to the following formula: 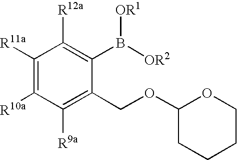
wherein R¹ and R² are members independently selected from H, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. R¹ and R², together with the atoms to which they are attached, can be optionally joined to form a 4- to 7-membered ring R^{9a}, R^{10a}, R^{11a} and R^{12a} are members independently selected from H, OR*, NR*R**, SR*, —S(O)R*, —S(O)₂R*, —S(O)₂NR*R**, nitro, halogen, cyano, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. R* and R** is a member selected from H, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. The method comprises: a) subjecting a first compound to Grignard or organolithium conditions, said first compound having a structure according to the following formula: 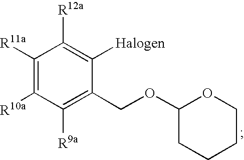
b) contacting the product of step a) with a borate ester, thereby forming said tetrahydropyran-containing boronic ester. In an exemplary embodiment, halogen is a member selected from iodo and bromo. In another exemplary embodiment, the borate ester is a member selected from B(OR¹)₂(OR²), wherein R¹ and R² are each members independently selected from H, substituted or unsubstituted methyl, substituted or unsubstituted ethyl, substituted or unsubstituted propyl, substituted or unsubstituted isopropyl, substituted or unsubstituted butyl, substituted or unsubstituted t-butyl, substituted or unsubstituted phenyl and substituted or unsubstituted benzyl. R¹ and R², together with the atoms to which they are joined, can optionally form a member selected from substituted or unsubstituted dioxaborolane, substituted or unsubstituted dioxaborinane and substituted or unsubstituted dioxaborepane. In another exemplary embodiment, the borate ester is a member selected from B(OR¹)₂(OR²), wherein R¹ and R², together with the atoms to which they are joined, form a member selected from dioxaborolane, substituted or unsubstituted tetramethyldioxaborolane, substituted or unsubstituted phenyldioxaborolane, dioxaborinane, dimethyldioxaborinane and dioxaborepane. In another exemplary embodiment, the Grignard or organolithium conditions further comprise diisobutyl aluminum hydride. In another exemplary embodiment, the temperature of the Grignard reaction does not exceed about 35° C. In another exemplary embodiment, the temperature of the Grignard reaction does not exceed about 40° C. In another exemplary embodiment, the temperature of the Grignard reaction does not exceed about 45° C. In an exemplary embodiment, step (b) is performed at a temperature of from about −30° C. to about −20° C. In another exemplary embodiment, step (b) is performed at a temperature of from about −35° C. to about −25° C. In another exemplary embodiment, step (b) is performed at a temperature of from about −50° C. to about −0° C. In another exemplary embodiment, step (b) is performed at a temperature of from about −40° C. to about −20° C. In another exemplary embodiment, the tetrahydropyran-containing boronic ester is

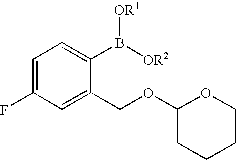

In another aspect, the invention provides a method of making a compound having a structure according to the following formula 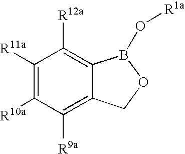
said method comprising: a) subjecting a first compound to Grignard or organolithium conditions, said first compound having a structure according to the following formula: 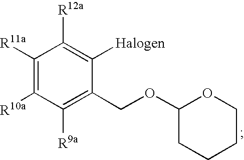
b) quenching said subjecting reaction with water and a organic acid, thereby forming said compound. In an exemplary embodiment, wherein said organic acid is a member selected from acetic acid. In another exemplary embodiment, the quenching step is essentially not contacted with a strong acid. In another exemplary embodiment, the compound is 1,3-dihydro-5-fluoro-1-hydroxy-2,1-benzoxaborole. In another exemplary embodiment, the compound is purified by recrystallization from a recrystallization solvent, wherein said recrystallization solvent essentially does not contain acetonitrile. In an exemplary embodiment, the recrystallization solvent contains less than 2% acetonitrile. In an exemplary embodiment, the recrystallization solvent contains less than 1% acetonitrile. In an exemplary embodiment, the recrystallization solvent contains less than 0.5% acetonitrile. In an exemplary embodiment, the recrystallization solvent contains less than 0.1% acetonitrile. In an exemplary embodiment, the recrystallization solvent contains toluene and a hydrocarbon solvent. In an exemplary embodiment, the recrystallization solvent contains about 1:1 toluene:hydrocarbon solvent. In an exemplary embodiment, the recrystallization solvent contains about 2:1 toluene:hydrocarbon solvent. In an exemplary embodiment, the recrystallization solvent contains about 3:1 toluene:hydrocarbon solvent. In an exemplary embodiment, the recrystallization solvent contains about 4:1 toluene:hydrocarbon solvent. In an exemplary embodiment, the hydrocarbon solvent is a member selected from heptane, octane, hexane, pentane and nonane. In an exemplary embodiment, the recrystallization solvent is 3:1 toluene:heptane.

Boronic Acid Preparation Strategy #2

In Scheme 2, Step 6, compound 2 is converted into boronic acid (6) via a transition metal catalyzed cross-coupling reaction. Compound 2 is treated with 1 to 3 equivalents of bis(pinacolato)diboron or 4,4,5,5-tetramethyl-1,3,2-dioxaborolane in the presence of transition metal catalyst, with the use of appropriate ligand and base as necessary. Suitable transition metal catalysts include palladium(II) acetate, palladium(II) acetoacetonate, tetrakis(triphenylphosphine)palladium, dichlorobis(triphenylphosphine)palladium, [1,1′-bis(diphenylphosphino)ferrocen]dichloropalladium(II), combinations thereof and the like. The catalyst can be used in quantities ranging from 1 to 5 mol % relative to compound 2. Suitable ligands include triphenylphosphine, tri(o-tolyl)phosphine, tricyclohexylphosphine, combinations thereof and the like. The ligand can be used in quantities ranging from 1 to 5 equivalents relative to compound 2. Suitable bases include sodium carbonate, potassium carbonate, potassium phenoxide, triethylamine, combinations thereof and the like. The base can be used in quantities ranging from 1 to 5 equivalents relative to compound 2. Suitable solvents include N,N-dimethylformamide, dimethylsufoxide, tetrahydrofuran, 1,4-dioxane, toluene, combinations thereof and the like. Reaction temperatures range from 20° C. to the boiling point of the solvent used; preferably between 50 and 150° C.; reaction completion times range from 1 to 72 h.

Pinacol ester is then oxidatively cleaved to give compound 6. Pinacol ester is treated with sodium periodate followed by acid. Sodium periodate can be used in quantities ranging from 2 to 5 equivalents relative to compound 6. Suitable solvents include tetrahydrofuran, 1,4-dioxane, acetonitrile, methanol, ethanol, combinations thereof and the like. Suitable acids include hydrochloric acid, hydrobromic acid, sulfuric acid combinations thereof and the like. Reaction temperatures range from 0° C. to the boiling point of the solvent used; preferably between 0 and 50° C.; reaction completion times range from 1 to 72 h.

In Step 7, the carbonyl group of compound 6 is treated with a reducing agent in an appropriate solvent to give a compound of the invention. Suitable reducing agents include borane complexes, such as borane-tetrahydrofuran, borane-dimethylsulfide, combinations thereof and the like. Lithium aluminum hydride, or sodium borohydride can also be used as reducing agents. The reducing agents can be used in quantities ranging from 0.5 to 5 equivalents, relative to compound 6. Suitable solvents include lower alcohol, such as methanol, ethanol, and propanol, diethyl ether, tetrahydrofuran, 1,4-dioxane and 1,2-dimethoxyethane, combinations thereof and the like. Reaction temperatures range from 0° C. to the boiling point of the solvent used; reaction completion times range from 1 to 24 h.

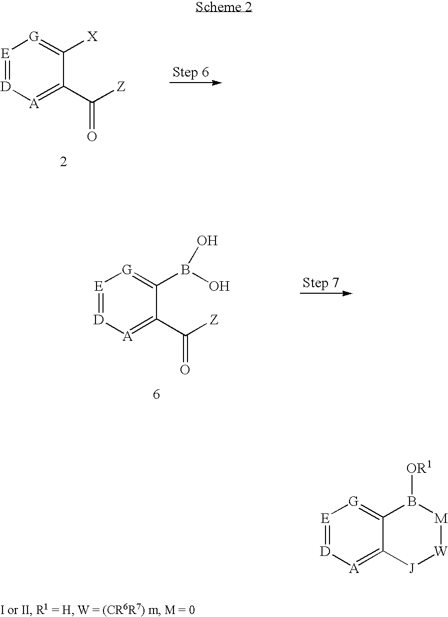

Boronic Acid Preparation Strategy #3

In Scheme 3, Step 8, compounds of the invention can be prepared in one step from compound 3. Compound 3 is mixed with trialkyl borate then treated with alkylmetal reagent. Suitable alkylmetal reagents include n-butyllithium, sec-butyllithium, tert-butyllithium combinations thereof and the like. Suitable trialkyl borates include trimethyl borate, triisopropyl borate, tributyl borate, combinations thereof and the like. The addition of butyllithium is carried out at between −100 and 0° C., preferably at between −80 and −40° C. The reaction mixture is allowed to warm to room temperature after the addition. Reaction completion times range from 1 to 12 h. The trialkyl borate can be used in quantities ranging from 1 to 5 equivalents relative to compound 3. The alkylmetal reagent can be used in quantities ranging from 1 to 2 equivalents relative to compound 3. Suitable solvents include tetrahydrofuran, ether, 1,4-dioxane, 1,2-dimethoxyethane, toluene, hexanes, combinations thereof and the like. Reaction completion times range from 1 to 12 h. Alternatively, a mixture of compound 3 and trialkyl borate can be refluxed for 1 to 3 h and the alcohol molecule formed upon the ester exchange can be distilled out before the addition of alkylmetal reagent.

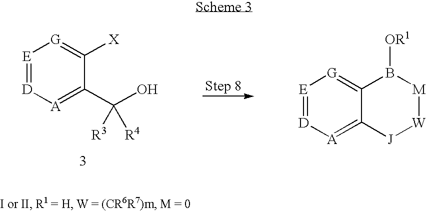

Boronic Acid Preparation Strategy #4

In Scheme 4, Step 10, the methyl group of compound 7 is brominated using N-bromosuccinimide. N-bromosuccinimide can be used in quantities ranging from 0.9 to 1.2 equivalents relative to compound 7. Suitable solvents include carbon tetrachloride, tetrahydrofuran, 1,4-dioxane, chlorobenzene, combinations thereof and the like. Reaction temperatures range from 20° C. to the boiling point of the solvent used; preferably between 50 and 150° C.; reaction completion times range from 1 to 12 h.

In Step 11, the bromomethylene group of compound 8 is converted to the benzyl alcohol 3. Compound 8 is treated with sodium acetate or potassium acetate. These acetates can be used in quantities ranging from 1 to 10 equivalents relative to compound 8. Suitable solvents include tetrahydrofuran, 1,4-dioxane, N,N-dimethylformamide, N,N-dimethylacetamide, N-methylpyrrolidone, dimethylsulfoxide, combinations thereof and the like. Reaction temperatures range from 20° C. to the boiling point of the solvent used; preferably between 50 and 100° C.; reaction completion times range from 1 to 12 h. The resulting acetate is hydrolyzed to compound 3 under basic conditions. Suitable bases include sodium hydroxide, lithium hydroxide, potassium hydroxide, combinations thereof and the like. The base can be used in quantities ranging from 1 to 5 equivalents relative to compound 8. Suitable solvents include methanol, ethanol, tetrahydrofuran, water, combinations thereof and the like. Reaction temperatures range from 20° C. to the boiling point of the solvent used; preferably between 50 and 100° C.; reaction completion times range from 1 to 12 h. Alternatively, compound 8 can be directly converted into compound 3 under the similar condition above.

Steps 3 through 5 convert compound 3 into a compound of the invention.

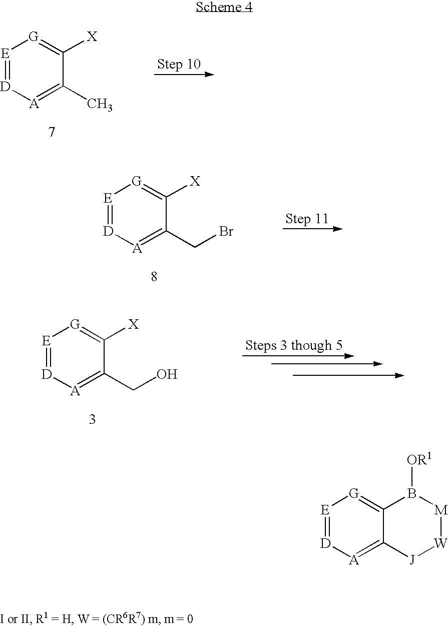

Boronic Acid Preparation Strategy #5

In Scheme 5, Step 12, compound 2 is treated with (methoxymethyl) triphenylphosphonium chloride or (methoxymethyl)triphenylphosphonium bromide in the presence of base followed by acid hydrolysis to give compound 9. Suitable bases include sodium hydride, potassium tert-butoxide, lithium diisopropylamide, butyllithium, lithium hexamethyldisilazane, combinations thereof and the like. The (methoxymethyl)triphenylphosphonium salt can be used in quantities ranging from 1 to 5 equivalents relative to compound 2. The base can be used in quantities ranging from 1 to 5 equivalents relative to compound 2. Suitable solvents include tetrahydrofuran, 1,2-dimethoxyethane, 1,4-dioxane, ether, toluene, hexane, N,N-dimethylformamide, combinations thereof and the like. Reaction temperatures range from 0° C. to the boiling point of the solvent used; preferably between 0 and 30° C.; reaction completion times range from 1 to 12 h. The enolether formed is hydrolyzed under acidic conditions. Suitable acids include hydrochloric acid, hydrobromic acid, sulfuric acid, and the like. Suitable solvents include tetrahydrofuran, 1,2-dimethoxyethane, 1,4-dioxane, methanol, ethanol, combination thereof and the like. Reaction temperatures range from 20° C. to the boiling point of the solvent used; preferably between 50 and 100° C.; reaction completion times range from 1 to 12 h.

Steps 2 through 5 convert compound 9 into a compound of the invention.

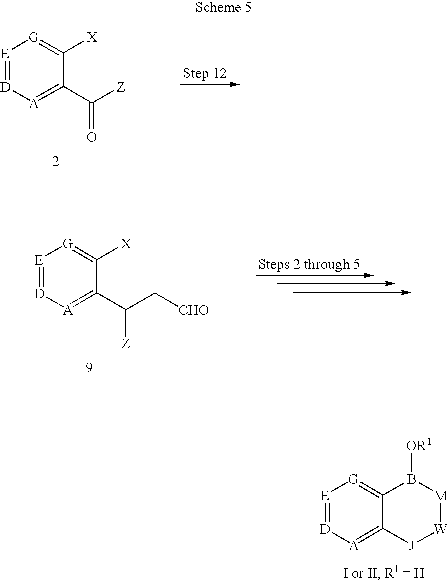

Boronic Acid Preparation Strategy #6

In Scheme 6, compound (I) wherein R¹ is H is converted into compound (I) wherein R¹ is alkyl by mixing with the corresponding alcohol, R¹OH. The suitable solvents include tetrahydrofuran, 1,2-dimethoxyethane, 1,4-dioxane, toluene, combinations thereof and the like. The alcohol (R¹OH) can be used as the solvent as well. Reaction temperatures range from 20° C. to the boiling point of the solvent used; preferably between 50 and 100° C.; reaction completion times range from 1 to 12 h.

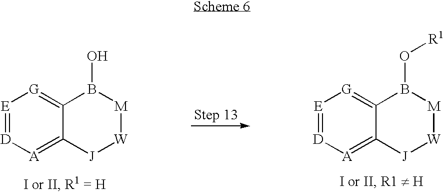

Boronic Acid Preparation Strategy #7

In Scheme 7, compound (Ia) is converted into its aminoalcohol complex (Ib). Compound (Ia) is treated with HOR¹NR^{1a}R^{1b}. The aminoalcohol can be used in quantities ranging from 1 to 10 equivalents relative to compound (Ia). Suitable solvents include methanol, ethanol, propanol, tetrahydrofuran, acetone, acetonitrile, 1,2-dimethoxyethane, 1,4-dioxane, toluene, N,N-dimethylformamide, water, combination thereof and the like. Reaction temperatures range from 20° C. to the boiling point of the solvent used; preferably between 50 and 100° C.; reaction completion times range from 1 to 24 h.

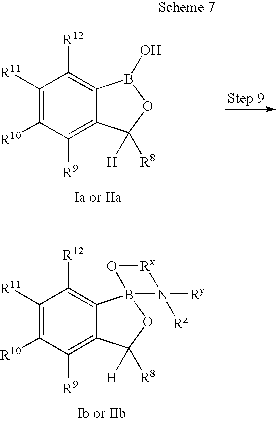

The compounds of the invention can be converted into hydrates and solvates by methods similar to those described above.

I. c.) Borinic Esters

In one aspect, the invention provides compounds useful in the methods which have a structure according to Formula XI: 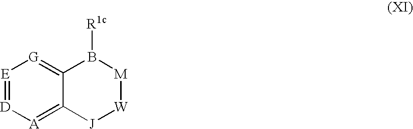
wherein the variables R^{1c}, A, D, E, G, J, W and M are described elsewhere herein.

In an exemplary embodiment of Formula (XI), R^{1c} is substituted or unsubstituted alkyl (C₁-C₄). In an exemplary embodiment of Formula (XI), R^{1c} is substituted or unsubstituted alkyloxy. In an exemplary embodiment of Formula (XI), R^{1c} is substituted or unsubstituted cycloalkyl (C₃-C₇). In an exemplary embodiment of Formula (XI), R^{1c} is substituted or unsubstituted alkenyl. In a further exemplary embodiment thereof, the substituted alkenyl has the structure 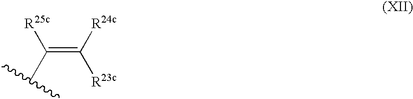
wherein R^{23c}, R^{24c}, and R^{25c} are each members independently selected from H, haloalkyl, aralkyl, substituted aralkyl, (CH₂)ᵣOH (where r=1 to 3), CH₂NR^{26c}R^{27c} (wherein R^{26c} and R^{27c} are independently selected from hydrogen and alkyl), CO₂H, CO₂alkyl, CONH₂, S-alkyl, S-aryl, SO₂alkyl, SO₃H, SCF₃, CN, halogen, CF₃, NO₂, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

In another exemplary embodiment of Formula (XI), R^{1c} is a substituted or unsubstituted alkynyl. In a further exemplary embodiment thereof, the substituted alkynyl has the structure 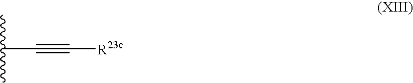
wherein R^{23c} is defined as before.

In an exemplary embodiment of Formula (XI), R^{1c} is substituted or unsubstituted aryl. In a further exemplary embodiment thereof the substituted aryl has the structure 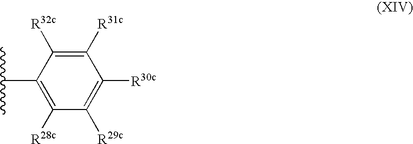
wherein R^{28c}, R^{29c}, R^{30c}, R^{31c} and R^{32c} are each members independently selected from H, aralkyl, substituted aralkyl, (CH₂)ₛOH (where s=1 to 3), CO₂H, CO₂alkyl, CONH₂, CONHalkyl, CON(alkyl)₂, OH, alkoxy, aryloxy, SH, S-alkyl, S-aryl, SO₂alkyl, SO₃H, SCF₃, CN, halogen, CF₃, NO₂, (CH₂)ₜNR²⁶R²⁷ (wherein R²⁶ and R²⁷ are independently selected from hydrogen, alkyl, and alkanoyl)(t=0 to 2), SO₂NH₂, OCH₂CH₂NH₂, OCH₂CH₂NHalkyl, OCH₂CH₂N(alkyl)₂, oxazolidin-2-yl, alkyl substituted oxazolidin-2-yl, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

In an exemplary embodiment of Formula (XI), R^{1c} is a substituted or unsubstituted aralkyl. In a further exemplary embodiment thereof the substituted aralkyl has the structure 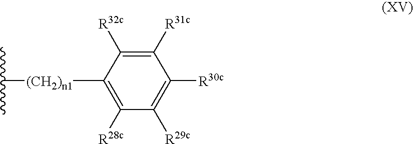
wherein R^{28c}, R^{29c}, R^{30c}, R^{31c} and R^{32c} are defined as before, and n1 is an integer selected from 1 to 15.

In an exemplary embodiment of Formula (XI), R^{1c} is a substituted or unsubstituted heteroaryl. In a further exemplary embodiment thereof, heteroaryl has the structure 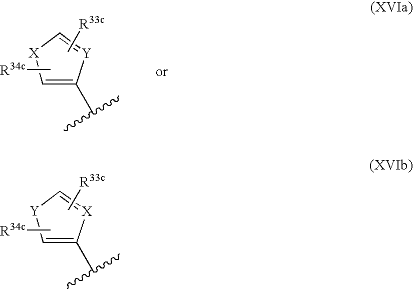
wherein X is a member selected from CH═CH, N═CH, NR^{35c} (wherein R^{35c}═H, alkyl, aryl or benzyl), O, or S. Y═CH or N. R^{33c} and R^{34c} are each members independently selected from H, haloalkyl, aralkyl, substituted aralkyl, (CH₂)ᵤOH (where u=1, 2 or 3), (CH₂)ᵥNR^{26c}R^{27c} (wherein R^{26c} and R^{27c} are independently selected from hydrogen, alkyl and alkanoyl)(v=0 to 3), CO₂H, CO₂alkyl, CONH₂, S-alkyl, S-aryl, SO₂alkyl, SO₃H, SCF₃, CN, halogen, CF₃, NO₂, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

The structures of the invention also permit solvent interactions that may afford structures (Formula XVII) that include atoms derived from the solvent encountered by the compounds of the invention during synthetic manipulations and therapeutic uses. Structure XVII arises from the formation of a dative bond between the solvent(s) with the Lewis acidic boron center. Thus, such solvent complexes could be stable entities with comparative bioactivities. Such structures are expressly contemplated by the present invention where R^{40c} is H or alkyl.

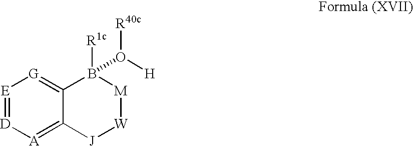

In an exemplary embodiment, the invention provides a structure which is a member selected from Formula (Ic), (IIc) and (IIIc): 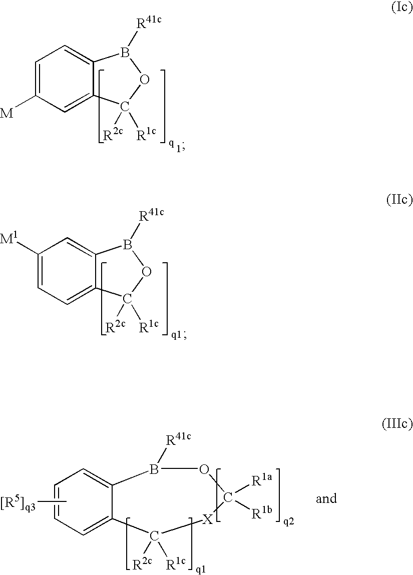
wherein B is boron. q1 and q2 are integers independently selected from 1 to 3. q3 is an integer selected from 0 to 4. M is a member selected from H, halogen, —OCH₃, and —CH₂—O—CH₂—O—CH₃. M¹ is a member selected from halogen, —CH₂OH, and —OCH₃. X is a member selected from O, S, and NR^{xc}. R^{xc} is a member selected from H and substituted or unsubstituted alkyl. R^{1c}, R^{3c}, R^{4c}, R^{2c} and R^{5c} are members independently selected from H, OH, NH₂, SH, CN, NO₂, SO₂, OSO₂OH, OSO₂NH₂, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. R^{41c} is a member selected from substituted or unsubstituted aryl, substituted or unsubstituted arylalkyl, substituted or unsubstituted heteroaryl, substituted or unsubstituted heteroarylalkyl and substituted or unsubstituted vinyl.

The compounds of the invention can form a hydrate with water, solvates with alcohols such as methanol, ethanol, propanol, and the like; adducts with amino compounds, such as ammonia, methylamine, ethylamine, and the like; adducts with acids, such as formic acid, acetic acid and the like; complexes with ethanolamine, quinoline, amino acids, and the like.

In an exemplary embodiment, the compound has a structure which is a member selected from 2-(3-Chlorophenyl)-[1,3,2]-dioxaborolane, (3-Chlorophenyl)(4′-fluoro-(2′-(methoxymethoxy)-methyl)-phenyl)-borinic acid, 1-(3-Chlorophenyl)-5-fluoro-1,3-dihydrobenzo[c][1,2]oxaborole, 1-(3-Chlorophenyl)-6-fluoro-1,3-dihydrobenzo[c][1,2]oxaborole, 1-(3-Chlorophenyl)-1,3-dihydrobenzo[c][1,2]oxaborole, 5-Chloro-1-(3-Fluorophenyl)-1,3-dihydrobenzo[c][1,2]oxaborole, 2-(3-fluorophenyl)-[1,3,2]-dioxaborolane, 3-(Benzo[c][1,2]oxaborol-1(3H)-yl)benzonitrile, 2-(3-cyanophenyl)-[1,3,2]-dioxaborolane, (3-Chlorophenyl)(5′-fluoro-(2′-(methoxymethoxy)methyl)-phenyl)-borinic acid, 1-(3-Chlorophenyl)-1,3-dihydro-3,3-dimethylbenzo[c][1,2]oxaborole, (3-Chlorophenyl)(2-(2-(methoxymethoxy)propan-2-yl)phenylborinic acid, 1-(3-Chlorophenyl)-1,3-dihydro-3,3-dimethylbenzo[c][1,2]oxaborole, 1-(4-Chlorophenyl)-1,3-dihydrobenzo[c][1,2]oxaborole, 2-(4-chlorophenyl)-[1,3,2]-dioxaborolane, 4-(Benzo[c][1,2]oxaborol-1(3H)-yl)benzonitrile, 2-(4-cyanophenyl)-[1,3,2]-dioxaborolane, 4-(5-Fluorobenzo[c][1,2]oxaborol-1(3H)-yl)benzonitrile, 2-(4-cyanophenyl)-[1,3,2]-dioxaborolane, 3-(5-Fluorobenzo[c][1,2]oxaborol-1(3H)-yl)benzonitrile, 2-(3-cyanophenyl)-[1,3,2]-dioxaborolane, 3-(6-Fluorobenzo[c][1,2]oxaborol-1(3H)-yl)benzonitrile, 2-(3-cyanophenyl)-[1,3,2]-dioxaborolane, 1-(3-Cyanophenyl)-5,6-dimethoxy-1,3-dihydrobenzo[c][1,2]-oxaborole, 2-(3-chlorophenyl)-[1,3,2]-dioxaborolane, (4-(5-(Fluorobenzo[c][1,2]oxaborol-1(3H)-yl)phenylmethanamine, 5-Fluoro-2-(methoxymethoxymethyl)phenyl]-[1,3,2]-dioxaborolane, 4-(5-(Fluorobenzo[c][1,2]oxaborol-1(3H)-yl)phenylmethanamine, (3-(5-(Fluorobenzo[c][1,2]oxaborol-1(3H)-yl)-phenylmethanamine, (4-(5-(Fluorobenzo[c][1,2]oxaborol-1(3H)-yl)phenyl)methanol, (3-(5-(Fluorobenzo[c][1,2]oxaborol-1(3H)-yl)phenyl)methanol, 3-(6-Fluorobenzo[c][1,2]oxaborol-1(3H)-yl)phenol, 3-(5-Fluorobenzo[c][1,2]oxaborol-1(3H)-yl)pyridine, (2-(Benzo[c][1,2]oxaborol-1(3H)-yl)phenyl)methanol, 2-[(Methoxymethoxy)methyl]phenyl boronic acid, 2-[(Methoxymethoxymethyl)pheny]-[1,3,2]-dioxaborolane, Bis[2-(methoxymethoxymethyl)phenyl]borinic acid, (2-(Benzo[c][1,2]oxaborol-1(3H)-yl)phenyl)methanol, (2-(Benzo[c][1,2]oxaborol-1(3H)-yl)phenyl)-N,N-dimethylmethanamine, (2-(Benzo[c][1,2]oxaborol-1(3H)-yl)-5-chlorophenyl)-N,N-dimethylmethanamine, (2-(Benzo[c][1,2]oxaborol-1(3H)-yl)-5-chlorophenyl)methanol, (2-(Benzo[c][1,2]oxaborol-1(3H)-yl)-5-chlorophenyl)methanol, (5-Chloro-2-(5-chlorobenzo[c][1,2]oxaborol-1(3H)-yl)phenyl)methanol, Bis[4-chloro-2-(methoxymethoxymethyl)phenyl]borinic acid, (5-Chloro-2-(5-chlorobenzo[c][1,2]oxaborol-1(3H)-yl)phenyl)methanol, (5-Chloro-2-(5-chlorobenzo[c][1,2]oxaborol-1(3H)-yl)phenyl-N,N-dimethylmethanamine, 1-(4-chloro-2-methoxyphenyl)-1,3-dihydrobenzo[c][1,2]benzoxaborole, 4-Chloro-2-methoxyphenylboronic acid ethylene glycol ester, 1-(4-chloro-2-methoxyphenyl)-1,3-dihydrobenzo[c][1,2]benzoxaborole, 2-(Benzo[c][1,2]oxaboral-1(3H)-yl)-5-chlorophenol, 2-(3-(Benzo[c][1,2]oxaborol-1(3H)-yl)phenoxy)-5-chlorophenol, 2-(3-(Benzo[c][1,2]oxaborol-1(3H)-yl)Phenoxy)-5-chlorophenol 4-((3-(5-Fluorobenzo[c][1,2]oxaborol-1(3H)-yl)phenyl)methyl)morpholine, 3-(5-Fluorobenzo[c][1,2]oxaborol-1(3H)-yl]phenyl)-methyl 8-hydroxy-quinoline-2-carboxylate, 1-(3-Chlorophenyl)-2,3-dihydro-2-(methoxymethy)-1H-benzo[c][1,2]azaborole, 3-Chlorophenyl 2-[N,N-bis(methoxymethyl)aminomethyl]phenylborinic acid, 1-(3-Chlorophenyl)-2,3-dihydro-2-(methoxymethy)-1H-benzo[c][1,2]azaborole, 1-(3-Chlorophenyl)-1,3,4,5-tetrahydrobenzo-[c][1,2]-oxaborepine, 1-(3-Chlorophenyl)-1,3,4,5-tetrahydrobenzo[c][1,2]oxaborepine, 1-(3-Chlorophenyl)-3,4-dihydro-1H-benzo[c][1,2]-oxaborinine, 2-(3-Chlorophenyl)-[1,3,2]dioxaborolane, (3-Chlorophenyl)(2′-(2-(methoxymethoxy)ethyl)phenyl)borinic acid, and 1-(3-Chlorophenyl)-3,4-dihydro-1H-benzo[c][1,2]oxaborinine.

I. d.) Preparation of Boron-Containing Compounds

Compounds of use in the present invention can be prepared using commercially available starting materials, known intermediates, or by using the synthetic methods published in references described and incorporated by reference herein.

I. e.) Boronic Esters

Methods of making boronic esters are known in the art, and it is within the knowledge of one skilled in the art to use these methods in order to make the boronic esters described herein. Examples include U.S. patent Ser. Nos. 10/740,304, 10/867,465, 11/152,959, 11/153,765, 11/153,010, 11/389,605, 11/357,687, 11/357,687 and U.S. Prov. Pat. Nos. 60/754,750, 60/774,532 and 60/746,361, which are herein incorporated by reference. Another example of a synthetic pathway for the preparation of compounds of use in the invention is shown below: 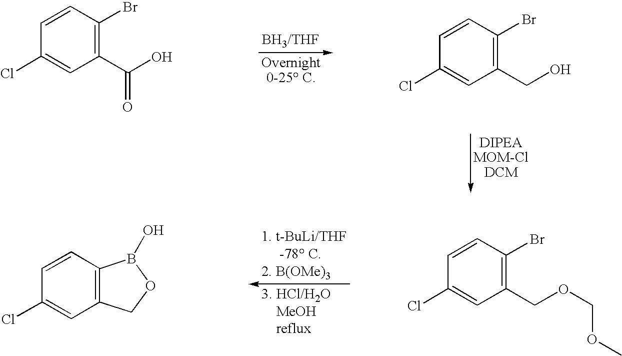
I. f) Borinic Esters

Methods of making borinic esters are known in the art, and it is within the knowledge of one skilled in the art to use these methods in order to make the boronic esters described herein. Examples include U.S. patent Ser. Nos. 10/868,268, and 11/743,665 which are herein incorporated by reference.

II. Assays for Periodontal Disease Inhibition

Art-recognized techniques of genetics and molecular/cell biology are of use to identify compounds that are appropriate for periodontal disease inhibition. Examples of assays used for this determination are provided herein.

III. Oral Care Compositions

In another aspect, the present invention provides an oral care composition comprising a compound of the invention. In an exemplary embodiment, the compound is a boron-containing compound described herein. In another exemplary embodiment, the compound is a member selected from 3-hydroxypyridine-2-carbonyloxy-bis(3-chloro-4-methylphenyl)-borane (bis(3-Chloro4-methylphenyl)borinic acid 3-hydroxypicolinate ester), 1,3-dihydro-5-fluoro-1-hydroxy-2,1-benzoxaborole, and 5-(4-cyanophenoxy)-1,3-dihydro-1-hydroxy-2,1-benzoxaborole. In another exemplary embodiment, the compound is 3-hydroxypyridine-2-carbonyloxy-bis(3-chloro-4-methylphenyl)-borane (bis(3-Chloro4-methylphenyl)borinic acid 3-hydroxypicolinate ester). These oral care compositions are of use in the methods of the invention.

An oral care composition of the present invention can take any physical form suitable for application to an oral surface. In various illustrative embodiments the composition can be a liquid solution suitable for irrigating, rinsing or spraying; a dentifrice such as a powder, toothpaste or dental gel; a liquid suitable for painting a dental surface (e.g., a liquid whitener); a chewing gum; a dissolvable, partially dissolvable or non-dissolvable film or strip (e.g., a whitening strip); a wafer; a wipe or towelette; an implant; a dental floss; toothpastes, prophylactic pastes, tooth polishes, gels, professional gels and other related products applied by dentists, as well as mouth washes, mouth rinses, dental flosses, chewing gums, lozenges, tablets, edible food products, Periochips for insertion into periodontal pockets (made of material such as chlorihexidine gluconate) and the like. The composition can contain active and/or carrier ingredients additional to those recited above.

In certain embodiments of the invention, a compound described herein in the oral care composition can be encapsulated in a material called a microsphere. This material can act as a slow-release mechanism for the compound. In an exemplary embodiment, the microsphere is at least partially constructed from chitosan. In an exemplary embodiment, 3-hydroxypyridine-2-carbonyloxy-bis(3-chloro-4-methylphenyl)-borane is encapsulated in a microsphere in an oral care composition. Additional descriptions of microencapsulated materials are described in Govender et al., Journal of Microencapsulation, 23(7): November 2006, pp 750-761, which is herein incorporated by reference.

In certain embodiments the composition is adapted for application to an oral surface of a small domestic animal, for example a cat or a dog. Such a composition is typically edible or chewable by the animal, and can take the form, for example, of a cat or dog food, treat or toy.

Illustratively, the composition of any of the embodiments described above is a mouthwash or rinse, an oral spray, a dentifrice, an oral strip, a liquid whitener or a chewing gum. Rinses include liquids adapted for irrigation by means of devices such as high-pressure water jets. Dentifrices include without limitation toothpastes, gels and powders. A “liquid whitener” herein encompasses semi-liquid compositions such as gels as well as flowable liquids, so long as the composition is capable of application to a dental surface by painting with a brush or other suitable device. “Painting” in the present context means application of a thin layer of the composition to the dental surface. In one embodiment the composition is a toothpaste or gel dentifrice.

A composition of the invention can comprise, in addition to the boron-containing compound described herein, a vitamin or vitamin derivative or antioxidant component or one or more active agents (“actives”).

Among useful actives are those addressing, without limitation, appearance and structural changes to teeth, treatment and prevention of plaque, calculus, dental caries, cavities, abscesses, inflamed and/or bleeding gums, gingivitis, oral infective and/or inflammatory conditions in general, tooth sensitivity, halitosis and the like. Thus, a composition of the invention can contain one or more actives such as whitening agents, fluoride ion sources, antimicrobial agents additional to the boron-containing compound described herein, desensitizing agents, anticalculus (tartar control) agents, stannous ion sources, zinc ion sources, sialagogues, breath-freshening agents, antiplaque agents, anti-inflammatory agents additional to any boron-containing compound that has anti-inflammatory properties, periodontal agents, analgesics and nutrients. Actives should be selected for compatibility with each other and with other ingredients of the composition.

Actives useful herein are normally present in the composition in amounts selected to be safe and effective, i.e., amount sufficient to provide a desired benefit, for example a therapeutic, prophylactic, nutritive or cosmetic effect, when the composition is used repeatedly as described herein, without undue side effects such as toxicity, irritation or allergic reaction, commensurate with a reasonable benefit/risk ratio. Such a safe and effective amount will usually, but not necessarily, fall within ranges approved by appropriate regulatory agencies. A safe and effective amount in a specific case depends on many factors, including the particular benefit desired or condition being treated or sought to be prevented, the particular subject using, or being administered, the composition, the frequency and duration of use, etc. Actives are typically present in a total amount of about 0.01% to about 80%, for example about 0.05% to about 60%, about 0.1% to about 50%, or about 0.5% to about 40%, by weight of the composition.

One or more actives, including the boron-containing compound described herein, can optionally be present in encapsulated form in the composition. For example, beads containing one or more actives can be adapted to rupture during brushing or chewing to release the active(s) to the oral surface.

Additionally, the composition of the invention may include any of the components conventionally present or desirable in an oral care product. For example, the composition may include a whitening agent, such as peroxy compounds, chlorine dioxide, chlorites and hypochlorites, a polymer-peroxide complex, polyvinylpyrrolidone-hydrogen peroxide (PVP—H₂O₂) complex; a source of fluoride ions (monofluorophosphate and fluorosilicate salts, antibacterial agents. Active agents such as antibacterial agents may be includes, including, for example, those listed in U.S. Pat. No. 5,776,435 to Gaffar et al., the contents of which are incorporated herein by reference. The composition may further include a tooth anti-sensitivity agent, a sialagogue (saliva stimulating agent), a breath-freshening agent, an antiplaque or plaque disrupting agent.

Among useful carriers for optional inclusion in a composition of the invention are diluents, abrasives, bicarbonate salts, pH modifying agents, surfactants, foam modulators, thickening agents, viscosity modifiers, humectants, sweeteners, flavorants and colorants. One carrier material, or more than one carrier material of the same or different classes, can optionally be present. Water is a preferred diluent and in some compositions such as mouthwashes and whitening liquids may be accompanied by an additional solvent, such as an alcohol, e.g., ethanol.

The composition may contain abrasives, pH modifying agents, surfactants, foam modulators, thickening agents, viscosity modifiers, humectants, sweeteners, flavorants, colorants.

The invention further provides a method of oral care comprising a step of applying a composition as described herein to an oral surface of a subject. In one embodiment the composition is a toothpaste or gel dentifrice, and the applying step comprises brushing the surface, for example a dental surface and a periodontal surface adjacent thereto, with the dentifrice.

According to an embodiment of the invention, there is still further provided a method of inhibiting inflammation in an oral tissue of a subject. The method of this embodiment comprises applying to an oral surface proximal to the tissue a compound of the invention. In an examplary embodiment, the compound is a member selected from 3-hydroxypyridine-2-carbonyloxy-bis(3-chloro-4-methylphenyl)-borane (bis(3-Chloro-4-methylphenyl)borinic acid 3-hydroxypicolinate ester), 1,3-dihydro-5-fluoro-1-hydroxy-2,1-benzoxaborole, and 5-(4-cyanophenoxy)-1,3-dihydro-1-hydroxy-2,1-benzoxaborole. In another examplary embodiment, the compound is 3-hydroxypyridine-2-carbonyloxy-bis(3-chloro-4-methylphenyl)-borane. In another embodiment, there is provided a method of promoting oral health in a subject. The method of this embodiment comprises applying to an oral surface of the subject a compound of the inventions.

Practice of a method of the invention can promote any aspect or aspects of oral health. As one example, such a method can promote periodontal and/or gingival health, for instance by reducing bacterial infection and/or inflammation. As another example, such a method can provide a breath-freshening benefit, for instance through antibacterial and/or antioxidant activity. As yet another example, such a method can promote tooth retention, for instance by reducing or preventing dental caries and preventing destruction of the bone matrix that holds the tooth in place. As yet another example, such a method can provide an anti-plaque benefit. As yet another example, such a method can reduce damage to oral tissues from free radicals, including those occurring as a result of contact with tobacco smoke or polluted air.

It is well known that enhanced oral health, in particular improved periodontal and/or gingival health associated with reduced bacterial infection and/or inflammation, can lead to systemic or whole-body health benefits. Delivery of vitamins via an oral surface as provided herein can further enhance general health by supplementing the vitamins ingested with food.

Among systemic conditions that can be ameliorated as a result of improved oral health following practice of a method of the invention are cardiovascular disease including atherosclerosis, coronary heart disease (CHD) and stroke; diabetes; respiratory infections including bacterial pneumonia; preterm low birth weight; stomach ulcers; bacteremia; infective endocarditis; prosthetic device infection; chronic obstructive pulmonary disease (COPD); and brain abscesses.

Practice of the methods can consist of a single application as described herein, or can comprise repeated such applications. In one embodiment a method as described herein is repeated at regular intervals, for example twice or once daily, twice or once weekly, twice or once monthly, in a program or regimen conducted at home and/or in a professional or clinical setting.

The subject in any of the above methods can be a human or non-human mammal, for example a dog, cat, horse or exotic mammal. In certain embodiments the subject is a small domestic animal, for example a cat or a dog, and the composition, in the form of a food, treat or toy, is given to the animal to chew.

Oral care compositions of this invention can further include a variety of other components, including hydrophilic liquid vehicles, including but not limited to glycerin, propylene glycol, polyethylene glycol, and hydrophobic liquid vehicles such as triglyceride, diglyceride, and organic oils including mineral oil, essential oils, and fatty vegetable oils. These hydrophilic and hydrophobic liquid vehicles can be used either singly or in combination and preferably, can be added in a proportion of from about 2 to about 50 wt. % (in the case of compositions comprising liquid vehicles), especially from about 10 to about 35 wt. % based on the whole composition. Using one or more of these liquid vehicles, the composition of the present invention for the oral cavity may preferably be formulated into a use form such as gel, liquid, or paste.

The oral care compositions of the present invention can also contain flavor components, typically in the form of natural flavors or aroma oils and/or herbal extracts and oils. These flavor components can serve not only to give a palatable flavor to the oral care composition, but can act as natural antibacterial agents and preservatives at the same time. The oils suitable for use in the present invention include but are not limited to citric oil, lemon oil, lime oil, lemongrass oil, orange oil, sweet orange oil, grapefruit oil, pomegranate oil, apricot oil extract, tangerine extract, tangelo oil, peppermint oil, spearmint oil, sage oil, rosemary oil, cinnamon oil, winter green oil, clove oil, eucalyptus oil, ginger oil, sassafras oil, menthol, arvensis mint oil, synthetic mint flavors and oils, carvone, eugenol, methyleugenol, methyl salicylate, methyl eugenol, thymol, anethole, millefolium extract, chamomile, lavender oil, myrrh, eugenol, tea tree oil, sage oil, mallow, limonene, ocimene, n-decyl alcohol, citronellol, α-terpineol, linalol, ethyllinalol, thyme, almond oil, nutmeg, and vanillin. Either one of these flavors or a mixture of two or more of these flavors can be used in the dentifrice composition. The content thereof ranges from about 3% to about 20% by weight, such as from about 4% to about 15% by weight, based on the whole composition.

Silica abrasives can also be incorporated into the oral care composition of the present invention, without detracting from the scope of the invention. Specific silica abrasives suitable for use with the present invention include but are not limited to silica gels, precipitated silicas, silicates, and hydrated silica. Silica gels suitable for use with the present invention are hydrogels, hydrous gels, xerogels, and aerogels, such as those known in the art and described in U.S. Pat. No. 6,440,397. Precipitated silicas are those known in the art, such as the suitable oral care-type precipitated silicas described in U.S. Pat. No. 5,589,160. Suitable silicates are any of those naturally occurring or synthetic silicates suitable for use with oral care compositions. These silica abrasives can be used singly or in combination. An exemplary silica abrasive for use with the present invention includes silica gels. The silica abrasives can be used together with the calcium salt or in lieu of the calcium salt component.

Water can optionally be incorporated into the oral care compositions of the present invention, such as toothpastes and mouthwashes. Water used in the preparation of commercially suitable oral care compositions should preferably be deionized and free of organic impurities. Water can generally comprise about 0% to about 40% by weight of the toothpaste compositions herein.

In addition to the above-described components, the oral care composition of the present invention can further contain a variety of optional ingredients and vehicles generally used for preparations for use in the oral cavity, such as toothpastes and mouthwashes. These optional components include, but are not limited to, such components as abrasives, surfactants, thickening agents, buffers, humectants, preservatives, and antibiotic and anti-caries agents. All of these additives, described in further detail below, are generally usual and would be known to one of skill in the art.

Dental abrasives useful in the dentifrice compositions of the present invention include a variety of different materials known in the art. Preferably, the abrasive material should be one which is compatible with the composition of interest and does not excessively abrade dentin. Suitable abrasives include for example, silicas including gels and precipitates; insoluble polymetaphosphate, hydrated alumina, resinous abrasives such as polymerized resins (e.g. ureas, melamines, cross-linked epoxides, phenolics, and the like), and mixtures thereof.

Another optional component of the oral care compositions of the present invention is a humectant. The humectant serves to keep compositions such as toothpaste compositions from hardening upon exposure to air, and to give mouthwash and toothpaste compositions a moist feel to the mouth. Certain humectants can also impart desirable sweetness of flavor to toothpaste and mouthwash compositions. Suitable humectants for use in compositions of the present invention include edible polyhydric alcohols such as glycerin, sorbitol, xylitol, polyethylene glycol, and propylene glycol.

The oral care compositions of the present invention can also optionally contain sweeteners such as saccharin sodium, acesulfame potassium, glycyrrhizin, perillartine, thaumatin, aspartylphenylalanyl methyl ester and xylitol.

Buffering agents are another optional component of the oral care compositions of the present invention. The buffering agents serve to retain the pH of the compositions within the preferred range. Suitable buffering agents for use in dentifrice compositions of the present invention include soluble phosphate salts.

Other optional components of the oral care compositions of the present invention are preservatives, such as those that prevent microbial growth in the oral care compositions. Suitable preservatives include but are not limited to methylparaben, propylparaben, bezoates, and alcohols such as ethanol.

Binders and thickeners can also optionally be used in the oral care compositions of the present invention, particularly in toothpaste compositions. Preferred binders and thickening agents include, but are not limited to, carrageenan (e.g. Viscarin, Irish moss, and the like); cellulose derivatives such as hydroxyethyl cellulose, sodium carboxymethyl cellulose, and sodium carboxymethyl hydroxypropyl cellulose, carboxyvinyl polymers; natural gums such as karaya gum, gum Arabic, and tragacanth; polysaccharide gums such as xanthan gum; fumed silica; and colloidal magnesium aluminum silicate.

Compositions of the present oral care compositions can also optionally contain a surfactant. Suitable surfactants are those which are reasonably stable and preferably form suds (bubbles) throughout the pH range of the dentifrice compositions. Surfactants can also be added to act as solubilizing agents to help retain sparingly soluble components in solutions or mixtures. Surfactants useful in the dentifrice compositions as sudsing agents can be soaps, polysorbates, poloxamers, and synthetic detergents that are anionic, nonionic, cationic, zwitterionic, or amphoteric, and mixtures thereof.

The oral care compositions of the present invention can also optionally comprise anti-caries agents. Preferred anti-caries agents are water-soluble fluoride ion sources. The number of such fluoride ions sources is great and well known to those of skill in the art, and includes those disclosed in U.S. Pat. No. 3,535,421. Exemplary fluoride ion source materials include sodium fluoride, potassium fluoride, sodium monofluorophosphate and mixtures thereof.

Antimicrobial and anti-plaque agents can also optionally be present in the oral care compositions of the present condition. Such agents may include: triclosan (5-chloro-2-(2,4-dichlorophenoxy)-phenol); chlorhexidine; chlorhexidine digluconate (CHX); alexidine, hexetidine (HEX); sanguinarine (SNG); benzalkonium chloride; salicylanilide; domiphen bromide; cetylpyridiniumchloride (CPC); tetradecylpyridinium chloride (TPC); N-tetra-decyl-4-ethylpyridinium chloride (TDEPC); octenidine; delmopinol; octapinol, and other piperidino derivatives; nicin preparations; zinc/stannous ion agents; antibiotics such as augmentin, amoxicillin, tetracycline, deoxycycline, minocycline, and metronidazole; peroxide, such as cylium peroxide, hydrogen peroxide, and magnesium monoperthalate an its analogs; and analogs and salts of the above listed antimicrobial and antiplaque agents.

Oral care compositions of the present invention can also optionally include one or more anticalculus (anti-tartar) agents. Anticalculus agents which may be useful in the dentifrice compositions of the present invention include antimicrobials such as chlorhexidine, niddamycin, and triclosan, metals and metal salts such as zinc citrate, Vitamin C, bisphosphonates, triclosanpyrophosphates, pyrophosphates, polyphosphates, polyacrylates and other polycarboxylates, polyepoxysuccinates, ethyenediaminetetraacetic acid (EDTA), nitrilotriacetic acid and related compounds, polyphosphonates, and polypyrophosphates such as sodium hexametaphosphate, as well as other anticalculus agents known to those of skill in the art, such as those described in K. J. Fairbrother et al., “Anticalculus agents,” Journal of Clinical Periodontology Vol. 27, pp. 285 301 (2000).

Nutrients and vitamins can also optionally be added to the oral care compositions of the present invention. Such agents can include folates, retinoids (Vitamin A), Vitamin B (B₁-thyamin, B₂-riboflavin, B₃-niacine, B₅-pantothenic acid, B₆-pyridoxine, B₇-biotin, B₈/B₉/Bc-folic acid, B₁₂-cyanocobalamin), Vitamin C (ascorbic acid, sodium ascorbate), Vitamin E, Vitamin E analogs (dl-α-tocopherol acetate, tocopherol succinate, tocopherol nicotinate) and zinc.

A variety of miscellaneous additives can also be optionally formulated into the oral care compositions of the present invention, such as tooth desensitizing agents (e.g. potassium and strontium salts), condensed anti-tartar agents such as sodium and potassium tetrapyrophosphate, whitening agents such as aluminum oxide and calcium peroxide, debriding agents such as sodium bicarbonate, pigments and dyes, such as Blue 15-C174160, Green 7-C174260, Reds 4-CI12085 and 40 CI16035, Yellows 115 CI47005:1 and 5 CI19140, and Carmine 5 CI16035), as well as additives such as mica and sparkles.

As with the other optional oral care additives, use can be made of either one of these ingredients or a mixture of two or more of these ingredients in amounts appropriate for the oral care composition.

IV. Methods of Inhibiting Microorganism Growth or Killing Microorganisms

In a further aspect, the invention provides a method for inhibiting the growth, or killing, a microorganism, preferably a bacteria, fungus, virus, yeast or parasite, comprising contacting the organism with a compound of the invention. In an exemplary embodiment, the compound is a boron-containing compound described herein. In an exemplary embodiment, the microorganism is in the oral cavity of an animal, which is a member selected from human, cattle, deer, reindeer, goat, honey bee, pig, sheep, horse, cow, bull, dog, guinea pig, gerbil, rabbit, cat, camel, yak, elephant, ostrich, otter, chicken, duck, goose, guinea fowl, pigeon, swan, and turkey. In another exemplary embodiment, the animal is a human. Alternatively, the method is used in vitro, for example, to eliminate microbial contaminants in a cell culture. In another examplary embodiment, the compound is a member selected from 3-hydroxypyridine-2-carbonyloxy-bis(3-chloro-4-methylphenyl)-borane (bis(3-Chloro-4-methylphenyl)borinic acid 3-hydroxypicolinate ester), 1,3-dihydro-5-fluoro-1-hydroxy-2,1-benzoxaborole, and 5-(4-cyanophenoxy)-1,3-dihydro-1-hydroxy-2,1-benzoxaborole. In another examplary embodiment, the compound is 3-hydroxypyridine-2-carbonyloxy-bis(3-chloro-4-methylphenyl)-borane.

IV. a) Methods Involving Fungi or Yeast

In an exemplary embodiment, the microorganism is a member selected from a fungus and a yeast. In another exemplary embodiment, the fungus or yeast is a member selected from Candida species, Trichophyton species, Microsporium species, Aspergillus species, Cryptococcus species, Blastomyces species, Cocciodiodes species, Histoplasma species, Paracoccidiodes species, Phycomycetes species, Malassezia species, Fusarium species, Epidermophyton species, Scytalidium species, Scopulariopsis species, Alternaria species, Penicillium species, Phialophora species, Rhizopus species, Scedosporium species and Zygomycetes class. In another exemplary embodiment, the fungus or yeast is a member selected from Aspergillus fumigatus (A. fumigatus), Blastomyces dermatitidis, Candida Albicans (C. albicans, both fluconazole sensitive and resistant strains), Candida glabrata (C. glabrata), Candida krusei (C. krusei), Cryptococcus neoformans (C. neoformans), Candida parapsilosis (C. parapsilosis), Candida tropicalis (C. tropicalis), Cocciodiodes immitis, Epidermophyton floccosum (E. floccosum), Fusarium solani (F. solani), Histoplasma capsulatum, Malassezia furfur (M. furfur), Malassezia pachydermatis (M. pachydermatis), Malassezia sympodialis (M. sympodialis), Microsporum audouinii (M. audouinii), Microsporum canis (M. canis), Microsporum gypseum (M. gypseum), Paracoccidiodes brasiliensis and Phycomycetes spp, Trichophyton mentagrophytes (T. mentagrophytes), Trichophyton rubrum (T. rubrum), Trichophyton tonsurans (T. tonsurans). In another exemplary embodiment, the fungus or yeast is a member selected from Trichophyton concentricum, T. violaceum, T. schoenleinii, T. verrucosum, T. soudanense, Microsporum gypseum, M. equinum, Candida guilliermondii, Malassezia globosa, M. obtuse, M. restricta, M. slooffiae, and Aspergillus flavus. In another exemplary embodiment, the fungus or yeast is a member selected from dermatophytes, Trichophyton, Microsporum, Epidermophyton Aspergillus, Blastomyces, Candida, Coccidioides, Cryptococcus, Hendersonula, Histoplasma, Paecilomyces, Paracoccidioides, Pneumocystis, Trichosporium and yeast-like fungi.

IV. b) Methods Involving Bacteria

In an exemplary embodiment, the microorganism is a bacteria. In an exemplary embodiment, the bacteria is a gram-positive bacteria. In another exemplary embodiment, the gram-positive bacteria is a member selected from Staphylococcus species, Streptococcus species, Bacillus species, Mycobacterium species, Corynebacterium species (Propionibacterium species), Clostridium species, Actinomyces species, Enterococcus species and Streptomyces species. In another exemplary embodiment, the bacteria is a gram-negative bacteria. In another exemplary embodiment, the gram-negative bacteria is a member selected from Acinetobacter species, Neisseria species, Pseudomonas species, Brucella species, Agrobacterium species, Bordetella species, Escherichia species, Shigelia species, Yersinia species, Salmonella species, Klebsiella species, Enterobacter species, Haemophilus species, Pasteurella species, Streptobacillus species, spirochetal species, Campylobacter species, Vibrio species and Helicobacter species. In another exemplary embodiment, the bacterium is a member selected from Propionibacterium acnes; Staphylococcus aureus; Staphylococcus epidermidis, Staphylococcus saprophyticus; Streptococcus pyogenes; Streptococcus agalactiae; Streptococcus pneumoniae; Enterococcus faecalis; Enterococcus faecium; Bacillus anthracis; Mycobacterium avium-intracellulare; Mycobacterium tuberculosis, Acinetobacter baumanii; Corynebacterium diphtheria; Clostridium perfringens; Clostridium botulinum; Clostridium tetani; Neisseria gonorrhoeae; Neisseria meningitidis; Pseudomonas aeruginosa; Legionella pneumophila; Escherichia coli; Yersinia pestis; Haemophilus influenzae; Helicobacter pylori; Campylobacter fetus; Campylobacter jejuni; Vibrio cholerae; Vibrio parahemolyticus; Trepomena pallidum; Actinomyces israelii; Rickettsia prowazekii; Rickettsia rickettsii; Chlamydia trachomatis; Chlamydia psittaci; Brucella abortus; Agrobacterium tumefaciens; and Francisella tularensis.

In an exemplary embodiment, the bacteria is a member selected from acid-fast bacterium, including Mycobacterium species; bacilli, including Bacillus species, Corynebacterium species (also Propionibacterium) and Clostridium species; filamentous bacteria, including Actinomyces species and Streptomyces species; bacilli, such as Pseudomonas species, Brucella species, Agrobacterium species, Bordetella species, Escherichia species, Shigella species, Yersinia species, Salmonella species, Klebsiella species, Enterobacter species, Haemophilus species, Pasteurella species, and Streptobacillus species; spirochetal species, Campylobacter species, Vibrio species; and intracellular bacteria including Rickettsiae species and Chlamydia species.

The compounds of use in the invention are active against a variety of bacterial organisms. They are active against both Gram positive and Gram negative aerobic and anaerobic bacteria, including staphylococci, for example S. aureus; enterococci, for example E. faecalis; streptococci, for example S. pneumoniae; haemophili, for example H. influenza; Moraxella, for example M. catarrhalis; and Escherichia, for example E. coli. The compounds of use in the present invention are also active against mycobacteria, for example M. tuberculosis. The compounds of use in the present invention are also active against intercellular microbes, for example Chlamydia and Rickettsiae. The compounds of use in the present invention are also active against mycoplasma, for example M. pneumoniae.

In addition, compounds of use in this invention are active against staphylococci organisms such as S. aureus and coagulase negative strains of staphylocci such as S. epidermidis which are resistant (including multiply-resistant) to other anti-bacterial agents, for instance, β-lactam antibiotics such as, for example, methicillin, acrolides, aminoglycosides, and lincosamides. Compounds of use in the present invention are therefore useful in the treatment of MRSA, MRCNS and MRSE. Compounds of use in the present invention are also active against vancomycin resistant strains of strains of E. faecalis and, therefore, of use in treating infections associated with VRE organisms. Furthermore, compounds of use in the present invention are useful in the treatment of staphylococci organisms which are resistant to mupirocin.

In another exemplary embodiment, the bacteria is a member selected from Actinobacillus species, Porphyromonas species, Tannerella species, Prevotella species, Eubacterium species, Treponema species, Bulleidia species, Mogibacterium species, Slackia species, Campylobacter species, Eikenella species, Peptostreptococcus species, Peptostreptococcus species, Capnocytophaga species, Fusobacterium species, Porphyromonas species and Bacteroides species. In yet another exemplary embodiment, the bacteria is a member selected from Actinobacillus actinomycetemcomitans, Porphyromonas gingivalis, Tannerella forsythensis, Prevotella intermedia, Eubacterium nodatum, Treponema denticola, Bulleidia extructa, Mogibacterium timidum Slackia exigua, Campylobacter rectus, Eikenella corrodens, Peptostreptococcus micros, Peptostreptococcus anaerobius, Capnocytophaga ochracea, Fusobacterium nucleatum, Porphyromonas asaccharolytica and Bacteroides forsythus. In another examplary embodiment, the compound is a member selected from 3-hydroxypyridine-2-carbonyloxy-bis(3-chloro-4-methylphenyl)-borane (bis(3-Chloro4-methylphenyl)borinic acid 3-hydroxypicolinate ester), 1,3-dihydro-5-fluoro-1-hydroxy-2,1-benzoxaborole, and 5-(4-cyanophenoxy)-1,3-dihydro-1-hydroxy-2,1-benzoxaborole. In another examplary embodiment, the compound is 3-hydroxypyridine-2-carbonyloxy-bis(3-chloro-4-methylphenyl)-borane.

IV. c) Methods Involving Viruses

The compounds of the invention are useful for the treatment of diseases of both animals and humans, involving viruses. In an exemplary embodiment, the microorganism is a virus. In an exemplary embodiment, the virus is a member selected from hepatitis A-B-C, human rhinoviruses, Yellow fever virus, human respiratory coronaviruses, Severe acute respiratory syndrome (SARS), respiratory syncytial virus, influenza viruses, parainfluenza viruses 1-4, human immunodeficiency virus 1 (HIV-1), human immunodeficiency virus 2 (HIV-2), Herpes simplex virus 1 (HSV-1), Herpes simplex virus 2 (HSV-2), human cytomegalovirus (HCMV), Varicella zoster virus, Epstein-Barr (EBV), polioviruses, coxsackieviruses, echoviruses, rubella virus, neuroderma-tropic virus, variola virus, papoviruses, rabies virus, dengue virus, and West Nile virus. In another exemplary embodiment, the virus is a member selected from picornaviridae, flaviviridae, coronaviridae, paramyxoviridae, orthomyxoviridae, retroviridae, herpesviridae and hepadnaviridae. In another exemplary embodiment, the virus is a member selected from a virus included in the following table:

TABLE A
Viruses

| | |
|---|---|
| Virus Category | Pertinent Human Infections |

RNA Viruses

| | |
|---|---|
| Picomaviridae | Polio |
| | Human hepatitis A |
| | Human rhinovirus |
| Togaviridae and | Rubella - German measles |
| Flaviviridae | Yellow fever |
| Coronaviridae | Human respiratory coronavirus (HCV) |
| | Severe acute respiratory syndrome (SAR) |
| Rhabdoviridae | Lyssavirus - Rabies |
| Paramyxoviridae | Paramyxovirus - Mumps |
| | Morbillvirus - measles |
| | Pneumovirus - respiratory syncytial virus |
| Orthomyxoviridae | Influenza A-C |
| Bunyaviridae | Bunyavirus - Bunyamwera (BUN) |
| | Hantavirus - Hantaan (HTN) |
| | Nairevirus - Crimean-Congo hemorrhagic |
| | fever (CCHF) |
| | Phlebovirus - Sandfly fever (SFN) |
| | Uukuvirus - Uukuniemi (UUK) |
| | Rift Valley Fever (RVFN) |
| Arenaviridae | Junin - Argentine hemorrhagic fever |
| | Machupo - Bolivian hemorrhagic fever |
| | Lassa - Lassa fever |
| | LCM - aseptic lymphocyctic choriomeningitis |
| Reoviridae | Rotovirus |
| | Reovirus |
| | Orbivirus |
| Retroviridae | Human immunodeficiency virus 1 (HIV-1) |
| | Human immunodeficiency virus 2 (HIV-2) |
| | Simian immunodeficiency virus (SIV) |

DNA Viruses

| | |
|---|---|
| Papovaviridae | Pediatric viruses that reside in kidney |
| Adenoviridae | Human respiratory distress and some deep-seated |
| | eye infections |
| Parvoviridae | Human gastro-intestinal distress (Norwalk Virus) |
| Herpesviridae | Herpes simplex virus 1 (HSV-1) |
| | Herpes simplex virus 2 (HSV-2) |
| | Human cytomegalovirus (HCMV) |
| | Varicella zoster virus (VZV) |
| | Epstein-Barr virus (EBV) |
| | Human herpes virus 6 (HHV6) |
| Poxviridae | Orthopoxvirus is sub-genus for smallpox |
| Hepadnaviridae | Hepatitis B virus (HBV) |
| | Hepatitis C virus (HCV) |

IV. d) Methods of Treating Diseases Involving Parasites

The compounds of the invention are useful for the treatment of diseases of both animals and humans, involving parasites, including protozoa and helminths. Examples of such parasitic species include, among others, Entamoeba, Leishmania, Toxoplasma, Trichinella and Schistosoma. In an exemplary embodiment, the parasite is a member selected from Plasmodium falciparum, P. vivax, P. ovale P. malariae, P. berghei, Leishmania donovani, L. infantum, L. chagasi, L. mexicana, L. amazonensis, L. venezuelensis, L. tropics, L. major, L. minor, L. aethiopica, L. Biana braziliensis, L. (V.) guyanensis, L. (V.) panamensis, L. (V.) peruviana, Trypanosoma brucei rhodesiense, T. brucei gambiense, T. cruzi, Giardia intestinalis, G. lambda, Toxoplasma gondii, Entamoeba histolytica, Trichomonas vaginalis, Pneumocystis carinii, and Cryptosporidium parvum. In an exemplary embodiment, the disease caused by the parasite is a member selected from malaria, Chagas' disease, Leishmaniasis, African sleeping sickness (African human trypanosomiasis), giardiasis, toxoplasmosis, amebiasis and cryptosporidiosis.

V. Methods of Treating or Preventing Periodontal Disease

In another aspect, the invention provides a method of treating or preventing periodontal disease, or both. The method includes administering to the animal a therapeutically effective amount of a compound of the invention. In an exemplary embodiment, the compound is a member selected from a boron-containing compound described herein, sufficient to treat or prevent said disease. In another examplary embodiment, the compound is a member selected from 3-hydroxypyridine-2-carbonyloxy-bis(3-chloro-4-methylphenyl)-borane (bis(3-Chloro4-methylphenyl)borinic acid 3-hydroxypicolinate ester), 1,3-dihydro-5-fluoro-1-hydroxy-2,1-benzoxaborole, and 5-(4-cyanophenoxy)-1,3-dihydro-1-hydroxy-2,1-benzoxaborole. In another examplary embodiment, the compound is 3-hydroxypyridine-2-carbonyloxy-bis(3-chloro-4-methylphenyl)-borane. In another exemplary embodiment, the animal is a member selected from human, cattle, deer, reindeer, goat, honey bee, pig, sheep, horse, cow, bull, dog, guinea pig, gerbil, rabbit, cat, camel, yak, elephant, ostrich, otter, chicken, duck, goose, guinea fowl, pigeon, swan, and turkey. In another exemplary embodiment, the animal is a human. In another exemplary embodiment, the animal is a member selected from a human, cattle, goat, pig, sheep, horse, cow, bull, dog, guinea pig, gerbil, rabbit, cat, chicken and turkey. In another exemplary embodiment, the infection is a member selected from a gingivitis, periodontitis or juvenile/acute periodontitis.

The invention is further illustrated by the Examples that follow. The Examples are not intended to define or limit the scope of the invention.

### EXAMPLES

General: Melting points were obtained using a MeI-Temp-II melting point apparatus and are uncorrected. ¹H NMR spectra were recorded on Oxford 300 (300 MHz) spectrometer (Varian). Mass spectra were determined on API 3000 (Applied Biosystems). Purity by HPLC (relative area) was determined using ProStar Model 330 (PDA detector, Varian), Model 210 (pump, Varian), and a BetaBasic-18 4.6×150 mm column (Thermo Electron Corporation) with a linear gradient of 0 to 100% MeCN in 0.01% H₃PO₄ over 10 min followed by 100% MeCN for another 10 min at 220 nm.

### Example 1

### Preparation of 3 from 1

1.1 Reduction of Carboxylic Acid

To a solution of 1 (23.3 mmol) in anhydrous THF (70 mL) under nitrogen was added dropwise a BH₃ THF solution (1.0 M, 55 mL, 55 mmol) at 0° C. and the reaction mixture was stirred overnight at room temperature. Then the mixture was cooled again with ice bath and MeOH (20 mL) was added dropwise to decompose excess BH₃. The resulting mixture was stirred until no bubble was released and then 10% NaOH (10 mL) was added. The mixture was concentrated and the residue was mixed with water (200 mL) and extracted with EtOAc. The residue from rotary evaporation was purified by flash column chromatography over silica gel to give 20.7 mmol of 3.

1.2 Results

Exemplary compounds of structure 3 prepared by the method above are provided below.

### 1.2.a 2-Bromo-5-chlorobenzyl Alcohol

¹H NMR (300 MHz, DMSO-d₆): δ 7.57 (d, J=8.7 Hz, 1H), 7.50-7.49 (m, 1H), 7.28-7.24 (m, 1H), 5.59 (t, J=6.0 Hz, 1H) and 4.46 (d, J=6.0 Hz, 2H) ppm.

### 1.2.b 2-Bromo-5-methoxybenzyl Alcohol

¹H NMR (300 MHz, DMSO-d₆): δ 7.42 (d, J=8.7 Hz, 1H), 7.09 (d, J=2.4 Hz, 1H), 6.77 (dd, J₁=3 Hz, J₂=3 Hz, 1H), 5.43 (t, J=5.7 Hz, 1H), 4.44 (d, J=5.1 Hz, 2H), 3.76 (s, 3H).

### Example 2

### Preparation of 3 from 2

2.1. Reduction of Aldehyde

To a solution of 2 (Z=H, 10.7 mmol) in methanol (30 mL) was added sodium borohydride (5.40 mol), and the mixture was stirred at room temperature for 1 h. Water was added, and the mixture was extracted with ethyl acetate. The organic layer was washed with brine and dried on anhydrous sodium sulfate. The solvent was removed under reduced pressure to afford 9.9 mmol of 3.

2.2 Results

Exemplary compounds of structure 3 prepared by the method above are provided below.

### 2.2.a 2-Bromo-5-(4-cyanophenoxy)benzyl Alcohol

¹H-NMR (300 MHz, CDCl₃) δ (ppm) 2.00 (br s, 1H), 4.75 (s, 2H), 6.88 (dd, J=8.5, 2.9 Hz, 1H), 7.02 (d, J=8.8 Hz, 1H), 7.26 (d, J=2.6 Hz, 1H), 7.56 (d, J=8.5 Hz, 1H), 7.62 (d, J=8.8 Hz, 2H).

### 2.2.b 2-Bromo-4-(4-cyanophenoxy)benzyl Alcohol

¹H NMR (300 MHz, DMSO-d₆): δ 7.83 (d, 2H), 7.58 (d, 1H), 7.39 (d, 1H), 7.18 (dd, 1H), 7.11 (d, 2H), 5.48 (t, 1H) and 4.50 (d, 2H) ppm.

### 2.2.c 5-(4-Cyanophenoxy)-1-Indanol

M.p. 50-53° C. MS (ESI+): m/z=252 (M+1). HPLC: 99.7% purity at 254 nm and 99.0% at 220 nm. ¹H NMR (300 MHz, DMSO-d₆): δ 7.80 (d, 2H), 7.37 (d, 1H), 7.04 (d, 2H), 6.98-6.93 (m, 2H), 5.27 (d, 1H), 5.03 (q, 1H), 2.95-2.85 (m, 1H), 2.75-2.64 (m, 1H), 2.39-2.29 (m, 1H) and 1.85-1.74 (m, 1H) ppm.

### 2.2.d 2-Bromo-5-(tert-butyldimethylsiloxy)benzyl Alcohol

¹H-NMR (300 MHz, CDCl₃) δ (ppm) 0.20 (s, 6H), 0.98 (s, 9H), 4.67 (br s, 1H), 6.65 (dd, J=8.2, 2.6 Hz, 1H), 6.98 (d, J=2.9 Hz, 1H), 7.36 (d, J=8.8 Hz, 1H).

Additional examples of compounds which can be produced by this method include 2-bromo-4-(3-cyanophenoxy)benzyl alcohol; 2-bromo-4-(4-chlorophenoxy)benzyl alcohol; 2-bromo-4-phenoxybenzyl alcohol; 2-bromo-5-(3,4-dicyanophenoxy)benzyl alcohol; 2-(2-bromo-5-fluorophenyl)ethyl alcohol; 2-bromo-5-fluorobenzyl alcohol; and 1-bromo-2-naphthalenemethanol.

### Example 3

### Preparation of 4 from 3

3.1 Protective Alkylation

Compound 3 (20.7 mmol) was dissolved in CH₂Cl₂ (150 mL) and cooled to 0° C. with ice bath. To this solution under nitrogen were added in sequence N,N-di-isopropyl ethyl amine (5.4 mL, 31.02 mmol, 1.5 eq) and chloromethyl methyl ether (2 mL, 25.85 mmol, 1.25 eq). The reaction mixture was stirred overnight at room temperature and washed with NaHCO₃-saturated water and then NaCl-saturated water. The residue after rotary evaporation was purified by flash column chromatography over silica gel to give 17.6 mmol of 4.

3.2 Results

Exemplary compounds of structure 4 prepared by the method above are provided below.

### 3.2.a 2-Bromo-5-chloro-1-(methoxymethoxymethyl)benzene

¹H NMR (300 MHz, DMSO-d₆): δ 7.63 (d, J=8.7 Hz, 1H), 7.50 (dd, J=2.4 & 0.6 Hz, 1H), 7.32 (dd, J=8.4 & 2.4 Hz, 1H), 4.71 (s, 2H), 4.53 (s, 2H) and 3.30 (s, 3H) ppm.

### 3.2.b 2-Bromo-5-fluoro-1-[1-(methoxymethoxy)ethyl]benzene

¹H-NMR (300.058 MHz, CDCl₃) δ ppm 1.43 (d, J=6.5 Hz, 3H), 3.38 (s, 3H), 4.55 (d, J=6.5 Hz, 1H), 4.63 (d, J=6.5 Hz, 1H), 5.07 (q, J=6.5 Hz, 1H), 6.85 (m, 1H), 7.25 (dd, J=9.7, 2.6 Hz, 1H), 7.46 (dd, J=8.8, 5.3 Hz, 1H).

### 3.2.c 2-Bromo-5-fluoro-1-[2-(methoxymethoxy)ethyl]benzene

¹H-NMR (300.058 MHz, CDCl₃) δ ppm 3.04 (t, J=6.7 Hz, 2H), 3.31 (s, 3H), 3.77 (t, J=6.7 Hz, 2H), 4.62 (s, 2H), 6.82 (td, J=8.2, 3.2 Hz, 1H), 7.04 (dd, J=9.4, 2.9 Hz, 1H), 7.48 (dd, J=8.8, 5.3 Hz, 1H).

### 3.2.d 2-Bromo-4,5-difluoro-1-(methoxymethoxymethyl)benzene

¹H-NMR (300.058 MHz, CDCl₃) δ ppm 3.42 (s, 3H), 4.57 (d, J=1.2 Hz, 2H), 4.76 (s, 2H), 7.3-7.5 (m, 2H).

### 3.2.e 2-Bromo-5-cyano-1-(methoxymethoxymethyl)benzene

¹H-NMR (300.058 MHz, CDCl₃) δ ppm 3.43 (s, 3H), 4.65 (s, 2H), 4.80 (s, 2H), 7.43 (dd, J=8.2, 4.1 Hz, 1H), 7.66 (d, J=8.2 Hz, 1H), 7.82 (d, J=4.1 Hz, 1H).

### 3.2.f 2-Bromo-5-methoxy-1-(methoxymethoxymethyl)benzene

¹H NMR (300 MHz, DMSO-d₆): δ 7.48 (dd, J₁=1.2 Hz, J₂=1.2 Hz, 1H), 7.05 (d, J=2.7 Hz, 1H), 6.83 (dd, J₂, =3 Hz, J₂=3 Hz, 1H), 4.69 (d, J=1.2 Hz, 2H), 4.5 (s, 2H), 3.74 (d, J=1.5 Hz, 3H), 3.32 (d, J=2.1 Hz, 3H) ppm.

### 3.2.g 1-Benzyl-1-(2-bromophenyl)-1-(methoxymethoxy)ethane

¹H NMR (300 MHz, DMSO-d₆): δ 7.70-7.67 (m, 1H), 7.25-7.09 (m, 6H), 6.96-6.93 (m, 2H), 4.61 (d, 1H), 4.48 (d, 1H), 3.36-3.26 (m, 2H), 3.22 (s, 3H) and 1.63 (s, 3H) ppm.

### 3.2.h 2-Bromo-6-fluoro-1-(methoxymethoxymethyl)benzene

¹H-NMR (300 MHz, CDCl₃) δ (ppm) 3.43 (s, 3H), 4.74 (s, 2H), 4.76 (d, J=2.1 Hz, 2H), 7.05 (t, J=9.1 Hz, 1H), 7.18 (td, J=8.2, 5.9 Hz, 1H), 7.40 (d, J=8.2 Hz, 1H).

### 3.2.i 2-Bromo-4-(4-cyanophenoxy)-1-(methoxymethoxymethyl)benzene

¹H NMR (300 MHz, DMSO-d₆): δ 7.84 (d, 2H), 7.56 (d, 1H), 7.44 (d, 1H), 7.19-7.12 (m, 3H), 4.69 (s, 2H), 4.56 (s, 2H) and 3.31 (s, 3H) ppm.

### 3.2.j 2-Bromo-5-(tert-butyldimethylsiloxy)-1-(methoxymethoxymethyl)benzene

¹H-NMR (300 MHz, CDCl₃) δ (ppm) 0.19 (s, 6H), 0.98 (s, 9H), 3.43 (s, 3H), 4.59 (s, 2H), 4.75 (s, 2H), 6.64 (dd, J=8.5, 2.9 Hz, 1H), 6.98 (d, J=2.9 Hz, 1H), 7.36 (d, J=8.5 Hz, 1H).

### 3.2.k 2-Bromo-5-(2-cyanophenoxy)-1-(methoxymethoxymethyl)benzene

¹H-NMR (300 MHz, CDCl₃) δ (ppm) 3.41 (s, 3H), 4.64 (s, 2H), 4.76 (s, 2H), 6.8-6.9 (m, 2H), 7.16 (td, J=7.6, 0.9 Hz, 1H), 7.28 (d, J=2.9 Hz, 1H), 7.49 (ddd, J=8.8, 7.6, 1.8 Hz, 1H), 7.56 (d, J=8.5 Hz, 1H), 7.67 (dd, J=7.9, 1.8 Hz, 1H).

### 3.2.l 2-Bromo-5-phenoxy-1-(methoxymethoxymethyl)benzene

¹H-NMR (300 MHz, CDCl₃) δ (ppm) 3.40 (s, 3H), 4.62 (s, 2H), 4.74 (s, 2H), 6.80 (dd, J=8.8, 2.9 hz, 1H), 7.01 (d, J=8.5 Hz, 2H), 7.12 (t, J=7.9 Hz, 1H), 7.19 (d, J=2.9 hz, 1H), 7.35 (t, J=7.6 Hz, 2H), 7.48 (d, J=8.5 Hz, 1H).

Additional examples of compounds which can be produced by this method include 2-bromo-1-(methoxymethoxymethyl)benzene; 2-bromo-5-methyl-1-(methoxymethoxymethyl)benzene; 2-bromo-5-(methoxymethoxymethyl)-1-(methoxymethoxymethyl)benzene; 2-bromo-5-fluoro-1-(methoxymethoxymethyl)benzene; 1-bromo-2-(methoxymethoxymethyl)naphthalene; 2-bromo-4-fluoro-1-(methoxymethoxymethyl)benzene; 2-phenyl-1-(2-bromophenyl)-1-(methoxymethoxy)ethane; 2-bromo-5-(4-cyanophenoxy)-1-(methoxymethoxy methyl)benzene; 2-bromo-4-(3-cyanophenoxy)-1-(methoxymethoxymethyl)benzene; 2-bromo-4-(4-chlorophenoxy)-1-(methoxymethoxymethyl)benzene; 2-bromo-4-phenoxy-1-(methoxymethoxymethyl)benzene; 2-bromo-5-(3,4-dicyanophenoxy)-1-(methoxymethoxymethyl)benzene.

### Example 4

### Preparation of I from 4 via 5

4.1 Metallation and Boronylation

To a solution of 4 (17.3 mmol) in anhydrous THF (80 mL) at −78° C. under nitrogen was added dropwise tert-BuLi or n-BuLi (11.7 mL) and the solution became brown colored. Then, B(OMe)₃ (1.93 mL, 17.3 mmol) was injected in one portion and the cooling bath was removed. The mixture was warmed gradually with stirring for 30 min and then stirred with a water bath for 2 h. After addition of 6N HCl (6 mL), the mixture was stirred overnight at room temperature and about 50% hydrolysis has happened as shown by TLC analysis. The solution was rotary evaporated and the residue was dissolved in MeOH (50 mL) and 6N HCl (4 mL). The solution was refluxed for 1 h and the hydrolysis was completed as indicated by TLC analysis. Rotary evaporation gave a residue which was dissolved in EtOAc, washed with water, dried and then evaporated. The crude product was purified by flash column chromatography over silica gel to provide a solid with 80% purity. The solid was further purified by washing with hexane to afford 7.2 mmol of I.

4.2 Results

Analytical data for exemplary compounds of structure I are provided below.

### 4.2.a 5-Chloro-1,3-dihydro-1-hydroxy-2,1-benzoxaborole

### 5-chlorobenzo[c][1,2]oxaborol-1(3H)-ol (C1)

M.p. 142-150° C. MS (ESI): m/z=169 (M+1, positive) and 167 (M−1, negative). HPLC (220 nm): 99% purity. ¹H NMR (300 MHz, DMSO-d₆): δ 9.30 (s, 1H), 7.71 (d, J=7.8 Hz, 1H), 7.49 (s, 1H), 7.38 (d, J=7.8 Hz, 1H) and 4.96 (s, 2H) ppm.

### 4.2.b 1,3-Dihydro-1-hydroxy-2,1-benzoxaborole

### benzo[c][1,2]oxaborol-1(3H)-ol (C2)

M.p. 83-86° C. MS (ESI): m/z=135 (M+1, positive) and 133 (M−1, negative). HPLC (220 nm): 95.4% purity. ¹H NMR (300 MHz, DMSO-d₆): δ 9.14 (s, 1H), 7.71 (d, J=7.2 Hz, 1H), 7.45 (t, J=7.5 Hz, 1H), 7.38 (d, J=7.5 Hz, 1H), 7.32 (t, J=7.1 Hz, 1H) and 4.97 (s, 2H) ppm.

### 4.2.c 5-chloro-3-methylbenzo[c][1,2]oxaborol-1(3H)-ol (C3)

¹H-NMR (300 MHz, DMSO-d₆) δ ppm 1.37 (d, J=6.4 Hz, 3H), 5.17 (q, J=6.4 Hz, 1H), 7.14 (m, 1H), 7.25 (dd, J=9.7, 2.3 Hz, 1H), 7.70 (dd, J=8.2, 5.9 Hz, 1H), 9.14 (s, 1H).

### 4.2.d 6-Fluoro-1-hydroxy-1,2,3,4-tetrahydro-2,1-benzoxaborine

### 6-fluoro-3,4-dihydrobenzo[c][1,2]oxaborinin-1-ol (C4)

¹H-NMR (300 MHz, DMSO-d₆) δ ppm 2.86 (t, J=5.9 Hz, 2H), 4.04 (t, J=5.9 Hz, 2H), 7.0-7.1 (m, 2H), 7.69 (dd, J=8.2, 7.2 Hz, 1H), 8.47 (s, 1H).

### 4.2.e 5,6-Difluoro-1,3-dihydro-1-hydroxy-2,1-benzoxaborole

### 5,6-difluorobenzo[c][1,2]oxaborol-1(3H)-ol (C5)

¹H-NMR (300 MHz, DMSO-d₆) δ ppm 4.94 (s, 2H), 7.50 (dd, J=10.7, 6.8 Hz, 1H), 7.62 (dd, J=9.7, 8.2 Hz, 1H), 9.34 (s, 1H).

### 4.2.f 5-Cyano-1,3-dihydro-1-hydroxy-2,1-benzoxaborole

### 1-hydroxy-1,3-dihydrobenzo[c][1,2]oxaborole-5-carbonitrile (C6)

¹H-NMR (300 MHz, DMSO-d₆) δ ppm 5.03 (s, 2H), 7.76 (d, J=8.2 Hz, 1H), 7.89 (d, J=8.2 Hz, 1H), 7.90 (s, 1H), 9.53 (s, 1H).

### 4.2.g 1,3-Dihydro-1-hydroxy-5-methoxy-2,1-benzoxaborole

### 5-methoxybenzo[c][1,2]oxaborol-1(3H)-ol (C7)

M.p. 102-104° C. MS ESI: m/z=165.3 (M+1) and 162.9 (M−1). ¹H NMR (300 MHz, DMSO-d₆): δ 8.95 (s, 1H), 7.60 (d, J=8.1 Hz, 1H), 6.94 (s, 1H), 6.88 (d, J=8.1 Hz, 1H), 4.91 (s, 2H), 3.77 (s, 3H) ppm.

### 4.2.h 1,3-Dihydro-1-hydroxy-5-methyl-2,1-benzoxaborole

### 5-methylbenzo[c][1,2]oxaborol-1(3H)-ol (C8)

M.p. 124-128° C. MS ESI: m/z=148.9 (M+1) and 146.9 (M−1). ¹H NMR (300 MHz, DMSO-d₆): δ 9.05 (s, 1H), 7.58 (d, J=7.2 Hz, 1H), 7.18 (s, 1H), 7.13 (d, J=7.2 Hz, 2H), 4.91 (s, 2H), 2.33 (s, 3H) ppm.

### 4.2.i 1,3-Dihydro-1-hydroxy-5-hydroxymethyl-2,1-benzoxaborole

### 5-(hydroxymethyl)benzo[c][1,2]oxaborol-1(3H)-ol (C9)

MS: m/z=163 (M−1, ESI−). ¹H NMR (300 MHz, DMSO-d₆): δ 9.08 (s, 1H), 7.64 (d, 1H), 7.33 (s, 1H), 7.27 (d, 1H), 5.23 (t, 1H), 4.96 (s, 2H), 4.53 (d, 2H) ppm.

### 4.2.j 1,3-Dihydro-5-fluoro-1-hydroxy-2,1-benzoxaborole

### 5-fluorobenzo[c][1,2]oxaborol-1(3H)-ol (C10)

M.p. 110-114° C. MS ESI: m/z=150.9 (M−1). ¹H NMR (300 MHz, DMSO-d₆): δ 9.20 (s, 1H), 7.73 (dd, J₁=6 Hz, J₂=6 Hz, 1H), 7.21 (m, 1H), 7.14 (m, 1H), 4.95 (s, 2H) ppm.

### 4.2.k 1,3-Dihydro-2-oxa-1-cyclopenta[\acute{α}]naphthalene

### naphtho[1,2-c][1,2]oxaborol-1(3H)-ol (C11)

M.P. 139-143° C. MS ESI: m/z=184.9 (M+1). ¹H NMR (300 MHz, DMSO-d₆): δ 9.21 (s, 1H), 8.28 (dd, J₁=6.9 Hz, J₂=0.6 Hz, 1H), 7.99 (d, J=8.1 Hz, 1H), 7.95 (d, J=7.5 Hz, 1H), 7.59-7.47 (m, 3H), 5.09 (s, 2H) ppm.

### 4.2.m 1,3-Dihydro-6-fluoro-1-hydroxy-2,1-benzoxaborole

### 6-fluorobenzo[c][1,2]oxaborol-1(3H)-ol (C13)

M.p. 110-117.5° C. MS (ESI): m/z=151 (M−1, negative). HPLC (220 nm): 100% purity. ¹H NMR (300 MHz, DMSO-d₆): δ 9.29 (s, 1H), 7.46-7.41 (m, 2H), 7.29 (td, 1H) and 4.95 (s, 2H) ppm.

### 4.2.n 3-Benzyl-1,3-dihydro-1-hydroxy-3-methyl-2,1-benzoxaborole

### 3-benzyl-3-methylbenzo[c][1,2]oxaborol-1(3H)-ol (C14)

MS (ESI): m/z=239 (M+1, positive). HPLC: 99.5% purity at 220 nm and 95.9% at 254 nm. ¹H NMR (300 MHz, DMSO-d₆): δ 8.89 (s, 1H), 7.49-7.40 (m, 3H), 7.25-7.19 (m, 1H), 7.09-7.05 (m, 3H), 6.96-6.94 (m, 2H), 3.10 (d, 1H), 3.00 (d, 1H) and 1.44 (s, 3H) ppm.

### 4.2.o 3-Benzyl-1,3-dihydro-1-hydroxy-2,1-benzoxaborole

### 3-benzylbenzo[c][1,2]oxaborol-1(3H)-ol (C15)

MS (ESI+): m/z=225 (M+1). HPLC: 93.4% purity at 220 nm. ¹H NMR (300 MHz, DMSO-d₆): δ 9.08 (s, 1H), 7.63 (dd, 1H), 7.43 (t, 1H), 7.35-7.14 (m, 7H), 5.38 (dd, 1H), 3.21 (dd, 1H) and 2.77 (dd, 1H) ppm.

### 4.2.p 1,3-Dihydro-4-fluoro-1-hydroxy-2,1-benzoxaborole

### 4-fluorobenzo[c][1,2]oxaborol-1(3H)-ol (C16)

¹H-NMR (300 MHz, DMSO-d₆) δ (ppm) 5.06 (s, 2H), 7.26 (ddd, J=9.7, 7.9, 0.6 Hz, 1H), 7.40 (td, J=8.2, 4.7 Hz, 1H), 7.55 (d, J=7.0 Hz, 1H), 9.41 (s, 1H).

### 4.2.q 5-(4-Cyanophenoxy)-1,3-dihydro-1-hydroxy-2,1-benzoxaborole

### 4-(1-hydroxy-1,3-dihydrobenzo[c][1,2]oxaborol-5-yloxy)benzonitrile (C17)

¹H-NMR (300 MHz, DMSO-d₆) δ ppm 4.95 (s, 2H), 7.08 (dd, J=7.9, 2.1 Hz, 1H), 7.14 (d, J=8.8 Hz, 1H), 7.15 (d, J=2.1 Hz, 1H), 7.78 (d, J=7.9 Hz, 1H), 7.85 (d, J=9.1 Hz, 2H), 9.22 (s, 1H).

### 4.2.r 6-(4-Cyanophenoxy)-1,3-dihydro-1-hydroxy-2,1-benzoxaborole

### 4-(1-hydroxy-1,3-dihydrobenzo[c][1,2]oxaborol-6-yloxy)benzonitrile (C18)

M.p. 148-151° C. MS: m/z=252 (M+1) (ESI+) and m/z=250 (M−1) (ESI−). HPLC: 100% purity at 254 nm and 98.7% at 220 nm. ¹H NMR (300 MHz, DMSO-d₆): δ 9.26 (s, 1H), 7.82 (d, 2H), 7.50 (d, 1H), 7.39 (d, 1H), 7.26 (dd, 1H), 7.08 (d, 2H) and 4.99 (s, 2H) ppm

### 4.2.s 6-(3-Cyanophenoxy)-1,3-dihydro-1-hydroxy-2,1-benzoxaborole

### 3-(1-hydroxy-1,3-dihydrobenzo[c][1,2]oxaborol-6-yloxy)benzonitrile (C19)

M.p. 146-149° C. MS: m/z=252 (M+1) (ESI+) and m/z=250 (M−1) (ESI−). HPLC: 100% purity at 254 nm and 97.9% at 220 nm. ¹H NMR (300 MHz, DMSO-d₆): δ 9.21 (s, 1H), 7.60-7.54 (m, 2H), 7.50-7.45 (m, 2H), 7.34-7.30 (m, 2H), 7.23 (dd, 1H) and 4.98 (s, 2H) ppm.

### 4.2.t 6-(4-Chlorophenoxy)-1,3-dihydro-1-hydroxy-2,1-benzoxaborole

### 6-(4-chlorophenoxy)benzo[c][1,2]oxaborol-1(3H)-ol (C20)

M.p. 119-130° C. MS: m/z=261 (M+1) (ESI+) and m/z=259 (M−1) (ESI−). HPLC: 100% purity at 254 nm and 98.9% at 220 nm. ¹H NMR (300 MHz, DMSO-d₆): δ 9.18 (s, 1H), 7.45-7.41 (m, 3H), 7.29 (d, 1H), 7.19 (dd, 1H), 7.01 (d, 2H) and 4.96 (s, 2H) ppm.

### 4.2.u 6-Phenoxy-1,3-dihydro-1-hydroxy-2,1-benzoxaborole

### 6-phenoxybenzo[c][1,2]oxaborol-1(3H)-ol (C21)

M.p. 95-99° C. MS: m/z=227 (M+1) (ESI+) and m/z=225 (M−1) (ESI−). HPLC: 100% purity at 254 nm and 98.4% at 220 nm. ¹H NMR (300 MHz, DMSO-d₆): δ 9.17 (s, 1H), 7.43-7.35 (m, 3H), 7.28 (s, 1H), 7.19-7.09 (m, 2H), 6.99 (d, 2H) and 4.96 (s, 2H) ppm.

### 4.2.v 5-(4-Cyanobenzyloxy)-1,3-dihydro-1-hydroxy-2,1-benzoxaborole

### 4-((1-hydroxy-1,3-dihydrobenzo[c][1,2]oxaborol-5-yloxy)methyl)benzonitrile (C22)

¹H-NMR (300 MHz, DMSO-d₆) δ (ppm) 4.90 (s, 2H), 5.25 (s, 2H), 6.98 (dd, J=7.9, 2.1 Hz, 1H), 7.03 (d, J=1.8 Hz, 1H), 7.62 (d, J=7.9 Hz, 1H), 7.64 (d, J=8.5 Hz, 2H), 7.86 (d, J=8.5 Hz, 1H), 9.01 (s, 1H).

### 4.2.w 5-(2-Cyanophenoxy)-1,3-dihydro-1-hydroxy-2,1-benzoxaborole

### 2-(1-hydroxy-1,3-dihydrobenzo[c][1,2]oxaborol-5-yloxy)benzonitrile (C23)

¹H-NMR (300 MHz, DMSO-d₆) δ (ppm) 4.95 (s, 2H), 7.0-7.2 (m, 3H), 7.32 (td, J=7.6, 1.2 Hz, 1H), 7.68 (ddd, J=9.1, 7.6, 1.8 Hz, 1H), 7.77 (d, J=7.9 Hz, 1H), 7.91 (dd, J=7.9, 1.8 Hz, 1H).

### 4.2.x 5-Phenoxy-1,3-dihydro-1-hydroxy-2,1-benzoxaborole

### 5-phenoxybenzo[c][1,2]oxaborol-1(3H)-ol (C24)

¹H-NMR (300 MHz, DMSO-d₆) δ (ppm) 4.91 (s, 2H), 6.94 (s, 1H), 6.96 (d, J=8.8 Hz, 1H), 7.05 (d, J=7.6 Hz, 2H), 7.17 (t, J=7.3 Hz, 1H), 7.41 (t, J=7.3 Hz, 2H), 7.70 (d, J=8.5 Hz, 1H), 9.11 (s, 1H).

### 4.2.y 5-[4-(N,N-Diethylcarbamoyl)phenoxy]-1,3-dihydro-1-hydroxy-2,1-benzoxaborole

### N,N-diethyl-4-(1-hydroxy-1,3-dihydrobenzo[c][1,2]oxaborol-5-yloxy)benzamide (C25)

¹H-NMR (300 MHz, DMSO-d₆) δ (ppm) 1.08 (br s, 6H), 3.1-3.5 (m, 4H), 4.93 (s, 2H), 7.0-7.1 (m, 4H), 7.37 (d, J=8.5 Hz, 2H), 7.73 (d, J=7.9 Hz, 1H), 9.15 (s, 1H).

### 4.2.z 1,3-Dihydro-1-hydroxy-5-[4-(morpholinocarbonyl)phenoxy]-2,1-benzoxaborole

### (4-(1-hydroxy-1,3-dihydrobenzo[c][1,2]oxaborol-5-yloxy)phenyl (morpholino)methanone (C26)

¹H-NMR (300 MHz, DMSO-d₆) δ (ppm) 3.3-3.7 (m, 8H), 4.93 (s, 2H), 7.0-7.1 (m, 4H), 7.44 (d, J=8.8 Hz, 2H), 7.73 (d, J=7.9 Hz, 1H), 9.16 (s, 1H).

### 4.2.aa 5-(3,4-Dicyanophenoxy)-1,3-dihydro-1-hydroxy-2,1-benzoxaborole

### 4-(1-hydroxy-1,3-dihydrobenzo[c][1,2]oxaborol-5-yloxy)phthalonitrile (C27)

¹H-NMR (300 MHz, DMSO-d₆) δ (ppm) 4.97 (s, 2H), 7.13 (dd, J=7.9, 2.1 Hz, 1H), 7.21 (d, J=1.5 Hz, 1H), 7.43 (dd, J=8.8, 2.6 Hz, 1H), 7.81 (d, J=7.9 Hz, 1H), 7.82 (d, J=2.6 Hz, 1H), 8.11 (d, J=8.5 Hz, 1H), 9.26 (s, 1H).

### 4.2.ab 6-Phenylthio-1,3-dihydro-1-hydroxy-2,1-benzoxaborole 6-(phenylthio)benzo[c][1,2]oxaborol-1(3H)-ol (C28)

M.p. 121-124° C. MS: m/z=243 (M+1) (ESI+) and m/z=241 (M−1) (ESI−). HPLC: 99.6% purity at 254 nm and 99.6% at 220 nm. ¹H NMR (300 MHz, DMSO-d₆): δ 9.25 (s, 1H), 7.72 (dd, 1H), 7.48 (dd, 1H), 7.43 (dd, 1H), 7.37-7.31 (m, 2H), 7.29-7.23 (m, 3H), and 4.98 (s, 2H) ppm.

### 4.2.ac 6-(4-trifluoromethoxyphenoxy)-1,3-dihydro-1-hydroxy-2,1-benzoxaborole

### 6-(4-(trifluoromethoxy)phenoxy)benzo[c][1,2]oxaborol-1(3H)-ol (C29)

M.p. 97-101° C. MS: m/z=311 (M+1) (ESI+) and m/z=309 (M−1) (ESI−). HPLC: 100% purity at 254 nm and 100% at 220 nm. ¹H NMR (300 MHz, DMSO-d₆): δ 9.20 (s, 1H), 7.45 (d, 1H), 7.37 (d, 2H), 7.33 (d, 1H), 7.21 (dd, 1H), 7.08 (d, 2H), and 4.97 (s, 2H) ppm.

### 4.2.ad 5-(N-Methyl-N-phenylsulfonylamino)-1,3-dihydro-1-hydroxy-2,1-benzoxaborole

### N-(1-hydroxy-1,3-dihydrobenzo[c][1,2]oxaborol-5-yl)-N-methylbenzenesulfonamide (C30)

M.p. 85-95° C. MS: m/z=304 (M+1) (ESI+) and m/z=302 (M−1) (ESI−). HPLC: 96.6% purity at 254 nm and 89.8% at 220 nm. ¹H NMR (300 MHz, DMSO-d₆): δ 9.23 (s, 1H), 7.72-7.63 (m, 2H), 7.56 (t, 2H), 7.50 (d, 2H), 7.16 (s, 1H), 7.03 (d, 1H), 4.91 (s, 2H) and 3.14 (s, 3H) ppm.

### 4.2.ae 6-(4-Methoxyphenoxy)-1,3-dihydro-1-hydroxy-2,1-benzoxaborole

### 6-(4-methoxyphenoxy)benzo[c][1,2]oxaborol-1(3H)-ol (C31)

M.p. 126-129° C. MS: m/z=257 (M+1) (ESI+) and m/z=255 (M−1) (ESI−). HPLC: 98.4% purity at 254 nm and 98.4% at 220 nm. ¹H NMR (300 MHz, DMSO-d₆): δ 9.14 (s, 1H), 7.36 (d, 1H), 7.19 (s, 1H), 7.12 (d, 1H), 6.98 (d, 2H), 6.95 (d, 2H), 4.93 (s, 2H) and 3.73 (s, 3H) ppm.

### 4.2.af 6-(4-Methoxyphenylthio)-1,3-dihydro-1-hydroxy-2,1-benzoxaborole

### 6-(4-methoxyphenylthio)benzo[c][1,2]oxaborol-1(3H)-ol (C32)

M.p. 95-100° C. MS: m/z=272 (M+), 273 (M+1) (ESI+) and m/z=271 (M−1) (ESI−). HPLC: 100% purity at 254 nm and 99.2% at 220 nm. ¹H NMR (300 MHz, DMSO-d₆): δ 9.20 (s, 1H), 7.51 (d, 1H), 7.39-7.28 (m, 4H), 6.98 (d, 2H), 4.93 (s, 2H) and 3.76 (s, 3H) ppm.

### 4.2.ag 6-(4-Methoxyphenylsulfonyl)-1,3-dihydro-1-hydroxy-2,1-benzoxaborole

### 6-(4-methoxyphenylsulfonyl)benzo[c][1,2]oxaborol-1(3H)-ol (C33)

M.p. 180-192° C. MS: m/z=305 (M+1) (ESI+) and m/z=303 (M−1) (ESI−). HPLC: 96.8% purity at 254 nm and 95.5% at 220 nm. ¹H NMR (300 MHz, DMSO-d₆): δ 9.46 (s, 1H), 8.28 (s, 1H), 7.99 (d, 1H), 7.85 (d, 2H), 7.61 (d, 1H), 7.11 (d, 2H), 5.02 (s, 2H) and 3.80 (s, 3H) ppm.

### 4.2.ah 6-(4-Methoxyphenylsulfinyl)-1,3-dihydro-1-hydroxy-2,1-benzoxaborole

### 6-(4-methoxyphenylsulfinyl)benzo[c][1,2]oxaborol-1(3H)-ol (C34)

¹H NMR (300 MHz, DMSO-d₆): δ 9.37 (s, 1H), 8.02 (d, 1H), 7.71 (dd, 1H), 7.59 (d, 2H), 7.53 (d, 1H), 7.07 (d, 2H), 5.00 (s, 2H) and 3.76 (s, 3H) ppm.

### 4.2.ai 5-Trifluoromethyl-1,3-dihydro-1-hydroxy-2,1-benzoxaborole

### 5-(trifluoromethyl)benzo[c][1,2]oxaborol-1(3H)-ol (C35)

M.p. 113-118° C. MS: m/z=203 (M+1) (ESI+) and m/z=201 (M−1) (ESI−). HPLC: 100% purity at 254 nm and 100% at 220 nm. ¹H NMR (300 MHz, DMSO-d₆): δ 9.48 (s, 1H), 7.92 (d, 1H), 7.78 (s, 1H), 7.67 (d, 1H) and 5.06 (s, 2H) ppm.

### 4.2.aj 4-(4-Cyanophenoxy)-1,3-dihydro-1-hydroxy-2,1-benzoxaborole

### 4-(1-hydroxy-1,3-dihydrobenzo[c][1,2]oxaborol-4-yloxy)benzonitrile (C36)

For coupling reaction between 4-fluorobenzonitrile and substituted phenol to give starting material 2, see Igarashi, S.; et al. Chemical & Pharmaceutical Bulletin (2000), 48(11), 1689-1697.

¹H-NMR (300 MHz, DMSO-d₆) (ppm) 4.84 (s, 2H), 7.08 (d, J=8.2 Hz, 2H), 7.18 (d, J=7.9 Hz, 1H), 7.45 (t, J=7.3 Hz, 1H), 7.63 (d, J=7.3 Hz, 1H), 7.82 (d, J=8.5 Hz, 2H).

### 4.2.ak 5-(3-Cyanophenoxy)-1,3-dihydro-1-hydroxy-2,1-benzoxaborole

### 3-(1-hydroxy-1,3-dihydrobenzo[c][1,2]oxaborol-5-yloxy)benzonitrile (C37)

For coupling between 3-fluorobenzonitrile and substituted phenol to give starting material 2: Li, F. et al., Organic Letters (2003), 5(12), 2169-2171.

¹H-NMR (300 MHz, DMSO-d₆) (ppm) 4.93 (s, 2H), 7.0-7.1 (m, 2H), 7.3-7.4 (m, 1H), 7.5-7.7 (m, 3H), 7.75 (d, J=8.2 Hz, 1H).

### 4.2.al 5-(4-Carboxyphenoxy)-1,3 dihydro-1-hydroxy-2,1-benzoxaborole

### 4-(1-hydroxy-1,3-dihydrobenzo[c][1,2]oxaborol-5-yloxy)benzoic acid (C38)

To a solution of 5-(4-cyanophenoxy)-1-hydroxy-2,1-benzoxaborole obtained in C17 (430 mg, 1.71 mmol) in ethanol (10 mL) was added 6 mol/L sodium hydroxide (2 mL), and the mixture was refluxed for 3 hours. Hydrochloric acid (6 mol/L, 3 mL) was added, and the mixture was extracted with ethyl acetate. The organic layer was washed with brine and dried on anhydrous sodium sulfate. The solvent was removed under reduced pressure, and the residue was purified by silica gel column chromatography (ethyl acetate) followed by trituration with diisopropyl ether to give the target compound (37 mg, 8%).

¹H-NMR (300 MHz, DMSO-d₆) δ (ppm) 4.94 (s, 2H), 7.0-7.1 (m, 4H), 7.76 (d, J=7.9 Hz, 1H), 7.94 (d, J=8.8 Hz, 2H), 9.19 (s, 1H), 12.8 (br s, 1H).

### 4.2.am 1-Hydroxy-1,3 dihydro-5-[4-(tetrazole-1-yl)phenoxy]-2,1-benzoxaborole

### 5-(4-(1H-tetrazol-5-yl)phenoxy)benzo[c][1,2]oxaborol-1(3H)-ol (C39)

A mixture of 5-(4-cyanophenoxy)-1-hydroxy-2,1-benzoxaborole (200 mg, 0.797 mmol), sodium azide (103 mg, 1.59 mmol), and ammonium chloride (85 mg, 1.6 mmol) in N,N-dimethylformamide (5 mL) was stirred at 80° C. for two days. Water was added, and the mixture was extracted with ethyl acetate. The organic layer was washed with water and brine, and dried on anhydrous sodium sulfate. The solvent was removed under reduced pressure, and the residue was purified by silica gel column chromatography (ethyl acetate) followed by trituration with ethyl acetate to give the target compound (55 mg, 23%).

¹H-NMR (300 MHz, DMSO-d₆) δ (ppm) 4.95 (s, 2H), 7.0-7.1 (m, 2H), 7.23 (d, J=8.8 Hz, 2H), 7.76 (d, J=7.9 Hz, 1H), 8.05 (d, J=8.5 Hz, 2H), 9.18 (br s, 1H).

### Example 5

### Preparation of I from 2 via 6

5.1 Catalytic Boronylation, Reduction and Cyclization

A mixture of 2 (10.0 mmol), bis(pinacolato)diboron (2.79 g, 11.0 mmol), PdCl₂(dppf) (250 mg, 3 mol %), and potassium acetate (2.94 g, 30.0 mmol) in 1,4-dioxane (40 mL) was stirred at 80° C. for overnight. Water was added, and the mixture was extracted with ethyl acetate. The organic layer was washed with brine and dried on anhydrous sodium sulfate. The solvent was removed under reduced pressure. The crude product was dissolved in tetrahydrofuran (80 mL), then sodium periodate (5.56 g, 26.0 mmol) was added. After stirring at room temperature for 30 min, 2N HCl (10 mL) was added, and the mixture was stirred at room temperature for overnight. Water was added, and the mixture was extracted with ethyl acetate. The organic layer was washed with brine and dried on anhydrous sodium sulfate. The solvent was removed under reduced pressure, and the residue was treated with ether to afford 6.3 mmol of the corresponding boronic acid. To the solution of the obtained boronic acid (0.595 mmol) in methanol (5 mL) was added sodium borohydride (11 mg, 0.30 mmol), and the mixture was stirred at room temperature for 1 h. Water was added, and the mixture was extracted with ethyl acetate. The organic layer was washed with brine and dried on anhydrous sodium sulfate. The solvent was removed under reduced pressure, and the residue was purified by silica gel column chromatography to give 0.217 mmol of I.

5.2 Results

Analytical data for exemplary compounds of structure I are provided below.

### 5.2.a 1,3-Dihydro-5-fluoro-1-hydroxy-2,1-benzoxaborole (C10)

Analytical data for this compound is listed in 4.2.j.

### Example 6

### Preparation of I from 3

6.1 One-pot Boronylation and Cyclization

To a solution of 3 (4.88 mmol) and triisopropyl borate (1.35 mL, 5.86 mmol) in tetrahydrofuran (10 mL) was added n-butyllithium (1.6 mol/L in hexanes; 6.7 mL, 10.7 mmol) dropwise over 15 min at −78° C. under nitrogen atmosphere, and the mixture was stirred for 2 h while allowing to warm to room temperature. The reaction was quenched with 2N HCl, and extracted with ethyl acetate. The organic layer was washed with brine and dried on anhydrous sodium sulfate. The solvent was removed under reduced pressure, and the residue was purified by silica gel column chromatography and treated with pentane to give 0.41 mmol of I.

6.2 Results

Analytical data for exemplary compounds of structure I are provided below.

### 6.2.a 1,3-Dihydro-5-fluoro-1-hydroxy-2,1-benzoxaborole (C10)

Analytical data for this compound is listed in 4.2.j.

### Example 7

### Preparation of I from 3

7.1 One-pot Boronylation and Cyclization with Distillation

To a solution of 3 (4.88 mmol) in toluene (20 mL) was added triisopropyl borate (2.2 mL, 9.8 mmol), and the mixture was heated at reflux for 1 h. The solvent, the generated isopropyl alcohol and excess triisopropyl borate were removed under reduced pressure. The residue was dissolved in tetrahydrofuran (10 mL) and cooled to −78° C. n-Butyllithium (3.2 mL, 5.1 mmol) was added dropwise over 10 min, and the mixture was stirred for 1 h while allowing to warm to room temperature. The reaction was quenched with 2N HCl, and extracted with ethyl acetate. The organic layer was washed with brine and dried on anhydrous sodium sulfate. The solvent was removed under reduced pressure, and the residue was purified by silica gel column chromatography to give 1.54 mmol of I.

7.2 Results

Analytical data for exemplary compounds of structure I are provided below.

### 7.2.a 1,3-Dihydro-5-fluoro-1-hydroxy-2,1-benzoxaborole (C10)

Analytical data for this compound is listed in 4.2.j.

### Example 8

### Preparation of 8 from 7

8.1 Bromination

To a solution of 7 (49.5 mmol) in carbon tetrachloride (200 mL) were added N-bromosuccinimide (8.81 g, 49.5 mmol) and N,N-azoisobutylonitrile (414 mg, 5 mol %), and the mixture was heated at reflux for 3 h. Water was added, and the mixture was extracted with chloroform. The organic layer was washed with brine and dried on anhydrous sodium sulfate. The solvent was removed under reduced pressure to give the crude methyl-brominated intermediate 8.

### Example 9

### Preparation of 3 from 8

9.1 Hydroxylation

To crude 8 (49.5 mmol) were added dimethylformamide (150 mL) and sodium acetate (20.5 g, 250 mmol), and the mixture was stirred at 80° C. for overnight. Water was added, and the mixture was extracted with ether. The organic layer was washed with water and brine, and dried on anhydrous sodium sulfate. The solvent was removed under reduced pressure. To the residue was added methanol (150 mL) and 1N sodium hydroxide (50 mL), and the mixture was stirred at room temperature for 1 h. The reaction mixture was concentrated to about a third of volume under reduced pressure. Water and hydrochloric acid were added, and the mixture was extracted with ethyl acetate. The organic layer was washed with water and brine, and dried on anhydrous sodium sulfate. The solvent was removed under reduced pressure, and the residue was purified by silica gel column chromatography followed by trituration with dichloromethane to give 21.8 mmol of 3.

9.2 Results

Exemplary compounds of structure 3 prepared by the method above are provided below.

### 9.2.a 2-Bromo-5-cyanobenzyl Alcohol

¹H-NMR (300 MHz, DMSO-d₆) δ ppm 4.51 (d, J=5.9 Hz, 2H), 5.67 (t, J=5.6 Hz, 1H), 7.67 (dd, J=8.2, 2.0 Hz, 1H), 7.80 (s, J=8.2 Hz, 1H), 7.83 (d, J=2.0 Hz, 1H).

Additional examples of compounds which can be produced by this method include 2-bromo-5-(4-cyanophenoxy)benzyl alcohol.

### Example 10

### Preparation of 9 from 2

10.1 Reaction

A mixture of 2 (20.0 mmol), (methoxymethyl)triphenylphosphonium chloride (8.49 g, 24.0 mmol), and potassium tert-butoxide (2.83 g, 24.0 mol) in N,N-dimethylformamide (50 mL) was stirred at room temperature for overnight. The reaction was quenched with 6 N HCl, and the mixture was extracted with ethyl acetate. The organic layer was washed with water (×2) and brine, and dried on anhydrous sodium sulfate. The solvent was removed under reduced. To the residue were added tetrahydrofuran (60 mL) and 6 N HCl, and the mixture was heated at reflux for 8 h. Water was added, and the mixture was extracted with ether. The organic layer was washed with brine and dried on anhydrous sodium sulfate. The solvent was removed under reduced pressure to afford 16.6 mmol of 9.

### Example 11

### Preparation Method of Step 13

11.1 Reaction

A solution of I in an appropriate alcohol solvent (R¹—OH) was refluxed under nitrogen atmosphere and then distilled to remove the alcohol to give the corresponding ester.

### Example 12

### Preparation of Ib from Ia

12.1 Reaction

To a solution of Ia in toluene was added amino alcohol and the participated solid was collected to give Ib.

12.2 Results

(500 mg, 3.3 mmol) was dissolved in toluene (37 mL) at 80° C. and ethanolamine (0.20 mL, 3.3 mmol) was added. The mixture was cooled to room temperature, then ice bath, and filtered to give C40 as a white powder (600.5 mg, 94%).

### 12.2a Ethanolamine adduct of 1,3-Dihydro-5-fluoro-1-hydroxy-2,1-benzoxaborole (C40)

¹H-NMR (300 MHz, DMSO-d₆) δ (ppm) 2.88 (t, J=6.2 Hz, 2H), 3.75 (t, J=6.3 Hz, 2H), 4.66 (s, 2H), 5.77 (br, 2H), 6.85-6.91 (m, 2H), 7.31 (td, J=7.2, 1.2 Hz, 1H).

### Example 13

### Preparation of Pyridinyloxaboroles

### 14a. Metallation and boronylation

To a solution of 3-bromo-4-hydroxymethylpyridine (10.7 mmol) and B(OMe)₃ (2.73 mL, 11.9 mmol) in anhydrous THF (20 mL) at −78° C. under nitrogen was added dropwise n-BuLi (13.6 mL, 21.8 mmol). The cooling bath was then removed. The mixture was warmed gradually with stirring for 30 min and then stirred with a water bath for 2 h. Brine was then added and the pH adjusted to 7 using 6N HCl. The mixture was washed with THF (×2) and the aqueous layer (containing product) was evaporated to dryness. The residue was washed with THF and the product was extracted into ethanol (×2). Ethanol was removed in vacuo, water was added to the residue and removed in vacuo. Toluene was added and removed in vacuo. The resulting residue was triturated with diethyl ether and the product was collected by filtration to afford C12.

### 14b. 7-Hydroxy-2,1-oxaborolano[5,4-c]pyridine[[1,2]oxaborolo[3,4-c]pyridin-1(3H)-ol](C12)

¹H-NMR (300 MHz, DMSO-d₆): 6 ppm 5.00 (s, 2H), 7.45 (d, J=5.0 Hz, 1H), 8.57 (d, J=5.3 Hz, 1H), 8.91 (s, 1H), 9.57 (s, 1H). ESI-MS m/z 134 (M−H) C₆H₆BNO₂=135.

### Example 14

### Cyclic Borinic Esters

Additional compounds can be produced by the methods described herein. By choosing the appropriate starting material such as 1 or 3, Examples 1-7 can be used to formulate the following compounds. Where available, melting point characterization is provided for these compounds.

14. Results

Analytical data for exemplary compounds of structure I are provided below.

### 14a Ethyl 2-(1-hydroxy-1,3-dihydrobenzo[c][1,2]oxaborol-5-yloxy)acetate (C41)

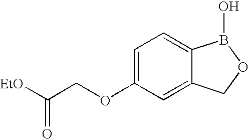

M.P. 134-137° C. Exemplary starting material: ethyl 2-(4-bromo-3-(hydroxymethyl)phenoxy)acetate.

### 14b 2-(1-hydroxy-1,3-dihydrobenzo[c][1,2]oxaborol-5-yloxy)acetic acid (C42)

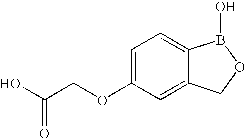

M.P. 163-166° C. Exemplary starting material: ethyl 2-(4-bromo-3-(hydroxymethyl)phenoxy)acetate. The title compound is obtained after saponification of the corresponding ester.

### 14c 6-(thiophen-2-ylthio)benzo[c][1,2]oxaborol-1(3H)-ol (C43)

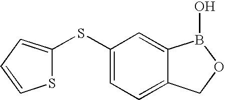

M.P. 99-104° C. Exemplary starting material: (2-bromo-4-(thiophen-2-ylthio)phenyl)methanol.

### 14d 6-(4-fluorophenylthio)benzo[c][1,2]oxaborol-1(3H)-ol (C44)

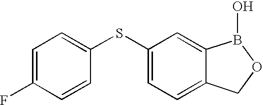

M.P. 135-138° C. Exemplary starting material: (2-bromo-4-(4-fluorophenylthio)phenyl)methanol.

### 14e 1-(3-((1-hydroxy-1,3-dihydrobenzo[c][1,2]oxaborol-5-yloxy)methyl)phenyl)pentan-1-one (C45)

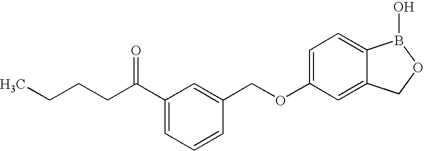

M.P. 96-98° C. Exemplary starting material: 1-(3-((4-bromo-3-(hydroxymethyl)phenoxy)methyl)phenyl)pentan-1-one.

### 14f 2-(1-hydroxy-1,3-dihydrobenzo[c][1,2]oxaborol-5-yloxy)-1-(pieridin-1-yl)ethanone (C46)

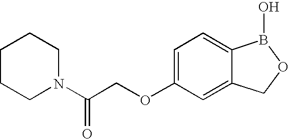

M.P. 158-163° C. Exemplary starting material: 2-(4-bromo-3-(hydroxymethyl)phenoxy)-1-(piperidin-1-yl)ethanone.

### 14g 2-(1-hydroxy-1,3-dihydrobenzo[c][1,2]oxaborol-5-yloxy)-1-(4-(pyrimidin-2-yl)piperazin-1-yl)ethanone (C47)

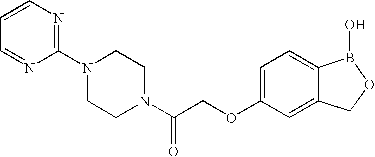

M.P. 190-195° C. Exemplary starting material: 2-(4-bromo-3-(hydroxymethyl)phenoxy)-1-(4-(pyrimidin-2-yl)piperazin-1-yl)ethanone.

### 14h 6-(4-(pyridin-2-yl)piperazin-1-yl)benzo[c][1,2]oxaborol-1(3H)-ol (C48)

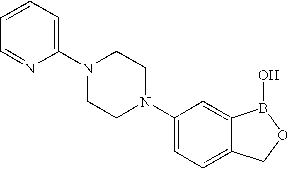

M.P. 135-138° C. Exemplary starting material: (2-bromo-4-(4-(pyridin-2-yl)piperazin-1-yl)phenyl)methanol.

### 14i 6-nitrobenzo[c][1,2]oxaborol-1(3H)-ol (C49)

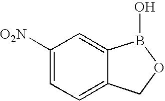

M.P. 163-171° C. Exemplary starting material: benzo[c][1,2]oxaborol-1(3H)-ol. See JACS 82, 2172, 1960 for preparation.

### 14j 6-aminobenzo[c][1,2]oxaborol-1(3H)-ol (C50)

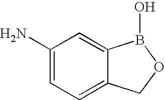

M.P. 145-148° C. Exemplary starting material: 6-nitrobenzo[c][1,2]oxaborol-1(3H)-ol.

### 14k 6-(dimethylamino)benzo[c][1,2]oxaborol-1(3H)-ol (C51)

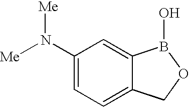

M.P. 120-123° C. Exemplary starting material: 6-aminobenzo[c][1,2]oxaborol-1(3H)-ol.

### 14l N-(1-hydroxy-1,3-dihydrobenzo[c][1,2]oxaborol-6-yl)benzamide (C52)

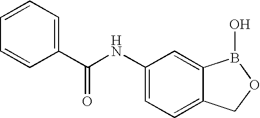

M.P. 186-193° C. Exemplary starting material: 6-aminobenzo[c][1,2]oxaborol-1(3H)-ol.

### 14m 6-(4-phenylpiperazin-1-yl)benzo[c][1,2]oxaborol-1(3H)-ol (C53)

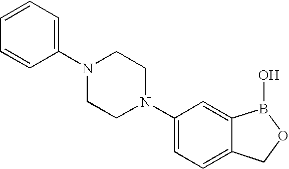

M.P. 159-161° C. Exemplary starting material: (2-bromo-4-(4-phenylpiperazin-1-yl)phenyl)methanol.

### 14o 6-(1H-indol-1-yl)benzo[c][1,2]oxaborol-1(3H)-ol (C55)

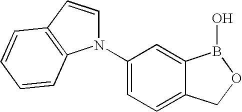

M.P. 135-140° C. Exemplary starting material: (2-bromo-4-(1H-indol-1-yl)phenyl)methanol.

### 14p 6-morpholinobenzo[c][1,2]oxaborol-1(3H)-ol (C56)

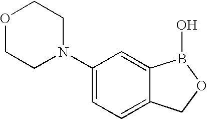

M.P. 128-132° C. Exemplary starting material: (2-bromo-4-morpholinophenyl)methanol.

### 14q 6-(1-hydroxy-1,3-dihydrobenzo[c][1,2]oxaborol-5-yloxy)nicotinonitrile (C57)

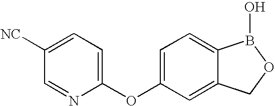

M.P. 193-198° C. Exemplary starting material: 6-(4-bromo-3-(hydroxymethyl)phenoxy)nicotinonitrile.

### 14r 5-fluoro-6-nitrobenzo[c][1,2]oxaborol-1(3H)-ol (C58)

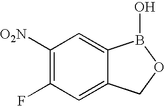

M.P. 162-167° C. Exemplary starting material: 5-fluorobenzo[c][1,2]oxaborol-1(3H)-ol.

### 14s 5-bromo-6-hydroxymethyl)benzo[c][1,2]oxaborol-1(3H)-ol (C59)

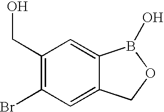

M.P. >257° C. Exemplary starting material: (2,5-dibromo-4-(methoxymethyl)phenyl)methanol.

### 14t 3,7-dihydro-1,5-dihydroxy-1H,3H-Benzo[1,2-c:4,5-c′]bis[1,2]oxaborole (C60)

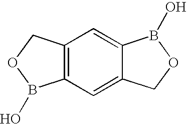

M.P. >250° C. Exemplary starting material: (2,5-dibromo-1,4-phenylene)dimethanol.

### 14u 1-(1-hydroxy-1,3-dihydrobenzo[c][1,2]oxaborol-6-yl)-3-phenylurea (C61)

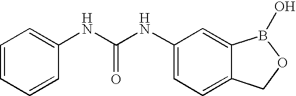

M.P. 213-215° C. Exemplary starting material: 6-aminobenzo[c][1,2]oxaborol-1(3H)-ol.

### 14v N-(1-hydroxy-1,3-dihydrobenzo[c][1,2]oxaborol-6-yl)benzenesulfonamide (C62)

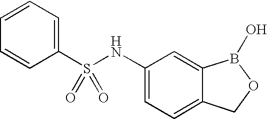

M.P. 175-184° C. Exemplary starting material: 6-aminobenzo[c][1,2]oxaborol-1(3H)-ol.

### 14w N-(1-hydroxy-1,3-dihydrobenzo[c][1,2]oxaborol-6-yl)acetamide (C63)

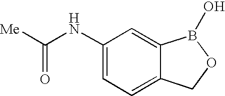

M.P. 176-185° C. Exemplary starting material: 6-aminobenzo[c][1,2]oxaborol-1(3H)-ol.

### 14x 7-(hydroxymethyl)benzo[c][1,2]oxaborol-1(3H)-ol (C64)

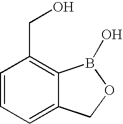

M.P. 241-250° C. Exemplary starting material: (2-bromo-1,3-phenylene)dimethanol.

### 14y 7-methylbenzo[c][1,2]oxaborol-1(3H)-ol (C65)

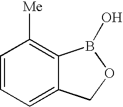

M.P. 107-111° C. Exemplary starting material: (2-bromo-3-methylphenyl)methanol.

### 14z 6-(3-(phenylthio)-1H-indol-1-yl)benzo[c][1,2]oxaborol-1(3H)-ol (C66)

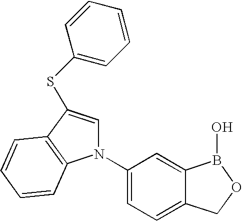

M.P. 159-163° C. Exemplary starting material: (2-bromo-4-(3-(phenylthio)-1H-indol-1-yl)phenyl)methanol.

### 14aa 3-(1-(1-hydroxy-1,3-dihydrobenzo[c][1,2]oxaborol-6-yl)-1H-indol-3-ylthio)propanenitrile (C67)

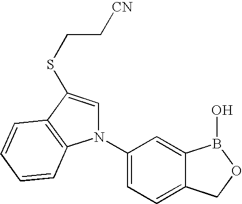

M.P. 135-141° C. Exemplary starting material: 3-(1-(3-bromo-4-(hydroxymethyl)phenyl)-1H-indol-3-ylthio)propanenitrile.

### 14bb 6-(5-methoxy-1H-indol-1-yl)benzo[c][1,2]oxaborol-1(3H)-ol (C68)

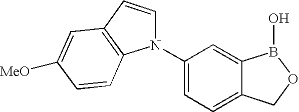

M.P. 120-124° C. Exemplary starting material: (2-bromo-4-(5-methoxy-1H-indol-1-yl)phenyl)methanol.

### 14cc 5,6-methylenedioxybenzo[c][1,2]oxaborol-1(3H)-ol (C69)

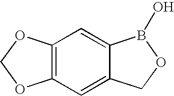

M.P. 185-189° C. Exemplary starting material: (6-bromobenzo[d][1,3]dioxol-5-yl)methanol.

### 14dd 6-amino-5-fluorobenzo[c][1,2]oxaborol-1(3H)-ol (C70)

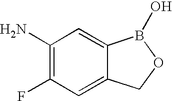

M.P. 142-145° C. Exemplary starting material: 6-nitro-5-fluorobenzo[c][1,2]oxaborol-1(3H)-ol.

### 14ee 6-(benzylamino)-5-fluorobenzo[c][1,2]oxaborol-1(3H)-ol (C71)

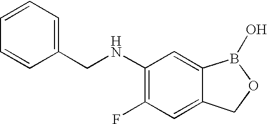

M.P. 159-164° C. Exemplary starting material: 6-amino-5-fluorobenzo[c][1,2]oxaborol-1(3H)-ol.

### 14ff 6-(5-methoxy-3-(phenylthio)-1H-indol-1-yl)benzo[c][1,2]oxaborol-1(3H)-ol (C72)

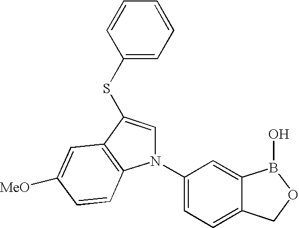

M.P. 135-141° C. Exemplary starting material: (2-bromo-4-(5-methoxy-3-(phenylthio)-1H-indol-1-yl)phenyl)methanol.

### 14gg 3-(1-(1-hydroxy-1,3-dihydrobenzo[c][1,2]oxaborol-6-yl)-5-methoxy-1H-indol-3-ylthio)propanenitrile (C73)

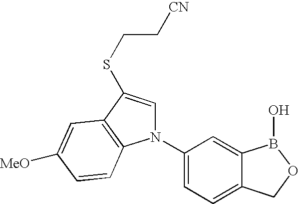

M.P. 149-154° C. Exemplary starting material: 3-(1-(3-bromo-4-(hydroxymethyl)phenyl)-5-methoxy-1H-indol-3-ylthio)propanenitrile.

### 14hh 4-(1-hydroxy-1,3-dihydrobenzo[c][1,2]oxaborol-7-yloxy)benzonitrile (C74)

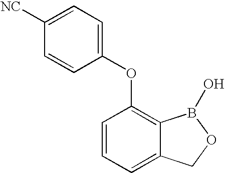

M.P. 148-153° C. Exemplary starting material: 4-(2-bromo-3-(hydroxymethyl)phenoxy)benzonitrile.

### 14ii 6-(5-chloro-1H-indol-1-yl)benzo[c][1,2]oxaborol-1(3H)-ol (C75)

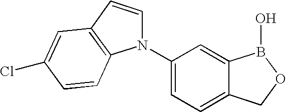

M.P. 149-154° C. Exemplary starting material: (2-bromo-4-(5-chloro-1H-indol-1-yl)phenyl)methanol.

### 14jj 3-(5-chloro-1-(1-hydroxy-1,3-dihydrobenzo[c][1,2]oxaborol-6-yl)-1H-indol-3-ylthio)propanenitrile (C76)

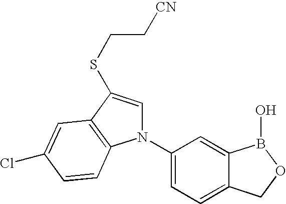

M.P. >225° C. Exemplary starting material: 3-(1-(3-bromo-4-(hydroxymethyl)phenyl)-5-chloro-1H-indol-3-ylthio)propanenitrile.

### 14kk 6-(benzylamino)benzo[c][1,2]oxaborol-1(3H)-ol (C77)

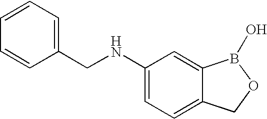

M.P. 126-133° C. Exemplary starting material: 6-aminobenzo[c][1,2]oxaborol-1(3H)-ol.

### 14ll 6-(dibenzylamino)benzo[c][1,2]oxaborol-1(3H)-ol (C78)

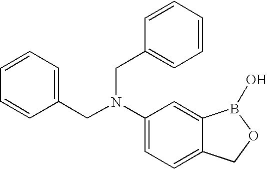

M.P. 115-123° C. Exemplary starting material: 6-aminobenzo[c][1,2]oxaborol-1(3H)-ol.

### 14mm 7-(4-(1H-tetrazol-5-yl)phenoxy)benzo[c][1,2]oxaborol-1(3H)-ol (C79)

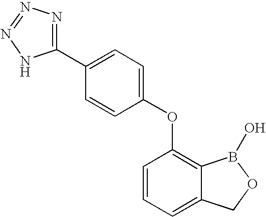

M.P. decomposition at 215° C. Exemplary starting material: 4-(1-hydroxy-1,3-dihydrobenzo[c][1,2]oxaborol-7-yloxy)benzonitrile.

### 14nn 6-(5-chloro-3-(phenylthio)-1H-indol-1-yl)benzo[c][1,2]oxaborol-1(3H)-ol (C80)

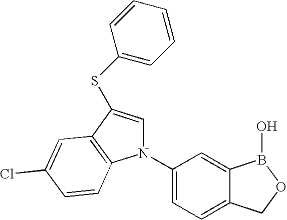

M.P. 145-151° C. Exemplary starting material: (2-bromo-4-(5-chloro-3-(phenylthio)-1H-indol-1-yl)phenyl)methanol.

### 14pp 6-(4-(pyrimidin-2-yl)piperazin-1-yl)benzo[c][1,2]oxaborol-1(3H)-ol (C82)

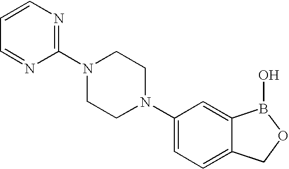

M.P. NA ° C. Exemplary starting material: (2-bromo-4-(4-(pyrimidin-2-yl)piperazin-1-yl)phenyl)methanol.

### 14qq 7-(benzyloxy)benzo[c][1,2]oxaborol-1(3H)-ol (C83)

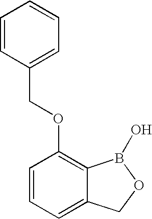

M.P. NA ° C. Exemplary starting material: (3-(benzyloxy)-2-bromophenyl)methanol.

### 14rr 4-(1-hydroxy-1,3-dihydrobenzo[c][1,2]oxaborol-6-ylthio)pyridinium chloride (C84)

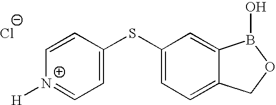

M.P. NA ° C. Exemplary starting material: (2-bromo-4-(pyridin-4-ylthio)phenyl)methanol.

### 14ss 6-(pyridin-2-ylthio)benzo[c][1,2]oxaborol-1(3H)-ol (C85)

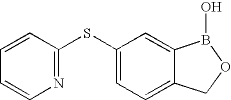

M.P. NA OC. Exemplary starting material: (2-bromo-4-(pyridin-2-ylthio)phenyl)methanol.

### 14tt 7-fluorobenzo[c][1,2]oxaborol-1(3H)-ol (C86)

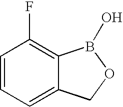

M.P. 120-124° C. Exemplary starting material: (2-bromo-3-fluorophenyl)methanol.

### 14uu 6-(4-(trifluoromethyl)phenoxy)benzo[c][1,2]oxaborol-1(3H)-ol (C87)

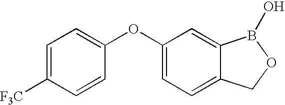

M.P. 98-105° C. Exemplary starting material: (2-bromo-4-(4-(trifluoromethyl)phenoxy)phenyl)methanol.

### 14vv 6-(4-chlorophenylthio)benzo[c][1,2]oxaborol-1(3H)-ol (C88)

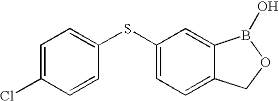

M.P. 157-161° C. Exemplary starting material: (2-bromo-4-(4-chlorophenylthio)phenyl)methanol.

### 14ww 6-(4-chlorophenylsulfinyl)benzo[c][1,2]oxaborol-1(3H)-ol (C89)

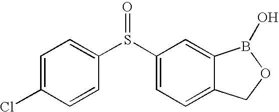

M.P. 154-161° C. Exemplary starting material: 6-(4-chlorophenylthio)benzo[c][1,2]oxaborol-1(3H)-ol.

### 14xx 6-(4-chlorophenylsulfonyl)benzo[c][1,2]oxaborol-1(3H)-ol (C90)

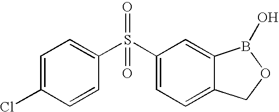

M.P. 157-163° C. Exemplary starting material: 6-(4-chlorophenylthio)benzo[c][1,2]oxaborol-1(3H)-ol.

### 14yy N-(1-hydroxy-1,3-dihydrobenzo[c][1,2]oxaborol-5-yl)-N-(phenylsulfonyl)benzenesulfonamide (C91)

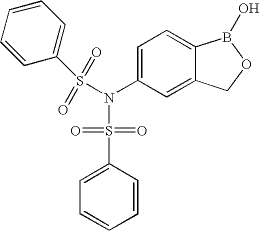

M.P. 142-152° C. Exemplary starting material: N-(4-bromo-3-(hydroxymethyl)phenyl)-N-(phenylsulfonyl)benzenesulfonamide.

### 14zz 6-(4-(trifluoromethyl)phenylthio)benzo[c][1,2]oxaborol-1(3H)-ol (C92)

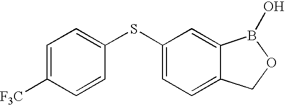

M.P. 111-113° C. Exemplary starting material: (2-bromo-4-(4-(trifluoromethyl)phenylthio)phenyl)methanol.

### 14aaa 6-(4-(trifluoromethyl)phenylsulfinyl)benzo[c][1,2]oxaborol-1(3H)-ol (C93)

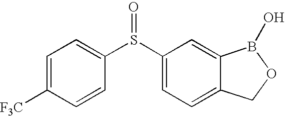

M.P. 79-88° C. Exemplary starting material: 6-(4-(trifluoromethyl)phenylthio)benzo[c][1,2]oxaborol-1(3H)-ol.

### 14bbb 6-(4-(methylthio)phenylthio)benzo[c][1,2]oxaborol-1(3H)-ol (C94)

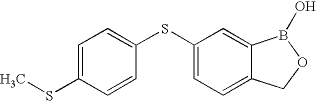

M.P. 117-120° C. Exemplary starting material: (2-bromo-4-(4-(methylthio)phenylthio)phenyl)methanol.

### 14ccc 6-(p-tolylthio)benzo[c][1,2]oxaborol-1(3H)-ol (C95)

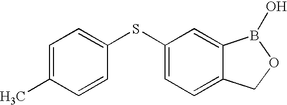

M.P. 139-144° C. Exemplary starting material: (2-bromo-4-(p-tolylthio)phenyl)methanol.

### 14ddd 3-((1-hydroxy-1,3-dihydrobenzo[c][1,2]oxaborol-5-yloxy)methyl)benzonitrile (C96)

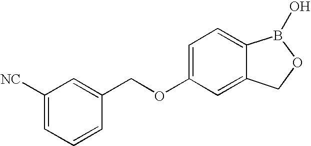

M.P. 147-150° C. Exemplary starting material: 3-((4-bromo-3-(hydroxymethyl)phenoxy)methyl)benzonitrile.

### Example 15

### Precursors to CBOs and CBEs

### 15.1 2-Bromo-5-fluoro-[1-(methoxymethoxy)methyl]benzene (5b)

To a solution of 3 (62.0 g, 293 mmol) in MeOH (400 mL) was added NaBH₄ (5.57 g, 147 mmol) portionwise at 0° C., and the mixture was stirred at room temperature for 1 h. Water was added, and the solvent was removed under reduced pressure to about a half volume. The mixture was poured into EtOAc and water. The organic layer was washed with brine and dried over anhydrous Na₂SO₄. The solvent was removed under reduced pressure to afford 4b, which was used for the next step without purification. To a solution of 4b (60.8 g, 293 mmol) and i-Pr₂NEt (61 mL, 0.35 mol) in CH₂Cl₂ was added chloromethyl methyl ether (27 mL, 0.35 mmol) at 0° C., and the mixture was stirred at room temperature overnight. Water was added, and the mixture was extracted with CHCl₃. The organic layer was washed with brine and dried over anhydrous Na₂SO₄. The solvent was removed under reduced pressure to afford 5b (73.2 g, quant). ¹H NMR (300 MHz, CDCl₃) δ (ppm) 3.43 (s, 3H), 4.62 (s, 2H), 4.78 (s, 2H), 6.88 (td, J=8.5, 3.2 Hz, 1H), 7.25 (dd, J=9.6, 3.1 Hz, 1H), 7.48 (dd, J=8.8, 5.3 Hz, 1H).

### 15.2 2-Bromo-[1-(methoxymethoxy)methyl]benzene (5a)

This compound was made from 2-bromobenzylalcohol in the same manner as compound 5b and used for the next step without purification.

### 15.3 2-[4-Fluoro-2-[(methoxymethoxy)methyl]phenyl]-[1,3,2]dioxaborolane (6)

To a solution of 5b (16.2 g, 65.1 mmol) in THF (130 mL) were added sec-BuLi (1.4 M, 56 mL) and (MeO)₃B (14.5 mL, 130 mmol) at −78° C. under nitrogen atmosphere, and the mixture was allowed to warm to room temperature and stirred for 2 h. Water and 1 N NaOH were added to the mixture, which was washed with Et₂O. Then the pH was adjusted to 4 with 1 N HCl, and the mixture was extracted with EtOAc. The organic layer was washed with brine and dried over anhydrous Na₂SO₄. Then the solvent was removed under reduced pressure to give boronic acid, which was used for the next step without purification. To a solution of the boronic acid in toluene (300 mL) was added ethylene glycol (3.29 g, 53 mmol), and the mixture was refluxed for 3 h with a Dean-Stark trap. The solvent was removed under reduced pressure to afford 6 (12.1 g, 77%). ¹H-NMR (300 MHz, CDCl₃) δ (ppm) 3.42 (s, 3H), 4.36 (s, 4H), 4.76 (s, 2H), 4.87 (s, 2H), 6.96 (td, J=8.2, 2.6 Hz, 1H), 7.26 (dd, J=10.6, 2.6 Hz, 1H), 7.83 (dd, J=8.2, 6.4 Hz, 1H).

### 15.4 2-(3-Chlorophenyl)[1,3,2]dioxaborolane (7b; Rⁱⁱⁱ=3-Cl-Ph)

3-Chlorophenylboronic acid (3.041 g, 19.4 mmol) was dissolved in 75 mL of dry THF under nitrogen atmosphere. Ethylene glycol (1.32 g, 21.3 mmol) was added and the solution was refluxed for 18 h. The solution was allowed to cool and the THF was removed under reduced pressure to give 7b (3.55 g, 100%) as a brown oil that solidified upon cooling in the freezer. ¹H NMR (300 MHz, CDCl₃) δ (ppm) 4.39 (s, 4H), 7.32 (t, J=7.9 Hz, 1H), 7.45 (dd, J=8.2, 1.2 Hz, 1H), 7.67 (d, J=7.0 Hz, 1H), 7.78 (br s, 1H).

Compounds 7a and 7c-k were synthesized in a similar manner to 7b.

### 15.5 2-Phenyl[1,3,2]dioxaborolane (7a; Rⁱⁱⁱ=Ph)

¹H NMR (300 MHz, DMSO-d₆) δ (ppm) 4.30 (s, 4H), 7.35-7.41 (t, J=8.2 Hz, 2H), 7.46-7.52 (m, 1H), 7.68-7.72 (dd, J=6.2, 2.6 Hz, 2H).

### 15.6 2-(4-Chlorophenyl) [1,3,2]dioxaborolane (7c; Rⁱⁱⁱ=4-Cl-Ph)

hu 1H NMR (300 MHz, CDCl₃) δ (ppm) 4.38 (s, 4H), 7.36 (d, J=6.7 Hz, 2H), 7.74 (d, J=7.0 Hz, 2H).

### 15.7 2-(3-Fluorophenyl) [1,3,2]dioxaborolane (7d; Rⁱⁱⁱ3-F-Ph)

¹H NMR (300 MHz, CDCl₃) δ (ppm) 4.39 (s, 4H), 7.1-7.2 (m, 1H), 7.36 (td, J=8.2, 5.6 Hz, 1H), 7.48 (dd, J=9.1, 2.6 Hz, 1H), 7.58 (d, J=7.0 Hz, 1H).

### 15.8 2-(4-Fluorophenyl)[1,3,2]dioxaborolane (7e; Rⁱⁱⁱ=4-F-Ph)

¹H NMR (300 MHz, DMSO-d₆) δ (ppm) 4.29 (s, 4H), 7.17-7.23 (t, J=8.5 Hz, 2H), 7.71-7.76 (dd, J=8.5, 6.1 Hz, 2H).

### 15.9 2-(3-Methylphenyl) [1,3,2]dioxaborolane (7f; Rⁱⁱⁱ=3-Me-Ph)

¹H NMR (300 MHz, DMSO-d₆) δ (ppm) 2.31 (s, 3H), 4.31 (s, 4H), 7.29-7.32 (m, 2H), 7.50-7.53 (m, 2H).

### 15.10 2-Styryl[1,3,2]dioxaborolane (7h; Rⁱⁱⁱ=styryl)

¹H NMR (300 MHz, DMSO-d₆) δ (ppm) 4.20 (s, 4H), 6.15 (d, J=18.5 Hz, 1H), 7.31-7.39 (m, 4H), 7.56 (dd, J=1.5, 7.6 Hz, 2H).

### 15.11 2-(Thiophen-3-yl)[1,3,2]dioxaborolane (7j; Rⁱⁱⁱ=thiophen-3-yl)

¹H NMR (300 MHz, DMSO-d₆) δ (ppm) 4.27 (s, 4H), 7.30 (dd, J=4.8, 0.9 Hz, 1H), 7.58 (dd, J=4.5, 2.4 Hz, 1H), 8.03 (dd, J=2.7, 1.2 Hz, 1H).

### 15.12 2-(4-Methylthiophen-3-yl)[1,3,2]dioxaborolane (7k; Rⁱⁱⁱ=4-methylthiophen-3-yl)

¹H NMR (300 MHz, DMSO-d₆) δ (ppm) 2.31 (s, 3H), 4.25 (s, 4H), 7.13-7.14 (m, 1H), 7.93 (d, J=3.0 Hz, 1H).

### Example 16

### CBEs

### 16.1 1-(3-Chlorophenyl)-1,3-dihydro-5-fluoro-2,1-benzoxaborole (9f)

Compound 5b (1.06 g, 4.20 mmol) was dissolved in 50 mL of dry THF under nitrogen atmosphere and cooled to −78° C. tert-BuLi (1.7M in pentane, 5.3 mL) was slowly added to the solution. After stirring for 10 minutes at −78° C., compound 7b (764 mg, 4.20 mmol) in 10 mL of dry THF was added and the solution was stirred for further 0.5 h. The solution was then allowed to warm to room temperature and stirred for 18 h. The solvent was removed under reduced pressure, and the residue was partitioned between 40 ml of H₂O and 80 mL of diethyl ether. The solution was vigorously stirred for several minutes then neutralized (pH 7) with 6 N HCl. The organic layer was separated and the aqueous solution extracted again with ether (2×80 mL). The ether extracts were combined, dried over MgSO₄, filtered and evaporated to give crude 8f (1.22 g) as a yellow oil, which was used for the next step without purification. Compound 8f (700 mg, 2.30 mmol) was dissolved in 46 mL of THF and 4 mL of concentrated HCl. The solution was stirred at room temperature for 12 h. Water (10 mL) was then added and the THF was removed under reduced pressure. This gave a suspension. The precipitates were filtered under vacuum and washed with water (10 mL) then with hexanes (5 mL) and dried to give compound 9f (334 mg, 59%) as a white solid: mp 112-114° C.; ¹H NMR (300 MHz, DMSO-d₆) δ (ppm) 5.15 (s, 2H), 7.02-7.08 (t, J=8.8 Hz, 1H), 7.14-7.17 (d, J=8.8 Hz, 1H), 7.23-7.33 (m, 2H), 7.65-7.72 (m, 3H); ESI-MS m/z 247.08, 249.03 (M−H)⁻; HPLC purity: 97.1%; Anal. (C₁₃H₉ClFO)C, H.

Compounds 9a-e, 9g-j, 10a, b, and 12-15 were synthesized in a similar manner to 9f.

### 16.1 1,3-Dihydro-1-phenyl-2,1-benzoxaborole (9a)

Colorless oil; ¹H NMR (300 MHz, DMSO-d₆) δ (ppm) 5.41 (s, 2H), 7.43-7.61 (m, 6H), 8.11 (d, J=9.4 Hz, 2H), 8.18 (d, J=8.2 Hz, 1H); ESI-MS m/z not observed; HPLC purity: 95.5%.

### 16.2 1,3-Dihydro-5-fluoro-1-phenyl-2,1-benzoxaborole (9b)

mp 90-99° C.; ¹H NMR (300 MHz, DMSO-d₆) δ (ppm) 5.37 (s, 2H), 7.22 (dt, J=2.3, 8.9 Hz, 1H), 7.38 (dd, J=2.1, 9.4 Hz, 1H), 7.45-7.57 (m, 3H), 8.06 (dd, J=1.8, 7.9 Hz, 2H), 8.16 (dd, J=5.9, 8.2 Hz, 1H); ESI-MS m/z 213 (M+H)⁺; HPLC purity: 95.1%.

### 16.3 1-(3-Chlorophenyl)-1,3-dihydro-2,1-benzoxaborole (9c)

colorless oil; ¹H NMR (300 MHz, DMSO-d₆) δ (ppm) 5.26 (s, 2H), 7.29-7.45 (m, 5H), 7.77-7.86 (m, 3H); ESI-MS m/z Not observed; HPLC purity: 96.0%; Anal (C₁₃H₁₀BClO) C, H.

### 16.4 1,3-Dihydro-1-(3-fluorophenyl)-2,1-benzoxaborole (9d)

colorless oil; ¹H NMR (300 MHz, DMSO-d₆) δ (ppm) 5.28 (s, 2H), 7.23 (m, 1H), 7.34 (m, 1H), 7.41-7.48 (m, 3H), 7.57-7.61 (dd, J=9.6, 2.6 Hz, 1H), 7.74-7.77 (d, J=7.3 Hz, 1H), 7.93-7.95 (d, J=7.3 Hz, 1H); ESI-MS m/z Not observed; HPLC purity: 98.3%; Anal (C₁₃H₁₀BFO)C, H.

### 16.5 1,3-Dihydro-1-(4-fluorophenyl)-2,1-benzoxaborole (9e)

mp 53-55° C.; ¹H NMR (300 MHz, DMSO-d₆) δ (ppm) 5.37 (s, 2H), 7.26-7.32 (m, 2H), 7.42 (m, 1H), 7.53-7.55 (m, 2H), 8.11-8.16 (m, 3H); ESI-MS m/z not observed; HPLC purity: 99.3%; Anal. (C₁₃H₁₀BFO)C, H.

### 16.6 1,3-Dihydro-5-fluoro-1-(3-fluorophenyl)-2,1-benzoxaborole (9g)

mp 80-82° C.; ¹H NMR (300 MHz, DMSO-d₆) δ (ppm) 5.20 (s, 2H), 7.06-7.18 (m, 2H), 7.22 (dd, J=9.6, 1.8 Hz, 1H), 7.39 (td, J=7.8, 5.4 Hz, 1H), 7.49 (dd, J=9.9, 2.7 Hz, 1H), 7.63 (dd, J=6.9, 0.9 Hz, 1H), 7.83 (dd, J=8.1, 5.7 Hz, 1H); ESI-MS m/z not observed; HPLC purity: 98.5%; Anal. (C₁₃H₉BF₂O)C, H.

### 16.7 1,3-Dihydro-5-fluoro-1-(4-fluorophenyl)-2,1-benzoxaborole (9h)

mp 75-77° C.; ¹H NMR (300 MHz, DMSO-d₆) δ (ppm) 5.33 (s, 2H), 7.19-7.30 (m, 3H), 7.36 (dd, J=9.9, 2.1 Hz, 1H), 8.05-8.14 (m, 3H).; ESI-MS m/z not observed; HPLC purity: 99.0%; Anal. (C₁₃H₉BF₂O)C, H.

### 16.8 1,3-Dihydro-5-fluoro-1-(3-methylphenyl)-2,1-benzoxaborole (91)

mp 48-49° C.; ¹H NMR (300 MHz, DMSO-d₆) δ (ppm) 2.37 (s, 3H), 5.36 (s, 2H), 7.25 (m, 1H), 7.3-7.5 (m, 3H), 7.8-7.9 (m, 2H), 8.20 (dd, J=7.9, 5.9 Hz, 1H); ESI-MS m/z 227 (M+H)⁺; HPLC purity: 99.8%; Anal. (C₁₄H₁₂BFO)C, H.

### 16.9 1,3-Dihydro-5-fluoro-1-(4-methylphenyl)-2,1-benzoxaborole (9j)

mp 48-49° C.; ¹H NMR (300 MHz, DMSO-d₆) δ (ppm) 2.36 (s, 3H), 5.35 (s, 2H), 7.25 (m, 1H), 7.29 (d, J=7.6 Hz, 2H), 7.40 (dd, J=9.4, 1.5 Hz, 1H), 7.99 (d, J=7.6 Hz, 2H), 8.20 (dd, J=7.9, 5.6 Hz, 1H); ESI-MS m/z 227 (M+H)⁺; HPLC purity: 98.9%; Anal. (C₁₄H₁₂BFO)C, H.

### 16.10 1,3-Dihydro-1-styryl-2,1-benzoxaborole (10a)

mp 57-59° C.; ¹H NMR (300 MHz, DMSO-d₆) δ (ppm) 5.33 (s, 2H), 6.85 (d, J=18.8 Hz, 1H), 7.38-7.46 (m, 4H), 7.56 (d, J=4.7 Hz, 2H), 7.64 (d, J=7.9 Hz, 2H), 7.83 (d, J=18.8 Hz, 1H), 8.14 (d, J=7.3 Hz, 1H); ESI-MS m/z 221 (M+H)⁺; HPLC purity: 98.5%; Anal. (C₁₃H₁₀BFO·0.1H₂O)C, H.

### 16.11 1,3-Dihydro-5-fluoro-1-styryl-2,1-benzoxaborole (10b)

mp 84-86° C.; ¹H NMR (300 MHz, DMSO-d₆) δ (ppm) 5.32 (s, 2H), 6.86 (d, J=18.8 Hz, 1H), 7.24 (td, J=2.3, 10.6 Hz, 1H), 7.38-7.47 (m, 4H), 7.74 (d, J=7.0 Hz, 2H), 7.83 (d, J=18.8 Hz, 1H), 8.19 (dd, J=5.9, 8.2, 1H); ESI-MS m/z 239 (M+H)⁺; HPLC purity: 99.1%; Anal. (C₁₃H₁₀BFO)C, H.

### 16.12 1,3-Dihydro-5-fluoro-1-(furan-3-yl)-2,1-benzoxaborole (12)

colorless oil; ¹H NMR (300 MHz, DMSO-d₆) δ (ppm) 5.34 (s, 2H), 6.84 (m, 1H), 7.24 (m, 1H), 7.37-7.40 (d, J=9.4 Hz, 1H), 7.83 (m, 1H), 8.14-8.18 (dd, J=8.2, 5.9 Hz, 1H), 8.49 (m, 1H); ESI-MS m/z 203 (M+H)⁺; HPLC purity: 96.9%; Anal. (C₁₁H₈BFO₂) C, H.

### 16.13 1,3-Dihydro-5-fluoro-1-(thiophen-3-yl)-2,1-benzoxaborole (13)

mp 33-35° C.; ¹H NMR (300 MHz, DMSO-d₆) δ (ppm) 5.33 (s, 2H), 7.24 (m, 1H), 7.35-7.38 (d, J=9.3 Hz, 1H), 7.65 (m, 2H), 8.17-8.22 (dd, J=8.4, 6.3 Hz, 1H), 8.48 (m, 1H); ESI-MS m/z 219 (M+H)⁺; HPLC purity: 97.8%; Anal. (C₁₁H₈BFOS) C, H.

### 16.14 1,3-Dihydro-5-fluoro-1-(4-methylthiophen-3-yl)-2,1-benzoxaborole (14)

mp 51-53° C.; ¹H NMR (300 MHz, DMSO-d₆) δ (ppm) 2.46 (s, 3H), 5.36 (s, 2H), 7.20-7.27 (m, 2H), 7.37-7.40 (dd, J=9.4, 2.1 Hz, 1H), 8.14-8.19 (dd, J=8.2, 5.9 Hz, 1H), 8.48-8.49 (d, J=2.6 Hz, 1H); ESI-MS m/z 233 (M+H)⁺; HPLC purity: 100%; Anal. (C₁₂H₁₀BFOS)C, H.

### 16.15 1,3-Dihydro-5-fluoro-1-vinyl-2,1-benzoxaborole (11)

Compound 5b (2.0 g, 8.0 mmol) in THF (30 mL) was cooled to −78° C. and tert-butyllithium (9.9 mL, 16.8 mmol) as 1.7 M solution in pentane was added slowly. After stirring at −78° C. for 30 min, dibutyl ester of vinyl boronic acid was added dropwise. The mixture was stirred at −78° C. for 1 h, then was warmed up to room temperature and stirred overnight. Concentrated HCl (4 mL) was added and was stirred at room temperature for 4 h. Water (10 mL) was added and THF was removed under reduced pressure. The residue was extracted with ethyl ether, washed with brine, dried over magnesium sulfate, and concentrated under reduced pressure. The crude product was purified by flash column chromatography (9:1 hexane/ethyl acetate) to give 11 (383 mg, 30%) as a yellowish oil; ¹H NMR (300 MHz, DMSO-d₆) δ (ppm) 5.27 (s, 2H), 6.25 (t, J=8.5 Hz, 1H), 6.50 (d, J=9.4 Hz, 2H), 7.06-7.15 (m, 2H), 7.89 (dd, J=5.6, 7.9 Hz, 1H); ESI-MS m/z (M+H)⁺; HPLC purity: 98.7%; Anal. (C₉H₈BFO·0.1H₂O)C, H.

### 16.16 3-(1,3-Dihydro-5-fluoro-2,1-benzoxaborol-1-yl)pyridine (15)

To a solution of 3-bromopyridine (731 mg, 4.63 mmol) in THF (5 mL) was added isopropylmagnesium chloride (1 M in THF; 2.3 mL) at room temperature under nitrogen atmosphere, and the mixture was stirred for 1 h. To the mixture was added compound 6 (1.11 g, 4.63 mmol) in THF (4 mL), and the mixture was stirred at room temperature overnight. Water was added and the pH was adjusted to 7 with 1 N HCl. Then the mixture was extracted with ethyl acetate. The solvent was removed under reduced pressure, and the residue was dissolved in THF (30 mL). To the mixture was added 1 N HCl (10 mL), and the mixture was refluxed overnight. The pH was adjusted to 7 with aqueous NaHCO₃ and the mixture was extracted with ethyl acetate. The organic layer was washed with brine and dried over anhydrous Na₂SO₄. The solvent was removed under reduced pressure and the residue was recrystallized from i-Pr₂O to afford compound 15 (76 mg, 7.7%): mp 210-212° C.; ¹H NMR (300 MHz, DMSO-d₆) δ 4.94 (s, 2H), 6.9-7.1 (m, 2H), 7.36 (br s, 1H), 7.66 (dd, J=6.7, 5.3 Hz, 1H), 8.19 (d, J=6.7 Hz, 1H), 8.24 (br s, 1H), 8.64 (d, J=5.3 Hz, 1H): ESI-MS m/z 214 (M+H)⁺; Anal (C₁₂H₉BFNO·0.6H₂O)C, H, N.

### Example 17

### Precursors for CBOs

### 17.1 2-Bromo-5-fluoro-[1-(methoxymethoxy)ethyl]benzene (18c)

To a solution of compound 3 (4.23 g, 20.0 mmol) in THF (30 mL) was added MeMgBr (1.4 mol/L in THF; 18 mL) at −78° C. under nitrogen atmosphere, and the mixture was stirred for 2 h while allowing to warm to room temperature. The reaction was quenched with 2 N HCl, and the mixture was extracted with EtOAc. The organic layer was washed with brine and dried over anhydrous Na₂SO₄. The solvent was removed under reduced pressure. To a solution of the residue (4.62 g) in CH₂Cl₂ (100 mL) were added i-Pr₂NEt (5.2 mL, 30 mmol) and chloromethyl methyl ether (2.0 mL, 26 mmol) at 0° C., and the reaction mixture was stirred at room temperature overnight. Water was added, and the mixture was extracted with CHCl₃. The organic layer was washed with brine and dried over anhydrous Na₂SO₄. The solvent was removed under reduced pressure. The residue was purified by silica gel column chromatography (15:1 hexane/ethyl acetate) to give 18c (4.97 g, 2 steps 94%):

¹H NMR (300 MHz, CDCl₃) δ (Ppm) 1.43 (d, J=6.5 Hz, 3H), 3.38 (s, 3H), 4.55 (d, J=6.5 Hz, 1H), 4.63 (d, J=6.5 Hz, 1H), 5.07 (q, J=6.5 Hz, 1H), 6.85 (m, 1H), 7.25 (dd, J=9.7, 2.6 Hz, 1H), 7.46 (dd, J=8.8, 5.3 Hz, 1H).

### 17.2 2-Bromo-5-chloro-1-(methoxymethoxymethyl)benzene (18d)

To a solution of 2-bromo-5-chlorobenzoic acid (5.49 g, 23.3 mmol) in anhydrous THF (70 mL) under nitrogen was added dropwise a BH₃ THF solution (1.0 M, 55 mL) at 0° C. and the reaction mixture was stirred overnight at room temperature. Then the mixture was cooled on an ice bath and MeOH (20 mL) was added dropwise to decompose excess BH₃. The resulting mixture was stirred until no bubble was released and then 10% NaOH (10 mL) was added. The mixture was concentrated and the residue was mixed with water (200 mL) and extracted with EtOAc. The residue from rotary evaporation was purified by silica gel column chromatography (5:1 hexane/EtOAc) to give 2-bromo-5-chlorobenzyl alcohol as a white solid (4.58 g, 88%): ¹H NMR (300 MHz, DMSO-d₆): δ (ppm) 7.57 (d, J=8.7 Hz, 1H), 7.50-7.49 (m, 1H), 7.28-7.24 (m, 1H), 5.59 (t, J=6.0 Hz, 1H), 4.46 (d, J=6.0 Hz, 2H).

2-Bromo-5-chlorobenzyl alcohol obtained above was dissolved in CH₂Cl₂ (150 mL) and cooled to 0° C. on an ice bath. To this solution under nitrogen were added in sequence i-Pr₂NEt (5.4 mL, 31 mmol) and chloromethyl methyl ether (2.0 mL, 26 mmol). The reaction mixture was stirred overnight at room temperature and washed with NaHCO₃-saturated water and then brine. The residue after rotary evaporation was purified by silica gel column chromatography (5:1 hexane/EtOAc) to give 18d (4.67 g, 85%) as a colorless oil: ¹H NMR (300 MHz, DMSO-d₆): δ (ppm) 3.30 (s, 3H), 4.53 (s, 2H), 4.71 (s, 2H), 7.32 (dd, J=8.4, 2.4 Hz, 1H), 7.50 (dd, J=2.4, 0.6 Hz, 1H), 7.63 (d, J=8.7 Hz, 1H).

### 17.3 4-Bromo-3-(methoxymethoxymethyl)toluene (18e)

This compound was made from 2-bromo-5-methylbenzoic acid in the same manner as compound 18d: ¹H NMR (300 MHz, DMSO-d₆) δ (ppm) 2.27 (s, 3H), 3.30 (s, 3H), 4.51 (s, 2H), 4.68 (s, 2H), 7.05 (dd, J=7.9, 2.3 Hz, 1H), 7.30 (d, J=1.5 Hz, 1H), 7.46 (d, J=8.2 Hz, 1H).

### 17.4 2-Bromo-5-methoxy-1-(methoxymethoxymethyl)benzene (18g)

This compound was made from 2-bromo-5-methoxybenzoic acid in the same manner as compound 18d: ¹H NMR (300 MHz, DMSO-d₆): δ 3.30 (s, 1H), 3.74 (s, 3H), 4.50 (s, 2H), 4.69 (s, 2H), 6.83 (dd, J=8.8, 2.9 Hz, 1H), 7.40 (d, J=2.9 Hz, 1H), 7.48 (d, J=8.8 Hz, 1H).

### 17.5 2-Bromo-1,5-bis(methoxymethoxymethyl)benzene (18h)

This compound was made from 4-bromo-1,3-phthalic acid in the same manner as compound 18d: ¹H NMR (300 MHz, CDCl₃) δ (ppm) 3.28 (s, 3H), 3.30 (s, 3H), 4.50 (s, 2H), 4.54 (s, 2H), 4.64 (s, 2H), 4.69 (s, 2H), 7.20 (dd, J=8.8, 2.5 Hz, 1H), 7.46 (d, J=2.5 Hz, 1H), 7.58 (d, J=8.8 Hz, 1H).

### 17.6 2-Bromo-4,5-difluoro-1-(methoxymethoxymethyl)benzene (18k)

This compound was made from 2-bromo-4,5-difluorobenzoic acid in the same manner as compound 18d: ¹H NMR (300 MHz, CDCl₃) δ (ppm) 3.42 (s, 3H), 4.57 (d, J=1.2 Hz, 2H), 4.76 (s, 2H), 7.3-7.5 (m, 2H).

### 17.7 2-Bromo-6-fluoro-1-(methoxymethoxymethyl)benzene (18l)

This compound was made from 2-bromo-6-fluorobenzoic acid in the same manner as compound 18d: ¹H NMR (300 MHz, CDCl₃) δ (ppm) 3.43 (s, 3H), 4.74 (s, 2H), 4.76 (d, J=2.1 Hz, 2H), 7.05 (t, J=9.1 Hz, 1H), 7.18 (td, J=8.2, 5.9 Hz, 1H), 7.40 (d, J=8.2 Hz, 1H).

### 17.8 2-Bromo-4-fluoro-1-(methoxymethoxymethyl)benzene (18m)

This compound was made from 2-bromo-4-fluorobenzoic acid in the same manner as compound 18d and was used for the next step without purification.

### 17.9 4-Bromo-3-(methoxymethoxymethyl)benzonitrile (18f)

To a solution of 17 (10.0 g, 49.5 mmol) in carbon tetrachloride (200 mL) were added N-bromosuccinimide (8.81 g, 49.5 mmol) and 2,2′-azobis(isobutyronitrile) (414 mg, 5 mol %), and the mixture was refluxed for 3 h. Water was added, and the mixture was extracted with chloroform. The organic layer was washed with brine and dried over anhydrous sodium sulfate. The solvent was removed under reduced pressure. To the residue were added dimethylformamide (150 mL) and sodium acetate (20.5 g, 250 mmol), and the mixture was stirred at 80° C. overnight. Water was added, and the mixture was extracted with ether. The organic layer was washed with water and brine, and dried over anhydrous sodium sulfate. The solvent was removed under reduced pressure. To the residue was added methanol (150 mL) and 1 mol/L sodium hydroxide (50 mL), and the mixture was stirred at room temperature for 1 h. The reaction mixture was concentrated to about a third of volume under reduced pressure. Water and hydrochloric acid were added, and the mixture was extracted with ethyl acetate. The organic layer was washed with water and brine, and dried over anhydrous sodium sulfate. The solvent was removed under reduced pressure, and the residue was purified by silica gel column chromatography (3:1 hexane/ethyl acetate) followed by trituration with dichloromethane to give 2-bromo-5-cyanobenzyl alcohol (4.63 g, overall 44%): ¹H NMR (300 MHz, DMSO-d₆) δ (ppm) 4.51 (d, J=5.9 hz, 2H), 5.67 (t, J=5.6 Hz, 1H), 7.67 (dd, J=8.2, 2.0 Hz, 1H), 7.80 (s, J=8.2 Hz, 1H), 7.83 (d, J=2.0 Hz, 1H).

To a solution of 2-bromo-5-cyanobenzyl alcohol (4.59 g, 21.7 mmol) in dichloromethane (80 mL) were added diisopropylethylamine (5.6 mL, 32 mmol) and chloromethyl methyl ether (2.3 mL, 30 mmol) at 0° C., and the reaction mixture was stirred at room temperature overnight. Water was added, and the mixture was extracted with chloroform. The organic layer was washed with brine and dried over anhydrous sodium sulfate. The solvent was removed under reduced pressure. The residue was purified by silica gel column chromatography (6:1 hexane/ethyl acetate) to give 18f (4.08 g, 71%): ¹H NMR (300 MHz, CDCl₃) δ (ppm) 3.43 (s, 3H), 4.65 (s, 2H), 4.80 (s, 2H), 7.43 (dd, J=8.2, 4.1 Hz, 1H), 7.66 (d, J=8.2 Hz, 1H), 7.82 (d, J=4.1 Hz, 1H).

### 17.10 2-Bromo-5-trifluoromethyl-1-(methoxymethoxymethyl)benzene (18i)

This compound was made from 2-bromo-5-trifluoromethylbenzaldehyde in the same manner as compound 5b and used for the next step without purification.

### 17.11 1-Bromo-2-(methoxymethoxymethyl)naphthalene (18j)

This compound was made from 1-bromonaphthaldehyde in the same manner as compound 5b: ¹H NMR (300 MHz, CDCl₃) δ (ppm) 3.42 (s, 3H), 4.75 (s, 2H), 4.81 (s, 2H), 7.5-7.7 (m, 3H), 7.99 (d, J=7.7 Hz, 2H), 8.22 (d, J=7.7 Hz, 1H).

### 17.12 1,3-Dihydro-1-hydroxy-2,1-benzoxaborole (19a)

This compound was purchased from Lancaster Synthesis.

### 17.13 1,3-Dihydro-5-fluoro-1-hydroxy-2,1-benzoxaborole (19b)

To a solution of 5b (73.2 g, 293 mmol) in dry THF (400 mL) was added n-butyllithium (1.6 M in hexanes; 200 mL) over 45 min at −78° C. under nitrogen atmosphere. Anion precipitated. After 5 min, (i-PrO)₃B (76.0 mL, 330 mmol) was added over 10 min, and the mixture was allowed to warm to room temperature over 1.5 h. Water and 6 N HCl (55 mL) were added, and the solvent was removed under reduced pressure to about a half volume. The mixture was poured into ethyl acetate and water. The organic layer was washed with brine and dried over anhydrous Na₂SO₄. The solvent was removed under reduced pressure. To a solution of the residue in tetrahydrofuran (360 mL) was added 6 N HCl (90 mL), and the mixture was stirred at 30° C. overnight. The solvent was removed under reduced pressure to about a half volume. The mixture was poured into ethyl acetate and water. The organic layer was washed with brine and dried over anhydrous Na₂SO₄. The solvent was removed under reduced pressure, and the residue was treated with i-Pr₂O/hexane to give 19b (26.9 g, 60%) as a white powder: mp 118-120° C.; ¹H NMR (300 MHz, DMSO-d₆) δ (ppm) 4.95 (s, 2H), 7.15 (m, 1H), 7.24 (dd, J=9.7, 1.8 Hz, 1H), 7.74 (dd, J=8.2, 6.2 Hz, 1H), 9.22 (s, 1H); ESI-MS m/z 151 (M−H)⁻; HPLC purity 97.8%; Anal (C₇H₆BFO₂) C, H.

### 17.14 1,3-Dihydro-5-fluoro-1-hydroxy-3-methyl-2,1-benzoxaborolane (19c)

This compound was made from 18c in the same manner as compound 19b: mp 72-76° C. ¹H NMR (300 MHz, DMSO-d₆) δ (ppm) 1.37 (d, J=6.4 Hz, 3H), 5.17 (q, J=6.4 Hz, 1H), 7.14 (m, 1H), 7.25 (dd, J=9.7, 2.3 Hz, 1H), 7.70 (dd, J=8.2, 5.9 Hz, 1H), 9.14 (s, 1H). ESI-MS m/z 165 (M−H)⁻; HPLC purity 95.2%; Anal (C₈H₉BO₂) C, H.

### 17.15 5-Chloro-1,3-dihydro-1-hydroxy-2,1-benzoxaborole (19d)

This compound was made from 18d in the same manner as compound 19b: mp 142-144° C. ¹H NMR (300 MHz, DMSO-d₆) δ (ppm) 4.96 (s, 2H), 7.38 (d, J=7.8 Hz, 1H), 7.49 (s, 1H), 7.71 (d, J=7.8 Hz, 1H), 9.30 (s, 1H); ESI-MS m/z 167 (M−H)⁻; HPLC purity 99.0%; Anal (C₇H₆BClO₂·0.1H₂O)C, H.

### 17.16 1,3-Dihydro-1-hydroxy-5-methyl-2,1-benzoxaborole (19e)

This compound was made from 18e in the same manner as compound 19b: mp 124-128° C.; ¹H NMR (300 MHz, DMSO-d₆) δ (ppm) 2.33 (s, 3H), 4.91 (s, 2H), 7.13 (d, J=7.2 Hz, 1H), 7.18 (s, 1H), 7.58 (d, J=7.2 Hz, 1H), 9.05 (s, 1H); ESI-MS m/z 147 (M−H)⁻; HPLC purity 99.0%; Anal (C₈H₉BO₂) C, H.

### 17.17 1,3-Dihydro-1-hydroxy-5-methoxy-2,1-benzoxaborole (19g)

This compound was made from 18g in the same manner as compound 19b: mp 102-104° C.; ¹H NMR (300 MHz, DMSO-d₆) δ (ppm) 3.77 (s, 3H), 4.91 (s, 2H), 6.88 (d, J=8.1 Hz, 1H), 6.94 (s, 1H), 7.60 (d, J=8.1 Hz, 1H), 8.95 (s, 1H); ESI-MS m/z 163 (M−H)⁻; HPLC purity 100%; Anal (C₈H₉BO₃) C, H.

### 17.18 1,3-Dihydro-1-hydroxy-5-hydroxymethyl-2,1-benzoxaborole (19h)

This compound was made from 18h in the same manner as compound 19b: mp 124-128° C.; ¹H NMR (300 MHz, DMSO-d₆) δ (ppm) 4.53 (d, 2H), 4.94 (s, 2H), 5.24 (t, 1H), 7.26 (d, 1H), 7.33 (s, 1H), 7.64 (d, 1H), 9.08 (s, 1H); ESI-MS m/z 163 (M−H)⁻; HPLC purity 100%.

### 17.19 1,3-Dihydro-1-hydroxy-5-trifluoromethoxy-benzoxaborole (19i)

This compound was made from 181 in the same manner as compound 19b: mp 113-118° C.; ¹H NMR (300 MHz, DMSO-d₆) δ (ppm) 5.05 (s, 2H), 7.65-7.68 (d, J=7.5 Hz, 1H), 7.78 (s, 1H), 7.90-7.93 (d, J=7.8 Hz, 1H), 9.47 (s, 1H); ESI-MS m/z 201 (M−H)⁻; HPLC purity 100%.

### 17.20 1,3-Dihydro-1-hydroxy-2,1-naphtho[2,1-d]oxaborole (19j)

This compound was made from 18j in the same manner as compound 19b: mp 139-143° C.; ¹H NMR (300 MHz, DMSO-d₆) δ (ppm) 5.09 (s, 2H), 7.59-7.47 (m, 3H), 7.95 (d, J=7.5 Hz, 1H), 7.99 (d, J=8.1 Hz, 1H), 8.28 (dd, J=6.9, 0.6 Hz, 1H), 9.21 (s, 1H); ESI-MS m/z 185 (M+H)⁺; Anal (C₁₁H₉BO₂) C, H.

### 17.21 1,3-Dihydro-4-fluoro-1-hydroxy-2,1-benzoxaborole (19l)

This compound was made from 18L in the same manner as compound 19b:

¹H NMR (300 MHz, DMSO-d₆) δ (ppm) 5.06 (s, 2H), 7.26 (ddd, J=9.7, 7.9, 0.6 Hz, 1H), 7.40 (td, J=8.2, 4.7 Hz, 1H), 7.55 (d, J=7.0 Hz, 1H), 9.41 (s, 1H); ESI-MS m/z 151 (M−H)⁻; HPLC purity 98.7%; Anal (C₇H₆BFO₂) C, H.

### 17.22 1,3-Dihydro-6-fluoro-1-hydroxy-2,1-benzoxaborole (19m)

This compound was made from 18m in the same manner as compound 19b: ¹H NMR (300 MHz, DMSO-d₆) δ (ppm) 4.95 (s, 2H), 7.29 (td, J=9.0, 2.7 Hz, 1H), 7.41-7.46 (m, 2H), 9.29 (s, 1H); ESI-MS m/z 151 (M−H)⁻; HPLC purity 100%; Anal (C₇H₆BFO₂) C, H.

### 17.23 5,6-Difluoro-1,3-dihydro-1-hydroxy-2,1-benzoxaborolane (19k)

To a solution of 18k (2.97 g, 11.1 mmol) and (1-PrO)₃B (2.8 mL, 12 mmol) in THF (30 mL) was added n-BuLi (1.6 mol/L in hexane; 7.5 mL) over 30 min at −78° C. under nitrogen atmosphere, and the mixture was stirred for 2 h while allowing to warm to room temperature. The reaction was quenched with 2 N HCl, and the mixture was extracted with EtOAc. The organic layer was washed with brine and dried over anhydrous Na₂SO₄. The solvent was removed under reduced pressure. To a solution of the residue in THF (25 mL) was added 6 N HCl (5 mL), and the mixture was stirred at room temperature overnight. Water was added and the mixture was extracted with EtOAc. The organic layer was washed with brine and dried over anhydrous Na₂SO₄. The solvent was removed under reduced pressure. Recrystallization from EtOAc/i-Pr₂O gave 19k (1.14 g, 60%) as a white powder: mp 134-140° C. ¹H NMR (300 MHz, DMSO-d₆) δ (ppm) 4.94 (s, 2H), 7.50 (dd, J=10.7, 6.8 Hz, 1H), 7.62 (dd, J=9.7, 8.2 Hz, 1H), 9.34 (s, 1H). ESI-MS m/z 169 (M−H)⁻; HPLC purity 96.6%; Anal (C₇H₅BF₂O₂) C, H.

### 17.24 5-Cyano-1,3-dihydro-1-hydroxy-2,1-benzoxaborolane (19f)

This compound was made from 18f in the same manner as compound 19k: mp 98-101° C. ¹H-NMR (300 MHz, DMSO-d₆) δ (ppm) 5.03 (s, 2H), 7.76 (d, J=8.2 Hz, 1H), 7.89 (d, J=8.2 Hz, 1H), 7.90 (s, 1H), 9.53 (s, 1H); ESI-MS m/z 158 (M−H)⁻; HPLC purity 97.7%.

### 17.25 1,3-Dihydro-7-fluoro-1-hydroxy-2,1-benzoxaborolane (19n)

To a solution of 20 (2.00 g, 15.9 mmol) and TMEDA (5.70 mL, 38.0 mmol) in THF (100 mL) was added sec-butyllithium (25 mL, 35.0 mmol) as 1.4 M solution at −78° C. The mixture was stirred at −78° C. for 1 h before (1-PrO)₃B (8.10 mL, 35.0 mmol) was added. The reaction was warmed up to room temperature very slowly, then was stirred overnight. Water was added, and the pH was adjusted to 12, then it was washed with ethyl ether. The aqueous layer was acidified to pH 2 using 6 N HCl, then extracted with ethyl ether, washed with brine, dried over Na₂SO₄ and concentrated under reduced pressure. The crude product was purified by flash column chromatography (2:1 hexane/ethyl acetate) to give 19n (270 mg) as a white solid: mp 120-124° C. ¹H NMR (300 MHz, DMSO-d₆) δ (ppm) 4.99 (s, 2H), 7.00 (t, J=8.7 Hz, 1H), 7.21 (d, J=7.8 Hz, 1H), 7.48 (td, J=5.1, 7.8 Hz, 1H), 9.25 (s, 1H); ESI-MS m/z 151 (M−H)⁻; HPLC purity 97.4%; Anal (C₈H₆BNO₂) C, H.

### Example 18

### Benzoxaborin

### 18.1 2-Bromo-5-fluorophenylacetaldehyde (21a)

A mixture of compound 3 (4.23 g, 20.0 mmol), (methoxymethyl)triphenylphosphonium chloride (8.49 g, 24.0 mmol), and potassium tert-butoxide (2.83 g, 24.0 mol) in N,N-dimethylformamide (50 mL) was stirred at room temperature overnight. The reaction was quenched with 6 N hydrochloric acid, and the mixture was extracted with ethyl acetate. The organic layer was washed with water twice and brine, and dried over anhydrous sodium sulfate. The solvent was removed under reduced. To the residue were added tetrahydrofuran (60 mL) and 6 N hydrochloric acid, and the mixture was heated at reflux for 8 h. Water was added, and the mixture was extracted with ether. The organic layer was washed with brine and dried over anhydrous sodium sulfate. The solvent was removed under reduced pressure to afford 21a (3.60 g, 83%): ¹H NMR (300 MHz, CDCl₃) δ (ppm) 3.86 (d, J=1.5 Hz, 2H), 6.9-7.1 (m, 2H), 7.57 (dd, J=8.8, 5.3 Hz, 1H), 9.76 (t, J=1.5 Hz, 1H).

### 18.2 1-Bromo-4-fluoro-2-[2-(methoxymethoxy)ethyl]benzene (22a)

To a solution of 21a (3.60 g, 16.6 mmol) in methanol (40 mL) was added sodium borohydride (640 mg, 16.6 mmol) at 0° C., and the mixture was stirred at room temperature for 1 h. Water was added, and the mixture was extracted with ethyl acetate. The organic layer was washed with brine and dried over anhydrous sodium sulfate. The solvent was removed under reduced pressure. To the residue were added dichloromethane (50 mL), diisopropylethylamine (3.5 mL, 20 mmol) and chloromethyl methyl ether (1.5 mL, 20 mmol) at 0° C., and the reaction mixture was stirred at room temperature overnight. Water was added, and the mixture was extracted with chloroform. The organic layer was washed with brine and dried over anhydrous sodium sulfate. The solvent was removed under reduced pressure. The residue was purified by silica gel column chromatography (15:1 hexane/ethyl acetate) to give 22a (2.99 g, 2 steps 68%) as a colorless oil: ¹H NMR (300 MHz, CDCl₃) δ (ppm) 3.04 (t, J=6.7 Hz, 2H), 3.31 (s, 3H), 3.77 (t, J=6.7 Hz, 2H), 4.62 (s, 2H), 6.82 (td, J=8.2, 3.2 Hz, 1H), 7.04 (dd, J=9.4, 2.9 Hz, 1H), 7.48 (dd, J=8.8, 5.3 Hz, 1H).

### 18.3 1-Bromo-2-[2-(methoxymethoxy)ethyl]benzene (22b)

This compound was synthesized from 21b in a similar manner to 22a and used for the next step without purification.

### 18.4 6-Fluoro-1-phenyl-1,2,3,4-tetrahydro-2,1-benzoxaborine (23a)

This compound was synthesized from 22a and 7a in a similar manner to compound 9f: colorless oil; ¹H NMR (300 MHz, CDCl₃) δ (ppm) 3.02 (t, J=6.1 Hz, 2H), 4.34 (t, J=6.1 Hz, 2H), 6.9-7.1 (m, 2H), 7.4-7.6 (m, 3H), 7.8-7.9 (m, 3H); ESI-MS m/z 227 (M+H)⁺; HPLC purity 95.3%; Anal (C₁₄H₁₂BFO·0.1H₂O)C, H.

### 18.5 1-Phenyl-1,2,3,4-tetrahydro-2,1-benzoxaborine (23b)

This compound was synthesized from 22b and 7a in a similar manner to compound 9f: colorless oil; ¹H NMR (300 MHz, DMSO-d₆) δ (ppm) 2.94 (t, J=5.9 Hz, 2H), 4.21 (t, J=5.9 Hz, 2H), 7.28 (t, J=7.9 Hz, 2H), 7.3-7.5 (m, 4H), 7.66 (d, J=7.0 Hz, 1H), 7.75 (d, J=7.6 Hz, 2H); ESI-MS m/z not observed; HPLC purity 96.0%; Anal (C₁₄H₁₃BO) C. H.

### 18.6 6-Fluoro-1-hydroxy-1,2,3,4-tetrahydro-2,1-benzoxaborine (24)

This compound was synthesized from 22a in a similar manner to compound 19b. Silica gel column chromatography (2:1 hexane/ethyl acetate) followed by trituration with pentane 24 as a white powder: mp 77-82° C.; ¹H NMR (300 MHz, DMSO-d₆) δ (ppm) 2.86 (t, J=5.9 Hz, 2H), 4.04 (t, J=5.9 Hz, 2H), 7.0-7.1 (m, 2H), 7.69 (dd, J=8.2, 7.2 Hz, 1H), 8.47 (s, 1H); ESI-MS m/z 165 (M−H)⁻; HPLC purity 99.0%; Anal (C₈H₈BFO₂) C, H.

### Example 19

### Formation of Ethylene Glycol Boronate Ester (3, T=Nothing)

General Procedure

Boronic acid was dissolved in dry THF, dry toluene or dry diethyl ether (about 10 mL/g) under nitrogen. Ethylene glycol (1 molar equivalent) was added to the reaction and the reaction was heated to reflux for 1 to 4 hours. Reaction was cooled to room temperature and solvent was removed under reduced pressure leaving the ethylene glycol ester as an oil or a solid. In cases where an oil was obtained or a solid that dissolved in hexane, dry hexane was added and removed under reduced pressure. The product was then placed under high vacuum for several hours. In cases where a solid was obtained that did not dissolve in hexane, the solid was collected by filtration and washed with cold hexane.

### 3-Cyanophenylboronic acid ethylene glycol ester (3a)

3-Cyanophenyl boronic acid (1 g, 6.8 mmol) was dissolved in dry THF (10 mL) under nitrogen. Ethylene glycol (379 .mu.L, 422 mg, 6.8 mmol) was added and the reaction was heated to reflux for 4 hours then cooled to room temperature. THF was removed by rotary evaporator to give a white solid. Cold hexane was added and the product was collected by filtration giving a white solid (1.18 g, quant. yield). .sup.1H-NMR (300.058 MHz, DMSO-d6) .delta. ppm 7.92-8.01 (3H, m), 7.50-7.64 (1H, m), 4.35 (4H, s)

### Thiophene 3-boronic acid ethylene glycol ester (3b)

Thiophene-3-boronic acid (1 g, 7.8 mmol) was dissolved in dry THF (10 mL) under nitrogen. Ethylene glycol (435 .mu.L, 484 mg, 7.8 mmol) was added and the reaction was heated to reflux for 1 hour then cooled to room temperature. THF was removed by rotary evaporator to give a white solid. Hexane was added, dissolving the solid and removed by rotary evaporation. The product was placed under high vacuum to yield a tan solid (1.17 g, 97%). .sup. ¹H-NMR (300.058 MHz, CDCl.sub.3) .delta. ppm 7.93 (1H, s), 7.3-7.4 (2H, m), 4.35 (4H, s).

### 3-Fluorophenylboronic acid ethylene glycol ester (3c)

A mixture of 3-fluorophenylboronic acid (7.00 g, 50.0 mmol) and ethylene glycol (2.8 mL, 50 mmol) in toluene (200 mL) was heated to reflux for 3 hours under Dean-Stark conditions. The solvent was removed under reduced pressure to afford 3-fluorophenylboronic acid ethylene glycol ester (7.57 g, 91%).

Formation of Unsymmetrical Borinic Acid (6) from Boronic Acid Ethylene Glycol Ester

General Procedure A: Grignard Methodology

Boronic acid ethylene glycol ester was dissolved in dry THF (10-20 mL/g) under nitrogen. Solution was cooled to −78° C. in an acetone/dry ice bath or to 0° C. in an ice/water bath. Grignard reagent (0.95 to 1.2 molar equivalent) was added dropwise to the cooled solution. The reaction was warmed to room temperature and stirred for 3-18 hours. 6N HCl (2 mL/g) was added and solvent was removed under reduced vacuum. Product was extracted into diethyl ether (40 mL/g) and washed with water (3.times.equal volume). Organic layer was dried (MgSO₄), filtered and the solvent was removed by rotary evaporation giving the crude product, which is either purified by column chromatography or taken onto the next step without purification. Alternative work-up: if the borinic acid product contained a basic group such as an amine or pyridine, then after stirring at room temperature for 3-18 hours, water (2 mL/g) was added and the pH adjusted to 5-8. Product was extracted into diethyl ether or ethyl acetate or THF up to three times (40 mL/g). Organic layer was dried (MgSO₄), filtered and the solvent was removed by rotary evaporation giving the crude product, which is either purified by column chromatography or taken onto the next step without purification.

### (4-Cyanophenyl)(3-fluorophenyl)borinic acid (6a)

4-Cyanophenyl boronic acid ethylene glycol ester (500 mg, 2.89 mmol) was dissolved in dry THF under nitrogen. The solution was cooled to −78° C. in an acetone/dry ice bath and 3-fluorophenylmagnesium bromide (1M in THF)(2.74 mL, 2.74 mmol, 0.95 molar equivalent) was added dropwise to the cold solution. The reaction was allowed to warm slowly to room temperature and stirred for 18 hours. 6N HCl (1 mL) was added to the reaction causing a cloudy appearance and the solvent was removed using a rotary evaporator. The product was extracted into diethyl ether (20 mL) and washed with water (3×20 mL). The organic layer was dried (MgSO₄), filtered and the solvent removed using a rotary evaporator to yield the crude product as an oily solid. This was taken onto the next step without purification.

General Procedure B: (Hetero)Aryl-Lithium Methodology

The (hetero)aryl-bromide or iodide was dissolved in dry THF (20-30 mL/g) under nitrogen and degassed. The solution was cooled to −78° C. in an acetone/dry ice bath and n-, sec- or tert-butyllithium in THF or other solvent (1.2-2.4 molar equivalents) was added to the cooled solution dropwise (generally causing the solution to turn deep yellow). The boronic acid ethylene glycol ester (1 molar equivalent) was dissolved in dry THF or diethyl ether (2-10 mL/g) under nitrogen. The boronic acid ethylene glycol ester in THF was added dropwise to the cooled aryl-lithium solution (generally causing the solution to turn pale yellow). The reaction was warmed to room temperature and stirred for 1-18 hours. 6N HCl (2-4 mL/g) was added and solvent was removed under reduced vacuum. Product was extracted into diethyl ether (40 mL/g) and washed with water (3.times.equal volume). Organic layer was dried (MgSO₄), filtered and the solvent was removed by rotary evaporation giving the crude product, which is either purified by column chromatography or taken onto the next step without purification. Alternative work-up: if the borinic acid product contained a basic group such as an amine or pyridine then after stirring at room temperature for 3-18 hours water (2 mL/g) was added and the pH adjusted to 5-8. Product was extracted into diethyl ether or ethyl acetate or THF up to three times (40 mL/g) and washed with water (3.times.equal volume). Organic layer was dried (MgSO₄), filtered and the solvent was removed by rotary evaporation giving the crude product, which is either purified by column chromatography or taken onto the next step without purification.

### (3-Thienyl)(3-chlorophenyl)borinic acid (6b)

3-Chloro-bromobenzene (447 .mu.L, 728 mg, 3.8 mmol) was dissolved in dry THF (15 mL) under nitrogen. The solution was degassed and cooled to −78° C. in an acetone/dry ice bath. tert-Butyllithium (1.7M in THF)(4.47 mL, 7.6 mmol, 2 molar equivalent) was added to the cooled solution dropwise causing the solution to turn deep yellow. The solution was stirred at −78° C. while 3-thiopheneboronic acid ethylene glycol ester (586 mg) was dissolved in dry diethyl ether (1 mL). The boronic ester solution was then added dropwise to the cooled solution causing the color to change to pale yellow. The reaction was warmed to room temperature and stirred for 18 hours. 6N HCl (2 mL) was added and the reaction was stirred for 1 hour. The solvent was removed using a rotary evaporator. The product was extracted into diethyl ether (10 mL) and washed with water (2.times. 10 mL). The organic layer was dried (MgSO₄), filtered and the solvent removed using a rotary evaporator to yield the crude product as an orange oil. The product was purified by column chromatography using silica gel and hexane:ethyl acetate 5:1 as eluent giving the pure product as a clear oil (614 mg, 73%).

### (3-Chlorophenyl)vinylborinic acid (6c)

This was prepared by a similar process as described for 6b by the reaction of 3-cyanophenyl boronic acid ethylene glycol ester with vinyllithium.

### (3-Fluoro-5-chlorophenyl)ethynylborinic acid (6d)

This was prepared by a similar process as described for 6b by the reaction of 3-fluoro-5-chlorophenyl boronic acid ethylene glycol ester with ethynyllithium.

### (4-Methyl-3-chlorophenyl)(2-thienyl)borinic acid (6e)

This was prepared by a similar process as described for 6b by the reaction of 2-thienylboronic acid ethylene glycol ester with 4-methyl-3-chlorophenyllithium.

### (4-Cyanophenyl)ethynylborinic acid (6f)

This was prepared by a similar process as described for 6b by the reaction of 4-cyanophenylboronic acid ethylene glycol ester with ethynyllithium.

### (3-Fluorophenyl)cyclopropylborinic acid (6q)

This was prepared by a similar process as described for 6b by the reaction of 3-fluorophenylboronic acid ethylene glycol ester with cyclopropyllithium.

### (3-Thienyl)methylborinic acid (6h)

This was prepared by a similar process as described for 6b by the reaction of 3-thienylboronic acid ethylene glycol ester with methyllithium.

### (4-Pyridyl)phenylborinic acid (6i)

This was prepared by a similar process as described for 6b by the reaction of phenylboronic acid ethylene glycol ester with 4-pyridyllithium.

### (3-Cyanophenyl)(2-fluorophenyl)borinic acid (6j)

This was prepared by a similar process as described for 6b by the reaction of 3-cyanophenylboronic acid ethylene glycol ester with 2-fluorophenyllithium.

### 4-(Dimethylaminomethyl)phenyl 3-fluorophenyl borinic acid (6k)

Sec-butyllithium (1.4 M in cyclohexane, 6.0 mL) was added to a solution of N,N-dimethyl-4-bromobenzylamine (1.50 g, 7.00 mmol) in THF (14 mL) at −78° C. under nitrogen atmosphere and the mixture was stirred for 15 min. 3-Fluorophenylboronic acid ethylene glycol ester (1.16 g, 7.00 mmol) in THF (7 mL) was added to the mixture. The reaction was allowed to warm to room temperature and stirred for 1 h. Water was added and the mixture was washed with ether. The pH was adjusted to 8 with 1M hydrochloric acid. The mixture was extracted with ethyl acetate twice. The organic layer was washed with brine and dried on anhydrous sodium sulfate. The solvent was removed under reduced pressure to afford the borinic acid (890 mg, 49%).

Formation of Symmetrical Borinic Acid (5) by Reaction of Organometallics with Trialkyl Borates.

### Bis(4-chlorophenyl)borinic acid (5a) (Procedure C)

A cold solution (−78° C.) of trimethyl borate (0.37 mL) in dry tetrahydrofuran (THF, 25 ml) was treated dropwise with 4-chlorophenylmagnesium bromide (6.75 ml, 1M solution in ether). The reaction mixture was stirred at −78° C. for 1 h and then stirred for 18 h at room temperature. The solvent was removed under reduced pressure. The resultant residue was stirred with 100 ml of ether and 15 ml of 6N hydrochloric acid. Organic layer was separated and aqueous layer was extracted with ether (2.times. 100 ml). The combined organic extract was washed with brine and dried over anhydrous magnesium sulfate. Solvent was removed to give light yellowish solid. The product was chromatographed over silica gel (Hex:Ether=1:1) to give 420 mg of borinic acid. ¹H NMR (400 MHz, CDCl₃) δ: 5.84 (s, OH), 7.46 (d, 4H, Ar—H), 7.72 (d, 4H, Ar—H).

### Bis(3-Chloro-4-methylphenyl)borinic acid (5b)

In a similar manner as for 5a, the titled compound was obtained from the reaction of 3-chloro-4-methylphenylmagnesium bromide with trimethyl borate. The product was obtained by chromatography over silica gel.

### Bis(3-Fluoro-4-methylphenyl)borinic acid (5c)

In a similar manner as for 5a, the titled compound was obtained from the reaction of 3-fluoro-4-methylphenyllithium with trimethyl borate. The product was obtained by chromatography over silica gel.

### Bis(3-Chloro-4-methoxyphenyl)borinic acid (5d)

In a similar manner as for 5a, the titled compound was obtained from the reaction of 3-chloro-4-methoxyphenyllithium with trimethyl borate. The product was obtained by chromatography over silica gel.

### Bis(3-Fluoro-4-methoxyphenyl)borinic acid (5e)

In a similar manner as for 5a, the titled compound was obtained from the reaction of 3-fluoro-4-methoxyphenyllithium with trimethyl borate. The product was obtained by chromatography over silica gel.

Formation of Unsymmetrical Borinic Acids (6) by Reaction of Organometallics with Alkyl (or Aryl or Alkenyl) Dialkoxyboranes.

### (4-Chloro-phenyl)methyl-borinic acid (6m) (Procedure D)

To 4-chlorophenylmagnesium bromide (5.5 ml, 1M solution in ether) at −78° C., di(isopropoxy)methylborane (1 ml, 0.78 g) was added dropwise via syringe. The reaction mixture was stirred at −78° C. for 1 h and then stirred overnight at ambient temperature. The reaction mixture was treated dropwise with 100 ml of ether and 15 ml of 6N hydrochloric acid, and stirred for 1 h. Organic layer was separated and aqueous layer was extracted with ether (2×100 ml). The combined organic extract was washed with brine and dried over anhydrous sodium sulfate. Solvent was removed under reduce pressure to give 1.1 g of oil. ¹H NMR of the product was consistent for (4-chlorophenyl)methyl borinic acid.

### (4-Fluorophenyl)methylborinic acid (6n)

In a similar manner as for 6m, the titled compound was obtained from the reaction of 4-fluorophenylmagnesium bromide with di(isopropoxy)methylborane. The product was obtained by chromatography over silica gel.

### (4-Biphenyl)methylborinic acid (6o)

In a similar manner as for 6m, the titled compound was obtained from the reaction of 4-biphenyllithium with di(isopropoxy)methylborane. The product was obtained by chromatography over silica gel.

### (3-Chloro-4-methylphenyl)methylborinic acid (6p)

In a similar manner as for 6m, the titled compound was obtained from the reaction of 3-chloro4-methylphenyllithium with di(isopropoxy)methylborane. The product was obtained by chromatography over silica gel.

### (3-Chloro-4-methoxyphenyl)methylborinic acid (6q)

In a similar manner as for 6m, the titled compound was obtained from the reaction of 3-chloro-4-methoxyphenyllithium with di(isopropoxy)methylborane. The product was obtained by chromatography over silica gel.

### (4-Dimethylaminophenyl)methylborinic acid (6r)

In a similar manner as for 6m, the titled compound was obtained from the reaction of 4-dimethylaminophenyllithium with di(isopropoxy)methylborane. The product was obtained by chromatography over silica gel.

### (3-Pyridyl)vinyl borinic acid (6s)

Isopropylmagnesium chloride (2.0 M in THF) (5.0 mL, 10 mmol) was added to a solution of 3-bromopyridine (1.60 g, 10.0 mmol) in THF (15 mL) under nitrogen atmosphere at room temperature and the mixture was stirred for 1 h. Vinylboronic acid dibutyl ester (3.4 mL) was added to the reaction dropwise and the mixture was stirred at room temperature for 18 h. Water was added and the pH was adjusted to 7 with 1 M hydrochloric acid. The mixture was extracted with ethyl acetate. The organic layer was washed with brine and dried on anhydrous sodium sulfate. The solvent was removed under reduced pressure to give the title compound (1.04 g, 78%).

### (3-Chloro-4-dimethylaminophenyl)vinylborinic acid (6t)

In a similar manner as for 6s, the titled compound was obtained from the reaction of 3-chloro4-dimethylaminophenyllithium with vinylboronic acid dibutyl ester. The product was obtained by chromatography over silica gel.

Borinic Acid-Alkylalcohol Derivatives

### Bis(3-Chlorophenyl)borinic acid 4-hydroxyethyl)imidazole ester (121)

To a solution of bis(3-chlorophenyl)borinic acid (0.4 g, 1.428 mmol) in ethanol (10 ml), 4-(hydroxyethyl)imidazole hydrochloride (0.191 g, 1.428 mmol), sodium bicarbonate (0.180 g, 2.143 mmol) were added and the reaction mixture was stirred at room temperature for 18 h. Salt was removed by filtration. Filtrate was concentrated and treated with hexane to afford the product as a solid and was collected by filtration. (450 mg, 84.9% yield). ¹H NMR (CD₃OD) δ (ppm) 2.92 (t, 2H), 3.82 (t, 2H), 7.0-7.2 (m, 9H), 7.90 (s, 1H); (ES.sup.−)(m/z) 343.11, MF C.sub.17H.sub. 15BCl.sub.2N.sub.2O

### Bis(4-Chlorophenyl)borinic acid 4-(hydroxymethyl)imidazole ester (126)

In a similar manner as in Example 121, the titled compound was obtained from the reaction of bis(4-chlorophenyl)borinic acid with 4-(hydroxymethyl)imidazole hydrochloride. The product was obtained as white crystals. (ES.sup.−)(m/z) 328.79, MF C.sub.16H.sub.13BCl.sub.2N.sub-.2O

### Bis(3-Chloro-4-methylphenyl)borinic acid 1-benzyl-4-(hydroxymethyl)-imidazo-le ester (127)

To a solution of 1-benzyl-4-(hydroxymethyl)imidazole (96 mg, 0.521 mmol) in methanol (5 ml), bis(3-chloro4-methylphenyl)borinic acid (121 mg, 0.521 mmol) was added and the reaction mixture was stirred at room temperature for 2 h. Solvent was removed under reduced pressure and the residue was treated with hexane to give a solid. The product was isolated by filtration and washed with hexane to give product (193 mg, 83%). ¹H NMR (400 MHz, CDCl₃) δ: 2.3 (s, 6H, 2.times.CH.sub.3), 4.8 (brs, 2H, CH.sub.2), 5.1 (brs, 2H, CH.sub.2), 6.9-7.4 (complex, 13H, Ar—H); MS (ES.sup.+)(m/z) 448.78, MF C.sub.25H.sub.23BCl.sub.2N.sub.2O.

### Bis(3-Chloro-4-methylphenyl)borinic acid 1-methyl-2-(hydroxymethyl)-imidazole ester (128)

In a similar manner as in Example 127, the titled compound was obtained from the reaction of bis(3-chloro4-methylphenyl)borinic acid with 1-methyl-2-(hydroxy-methyl)imidazole hydrochloride. The product was obtained as white crystals. (ES.sup.+)(m/z) 372.82, MF C.sub.19H.sub.21BCl.sub.2N.sub.2O

### Bis(3-Chloro-4-methylphenyl)borinic acid 1-ethyl-2-(hydroxymethyl)-imidazo-le ester (129)

In a similar manner as in Example 127, the titled compound was obtained from the reaction of bis(3-chloro-4-methylphenyl)borinic acid with 1-ethyl-2-(hydroxy-methyl)imidazole hydrochloride. The product was obtained as white crystals. (ES.sup.+)(m/z) 386.83, MF C.sub.20H.sub.23BCl.sub.2N.sub.2O

### Bis(3-Chloro-4-methylphenyl)borinic acid 1-methyl-4-(hydroxymethyl)-imidazo-le ester (130)

In a similar manner as in Example 127, the titled compound was obtained from the reaction of bis(3-chloro-4-methylphenyl)borinic acid with 1-methyl-4-(hydroxy-methyl)imidazole hydrochloride. The product was obtained as white crystals. (ES.sup.+)(m/z) 372.88, MF C.sub.19H.sub.21BCl.sub.2N.sub.2O

### Bis(3-Chloro-4-methylphenyl)borinic acid 2-pyridylethanol (131)

In a similar manner as in Example 121, the titled compound was obtained from the reaction of bis(3-chloro-4-methylphenyl)borinic acid with 2-pyridylethanol. The product was obtained as white crystals. (ES.sup.+)(m/z) 383.84, MF C.sub.21H.sub.20BCl.sub.2NO

Hydroxyquinoline Derivatives

### Bis(3-Chlorophenyl)borinic acid 5-cyano8-hydroxyquinoline ester (19)

To a solution of bis(3-chlorophenyl)borinic acid (0.25 g) in ethanol (5 ml) and water (2 ml) was added 5-cyano-8-hydroxyquinoline (0.15 g). The solution was stirred at room temperature for 21 hours. A yellow solid precipitate formed which was collected by filtration and washed with cold ethanol. The product was obtained as yellow crystals. ¹H NMR (DMSO) δ: (ppm) 7.24-7.35 (m, 8H), 7.38 (d, 1H), 8.18 (dd, 1H), 8.40 (d, 1H), 8.86 (d, 1H), 9.50 (d, 1H).

### (3-Chlorophenyl)(2-thienyl)borinic acid 8-hydroxyquinoline ester (36)

To a solution of (3-chlorophenyl)(2-thienyl)borinic acid (1.5 g) in ethanol (2 ml) was added 8-hydroxyquinoline (0.77 g) in hot ethanol (2 ml). The reaction was heated to reflux and cooled to room temperature. A yellow solid precipitated. The mixture was cooled in ice, the solid was collected by filtration and washed with cold ethanol. The product was obtained as a yellow solid (1.01 g). ¹H NMR (400 MHz, CDCl₃) δ: (ppm) 6.98-7.06 (m, 2H), 7.19-7.26 (m, 3H), 7.38-7.50 (m, 4H), 7.71 (t, 1H), 7.91 (dd, 1H), 8.80 (d, 1H), 9.18 (d, 1H); (ESI.sup.+)(m/z) 350.1, MF C.sub. 19H.sub. 13BClNOS

### (2-Thienyl)methylborinic acid 8-hydroxyquinoline ester (26)

In a similar manner as in Example 36, the titled compound was obtained from the reaction of (2-thienyl)methylborinic acid with 8-hydroxyquinoline. The product was obtained as yellow crystals.

### (3-Cyanophenyl)vinylborinic acid 8-hydroxyquinoline ester (40)

In a similar manner as in Example 36, the titled compound was obtained from the reaction of (3-cyanophenyl)vinylborinic acid with 8-hydroxyquinoline. The product was obtained as yellow crystals. (ESI.sup.+)(m/z) 285.1, MF C.sub. 18H.sub. 13BN.sub.2O

### (2-Chlorophenyl)ethynylborinic acid 8-Hydroxyquinoline ester (43)

In a similar manner as in Example 36, the titled compound was obtained from the reaction of (2-chlorophenyl)ethynylborinic acid with 8-hydroxyquinoline. The product was obtained as yellow crystals. (ESI.sup.+)(m/z) 292.1, MF C.sub.17H.sub.11BClNO

### Bis(ethynyl)borinic acid 8-Hydroxyquinoline (44)

In a similar manner as in Example 36, the titled compound was obtained from the reaction of bis(ethynyl)borinic acid with 8-hydroxyquinoline. The product was obtained as light yellow crystals. (ESI.sup.+)(m/z) 206.1, MF C.sub.13H.sub.8BNO

### (3-Fluorophenyl)cyclopropylborinic acid 8-hydroxyquinoline ester (70)

In a similar manner as in Example 36, the titled compound was obtained from the reaction of (3-fluorophenyl)cyclopropylborinic acid with 8-hydroxyquinoline. The product was obtained as light yellow crystals. (ES.sup.−)(m/z) 291.05, MF C.sub.18H.sub.15BFNO

### (3-Pyridyl)vinylborinic acid 8-hydroxyquinoline ester (99)

A solution of (3-pyridyl)vinyl borinic acid (1.04 g, 7.82 mmol) and 8-hydroxyquinoline (961 mg, 6.63 mmol) in ethanol was stirred at 40° C. for 20 min. The solvent was removed under reduced pressure and the residue was crystallized from diethyl ether/diisopropyl ether/hexane to afford the title product (99) as a light yellow powder (355 mg, 21%). ¹H NMR (DMSO₆) δ: (ppm) 5.23 (dd, 1H), 5.46 (dd, 1H), 6.43 (dd, 1H), 7.14 (d, 1H), 7.19 (dd, 1H), 7.41 (d, 1H), 7.6-7.8 (m, 2H), 7.88 (dd, 1H), 8.35 (dd, 1H), 8.57 (s, 1H), 8.76 (d, 1H), 9.00 (d, 1H); ESI.sup.+ (m/z) 261 MF C.sub. 16H.sub. 13BN.sub.2O.

### (4-(Dimethylaminomethyl)phenyl)(3-fluorophenyl)borinic acid 8-hydroxy-quinoline ester (100)

In a similar manner as in Example 99, the titled compound was obtained from the reaction of (4-(Dimethylaminomethyl)phenyl)(3-fluorophe-nyl)borinic acid with 8-hydroxyquinoline. The product was obtained as a light yellow powder. ESI.sup.+ (m/z) 385 MF C.sub.24H.sub.22BFN.sub.2O.

3-Hydroxypicolinic Acid Derivatives

### Bis(3-Chloro-4-methylphenyl)borinic acid 3-hydroxypicolinate ester (111)

Bis(3-chloro-4-methylphenyl)borinic acid (14.6 g) was dissolved in ethanol (120 ml) and heated to reflux. 3-Hydroxypicolinic acid (5.83 g) was added in portions to the hot solution. The reaction was stirred at reflux for 15 minutes after the addition of the last portion of 3-hydroxypicolinic acid was added and then cooled to room temperature. Reaction was concentrated by removal of some ethanol. Solid was removed by filtration. One recrystallization from ethanol afforded the title product as white crystals (13.4 g). MP=165.0-166.5° C.

### Example 20

### Anti-inflammatory data for 3-hydroxypyridine-2-carbonyloxy-bis(3-chloro-4-methylphenyl)-borane

Pro-inflammatory cytokines: THP-1 cells, LPS@ 1 mg/mL, 24 hr

TNF-α: 2.7 μM; IL-1β: 1.0 μM; IL-6: 5.3 μM; IL-8: 9.6 μM

TH1 cytokines: PBMC, PHA@ 20 mg/mL, 24 h

IFN-g: >25 μM; IL-2: >25 μM

TH2 cytokines: PBMC, PHA@ 20 mg/mL, 24 h

IL-4: >25 μM; IL-5: 9.3 μM; IL-10: 14.5 μM; IL-13: >10 μM

Regulatory cytokine: PBMC, PHA@ 10 mg/mL, 24 h

IL-3: >10 μM.

### Example 21

### Toothpaste Formulation Containing Calcium Sulfate Dihydrate and About 0.5 wt. % of 3-hydroxypyridine-2-carbonyl-bis(3-chloro-4-methylphenyl)-borane(bis(3-chloro-4-methylphenyl)boronic acid 3-hydroxypicolinate ester)

A toothpaste formulation according to the present invention is prepared as follows.

To an appropriate measuring container is added 6 mL of grapefruit oil, 2 mL of citric oil, 2 mL of sweet orange oil, 2 mL of peppermint oil, and 2 mL of eucalyptus oil, and the flavor oils are mixed at ambient temperature. Thick liquid paraffin [Food Grade] (also known as mineral oil) is then added in an amount sufficient to bring the total volume of the mixture up to 100 mL. The oil component is mixed so as to form a homogenous solution. This is the base flavoring oil component for use in the toothpaste examples described herein.

To 30 g of finely powdered gypsum (calcium sulfate dihydrate; pure for the food production industry), 0.2 g of 3-hydroxypyridine-2-carbonyl-bis(3-chloro-4-methylphenyl)-borane(bis(3-chloro-4-methylphenyl)boronic acid 3-hydroxypicolinate ester) is added as a dry, solid powder. The two solids are mixed to form a homogenous solid, and then 12 g of the flavoring oil component from above is added. The composition of the powders and oils are mixed together to form a smooth paste. The paste is then packed into a tube.

### Example 22

### Toothpaste Formulation Containing Calcium Sulfate Dihydrate and About 5.0 wt. % of 3-hydroxypyridine-2-carbonyl-bis(3-chloro-4-methylphenyl)-borane(bis(3-chloro-4-methylphenyl)boronic acid 3-hydroxypicolinate ester)

The paste composition in Example 21 is reformed, this time using 2.2 g of 3-hydroxypyridine-2-carbonyl-bis(3-chloro-4-methylphenyl)-borane(bis(3-chloro-4-methylphenyl)boronic acid 3-hydroxypicolinate ester), in the same manner as described. The paste is formed into a smooth paste, as before, and packed into a tube.

### Example 23

### Toothpaste Formulation Containing Calcium Sulfate Dihydrate, About 5.0 wt. % of 3-hydroxypyridine-2-carbonyl-bis(3-chloro-4-methylphenyl)-borane(bis(3-chloro-4-methylphenyl)boronic acid 3-hydroxypicolinate ester), and Glycerine

To 60 g of finely powdered gypsum (as in Example 21), 1.2 g of 3-hydroxypyridine-2-carbonyl-bis(3-chloro-4-methylphenyl)-borane(bis(3-chloro-4-methylphenyl)boronic acid 3-hydroxypicolinate ester) is added as a dry powder, and the two solids are mixed together to form a homogenous powder. This solid powder is then mixed with 32 g of the oil component (as prepared in Example 21) for 30 minutes. A smooth, half-liquid paste results. To this paste is added 4 g of glycerin (available from numerous commercial sources), and the mixing is continued for a further 30 minutes. The product paste is packed into aluminum tubes and is ready for use.

### Example 24

### Mouthwash Containing About 0.5 wt. % of 3-hydroxypyridine-2-carbonyl-bis (3-chloro-4-methylphenyl)-borane(bis(3-chloro-4-methylphenyl)boronic acid 3-hydroxypicolinate ester)

A mouthwash is prepared in conventional manner to the following composition:

| | % w/w |
|---|---|
| Sorbitol 70% solution (non-crystalline) | 5 |
| Ethanol 96% BP (% by volume) | 7 |
| Sodium saccharin BP Cryst (76% Sac) | 0.02 |
| Polyethylene glycol-40 hydrogenated castor oil | 0.15 |
| (available under the trade name Croduret 40 | |
| ET 0080 DF) | |
| Polyoxyethylene sorbitan monolaurate | 0.15 |
| (available under the trade name Tween 20) | |
| Sodium fluoride BP | 0.05 |
| Sodium benzoate BP | 0.1 |
| Blue 12401 Anst | 0.0006 |
| Yellow 2G Anst | 0.00055 |
| Bentonite BP 1 Mouth rinse flavour | 0.1 |
| (% by volume) | |
| Boron-containing compound (3-hydroxypyridine- | 0.5 |
| 2-carbonyl-bis(3-chloro-4-methylphenyl)- | |
| borane(bis(3-chloro-4-methylphenyl)boronic | |
| acid 3-hydroxypicolinate ester)) | |
| Purified water | Balance Total 100.0% |

The mouthwash is made by first mixing together, at room temperature, the ethanol and water, after which the additional components are admixed with the aqueous alcoholic medium the surfactants, flavor, humectants, copolymer, and boron-containing compound that are in the formula. The finished mouthwash is then filtered, if necessary.

### Example 25

### Toothpaste Containing About 0.3 wt. % of 3-hydroxypyridine-2-carbonyl-bis (3-chloro-4-methylphenyl)-borane(bis(3-chloro-4-methylphenyl)boronic acid 3-hydroxypicolinate ester)

A toothpaste is prepared in conventional manner to the following composition:

| | % wt |
|---|---|
| Precipitated silica | 25.0 |
| Gelling silica | 2.0 |
| Sorbitol | 20.0 |
| Propylene glycol | 2.5 |
| Sodium carboxymethyl cellulose | 1.0 |
| Lauryl diethanol amide | 1.0 |
| Sodium lauryl sulfate | 1.5 |
| Sodium lauroyl sarcosinate | 0.3 |
| Sodium saccharin | 0.1 |
| Ethyl p-oxybenzoate | 0.1 |
| Boron-containing compound (3-hydroxypyridine- | 0.3 |
| 2-carbonyl-bis(3-chloro-4-methylphenyl)- | |
| borane(bis(3-chloro-4-methylphenyl)boronic | |
| acid 3-hydroxypicolinate ester)) | |
| Stannous gluconate | 0.3 |
| Gelatin | 0.2 |
| Flavor | 0.8 |
| Purified water | Balance Total 100.0% |

The components are mixed at room temperature to form a homogenous smooth paste. The paste is then packed into a tube.

### Example 26

### MIC Testing

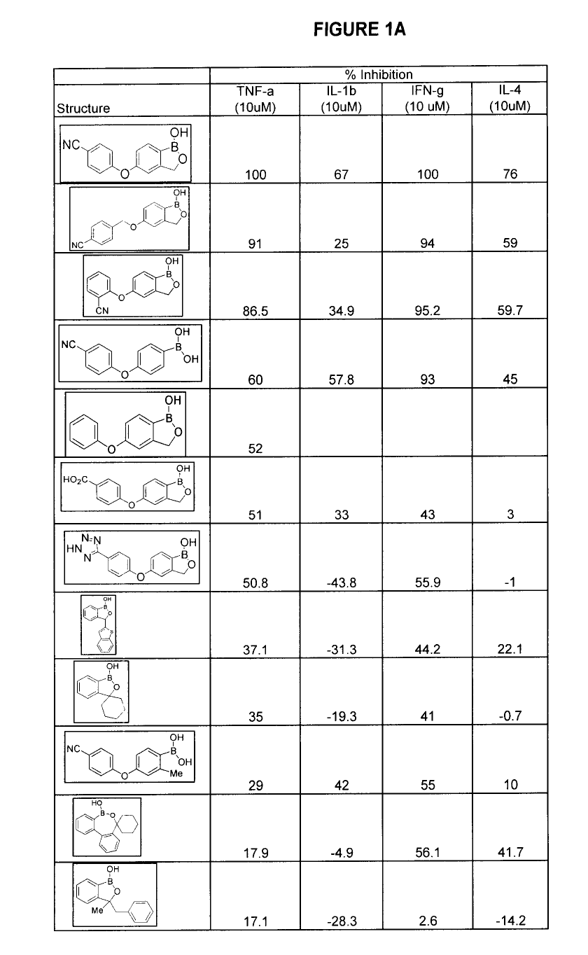

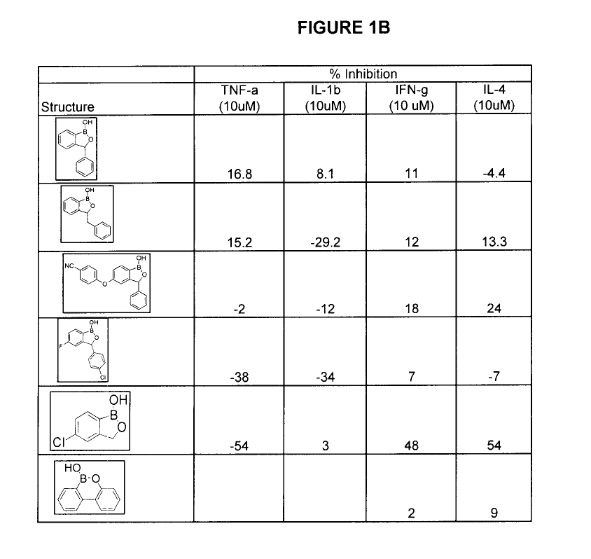

All MIC testing followed the National Committee for Clinical Laboratory Standards (NCCLS) guidelines for antimicrobial testing of yeasts (M27-A2 NCCLS) and filamentous fungi (Pfaller et al., NCCLS publication M38-A—Reference Method for Broth Dilution Antifungal Susceptibility Testing of Filamentous Fungi; Approved Standard. Wayne, Pa.: NCCLS; 2002 (Vol. 22, No. 16) except the Malassezia species which was incubated in a urea broth (Nakamura et al., Antimicrobial Agents And Chemotherapy, 2000, 44(8) p. 2185-2186). Results of the MIC testing is provided in FIG. 1.

The compounds of this invention are evaluated for their antibacterial activity as per the guidelines and procedures prescribed by the National Committee for Clinical Laboratory Standards (NCCLS) (cf., NCCLS Document M7-A3, 1993—Antimicrobial Susceptibility Testing).

Protocol for MIC Determination

A useful protocol for MIC determination is as follows:
1. Approximately 2.5 mg of the compounds to be tested was weighed into cryovials.
2. 5 mg/mL stock solutions were made by adding DMSO to the samples accordingly.
3. 256 μg/mL working solutions were made by using the 5 mg/mL stock solutions and adding sterile distilled water accordingly.
4. A Beckman 2000 Automated Workstation was programmed to load 96 well plates with broth and compounds as follows:
   100 μL of the appropriate broth was added to columns 1-11
   200 μL of the appropriate broth was added to column 12
   100 μL of compounds at the 256 μg/mL working solution were added to column 1 (one compound per row)
   Two-fold serial dilutions were done from column 1 to 10
   Column 11 served as the growth control
5. The 10 organism panel was plated from stock vials stored at −80° C. and incubated for 24 hours at 34° C. The organisms were then sub-cultured and incubated for 24 hours at 34° C.
The inoculums were first prepared in sterile distilled water with a target of 0.09-0.11 absorbance at 620 nm wavelength.
A 1/100 dilution was made into the appropriate broth
100 μL of broth with organism was added to columns 1-11
Column 12 served as the blank control
6. The completed 96 well plates were incubated for 24 hours at 34° C. The 96 well plates were then read using a Beckman Automated Plate Reader at 650 nm wavelength. The MIC was determined through calculations involving the growth control (column 11) and blank control (column 12).
   Protocol for Antifungal In Vitro MIC Determination

A useful protocol for antifungal activity determination is described below.

Preparation

Media is prepared 1-2 weeks before the start of the experiment. Media is stored in the cold room (4° C.) prior to use.

Sabouraud Dextrose Agar Plates:

1. Add 65 g of powdered of Sabouraud Dextrose Agar media into 1 L of dH₂O with gentle stirring

2. Autoclave at 121° C. and 22 psi for 15 minutes

3. Allow the media to cool to about 50° C.

4. Pour media into 100×15 mm sterile petri dishes with 20 mL aliquots

RPMI 1640+MOPS Broth:

1. Add 1 packet of powdered RPMI media to 1 L of dH₂O (15° C.-30° C.) with gentle stirring

2. Add 2 g of NaHCO₃

3. Add 34.5 g of MOPS

4. Adjust the pH to 7.0 using NaOH or HCl

5. Sterilize with membrane filtration (0.22 micron cellulose acetate filter)

Sterile Saline (0.9%)

1. Dissolve 9 g of NaCl to 1 L of dH₂O

2. Autoclave at 121° C. and 22 psi for 15 minutes

Sterile dH₂O

1. Autoclave dH₂O at 121° C. and 22 psi for 15 minutes

Procedure

1. The 10 organism panel is plated from stock vials stored at −80° C. (suspended in broth with 20% glycerol) and incubated at 37° C. for 24 hours. The organisms are then sub-cultured and incubated at 37° C. for 24 hours. These will be used to prepare fresh inoculums for Step 6.

2. Approximately 2.5 mg of the compounds to be tested are weighed into 2 mL cryovials. Fluconazole, Amphotericin B and Itraconazole are tested as reference compounds.

3. 5 mg/mL stock solutions are made by adding DMSO to the samples accordingly. Compounds insoluble with vortexing only are sonicated.

4 256 μg/mL working solutions are made by using the 5 mg/mL stock solutions and adding sterile distilled water accordingly.

5. 96-well plates are used for MIC determination. Each of the 8 rows can be used to test a different compound. Compounds are loaded into the first column and two-fold dilutions of are made from column 1 to 10. Column 11 is a growth control (no compound) and column 12 is a blank control (no compound or organism). Manual addition of broth and compounds is performed as follows:

100 μL of RPMI+MOPS broth is added to columns 1-11

200 μL of RPMI+MOPS broth is added to column 12

100 μL of compounds at the 256 μg/mL working solution are added to column 1 (one compound per row)

Two-fold serial dilutions are done from column 1 to 10

Column 11 serves as the growth control (media+organism only)

6. The sub-cultured organisms are used to prepare fresh inoculums for testing on the 96-well plates. Each 96-well plate will test a different organism.

Colonies from the sub-cultured organisms (Step 1) are used to prepare inoculums with sterile saline. The target is adjusted to 70-75% transmittance at 530 nm wavelength using a Novospec II spectrophotometer.

1/1000 dilution is made into RPMI+MOPS broth

100 μL of this broth with organism is added to columns 1-11 (column 12 serves as the blank control)

7. The completed 96-well plates are incubated at 37° C. for 24 hours. The 96 well plates are then read for absorbance at 650 nm wavelength using a Biomek Automated Plate Reader.

Calculations

The absorbance readings from the Biomek Automated Plate Reader are used to determine the percent inhibition for each test well. The formula used is as follows:
% Inhibition=[1−(ABSₜₑₛₜ−ABS_{blank})/(ABS_{mean growth}−ABS_{blank})]×100%
ABSₜₑₛₜ: Absorbance of the test well
ABS_{blank}: Absorbance of the blank well in the same row as the test well (column 12)
ABS_{mean growth}: Mean absorbance of the growth control wells (column 11)
The minimum inhibitory concentration (MIC) is found at the lowest concentration of compound where percent inhibition is greater than or equal to 80%.
Thus, the invention provides antibiotics that are generically called borinic acid complexes, most preferably derived from disubstituted borinic acids.

All patents, patent applications, and other publications cited in this application are incorporated by reference in the entirety.

What is claimed is:

1. An oral care composition comprising
a compound having a structure according to one of the following formulas: 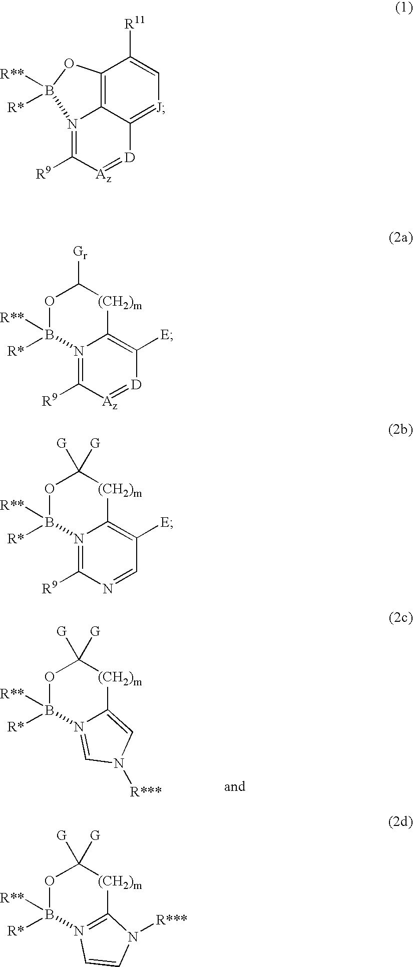
wherein B is boron, O is oxygen,
R* and R** are each independently selected from substituted or unsubstituted alkyl (C₁-C₄), substituted or unsubstituted cycloalkyl (C₃-C₇), substituted or unsubstituted alkenyl, substituted or unsubstituted alkynyl, substituted or unsubstituted aralkyl, substituted or unsubstituted phenyl, and substituted or unsubstituted heteroaryl;
z is 0 or 1 and when z is 1, A is CH, CR¹⁰ or N; D is N, CH, or CR¹²; E is H, OH, alkoxy or 2-(morpholino)ethoxy, CO₂H or CO₂alkyl;
m=0-2;
r is 1 or 2, and wherein when r is 1, G is ═O (double-bonded oxygen) and when r is 2, each G is independently H, methyl, ethyl or propyl;
R¹² is selected from (CH₂)ₖOH (where k=1, 2 or 3), CH₂NH₂, CH₂NH-alkyl, CH₂N(alkyl)₂, CO₂H, CO₂alkyl, CONH₂, OH, alkoxy, aryloxy, SH, S-alkyl, S-aryl, SO₂N(alkyl)₂, SO₂NHalkyl, SO₂NH₂, SO₂alkyl, SO₃H, SCF₃, CN, halogen, CF₃, NO₂, NH₂, 2^{o}-amino, 3^{o}-amino, NH₂SO₂ and CONH₂, and wherein J is CR¹⁰ or N;
R⁹, R¹⁰ and R¹¹ are each independently selected from the group consisting of hydrogen, alkyl, cycloalkyl, (CH₂)ₙOH (n=2 to 3), CH₂NH₂, CH₂NHalkyl, CH₂N(alkyl)₂, halogen, CHO, CH═NOH, CO₂H, CO₂-alkyl, S-alkyl, SO₂-alkyl, S-aryl, SO₂N(alkyl)₂, SO₂NHalkyl, SO₂NH₂, NH₂, alkoxy, CF₃, SCF₃, NO₂, SO₃H and OH, including salts thereof.

2. The oral care composition of claim 1, wherein said oral care composition is a member selected from a mouthwash, dentifrice, liquid whitener, chewing gum, dissolvable, partially dissolvable or non-dissolvable film or strip, wipe or towelette, implant and dental floss.

3. The oral care composition of claim 2, wherein said dentifrice is a member selected from a powder, toothpaste and dental gel.

4. The oral care composition of claim 1, wherein said compound is present in a therapeutically effective amount.

5. The oral care composition of claim 1, wherein said compound is present in an amount of from about 0.1% wgt/wgt to about 5% wgt/wgt.

6. The oral care composition of claim 1, wherein said compound is present in an amount of from about 0.3% wgt/wgt to about 0.6% wgt/wgt.

7. The oral care composition of claim 2, wherein said compound has a structure according to 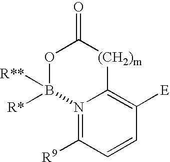
wherein m is 0.

8. The oral care composition of claim 2, wherein said compound has a structure according to

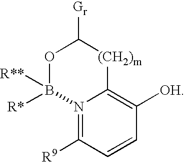

9. The oral care composition of claim 7, wherein E is OH, R⁹ is H and R* and R** are independently selected from substituted or unsubstituted phenyl.

10. The oral care composition of claim 9, wherein R* and R** are independently selected from 4-alkyl, 3-halogen phenyl and 4-halogen, 3-alkyl phenyl.

11. The oral care composition of claim 10, wherein R* and R** are 4-methyl, 3-chloro phenyl.

12. The oral care composition of claim 11, wherein said compound is present in an amount of from about 0.3% wgt/wgt to about 0.6% wgt/wgt.

13. A method for killing a microorganism or inhibiting the growth of a microorganism, comprising contacting said microorganism with a therapeutically effective amount of a compound having a structure according to one of the following formulas: 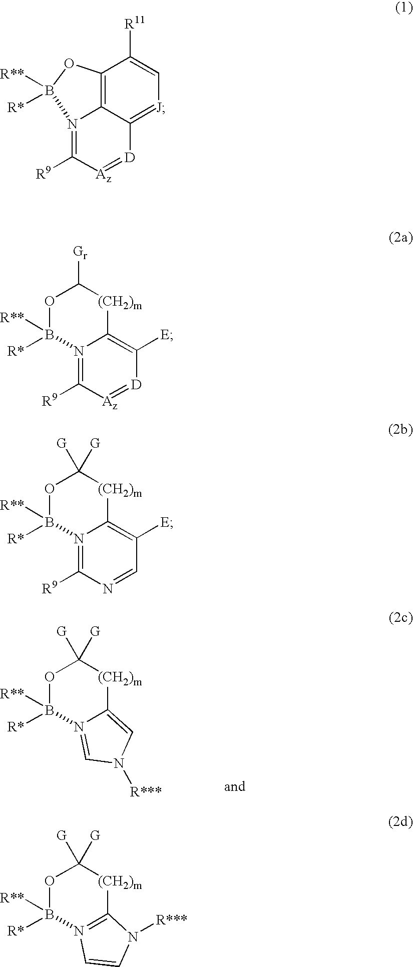
wherein B is boron, O is oxygen,
R* and R** are each independently selected from substituted or unsubstituted alkyl (C₁-C₄), substituted or unsubstituted cycloalkyl (C₃-C₇), substituted or unsubstituted alkenyl, substituted or unsubstituted alkynyl, substituted or unsubstituted aralkyl, substituted or unsubstituted phenyl, and substituted or unsubstituted heteroaryl;
z is 0 or 1 and when z is 1, A is CH, CR¹⁰ or N; D is N, CH, or CR¹²; E is H, OH, alkoxy or 2-(morpholino)ethoxy, CO₂H or CO₂alkyl;
m=0-2;
r is 1 or 2, and wherein when r is 1, G is ═O (double-bonded oxygen) and when r is 2, each G is independently H, methyl, ethyl or propyl;
R¹² is selected from (CH₂)ₖOH (where k=1, 2 or 3), CH₂NH₂, CH₂NH-alkyl, CH₂N(alkyl)₂, CO₂H, CO₂alkyl, CONH₂, OH, alkoxy, aryloxy, SH, S-alkyl, S-aryl, SO₂N(alkyl)₂, SO₂NHalkyl, SO₂NH₂, SO₂alkyl, SO₃H, SCF₃, CN, halogen, CF₃, NO₂, NH₂, 2^{o}-amino, 3^{o}-amino, NH₂SO₂ and CONH₂, and wherein J is CR¹⁰ or N;
R⁹, R¹⁰ and R¹¹ are each independently selected from the group consisting of hydrogen, alkyl, cycloalkyl, (CH₂)ₙOH (n=2 to 3), CH₂NH₂, CH₂NHalkyl, CH₂N(alkyl)₂, halogen, CHO, CH═NOH, CO₂H, CO₂-alkyl, S-alkyl, SO₂-alkyl, S-aryl, SO₂N(alkyl)₂, SO₂NHalkyl, SO₂NH₂, NH₂, alkoxy, CF₃, SCF₃, NO₂, SO₃H and OH, including salts thereof
wherein said microorganism is a member selected from Actinobacillus species, Porphyromonas species, Tannerella species, Prevotella species, Eubacterium species, Treponema species, Bulleidia species, Mogibacterium species, Slackia species, Campylobacter species, Eikenella species, Peptostreptococcus species, Peptostreptococcus species, Capnocytophaga species, Fusobacterium species, Porphyromonas species and Bacteroides species.

14. The method of claim 13, wherein said microorganism is a member selected from Actinobacillus actinomycetemcomitans, Porphyromonas gingivalis, Tannerella forsythensis, Prevotella intermedia, Eubacterium nodatum, Treponema denticola, Bulleidia extructa, Mogibacterium timidum Slackia exigua, Campylobacter rectus, Eikenella corrodens, Peptostreptococcus micros, Peptostreptococcus anaerobius, Capnocytophaga ochracea, Fusobacterium nucleatum, Porphyromonas asaccharolytica and Bacteroides forsythus.

15. The method of claim 13, wherein said compound has a structure according to 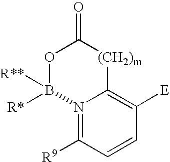
wherein m is 0.

16. The method of claim 15, wherein E is OH, R⁹ is H and R* and R** are independently selected from substituted or unsubstituted phenyl.

17. The method of claim 16, wherein R* and R** are independently selected from 4-alkyl, 3-halogen phenyl and 4-halogen, 3-alkyl phenyl.

18. The method of claim 17, wherein R* and R** are 4-methyl, 3-chloro phenyl.

19. A method of treating or preventing periodontal disease in a human or an animal, said method comprising administering to the human or the animal a therapeutically effective amount of a compound having a structure according to one of the following formulas: 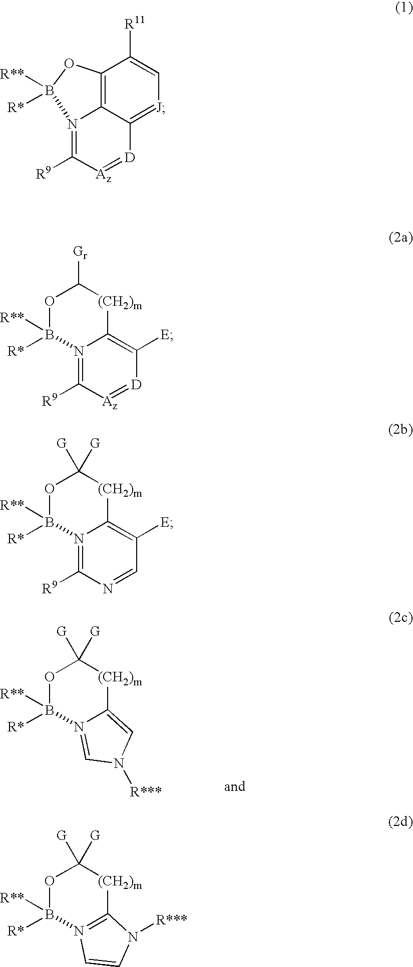
wherein B is boron, O is oxygen,
R* and R** are each independently selected from substituted or unsubstituted alkyl (C₁-C₄), substituted or unsubstituted cycloalkyl (C₃-C₇), substituted or unsubstituted alkenyl, substituted or unsubstituted alkynyl, substituted or unsubstituted aralkyl, substituted or unsubstituted phenyl, and substituted or unsubstituted heteroaryl;
z is 0 or 1 and when z is 1, A is CH, CR¹⁰ or N; D is N, CH, or CR¹²; E is H, OH, alkoxy or 2-(morpholino)ethoxy, CO₂H or CO₂alkyl;
m=0-2;
r is 1 or 2, and wherein when r is 1, G is ═O (double-bonded oxygen) and when r is 2, each G is independently H, methyl, ethyl or propyl;
R¹² is selected from (CH₂)ₖOH (where k=1, 2 or 3), CH₂NH₂, CH₂NH-alkyl, CH₂N(alkyl)₂, CO₂H, CO₂alkyl, CONH₂, OH, alkoxy, aryloxy, SH, S-alkyl, S-aryl, SO₂N(alkyl)₂, SO₂NHalkyl, SO₂NH₂, SO₂alkyl, SO₃H, SCF₃, CN, halogen, CF₃, NO₂, NH₂, 2^{o}-amino, 3^{o}-amino, NH₂SO₂ and CONH₂, and wherein J is CR¹⁰ or N;
R⁹, R¹⁰ and R¹¹ are each independently selected from the group consisting of hydrogen, alkyl, cycloalkyl, (CH₂)ₙOH (n=2 to 3), CH₂NH₂, CH₂NHalkyl, CH₂N(alkyl)₂, halogen, CHO, CH═NOH, CO₂H, CO₂-alkyl, S-alkyl, SO₂-alkyl, S-aryl, SO₂N(alkyl)₂, SO₂NHalkyl, SO₂NH₂, NH₂, alkoxy, CF₃, SCF₃, NO₂, SO₃H and OH, including salts thereof.

20. The method of claim 19, wherein said compound has a structure according to 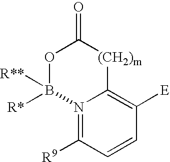
wherein m is 0.

21. The method of claim 20, wherein E is OH, R⁹ is H and R* and R** are independently selected from substituted or unsubstituted phenyl.

22. The method of claim 21, wherein R* and R** are independently selected from 4-alkyl, 3-halogen phenyl and 4-halogen, 3-alkyl phenyl.

23. The method of claim 22, wherein R* and R** are 4-methyl, 3-chloro phenyl.

24. The method of claim 19, wherein said periodontal disease is a member selected from gingivitis, periodontitis, and juvenile/acute periodontitis.
